(19) United States
(12) Patent Application Publication
(10) Pub. No.: US 2015/0337388 A1
(43) Pub. Date: Nov. 26, 2015

(54) METHODS AND COMPOSITIONS FOR IDENTIFYING GLOBAL MICROSATELLITE INSTABILITY AND FOR CHARACTERIZING INFORMATIVE MICROSATELLITE LOCI

(71) Applicant: VIRGINIA TECH INTELLECTUAL PROPERTIES, INC., Blacksburg,, VA (US)

(72) Inventors: Harold R. Garner, JR., Blacksburg, VA (US); Lauren J. McIver, Albuquerque, NM (US); Hongseok Tae, Phoenix, AZ (US)

(21) Appl. No.: 14/652,823

(22) PCT Filed: Dec. 17, 2013
(86) PCT No.: PCT/US13/75763

§ 371 Date: Jun. 17, 2015

Related U.S. Application Data
(60) Provisional application No. 61/737,919, filed on Dec. 17, 2012.

(51) Int. Cl. C12Q 1/68; G06F 19/22; C40B 30/02

(52) U.S. Cl. CPC C12Q 1/6886; C40B 30/02; G06F 19/22; C12Q 2600/106; C12Q 2600/156

(57) ABSTRACT

The disclosure provides methods and systems for assessing microsatellites, for identifying informative microsatellite loci, and for using microsatellite data. Microsatellite information has numerous uses including, for example, to characterize disease risk, to predict responsiveness to therapy, and to non-invasively diagnose subjects.

### RELATED APPLICATIONS

This application claims priority to and the benefit of the filing date of U.S. Provisional Application No. 61/737,919, filed Dec. 17, 2012, the disclosure of which is hereby incorporated by reference herein in its entirety.

### STATEMENT OF GOVERNMENT SUPPORT

This invention was made with government support under Grant U01-HG005719 awarded by The National Institutes of Health, National Human Genome Research Institute. The government has certain rights in the invention.

### BACKGROUND OF THE DISCLOSURE

Microsatellites are tandemly repeated units of 1-6 base pairs in length that comprise approximately 3% of the human genome. They are often highly variable with mutation rates dependent on several factors, including the length of the microsatellite and its location in the genome. Microsatellite mutations within genes have been shown to frequently affect gene expression and function. Microsatellite mutations are linked with more than 20 neurological disorders with associations to autism, Parkinson's disease, Huntington's disease, and attention-deficit/hyperactivity disorder. For example, the most common inherited form of intellectual disability, Fragile X Syndrome, is caused by an expansion in a CGG triplet repeat in the 5′UTR region of FMR1, fragile-X mental retardation 1.

However, microsatellites are highly polymorphic and difficult to analyze en masse. As a result, there has been significantly less reporting of microsatellite polymorphisms when compared to other genomic variations, such as single nucleotide polymorphisms (SNPs) and short insertions/deletions (indels). Therefore there is a need for systems and methods that can be used to analyze and interpret microsatellites on a genomic scale. Such systems may be used for identifying informative microsatellite loci suitable for, among other things, use as prognostic and diagnostic markers of disease and disease predisposition.

### SUMMARY OF THE DISCLOSURE

The disclosure is based, in part, on the improved ability to identify and characterize microsatellite loci, including improved ability to identify microsatellite loci informative for a particular disease state. This improved ability is based on an extensive set of systems and methods that permit accurate analysis of microsatellites across a variety of potentially different populations, as well as systems and methods that permit comparisons of microsatellites across different populations, to identify loci that are informative of a particular disease, condition or state of affairs. The systems and methods, as well as their application to identifying informative loci and using informative loci prognostically, diagnostically, and as a means for identifying potential targets for therapeutic intervention, are described in more detail herein.

In addition to the lack of sufficient tools for effectively analyzing microsatellites, three widely held myths have undermined their study and use. These widely held myths taught away from the exploration and use of microsatellites as markers for diseases and conditions.

Myth #1 is that accurate and efficient analysis of the ˜1 million microsatellites in the human genome is not possible. Myth #2 is that, given that microsatellites are hyper-variable, they will not be useful in genotype-phenotype association studies. Myth #3 is that SNPs are the drivers of disease, and thus, analysis of SNPs will explain both the heritable and spontaneous components of disease.

Our work demonstrates that these myths are incorrect. Moreover, we provide tools, including both computer implemented methods and physical reagents, that can be used to analyze microsatellites across populations and can also be applied to analyzing microsatellites in individual subjects as a diagnostic or risk assessment tool or as part of a treatment or monitoring regime. Specifically, with regard to myth #1, our previous work estimated that microsatellite data from the 1000 Genome Project and the Cancer Genome Atlas was only 20% accurate. Using the methods described herein, we're able to analyze microsatellites with 96% accuracy. Thus, accurate and efficient analysis of microsatellites is now possible. With regard to myth #2, our data analyzing approximately 1,200 genomes from purportedly healthy individuals demonstrated that 98% of the 150,000 microsatellites analyzed are, in fact, highly invariant. Thus, contrary to popular wisdom, microsatellite variation can be effectively used as a biomarker because the majority of loci are not highly variant in healthy populations. Finally, with regard to myth #3, recent reports by others suggest that in a study of over 200,000 subjects, known and new SNPs explained less than 50% of heritability in breast, ovarian, and prostate cancer.

It should be appreciated that the various method steps summarized below may be applied, for example, to methods of identifying increased risk of developing a disease or condition, such as cancer. Such methods may also be applied to methods of identifying microsatellite instability in a subject and methods of identifying variant genotypes in a subject, as well as methods of diagnosing a particular condition, distinguishing between conditions, and the like. The disclosure contemplates applying the various method steps to any of the foregoing, as well as to other applications described herein. Moreover, it should be noted that although, for convenience, many of the methods are indicated as including a step of obtaining a sample, particularly a simple non-invasive or minimally invasive sample indicative of germline nucleic acid, such a step need not be expressly included. For example, in the case of a computer-implement method or system, data or information reflecting nucleotide sequence from a sample or set of samples can be provided, such as inputted into or downloaded to, a computer. Accordingly, the disclosure expressly contemplates methods and uses that do not include such a step of obtaining a sample.

The disclosure also provides methods and systems for identifying informative microsatellite loci. In certain embodiments, these methods and systems are based on analysis of microsatellite loci in two populations, which can then be compared to each other to identify microsatellite loci where the distributions of sequence lengths or genotypes do not significantly overlap. In certain embodiments, sequence lengths, whether considered individually for each allele or considered as a genotype, are called using rule-based analysis or a Gaussian mixture model. Calling using criteria to eliminate suspect data is considered “reliably calling.” Once informative loci are identified, these loci, and information about these loci obtained from one or both of the population analyses, can be used as part of a diagnostic method to evaluate a new sample (e.g., a single patient sample). That new sample can then be evaluated, such as to determine if its genotype at callable informative loci differs from that of, for example, a healthy reference population. Certain steps of such a diagnostic or prognostic method can be implemented on a computer and involve the use of a computer system. In certain embodiments, the disclosure provides a system, such as a computer system, that implements all or a portion of the steps of any of the diagnostic or prognostic methods set forth herein.

It should also more generally be noted that, in certain embodiments, the present disclosure provides methods for identifying informative microsatellite loci and using those loci diagnostically and prognostically that is based on analysis of and comparisons to a reference population or between reference populations, where a reference population is based on information from a plurality of samples or genomes (e.g., members). In other words, rather than simply relying on a comparison between a test sample and a common reference based on a single sample (such as a reference created from analysis of a single sample and deposited in a sequence depository, such as GenBank), the present disclosure is based, in certain embodiments, on identifying informative microsatellite loci by analyzing microsatellite length and/or sequence across a population (e.g., a plurality of samples or genomes, such as a plurality of samples from purportedly healthy individuals indicative of the healthy population—obtained from subjects not diagnosed with a disease) and, optionally, comparing the length and sequence information to another population (such as a populations of individuals having a particular disease). Although alignment of sequence reads for a sample may utilize reference to a single reference sequence for purposes of determining coordinates in the genome, the identification of the informative loci themselves relies, in certain embodiments, on a population analysis. Further, in certain embodiments, when using informative loci diagnostically or prognostically to assess the condition of a particular subject, sequence information for the informative loci in that sample may be compared to information obtained from a population (e.g., the ultimate value or information to which a sample is compared is a value based on analysis of a population—rather than a value based on a single reference sample). However, the disclosure recognizes that, once again, when aligning sequence reads for the sample, a single reference sequence can be used.

Once a set of microsatellite loci (also referred to as a panel of loci or list of loci) informative for a particular disease, condition or trait is identified, future test samples (e.g., a sample from a patient or a test sample of known disease state intended to test the sensitivity and specificity of the identified informative loci can, in certain embodiments, be evaluated and compared to that of a reference population (e.g., a healthy population, a diseased population, or both). This comparison can be performed, for example, by determining if the patient's genotype (e.g., the unit of both alleles for the patient at a given loci) for one or more informative loci better fits into the distribution for the healthy population or the diseased population. Alternatively, the patient's genotype (e.g., the unit of two or more alleles for the patient at a given loci) can be compared to the modal genotype of the healthy population at one or more informative loci. In certain embodiments, a value corresponding to information about allelotype or genotype of a reference population is stored in a computer and used to compare future test samples.

In a first aspect, the disclosure provides a method of identifying an increased risk of developing cancer. The method comprises a series of steps, such as, (i) obtaining a sample of nucleic acid from a subject; (ii) determining a microsatellite profile for said sample for two or more microsatellite loci; and (iii) comparing the microsatellite profile from said sample to a reference microsatellite profile generated from nucleic acid from a reference population to identify an alteration at the two or more microsatellite loci in the sample from the subject relative to that of the reference population. An alteration at said two or more microsatellite loci indicates an increased risk of developing cancer. For a specific locus, the microsatellite profile includes information about the characteristics of that locus, such as sequence length and nucleotide sequence. This information (e.g., this profile) can be compared to a reference to identify whether and how the characteristics of the locus in the sample from the subject differ from the reference.

In certain embodiments, a method of identifying an increased risk of developing cancer is a computer-implemented method which comprises: receiving, at a host computer, a value and/or information representing a microsatellite profile determined by an analysis of nucleic acid obtained from a subject; and comparing, in the host computer, the value and/or information to a reference value and/or information, wherein the reference value and/or information represents a microsatellite profile generated from an analysis of nucleic acid obtained from a reference population of individuals identified as not having cancer, wherein, an alteration at said two or more microsatellite loci indicates an increased risk of developing cancer. It should be understood that the host computer may include a single processor or multiple processors, and that the host computer may be a plurality of computers which communicated, for example, via a network. Moreover, reference information may be stored as a database and used when making comparisons to one, two, or a plurality of microsatellite loci (e.g., including at least 10,000 or even all microsatellite loci for which reliable reference information is available. Further information regarding the generation of a database of microstallite information for a reference population is provided herein. In certain embodiments, the reference sample used for comparison is prepared using the methods described herein.

It should be understood that the foregoing method can also be applied to analyzing increased risk of developing another disease or disorder.

Genotyping is often used, here and in the art, to refer to analyzing information about either or both alleles for a sample. In the present disclosure, this information can be used in, at least, two ways for identifying informative microsatellite loci. First, in an approach based on sequence of each allele, distributions of sequence lengths are determined, and these distributions are then compared to other distributions. This is an alleles-based approach (allelotyping) for determining distributions. It does not account, for any particular sample, for the information at both alleles, for each loci, to be considered together as a unit (e.g., a genotype for a specific locus based on consideration of alleles as a unit). In an approach based on genotype, for each sample, information at both alleles is considered together to determine the genotype (based on at least two alleles; a unit) for a locus for a sample. The distribution of these genotypes is then determined across a population and compared. Although the term genotyping may be used generically to refer to both approaches, determining a genotype is generally used to describe this second approach where both alleles of the sample, at a particular locus, are considered together as a unit, and this unit is used for later comparison to determine a distribution. When the term genotyping can refer to gathering of callable information suitable for use in either approach, context will indicate which is intended. In certain embodiments, sequence length and/or sequence at one or more microsatellite loci is reliably called. From reliably called information, genotype for each sample, at each loci, can be determined and genotype distributions are assessed. In certain embodiments, a modal genotype is determined.

In certain embodiments, determining a genotype includes determining sequence length and/or actual sequence. In certain embodiments, determining sequence may reveal sequence polymorphisms, regardless of whether those polymorphisms impact length. In other embodiments, genotype across a population is determined based only on sequence length. When determining the genotype with either sequence length or actual sequence is discussed in the following embodiments, either or both could generally be used.

In a second aspect, the disclosure provides a method of identifying an increased risk of developing a disease. For example, the method comprises (i) obtaining a sample of nucleic acid from a subject; (ii) determining the sequence length of at least one informative microsatellite locus in said sample; and (iii) comparing the sequence length of the at least one informative microsatellite locus in said sample from the subject to a distribution of sequence lengths of the at least one informative microsatellite locus in nucleic acid obtained from a reference population of individuals identified as not having the disease. If the sequence length of the at least one informative microsatellite locus in said sample differs from the average sequence length of the at least one informative microsatellite locus in nucleic acid obtained from the disease-free reference population, then the subject is identified as being at an increased risk of developing the disease.

In certain embodiments, a method of identifying an increased risk of developing a disease is a computer-implemented method which comprises: receiving, at a host computer, a value representing the sequence length of at least one informative microsatellite locus determined by an analysis of nucleic acid obtained from a subject; and comparing, in the host computer, the value to a distribution of sequence lengths of the at least one informative microsatellite locus in nucleic acid obtained from a reference population of individuals identified as not having the disease, wherein if the sequence length of the at least one informative microsatellite locus in said sample differs from the average sequence length of the at least one informative microsatellite locus in nucleic acid obtained from the disease-free reference population, then the subject is identified as being at an increased risk of developing the disease. It is understood that these steps may be performed on the same computer or different computers, including across computers interconnected via a network or server or series of servers.

In a third aspect, the disclosure provides a method of identifying an increased risk of developing cancer, comprising: obtaining a sample of nucleic acid from a subject; determining the sequence length of at least one informative microsatellite locus in said sample; and comparing the sequence length of the at least one informative microsatellite locus in said sample from the subject to a distribution of sequence lengths of the at least one informative microsatellite locus in nucleic acid obtained from a reference population of individuals identified as not having cancer; wherein, if the sequence length of the at least one informative microsatellite locus in said sample differs from the average sequence length of the at least one informative microsatellite locus in nucleic acid obtained from the cancer-free reference population, then the subject is identified as being at an increased risk of developing cancer.

In certain embodiments, a method of identifying an increased risk of developing cancer is a computer-implemented method which comprises: receiving, at a host computer, a value representing the sequence length of at least one informative microsatellite locus determined by an analysis of nucleic acid obtained from a subject; and comparing, in the host computer, the value to a distribution of sequence lengths of the at least one informative microsatellite locus in nucleic acid obtained from a reference population of individuals identified as not having cancer, wherein if the sequence length of the at least one informative microsatellite locus in said sample differs from the average sequence length of the at least one informative microsatellite locus in nucleic acid obtained from the cancer-free reference population, then the subject is identified as being at an increased risk of developing cancer. It is understood that these steps may be performed on the same computer or different computers, including across computers interconnected via a network or server or series of servers.

In a fourth aspect, the disclosure provides a method of identifying the likelihood that a subject will respond to a particular treatment regimen, comprising: obtaining a sample of nucleic acid from a subject; determining the sequence length of at least one informative microsatellite locus in said sample; and comparing the sequence length of the at least one informative microsatellite locus in said sample from the subject to a distribution of sequence lengths of the at least one informative microsatellite locus in nucleic acid obtained from (i) a population of individuals identified as being poor-responders to the treatment regimen or (ii) a population of individuals identified as being responsive to the treatment regimen; wherein, (i) if the sequence length of the at least one informative microsatellite locus in said sample from the subject differs from the average sequence length of the at least one informative microsatellite locus in nucleic acid obtained from the poor-responders population, then the subject is identified as having increased likelihood for being responsive to the treatment regimen or (ii) if the sequence length of the at least one informative microsatellite locus in said sample from the subject differs from the average sequence length of the at least one informative microsatellite locus in nucleic acid obtained from the responsive population, then the subject is identified as having increased likelihood for being a poor responder to the treatment regimen.

In some embodiments, a method of identifying the likelihood that a subject will respond to a particular treatment regimen is a computer-implemented method which comprises: receiving, at a host computer, a value representing the sequence length of at least one informative microsatellite locus determined by an analysis of nucleic acid obtained from a subject; and comparing, in the host computer, the value to a distribution of sequence lengths of the at least one informative microsatellite locus in nucleic acid obtained from a reference population of individuals identified as (i) being poor-responders to the treatment regimen or (ii) being responsive to the treatment regimen, wherein (i) if the sequence length of the at least one informative microsatellite locus in said sample from the subject differs from the average sequence length of the at least one informative microsatellite locus in nucleic acid obtained from the poor-responders population, then the subject is identified as having increased likelihood for being responsive to the treatment regimen or (ii) if the sequence length of the at least one informative microsatellite locus in said sample from the subject differs from the average sequence length of the at least one informative microsatellite locus in nucleic acid obtained from the responsive population, then the subject is identified as having increased likelihood for being a poor responder to the treatment regimen. It is understood that any one or more of these steps may be performed on the same computer or different computers, including across computers interconnected via a network or server or series of servers.

In a fifth aspect, the disclosure provides a method of evaluating the aggressiveness of a particular tumor type in a subject, comprising: obtaining a sample of nucleic acid from a subject; determining the sequence length of at least one informative microsatellite locus in said sample; and comparing the sequence length of the at least one informative microsatellite locus in said sample from the subject to a distribution of sequence lengths of the at least one informative microsatellite locus in nucleic acid obtained from (i) a population of individuals identified as having an aggressive tumor of the particular tumor type or (ii) a population of individuals identified as having a non-aggressive tumor of the particular tumor type; wherein, (i) if the sequence length of the at least one informative microsatellite locus in said sample from the subject differs from the average sequence length of the at least one informative microsatellite locus in nucleic acid obtained from the population of individuals identified as having an aggressive tumor, then the subject is identified as having a non-aggressive or (ii) if the sequence length of the at least one informative microsatellite locus in said sample from the subject differs from the average sequence length of the at least one informative microsatellite locus in nucleic acid obtained from the population of individuals identified as having a non-aggressive tumor, then the subject is identified as having an aggressive tumor.

In certain embodiments, a method evaluating the aggressiveness of a particular tumor type in a subject is a computer-implemented method which comprises: receiving, at a host computer, a value representing the sequence length of at least one informative microsatellite locus determined by an analysis of nucleic acid obtained from a subject; and comparing, in the host computer, the value to a distribution of sequence lengths of the at least one informative microsatellite locus in nucleic acid obtained from (i) a population of individuals identified as having an aggressive tumor of the particular tumor type or (ii) a population of individuals identified as having a non-aggressive tumor of the particular tumor type; (i) if the sequence length of the at least one informative microsatellite locus in said sample from the subject differs from the average sequence length of the at least one informative microsatellite locus in nucleic acid obtained from the population of individuals identified as having an aggressive tumor, then the subject is identified as having a non-aggressive or (ii) if the sequence length of the at least one informative microsatellite locus in said sample from the subject differs from the average sequence length of the at least one informative microsatellite locus in nucleic acid obtained from the population of individuals identified as having a non-aggressive tumor, then the subject is identified as having an aggressive tumor. It is understood that any one or more of steps may be performed on the same computer or different computers, including across computers interconnected via a network or server or series of servers.

In certain embodiments of any of the foregoing or following aspects and embodiments, the at least one informative microsatellite locus is a locus that has been previously identified by a method comprising: (i) determining a distribution of sequence lengths for a plurality of microsatellite loci in nucleic acid obtained from a population of individuals identified as having the disease; (ii) determining a distribution of sequence lengths for a plurality of microsatellite loci in nucleic acid obtained from a population of individuals identified as not having the disease; (iii) comparing the distribution of sequence lengths for a first microsatellite locus in nucleic acid obtained from the disease population set forth in (i) to the distribution of sequence lengths for the same first microsatellite locus in nucleic acid obtained from the disease-free population set forth in (ii); (iv) repeating the comparing step (iii) for additional microsatellite loci; and (v) classifying as informative, any microsatellite locus whose distributions of sequence lengths do not significantly overlap between the population of individuals identified as having the disease and the population of individual identified as not having the diseases. In certain embodiments, previously determined information regarding informative loci is stored on a computer, such as a database. This information is available for use in a computer-implemented method of comparison when evaluating a new sample from a subject (e.g., performing a risk assessment, diagnostic, or prognostic method on a sample from a subject).

In certain embodiments of any of the foregoing or following aspects and embodiments, the nucleic acid being analyzed is DNA, such as genomic DNA. In other aspects, the nucleic acid being analyzed is RNA. In some aspects, the DNA, such as genomic DNA is non-tumor, germline DNA. Nucleic acid suitable for analysis may be tumor nucleic acid, or nucleic acid from non-tumor tissue indicative of the nucleic acid present in somatic and other non-tumor cells (e.g., germline nucleic acid). In certain embodiments, nucleic acid being analyzed in enriched. For example, nucleic acid may be exome enriched. Alternatively, an enrichment kit may be used to enrich for microsatellites, generally, or for specific microsatellite in a sample.

In certain embodiments, a sample is obtained. That sample may be a tissue sample from a subject or from a member of a population. Such a sample must be processed to obtain nucleic acid which can then be sequenced and analyzed. Alternatively, nucleic acid or nucleic acid information from a sample may be obtained directly, such as by providing sequence information to a computer, such as by downloading available sequence information.

In certain embodiments of any of the foregoing or following aspects and embodiments, the sample from the subject is a tumor sample. In other aspects, the sample from the subject is taken from normal margin cells adjacent to a tumor. In some aspects, the sample obtained from the subject is blood, skin cells, or an oral swab. The foregoing are examples of tissue samples comprising nucleic acid. Even when sequence information is obtained, such as by providing sequence information to a computer, that sequence information is generally from a tissue sample from a subject.

In certain embodiments of any of the forgoing or following aspects and embodiments, the reference population comprises at least 100 healthy subjects. In some aspects, the reference population comprises 100 healthy females. In some aspects, the reference population comprises at least 100 healthy males. In some embodiments, the individuals from the reference population are of the same age, sex, or ethnicity, or combinations thereof, as the test subject. In certain embodiments of any of the forgoing or following aspects and embodiments, the sequence length of at least one informative microsatellite locus in the sample is determined by amplifying the nucleotide sequence of said at least one locus by performing polymerase chain reaction (PCR) using primers flanking each of said at least one locus; and evaluating the amplified fragment by capillary electrophoresis or sequencing. In certain embodiments, an enrichment step is performed, such as by using an enrichment array, to enrich for informative loci in a sample prior to performing capillary electrophoresis or sequencing. It should be noted that amplification using, for example, PCR is optional, and analysis by sequencing (e.g., NextGen sequencing) can be performed without the need for prior amplification.

In certain embodiments of any of the forgoing or following aspects and embodiments, a method of the disclosure comprises determining the sequence length of at least two informative microsatellite loci. In some aspects, a method of the disclosure comprises determining the sequence length of at least five informative microsatellite loci. In some aspects, a method of the disclosure comprises determining the sequence length of at least ten informative microsatellite loci.

In certain embodiments of any of the forgoing or following aspects and embodiments, a method of the disclosure comprises determining the sequence length of at least one informative microsatellite locus selected from the group consisting of the loci 1-100 as set forth in Table 4. In other aspects, a method of the disclosure comprises determining the length of at least two microsatellite loci selected from the group consisting of the loci 1-100 as set forth in Table 4. In some aspects, a method of the disclosure comprises determining the length of at least one informative microsatellite locus selected from the group consisting of the microsatellite loci set forth in Table 2. In some aspects, a method of the disclosure comprises determining the length of at least two microsatellite loci selected from the group consisting of the microsatellite loci set forth in Table 2. In some aspects, a method of the disclosure comprises determining the length of at least one informative microsatellite locus selected from the group consisting of the microsatellite loci set forth in Table 5. In some aspects, a method of the disclosure comprises determining the length of at least two microsatellite loci selected from the group consisting of the microsatellite loci set forth in Table 5. In some aspects, a method of the disclosure comprises determining the length of at least one informative microsatellite locus selected from the group consisting of the microsatellite loci set forth in Tables 8 and/or 9. In some aspects, a method of the disclosure comprises determining the length of at least two microsatellite loci selected from the group consisting of the microsatellite loci set forth in Tables 8 and/or 9. In some aspects, a method of the disclosure comprises determining the length of at least one informative microsatellite locus selected from the group consisting of the microsatellite loci set forth in Table 7. In some aspects, a method of the disclosure comprises determining the length of at least two microsatellite loci selected from the group consisting of the microsatellite loci set forth in Table 7. In some aspects, a method of the disclosure comprises determining the length of at least one informative microsatellite locus selected from the group consisting of the microsatellite loci set forth in Table 10. In some aspects, a method of the disclosure comprises determining the length of at least two microsatellite loci selected from the group consisting of the microsatellite loci set forth in Table 10. Also contemplated are methods in which more than two informative loci are analyzed (e.g., 3, 4, 5, 6, 7, 8, 9, 10, or more than 10, or even all of the identified informative loci).

In certain embodiments of any of the forgoing or following aspects and embodiments, a method of the disclosure comprises determining the length of at least one informative microsatellite locus located in a gene selected from the group consisting of the genes set forth in Table 4. In some aspects, a method of the disclosure comprises determining the length of at least one informative microsatellite locus located in a gene selected from the group consisting of the genes set forth in Table 1. In some aspects, a method of the disclosure comprises determining the length of at least one informative microsatellite locus located in a gene selected from the group consisting of the genes set forth in Table 5. In some aspects, a method of the disclosure comprises determining the length of at least one informative microsatellite locus located in a gene selected from the group consisting of the genes set forth in Table 8 and/or 9. In some aspects, a method of the disclosure comprises determining the length of at least one informative microsatellite locus located in a gene selected from the group consisting of the genes set forth in Table 7. In some aspects, a method of the disclosure comprises determining the length of at least one informative microsatellite locus located in a gene selected from the group consisting of the genes set forth in Table 10. Also contemplated are methods in which more informative loci are analyzed (e.g., 2, 3, 4, 5, 6, 7, 8, 9, 10, or more than 10, or even all of the identified informative loci).

In certain embodiments of any of the forgoing or following aspects and embodiments, the cancer is selected from the group consisting of breast cancer, ovarian cancer, lung cancer, prostate cancer, colon cancer, or glioblastoma.

In certain embodiments of any of the forgoing or following aspects and embodiments, a method of the disclosure provides a sensitivity of at least 40% and a specificity of at least 90%. In some aspects, a method of the disclosure provides a sensitivity of at least 90% and a specificity of at least 90%.

The disclosure also provides a method of identifying an increased risk of developing cancer. Thus, in another aspect, the method comprising: obtaining a sample from a subject; extracting nucleic acid from the sample; analyzing the nucleic acid to determine a microsatellite profile for at least 10,000 microsatellite loci; and comparing the microsatellite profile from said sample to a reference microsatellite profile generated from nucleic acid obtained from a reference population to identify a difference between the subject's microsatellite profile and the reference microsatellite profile; wherein a difference is associated with an increased risk of developing cancer. This type of GMI analysis is itself a biomarker of increased cancer risk (e.g., increased predisposition to developing cancer), and can be used alone or in combination of any of the other methods provided herein. Alternatively, comparisons may be made between germline and tumor samples to identify microsatellite hot spots associated with changes between germline and tumor tissue. Such hotspots may be useful for identifying targets for therapeutic intervention, and the disclosure contemplates using these hotspots as target for drug discovery. In certain embodiments, the comparison is made between matched samples (e.g., a germline and tumor sample taken from the same patient. In other embodiments, the comparison is made between populations of samples (e.g., a plurality of gerline samples are compared to a plurality of tumor samples). Sequence lengths, such as average sequence lengths, for alleles may be compared, or genotypes may be compared.

In certain embodiments of any of the forgoing or following aspects and embodiments, a method of identifying an increased risk of developing cancer is a computer-implemented method which comprises: receiving, at a host computer, a value representing a microsatellite profile for at least 10,000 microsatellite loci determined by an analysis of nucleic acid obtained from a subject; and comparing, in the host computer, the value to a reference value representing a reference microsatellite profile generated from nucleic acid obtained from a reference population to identify a difference between the subject's microsatellite profile and the reference microsatellite profile; wherein a difference is associated with an increased risk of developing cancer. It is understood that any one or more of these steps may be performed on the same computer or different computers, including across computers interconnected via a network or server or series of servers.

The disclosure also provides a method of identifying global microsatellite instability (GMI) in a genome. Thus, in another aspect, the disclosure provides a method comprising: obtaining a sample from a subject; extracting nucleic acid from the sample; analyzing the nucleic acid to determine a microsatellite profile for at least 10,000 microsatellite loci; and comparing the microsatellite profile from said sample to a reference microsatellite profile generated from nucleic acid obtained from a reference population to identify a difference between the subject's microsatellite profile and the reference microsatellite profile; wherein a difference is associated with an increased risk of developing cancer. This type of GMI analysis is itself a biomarker of increased cancer risk (e.g., increased predisposition to developing cancer), and can be used alone or in combination of any of the other methods provided herein.

In certain embodiments of any of the foregoing or following aspects and embodiments, a method of identifying global microsatellite instability (GMI) in a genome is a computer-implemented method which comprises: receiving, at a host computer, a value representing a microsatellite profile for at least 10,000 microsatellite loci determined by an analysis of nucleic acid obtained from a subject; and comparing, in the host computer, the value to a reference value representing a reference microsatellite profile generated from nucleic acid obtained from a reference population to identify a difference between the subject's microsatellite profile and the reference microsatellite profile; wherein a difference is associated with an increased risk of developing cancer. It is understood that any one or more of these steps may be performed on the same computer or different computers, including across computers interconnected via a network or server or series of servers.

The disclosure also provides a method of identifying a subject at increased risk for developing ovarian cancer. Thus, in another aspect, the disclosure provides a method comprising: obtaining a sample from a subject; extracting nucleic acid from the sample; analyzing the nucleic acid in said sample from the subject to determine the sequence length of at least four microsatellite loci selected from the group consisting of loci 1-100 listed in Table 4; and comparing the sequence length of the at least four microsatellite loci in said sample from the subject to a distribution of sequence lengths of each of the at least four microsatellite locus in nucleic acid obtained from a reference population of individuals identified as not having ovarian cancer; wherein, if the sequence length of each of the at least four microsatellite loci in said sample from the subject differs from the average sequence length of the at least four microsatellite loci in nucleic acid obtained from the reference population, then the subject is identified as being at an increased risk of developing the ovarian cancer; wherein the method provides a sensitivity of at least 40% and a specificity of at least 90% for identifying subjects at increased risk of developing ovarian cancer.

In certain embodiments of any of the foregoing or following aspects and embodiments, a method for identifying a subject at increased risk of developing ovarian cancer, is a computer-implemented method which comprises: receiving, at a host computer, values representing the sequence length of at least four microsatellite loci selected from the group consisting of loci 1-100 listed in Table 4; and comparing, in the host computer, the values to reference values, wherein the reference values represents the average sequence length of each of the at least four microsatellite loci in a reference population of individuals identified as not having ovarian cancer, wherein, if the sequence length of each of the at least four microsatellite loci in said sample from the subject differs from the average sequence length of the at least four microsatellite loci in nucleic acid obtained from the reference population, then the subject is identified as being at an increased risk of developing the ovarian cancer; wherein the method provides a sensitivity of at least 40% and a specificity of at least 90% for identifying subjects at increased risk of developing ovarian cancer.

The disclosure also provides a method of identifying a subject at increased risk for developing breast cancer. Thus, in another aspect, the disclosure provides a method comprising: obtaining a sample from a subject; extracting nucleic acid from the sample; analyzing the nucleic acid in said sample to determine the sequence length of a microsatellite locus, wherein the locus is located in the CDC2L1/2 gene; and comparing the sequence length of the microsatellite locus in said sample to a distribution of sequence lengths of the microsatellite locus in nucleic acid obtained from a reference population of individuals identified as not having breast cancer; wherein, if the sequence length of the microsatellite loci in said sample differs from the average sequence length of the microsatellite locus in nucleic acid obtained from the reference population, then the subject is identified as being at an increased risk of developing the breast cancer; wherein the method provides a sensitivity of at least 90% and a specificity of at least 90% for identifying subjects at increased risk of developing breast cancer.

In certain embodiments of any of the foregoing or following aspects and embodiments, the method for identifying a subject at increased risk of developing breast cancer further comprises analyzing the nucleic acid in the sample from the subject to determine the sequence length of at least two additional microsatellite loci selected from the group consisting of the loci listed in Table 2 and comparing the sequence length of the at least two additional microsatellite loci in said sample from the subject to a distribution of sequence lengths of each of the at least two additional microsatellite locus in nucleic acid obtained from the reference population.

In certain embodiments of any of the foregoing or following aspects and embodiments, a method for identifying a subject at increased risk of developing breast cancer is a computer-implemented method comprises: receiving, at a host computer, a value representing the sequence length of a microsatellite locus, wherein the locus is located in the CDC2L1/2 gene; and comparing, in the host computer, the value to a reference value, wherein the reference value represents the average sequence length of the microsatellite locus in a reference population of individuals identified as not having breast cancer, wherein, if the sequence length of the microsatellite loci in said sample differs from the average sequence length of the microsatellite locus in nucleic acid obtained from the reference population, then the subject is identified as being at an increased risk of developing the breast cancer; wherein the method provides a sensitivity of at least 90% and a specificity of at least 90% for identifying subjects at increased risk of developing breast cancer.

The disclosure also provides a method of identifying subjects at increased risk for developing breast cancer. Thus, in another aspect the disclosure provides a method comprising: obtaining a sample from a subject; extracting nucleic acid from the sample; analyzing the nucleic acid in said sample from the subject to determine the sequence length of at least three microsatellite loci selected from group consisting of the microsatellites listed in Table 2; and comparing the sequence length of the at least three microsatellite loci in said sample to a distribution of sequence lengths of the at least three microsatellite loci in nucleic acid obtained from a reference population of individuals identified as not having breast cancer; wherein, if the sequence length of each of the at least three microsatellite loci in said sample differs from the average sequence length of the at least three microsatellite loci in nucleic acid obtained from the reference population, then the subject is identified as being at an increased risk of developing the breast cancer; wherein the method provides a sensitivity of at least 90% and a specificity of at least 90% for identifying subjects at increased risk of developing breast cancer. In some aspects, the length of at least four microsatellite loci is determined. In some aspects, the length of all five microsatellite loci is determined.

In certain embodiments of any of the foregoing or following aspects and embodiments, a method for identifying a subject at increased risk of developing breast cancer is a computer-implemented method which comprises: receiving, at a host computer, values representing the sequence length of at least three microsatellite loci selected from group consisting of the microsatellites listed in Table 2; and comparing, in the host computer, the values to reference values, wherein the reference values represents the average sequence length of each of the at least three microsatellite loci in a reference population of individuals identified as not having breast cancer, wherein, if the sequence length of the microsatellite loci in said sample differs from the average sequence length of the microsatellite locus in nucleic acid obtained from the reference population, then the subject is identified as being at an increased risk of developing the breast cancer; wherein the method provides a sensitivity of at least 90% and a specificity of at least 90% for identifying subjects at increased risk of developing breast cancer.

The present disclosure also provides a method of identifying a subject at increased risk of developing glioblastoma. Thus, in another aspect, the disclosure provides a method comprising obtaining a sample from a subject; extracting nucleic acid from the sample; analyzing the nucleic acid in said sample from the subject to determine the sequence length of at least three microsatellite loci selected from the group consisting of the loci listed in Table 5; and comparing the sequence length of the at least three microsatellite loci in said sample from the subject to a distribution of sequence lengths of each of the at least three microsatellite locus in nucleic acid obtained from a reference population of individuals identified as not having glioblastoma; wherein, if the sequence length of each of the at least three microsatellite loci in said sample from the subject differs from the average sequence length of the at least three microsatellite loci in nucleic acid obtained from the reference population, then the subject is identified as being at an increased risk of developing glioblastoma.

In certain embodiments of any of the foregoing or following aspects and embodiments, a method for identifying a subject at increased risk of developing glioblastoma is a computer-implemented method which comprises: receiving, at a host computer, values representing the sequence length of at least three microsatellite loci selected from group consisting of the microsatellites listed in Table 5; and comparing, in the host computer, the values to reference values, wherein the reference values represents the average sequence length of each of the at least three microsatellite loci in a reference population of individuals identified as not having glioblastoma, wherein, if the sequence length of the microsatellite loci in said sample differs from the average sequence length of the microsatellite locus in nucleic acid obtained from the reference population, then the subject is identified as being at an increased risk of developing glioblastoma.

The disclosure also provides a method of identifying a subject at increased risk for developing lung cancer. Thus, in another aspect, the disclosure provides a method comprising: obtaining a sample from a subject; extracting nucleic acid from the sample; analyzing the nucleic acid in said sample from the subject to determine the sequence length of at least three microsatellite loci selected from the group consisting of the loci listed in Tables 8 and/or 9; and comparing the sequence length of the at least three microsatellite loci in said sample from the subject to a distribution of sequence lengths of each of the at least three microsatellite locus in nucleic acid obtained from a reference population of individuals identified as not having lung cancer; wherein, if the sequence length of each of the at least three microsatellite loci in said sample from the subject differs from the average sequence length of the at least three microsatellite loci in nucleic acid obtained from the reference population, then the subject is identified as being at an increased risk of developing lung cancer. In certain embodiments, the method is a method of identifying subjects at increased risk of developing adenocarcinoma of the lung. In another aspect, the method is a method of identifying subjects at increased risk of developing squamous cell carcinoma.

In certain embodiments of any of the foregoing or following aspects and embodiments, a method for identifying a subject at increased risk of developing lung cancer is a computer-implemented method which comprises: receiving, at a host computer, values representing the sequence length of at least three microsatellite loci selected from group consisting of the microsatellites listed in Tables 8 and 9; and comparing, in the host computer, the values to reference values, wherein the reference values represents the average sequence length of each of the at least three microsatellite loci in a reference population of individuals identified as not having lung cancer, wherein, if the sequence length of the microsatellite loci in said sample differs from the average sequence length of the microsatellite locus in nucleic acid obtained from the reference population, then the subject is identified as being at an increased risk of developing lung cancer.

The disclosure also provides a method of identifying a subject at increased risk for developing prostate cancer. Thus, in another aspect, the disclosure provides a method comprising: obtaining a sample from a subject; extracting nucleic acid from the sample; analyzing the nucleic acid in said sample from the subject to determine the sequence length of at least three microsatellite loci selected from the group consisting of the loci listed in Table 10; and comparing the sequence length of the at least three microsatellite loci in said sample from the subject to a distribution of sequence lengths of each of the at least three microsatellite locus in nucleic acid obtained from a reference population of individuals identified as not having prostate cancer; wherein, if the sequence length of each of the at least three microsatellite loci in said sample from the subject differs from the average sequence length of the at least three microsatellite loci in nucleic acid obtained from the reference population, then the subject is identified as being at an increased risk of developing prostate cancer.

In certain embodiments of any of the foregoing or following aspects and embodiments, a method for identifying a subject at increased risk of developing prostate cancer is a computer-implemented method which comprises: receiving, at a host computer, values representing the sequence length of at least three microsatellite loci selected from group consisting of the microsatellites listed in Table 10; and comparing, in the host computer, the values to reference values, wherein the reference values represents the average sequence length of each of the at least three microsatellite loci in a reference population of individuals identified as not having prostate cancer, wherein, if the sequence length of the microsatellite loci in said sample differs from the average sequence length of the microsatellite locus in nucleic acid obtained from the reference population, then the subject is identified as being at an increased risk of developing prostate cancer.

The disclosure also provides a method of identifying a subject at increased risk for developing colon cancer. Thus, in another aspect, the disclosure provides a method comprising: obtaining a sample from a subject; extracting nucleic acid from the sample; analyzing the nucleic acid in said sample from the subject to determine the sequence length of at least three microsatellite loci selected from the group consisting of the loci listed in Table 7; and comparing the sequence length of the at least three microsatellite loci in said sample from the subject to a distribution of sequence lengths of each of the at least three microsatellite locus in nucleic acid obtained from a reference population of individuals identified as not having colon cancer; wherein, if the sequence length of each of the at least three microsatellite loci in said sample from the subject differs from the average sequence length of the at least three microsatellite loci in nucleic acid obtained from the reference population, then the subject is identified as being at an increased risk of developing colon cancer.

In certain embodiments of any of the foregoing or following aspects and embodiments, a method for identifying a subject at increased risk of developing colon cancer is a computer-implemented method which comprises: receiving, at a host computer, values representing the sequence length of at least three microsatellite loci selected from group consisting of the microsatellites listed in Table 7; and comparing, in the host computer, the values to reference values, wherein the reference values represents the average sequence length of each of the at least three microsatellite loci in a reference population of individuals identified as not having colon cancer, wherein, if the sequence length of the microsatellite loci in said sample differs from the average sequence length of the microsatellite locus in nucleic acid obtained from the reference population, then the subject is identified as being at an increased risk of developing colon cancer.

In certain embodiments of any of the foregoing or following aspects and embodiments, the sample from the subject comprises a blood sample, skin sample, or oral swab. In certain embodiments, the sample comprises tumor or cancer cells. In some aspects, the nucleic acid being analyzed is DNA, such as genomic DNA. In some aspects, the DNA, such as genomic DNA is non-tumor, germline DNA. In some aspects, extracting nucleic acid from the sample comprises preparing genomic DNA from the sample. In some aspects, extracting nucleic acid from the sample comprises preparing RNA from the sample.

In certain embodiments, the samples are from a human. In other embodiments, the samples are from a non-human animal. In yet other embodiments, the samples are from a plant. In methods involving plant samples, the condition analyzed may be a characteristic such as disease, pesticide or pest resistance.

In certain embodiments of any of the foregoing or following aspects and embodiments, analyzing nucleic acid comprises amplifying the nucleotide sequence of each of said loci by performing polymerase chain reaction (PCR) using primers flanking each of said loci; and evaluating the amplified fragment by capillary electrophoresis or sequencing. In other aspects, analyzing nucleic acid comprises performing next-generation sequencing. In certain embodiments, an enrichment step is performed, such as by using an enrichment array, to enrich for informative loci in a sample prior to performing capillary electrophoresis or sequencing. It should be noted that amplification using, for example, PCR is optional, and analysis by sequencing (e.g., NextGen sequencing) can be performed without the need for prior amplification. In certain embodiments, prior to sequencing, the nucleic acid is enriched using an enrichment kit. For example, an enrichment kit comprising one or more enrichment probes is used to enrich for microsatellite-containing sequence fragments. This can be done prior to sequencing to increase the proportion of the sample in the sequencing reaction containing a microsatellite. In certain embodiments, use of an enrichment array increases the callable microsatellite loci in the sample.

In certain embodiments of any of the foregoing or following aspects and embodiments, the average sequence length of a microsatellite locus in a population is determined by a method comprising: obtaining a nucleotide sequence of the locus from a first chromosome and a second chromosome in each individual in the population to generate a plurality of nucleotide sequences for the population; aligning the plurality of nucleotide sequences to a plurality of microsatellite loci identified from a reference genome; selecting sequence portions preceding and following the microsatellite locus; identifying a similarity between microsatellite locus and sequence portions and a portion of the reference genome; determining a length of the microsatellite locus for each individual in the population; forming a distribution of the lengths of the microsatellite locus; and determining a value based on the distribution, wherein the value is the average sequence length of the microsatellite locus in the population.

In certain embodiments of any of the foregoing or following aspects and embodiments, the genotype of a microsatellite locus is determined by a method comprising: obtaining a nucleotide sequence of the locus from a first chromosome and a second chromosome in each individual and assigning a genotype based on this information.

In certain embodiments of any of the foregoing or following aspects and embodiments, if the subject is identified as having an increased risk of developing cancer, then the subject is provided with a recommendation for prophylactic treatment of the cancer. In some aspects, if the subject is identified as having an increased risk of developing cancer, the subject is placed on a cancer monitoring regimen that exceeds the level of monitoring generally provided for subjects of comparable age and gender.

The present disclosure also provides a method of diagnosing ovarian cancer in a subject suspected of having cancer, comprising: obtaining a sample from the subject; extracting nucleic acid from the sample; analyzing the nucleic acid in said sample from the subject to determine the sequence length of at least four microsatellite loci selected from the group consisting of loci 1-100 listed in Table 4; comparing the sequence length of the at least four microsatellite loci in said sample to a distribution of sequence lengths of each of the at least four microsatellite loci in nucleic acid obtained from a reference population of individuals identified as not having ovarian cancer; and diagnosing the subject as having ovarian cancer if the sequence length of each of the at least 4 microsatellite loci in said sample from the subject differs from the average sequence length of the at least 4 microsatellite loci in nucleic acid obtained from the reference population; wherein the method provides a sensitivity of at least 40% and a specificity of at least 90% for diagnosing subjects having ovarian cancer.

In some aspects, a method of diagnosing ovarian cancer in a subject suspected of having cancer is a computer-implemented method which comprises: receiving, at a host computer, values representing the sequence length of at least four microsatellite loci selected from group consisting of the microsatellites listed in Table 4; and comparing, in the host computer, the values to a distribution of values representing the sequence lengths of each of the at least four microsatellite loci in nucleic acid obtained from a reference population of individuals identified as not having ovarian cancer; wherein, if the sequence length of each of the at least 4 microsatellite loci in said sample from the subject differs from the average sequence length of the at least 4 microsatellite loci in nucleic acid obtained from the reference population, then the subject is diagnosed as having ovarian cancer; wherein the method provides a sensitivity of at least 40% and a specificity of at least 90% for diagnosing subjects having ovarian cancer.

In some aspects, if the subject is diagnosed as having ovarian cancer, the method further comprises treating the subject for ovarian cancer. In some aspects, the subject was suspected of having cancer because the subject had one or more prior tests consistent with or suggestive of a diagnosis of cancer.

The present disclosure also provides a method for diagnosing breast cancer in a subject suspected of having breast cancer, comprising: obtaining a sample from a subject; extracting nucleic acid from the sample; analyzing the nucleic acid in said sample from the subject to determine the sequence length of a microsatellite locus located in the CDC2L1/2 gene; comparing the sequence length of the microsatellite locus in said sample from the subject to a distribution of sequence lengths of the microsatellite locus in the nucleic acid obtained from a reference population of individuals identified as not having breast cancer; and diagnosing the subject as having breast cancer if the sequence length of the microsatellite locus in said sample from the subject differs from the average sequence length of the microsatellite locus in nucleic acid obtained from the reference population, wherein the method provides a sensitivity of at least 90% and a specificity of at least 90% for diagnosing subjects having breast cancer.

In some aspects, a method of diagnosing breast cancer in a subject suspected of having cancer is a computer-implemented method which comprises: receiving, at a host computer, a value representing the sequence length of a microsatellite locus located in the CDC2L1/2 gene; and comparing, in the host computer, the value to a distribution of values representing the sequence lengths of the microsatellite locus in nucleic acid obtained from a reference population of individuals identified as not having breast cancer; wherein, if the sequence length of the microsatellite locus in said sample from the subject differs from the average sequence length of the microsatellite locus in nucleic acid obtained from the reference population, wherein the method provides a sensitivity of at least 90% and a specificity of at least 90% for diagnosing subjects having breast cancer, then the subject is diagnosed as having breast cancer; wherein the method provides a sensitivity of at least 90% and a specificity of at least 90% for diagnosing subjects having breast cancer.

In some aspects, if the subject is diagnosed as having breast cancer, the method further comprises treating the subject for breast cancer. In some aspects, the subject was suspected of having breast cancer because the subject had one or more prior tests consistent with or suggestive of a diagnosis of breast cancer.

In some aspects, the method of diagnosing breast cancer in a subject further comprises analyzing the nucleic acid to determine the sequence length of least two additional microsatellite loci selected from the group consisting of the loci listed in Table 2 and comparing the sequence length of the at least two additional microsatellite loci in said sample to a distribution of sequence lengths of the at least two additional microsatellite loci in nucleic acid obtained from the reference population; and diagnosing the subject as having breast cancer if the sequence length of the at least two additional microsatellite loci in said sample from the subject differs from the average sequence length of the at least two additional microsatellite loci in nucleic acid obtained from the reference population; wherein the method provides a sensitivity of at least 90% and a specificity of at least 90% for diagnosing subjects having breast cancer.

In some aspects, a method of diagnosing breast cancer in a subject suspected of having cancer is a computer-implemented method which comprises: receiving, at a host computer, values representing the sequence length of at least two microsatellite loci selected from group consisting of the microsatellites listed in Table 2; and comparing, in the host computer, the values to a distribution of values representing the sequence lengths of each of the at least two microsatellite loci in nucleic acid obtained from a reference population of individuals identified as not having breast cancer; wherein, if the sequence length of each of the at least two microsatellite loci in said sample from the subject differs from the average sequence length of the at least two microsatellite loci in nucleic acid obtained from the reference population, then the subject is diagnosed as having breast cancer; wherein the method provides a sensitivity of at least 40% and a specificity of at least 90% for diagnosing subjects having breast cancer.

The present disclosure also provides method for diagnosing breast cancer in a subject suspected of having breast cancer, comprising: obtaining a sample from a subject; extracting nucleic acid from the sample; analyzing the nucleic acid to determine the sequence length of at least three microsatellite loci located in genes selected from group consisting of MAPKAPK3, CABIN1, HSPA6, NSUN5 and CDC2L1; comparing the sequence length of the at least three microsatellite loci in said sample from the subject to a distribution of sequence lengths of each of the at least three microsatellite loci in the nucleic acid obtained from a reference population of individuals identified as not having breast cancer; and diagnosing the subject as having breast cancer if the sequence length of each of the at least three microsatellite loci in said sample differs from the average sequence length of the at least three microsatellite loci in nucleic acid obtained from the reference population, wherein the method provides a sensitivity of at least 90% and a specificity of at least 90% for diagnosing subjects having breast cancer.

In some aspects, a method of diagnosing breast cancer in a subject suspected of having breast is a computer-implemented method which comprises: receiving, at a host computer, values representing the sequence length of at least three microsatellite loci located in genes selected from group consisting of MAPKAPK3, CABIN1, HSPA6, NSUN5 and CDC2L1; and comparing, in the host computer, the values to a distribution of values representing the sequence lengths of each of the at least four microsatellite loci in nucleic acid obtained from a reference population of individuals identified as not having breast cancer; wherein, if the sequence length of each of the at least three microsatellite loci in said sample from the subject differs from the average sequence length of the at least three microsatellite loci in nucleic acid obtained from the reference population, then the subject is diagnosed as having breast cancer; wherein the method provides a sensitivity of at least 90% and a specificity of at least 90% for diagnosing subjects having breast cancer.

In some aspects, the length of at least four microsatellite loci located in genes selected from group consisting of MAPKAPK3, CABIN1, HSPA6, NSUN5 and CDC2L1 is determined. In some aspects, the length of all five microsatellite loci is determined.

In some aspects, if the subject is diagnosed as having breast cancer, the method further comprises treating the subject for breast cancer. In some aspects, the subject was suspected of having breast cancer because the subject had one or more prior tests consistent with or suggestive of a diagnosis of breast cancer.

The present disclosure also provides a method for diagnosing glioblastoma in a subject suspected of having glioblastoma, comprising: obtaining a sample from the subject; extracting nucleic acid from the sample; analyzing the nucleic acid in said sample from the subject to determine the sequence length of at least 3 microsatellite loci selected from the group consisting of the microsatellite loci listed in Table 5; comparing the sequence length of the at least 3 microsatellite loci in said sample to a distribution of sequence lengths of each of the at least 3 microsatellite loci in nucleic acid obtained from a reference population of individuals identified as not having glioblastoma; and diagnosing the subject as having glioblastoma if the sequence length of each of the at least 3 microsatellite loci in said sample from the subject differs from the average sequence length of the at least 3 microsatellite loci in nucleic acid obtained from the reference population.

In some aspects, a method of diagnosing glioblastoma in a subject suspected of having glioblastoma is a computer-implemented method which comprises: receiving, at a host computer, values representing the sequence length of at least three microsatellite loci selected from group consisting of the microsatellites listed in Table 5; and comparing, in the host computer, the values to a distribution of values representing the sequence lengths of each of the at least three microsatellite loci in nucleic acid obtained from a reference population of individuals identified as not having glioblastoma; wherein, if the sequence length of each of the at least three microsatellite loci in said sample from the subject differs from the average sequence length of the at least three microsatellite loci in nucleic acid obtained from the reference population, then the subject is diagnosed as having glioblastoma.

In some aspects, if the subject is diagnosed as having glioblastoma, the method further comprises treating the subject for glioblastoma. In some aspects, the subject was suspected of having glioblastoma because the subject had one or more prior tests consistent with or suggestive of a diagnosis of glioblastoma.

The present disclosure also provides a method for diagnosing lung cancer in a subject suspected of having lung cancer, comprising: obtaining a sample from the subject; extracting nucleic acid from the sample; analyzing the nucleic acid in said sample from the subject to determine the sequence length of at least 3 microsatellite loci selected from the group consisting of the microsatellite loci listed in Tables 8 and 9; comparing the sequence length of the at least 3 microsatellite loci in said sample to a distribution of sequence lengths of each of the at least 3 microsatellite loci in nucleic acid obtained from a reference population of individuals identified as not having lung cancer; and diagnosing the subject as having lung cancer if the sequence length of each of the at least 3 microsatellite loci in said sample from the subject differs from the average sequence length of the at least 3 microsatellite loci in nucleic acid obtained from the reference population.

In some aspects, a method of diagnosing lung cancer in a subject suspected of having lung cancer is a computer-implemented method which comprises: receiving, at a host computer, values representing the sequence length of at least three microsatellite loci selected from group consisting of the microsatellites listed in Tables 8 and 9; and comparing, in the host computer, the values to a distribution of values representing the sequence lengths of each of the at least three microsatellite loci in nucleic acid obtained from a reference population of individuals identified as not having lung cancer; wherein, if the sequence length of each of the at least three microsatellite loci in said sample from the subject differs from the average sequence length of the at least three microsatellite loci in nucleic acid obtained from the reference population, then the subject is diagnosed as having lung cancer.

In some aspects, if the subject is diagnosed as having lung cancer, the method further comprises treating the subject for lung cancer. In some aspects, the subject was suspected of having lung cancer because the subject had one or more prior tests consistent with or suggestive of a diagnosis of lung cancer.

The present disclosure also provides a method for diagnosing prostate cancer in a subject suspected of having prostate cancer, comprising: obtaining a sample from the subject; extracting nucleic acid from the sample; analyzing the nucleic acid in said sample from the subject to determine the sequence length of at least 3 microsatellite loci selected from the group consisting of the microsatellite loci listed in Table 10; comparing the sequence length of the at least 3 microsatellite loci in said sample to a distribution of sequence lengths of each of the at least 3 microsatellite loci in nucleic acid obtained from a reference population of individuals identified as not having prostate cancer; and diagnosing the subject as having prostate cancer if the sequence length of each of the at least 3 microsatellite loci in said sample from the subject differs from the average sequence length of the at least 3 microsatellite loci in nucleic acid obtained from the reference population.

In some aspects, a method of diagnosing prostate cancer in a subject suspected of having prostate cancer is a computer-implemented method which comprises: receiving, at a host computer, values representing the sequence length of at least three microsatellite loci selected from group consisting of the microsatellites listed in Tables 10; and comparing, in the host computer, the values to a distribution of values representing the sequence lengths of each of the at least three microsatellite loci in nucleic acid obtained from a reference population of individuals identified as not having prostate cancer; wherein, if the sequence length of each of the at least three microsatellite loci in said sample from the subject differs from the average sequence length of the at least three microsatellite loci in nucleic acid obtained from the reference population, then the subject is diagnosed as having prostate cancer.

In some aspects, if the subject is diagnosed as having prostate cancer, the method further comprises treating the subject for prostate cancer. In some aspects, the subject was suspected of having prostate cancer because the subject had one or more prior tests consistent with or suggestive of a diagnosis of prostate cancer.

The present disclosure also provides a method for diagnosing colon cancer in a subject suspected of having colon cancer, comprising: obtaining a sample from the subject; extracting nucleic acid from the sample; analyzing the nucleic acid in said sample from the subject to determine the sequence length of at least 3 microsatellite loci selected from the group consisting of the microsatellite loci listed in Table 7; comparing the sequence length of the at least 3 microsatellite loci in said sample to a distribution of sequence lengths of each of the at least 3 microsatellite loci in nucleic acid obtained from a reference population of individuals identified as not having colon cancer; and diagnosing the subject as having lung cancer if the sequence length of each of the at least 3 microsatellite loci in said sample from the subject differs from the average sequence length of the at least 3 microsatellite loci in nucleic acid obtained from the reference population.

In some aspects, a method of diagnosing colon cancer in a subject suspected of having colon cancer is a computer-implemented method which comprises: receiving, at a host computer, values representing the sequence length of at least three microsatellite loci selected from group consisting of the microsatellites listed in Tables 7; and comparing, in the host computer, the values to a distribution of values representing the sequence lengths of each of the at least three microsatellite loci in nucleic acid obtained from a reference population of individuals identified as not having colon cancer; wherein, if the sequence length of each of the at least three microsatellite loci in said sample from the subject differs from the average sequence length of the at least three microsatellite loci in nucleic acid obtained from the reference population, then the subject is diagnosed as having colon cancer.

In some aspects, if the subject is diagnosed as having colon cancer, the method further comprises treating the subject for colon cancer. In some aspects, the subject was suspected of having colon cancer because the subject had one or more prior tests consistent with or suggestive of a diagnosis of colon cancer.

In some aspects, the sample from the subject comprises a blood sample, skin sample, or oral swab. In some aspects, the nucleic acid being analyzed is DNA, such as genomic DNA. In some aspects, the DNA, such as genomic DNA, is non-tumor, germline DNA. In some aspects, extracting nucleic acid from the sample comprises preparing DNA, such as genomic DNA from the sample. In some aspects, extracting nucleic acid from the sample comprises preparing RNA from the sample. In certain embodiments, a benefit of the disclosure is the ability to accurately diagnose cancer or predict risk susceptibility of a disease or condition by analyzing a sample that can be obtained non-invasively or minimally invasively. For example, given that the subject methods can be robustly used to analyze microsatellite loci that differ in non-tumor tissues, not just in tumor cells, patients can be evaluated using simple blood sample or cheek swabs—rather than via a biopsy. This is particularly useful when obtaining a biopsy is itself painful and/or dangerous, such as for cancers located in the brain. In certain embodiments, the sample (e.g., tissue sample) was previously obtained and nucleic acid was previously isolated and processed. Thus, any of the methods provided herein may be performed using a fresh or frozen tissue sample, or using nucleic acid or nucleic acid sequence information previously obtained from a sample. For example, previously obtained nucleic acid may be provided and used as the basis for determining sequence. Alternatively, previously obtained sequence information may be provided to a host computer and used as the basis for analysis.

In certain aspects, analyzing nucleic acid comprises amplifying the nucleotide sequence of each of said loci by performing polymerase chain reaction (PCR) using primers flanking each of said loci; and evaluating the amplified fragment by capillary electrophoresis or sequencing. In other aspects, analyzing nucleic acid comprises performing next-generation sequencing. In certain embodiments, an enrichment step is performed, such as by using an enrichment array, to enrich for informative loci in a sample prior to performing capillary electrophoresis or sequencing. It should be noted that amplification using, for example, PCR is optional, and analysis by sequencing (e.g., NextGen sequencing) can be performed without the need for prior amplification. In certain embodiments, prior to performing sequencing to analyze one or more informative microsatellite loci, the sample is processed to enrich for microsatellite loci. Such enrichment may be with a general enrichment array or kit (e.g., set of reagents) that enriches generally for all or a subset of microsatellites in a sample prior to sequencing. Alternatively, such enrichment may be with a specific enrichment array or kit that enriches for one or more of the microsatellite loci that one ultimately wishes to analyze via sequencing (e.g., the enrichment kit enriches for one or more microsatellite loci that are informative for a disease, condition or trait). Either kit may be used to enrich the sample prior to sequencing. One benefit of using an enrichment kit is that it increases the number of callable allelotypes or genotypes in a read and increases the ability to analyze a larger percentage of informative loci for a given sample. General or specific enrichment kits comprise, in certain embodiments, probes, such as capture probes, that are hybridizable (intended to specifically hybridize to all or a portion of) for target sequence, such as target sequence that includes a microsatellite of interest and, optionally, flanking sequence (e.g., 1, 2, 3, 4, 5, 6, 7, 8, 9, 10 nucleotides or at least 1, 2, 3, 4, 5, 6, 7, 8, 9, or 10 nucleotides) on either or both sides of the microsatellite. The use of an enrichment kit, prior to analyzing a sample has numerous benefits. In certain embodiments, the inclusion of an enrichment step increases the number of callable genotypes (e.g., the number of callable genotypes for the informative microsatellite loci being evaluated in a given application), and thus, permits analysis of a larger percentage of informative loci per sample. In certain embodiments, the inclusion of an enrichment step increases the number of callable genotypes by at least 5%, 10%, 20%, 30%, 40%, 50%, 60%, 70%, 80%, 90%, 100% or more, as compared to the number of callable genotypes obtainable using, for example, a Next Generation sequence platform without an enrichment step. In certain embodiments, the inclusion of an enrichment step increases the number of callable genotypes by a factor of at least 2, 3, 4, 5, 6, 7, 8, 9, 10 or more, as compared to the number of callable genotypes obtainable using, for example, a Next Generation sequence platform without an enrichment step. In certain embodiments, the inclusion of an enrichment step permits analysis of loci that are otherwise difficult to assess because they are in a portion of the genome difficult to access, and thus underrepresented in reads that are not enriched. In certain embodiments, when calculating the increase in the number or percentage of callable loci, such as the increase in callable genotypes for the informative microsatellites being evaluated, the relevant comparison is made using the same sequencing platform, in the presence or absence of the enrichment step and reagents.

In certain embodiments, an enrichment step is used as part of the initial analysis of samples to generate information about a population. For example, enrichment with a general microsatellite array or kit that enriches for all or a subset of microsatellites may be used when initially generating information about one or more reference populations. In certain embodiments, this increases the loci available for analysis, and thus, may reveal informative loci that would otherwise not be considered because they would not be present with sufficient fidelity and depth to include in the analysis.

The present disclosure also provides a method for measuring propensity for polymorphism, comprising: (a) iteratively aligning a set of microsatellite data corresponding to a subject in a population, to a reference microsatellite loci dataset, comprising: (i) iteratively selecting a microsatellite and sequence portions flanking the selected microsatellite from said set of microsatellite data corresponding to the said subject; and (ii) identifying a similarity between the selected microsatellite and sequence portions and a first locus from said reference microsatellite loci dataset; (b) iteratively determining sequence lengths of the microsatellite loci to which similarities were identified from said set of microsatellite data corresponding to said subject; (c) forming a distribution of the sequence lengths associated with each microsatellite locus in the said reference microsatellite loci dataset; and (d) determining a value based on said microsatellite loci-specific sequence length distribution, wherein a selected group of said microsatellite loci-specific values is indicative of a propensity for polymorphism.

In certain aspects, the set of microsatellite data corresponding to the subject in the population is generated by locating repeating subsequences in a set of sequence reads corresponding to said subject. In certain aspects, the population includes humans associated with known physiological states.

In certain aspects, the method for measuring propensity for polymorphism further comprises assessing, for each microsatellite, a quality score indicative of an accuracy of the bases in the microsatellite; and discarding microsatellites that have quality scores below a first predetermined threshold. In certain aspects, the method further comprises assessing, for each microsatellite, an alignment quality score indicative of an accuracy of the alignment to said reference microsatellite loci dataset; and discarding microsatellites that have alignment quality scores below a second predetermined threshold. In certain aspects, the method further comprises ranking loci of the reference microsatellite loci dataset based on the values determined from the sequence length distributions associated with each microsatellite locus. In certain aspects, the method further comprises identifying each microsatellite locus as heterozygous or homozygous.

In certain aspects, the value is selected from the group consisting of width of the distribution, length of the repeating subsequence, average number of repetitions, purity of the microsatellite locus, and base composition of the subsequence.

In certain aspects, the method for measuring propensity for polymorphism further comprises iteratively training a classifier on the distribution; and using a selected group of classifiers to determine a likelihood of polymorphism. In some aspects, the method further comprises filtering of said set of microsatellite data corresponding to a subject in a population, after said alignment through said identifications of said similarities; generating a local mapping reference microsatellite loci dataset; realigning said set of microsatellite data to said local mapping reference; converting loci positions of said set of microsatellite data relative to said local mapping reference to loci positions relative to said reference microsatellite loci dataset, generating a second alignment; and revising the original alignment to said reference microsatellite loci dataset, based on a comparison of the original alignment to the second alignment.

In some aspects, the determination of the sequence lengths of the microsatellite loci to which similarities were identified, from said set of microsatellite data, requires a difference between percentages of microsatellite data supporting each said identified microsatellite loci be at most 30%. In some aspects, the classifier is selected from the group consisting of likelihood of a sequence length at a microsatellite loci, posterior probability of said sequence length, posterior distribution of sequence lengths at said microsatellite loci, the difference between said posterior distribution and a pre-defined distribution, and whether said microsatellite loci is heterozygous or homozygous.

In some aspects, the sequence lengths are determined by minimizing the mean square error between an observed proportion of reads containing the said microsatellite and Gaussian mixtures parameterized by allelotypes, further comprising: generating confidence scores for each sequence length; and comparing the confidence scores to a pre-defined threshold value to finalized the called sequence length.

In some aspects, the method for measuring propensity for polymorphism further comprises a display device configured to depict the sequence lengths and/or nucleotide sequences of the one or more microsatellites in the test set, and the sequence length and/or nucleotide sequences of the matching microsatellite loci in the reference set. In some aspects, the method for measuring propensity for polymorphism further comprises using a clustering algorithm to identify loci with co-varying distributions.

The present disclosure also provides a method for providing web-based database of microsatellite data, comprising: receiving a set of microsatellite data; identifying microsatellites loci in the set that are likely to be polymorphic; assessing, for each said microsatellite loci, a conservation score, an impact score, and a mutability score; and displaying an indication of the identified microsatellite loci, the conservation scores, the impact scores, and the mutability scores to a user.

The present disclosure also provides a user interface, comprising: (i) a receiver configured to: receive a reference set of microsatellite information for one or more microsatellite loci over a network, wherein the reference set includes reference values indicative of a propensity for polymorphism for each of said one or more microsatellite loci; and receive a test set of microsatellite data from a subject; (ii) a processor configured to: identify a matching microsatellite loci in the reference set corresponding to a microsatellite in the test set; determine sequence length of said matching microsatellite of the test set; and compare the sequence length to a reference value corresponding to the matching microsatellite loci in the reference set.

In certain aspects, the processor is further configured to compare the nucleotide sequence of the microsatellite in the test set to that of the microsatellite loci in the reference set.

The present disclosure also provides an apparatus for identifying an increased risk of developing cancer, comprising: a non-transitory memory; a sample receiver for obtaining a sample of nucleic acid from a subject; a microsatellite profiler for determining a profile for said sample for two or more microsatellite loci; and a comparator for comparing the microsatellite profile from said sample to a reference microsatellite profile generated from nucleic acid from a reference population to identify an alteration at the two or more microsatellite loci in the sample relative to that of the reference population; wherein the alteration at said two or more microsatellite loci is associated with an increased risk of developing cancer.

In a sixth aspect, the disclosure provides a method for identifying an informative microsatellite locus, comprising (i) determining a genotype for a microsatellite locus for each of a plurality of members of a population of individuals identified as having a disease or condition, wherein the genotype for the microsatellite locus for each said member is determined by reliably calling the genotype; (ii) determining a genotype for the same microsatellite locus determined in (i) for each of a plurality of members of a population of individuals identified as not having the disease or condition, wherein the genotype for the microsatellite locus for each said member is determined by reliably calling the genotype; (iii) determining a distribution of the genotypes determined in step (i), which distribution is the distribution of genotypes for the microsatellite locus from nucleic acid obtained from the population of individuals identified as having the disease or condition; (iv) determining a distribution of the genotypes determined in step (ii), which distribution is the distribution of genotypes for the microsatellite locus from nucleic acid obtained from the population of individuals identified as not having the disease or condition; (v) comparing the distribution of genotypes determined in step (iii) to the distribution of genotypes for the same microsatellite locus determined in step (iv); and (vi) classifying the microsatellite locus as informative for the disease or condition if the distribution of genotypes do not significantly overlap between the population of individuals identified as having the disease or condition and the population of individuals identified as not having the disease or condition.

In certain embodiments, a method identifying an informative microsatellite locus is a computer-implemented method which comprises: (i) determining, in a host computer, a genotype for a microsatellite locus for each of a plurality of members of a population of individuals identified as having a disease or condition, wherein the genotype for the microsatellite locus for each said member is determined by reliably calling the genotype; (ii) determining, in the host computer, a genotype for the same microsatellite locus determined in (i) for each of a plurality of members of a population of individuals identified as not having the disease or condition, wherein the genotype for the microsatellite locus for each said member is determined by reliably calling the genotype; (iii) determining, in the host computer, a distribution of the genotypes determined in step (i), which distribution is the distribution of genotypes for the microsatellite locus from nucleic acid obtained from the population of individuals identified as having the disease or condition; (iv) determining, in the host computer, a distribution of the genotypes determined in step (ii), which distribution is the distribution of genotypes for the microsatellite locus from nucleic acid obtained from the population of individuals identified as not having the disease or condition; (v) comparing, in the host computer, the distribution of genotypes determined in step (iii) to the distribution of genotypes for the same microsatellite locus determined in step (iv); and (vi) classifying, in the host computer, the microsatellite locus as informative for the disease or condition if the distribution of genotypes do not significantly overlap between the population of individuals identified as having the disease or condition and the population of individuals identified as not having the disease or condition. It is understood that any one or more of steps may be performed on the same computer or different computers, including across computers interconnected via a network or server or series of servers.

In certain embodiments of any of the foregoing or following aspects and embodiments, further comprises (vii) repeating steps (i) and (ii) for a plurality of microsatellite loci, thereby identifying a plurality of informative microsatellite loci.

In a seventh aspect, the disclosure provides a panel of informative microsatellite loci, identified by any of the foregoing or following aspects and embodiments.

In an eighth aspect, the disclosure provides a system that implements any of the foregoing or following aspects and embodiments.

In a ninth aspect, the disclosure provides a method of identifying condition-associated genotypes in a sample, comprising: (i) obtaining a sample comprising nucleic acid from a subject; (ii) analyzing the nucleic acid to determine a genotype for at least 30% of microsatellite loci from a panel of microsatellite loci identified as being informative for the condition, wherein each informative microsatellite locus is a locus whose distributions of genotypes do not significantly overlap between a population of a plurality of individuals identified as having the condition and a population of a plurality of individuals identified as not having the condition; (iii) comparing the genotype of a first microsatellite locus genotyped in (ii) to a genotype or distribution of genotypes, for that locus, of a reference population identified as having the condition and/or a genotype or distribution of genotypes of a reference population identified as not having the condition; and (iv) repeating step (iii) for one or more of the remaining genotyped microsatellite loci; thereby identifying condition-associated genotypes in a sample. In certain embodiments, analysis of the genotyped microsatellites identifies a condition-associated genotype in a sample with a specificity of at least 60% and a sensitivity of at least 60%.

In certain embodiments, a method identifying condition-associated genotypes in a sample is a computer-implemented method which comprises: (i) receiving, at a host computer, a value representing the genotype for at least 30% of microsatellite loci from a panel of microsatellite loci identified as being informative for the condition, determined by an analysis of nucleic acid obtained from a subject, wherein each informative microsatellite locus is a locus whose distributions of genotypes do not significantly overlap between a population of a plurality of individuals identified as having the condition and a population of a plurality of individuals identified as not having the condition; (ii) comparing, in a host computer, the value to a genotype or distribution of genotypes, for that locus, of a reference population identified as having the condition and/or a genotype or distribution of genotypes of a reference population identified as not having the condition; and (iii) repeating step (ii), in a host computer, for one or more of the remaining genotyped microsatellite loci; thereby identifying condition-associated genotypes in a sample. In certain embodiments, analysis of the genotyped microsatellites identifies a condition-associated genotype in a sample with a specificity of at least 60% and a sensitivity of at least 60%. It is understood that any one or more of steps may be performed on the same computer or different computers, including across computers interconnected via a network or server or series of servers.

In a tenth aspect, the disclosure provides a method of identifying an increased risk of developing a condition, comprising: (i) obtaining a sample comprising nucleic acid from a subject; (ii) analyzing the nucleic acid to determine a genotype for at least 30% of microsatellite loci from a panel of microsatellite loci identified as being informative for the condition, wherein each informative microsatellite locus is a locus whose distributions of genotypes do not significantly overlap between a population of a plurality of individuals identified as having the condition and a population of a plurality of individuals identified as not having the condition; (iii) comparing the genotype of a first microsatellite locus genotyped in (ii) to a genotype, for that locus, of a reference population identified as having the condition and/or a genotype or distribution of genotypes of a reference population identified as not having the condition; and (iv) repeating step (iii) for one or more of the remaining genotyped microsatellite loci; wherein, analysis of the genotyped microsatellites identifies an increased risk of developing a condition with a specificity of at least 60% and a sensitivity of at least 60%.

In certain embodiments, a method identifying an increased risk of developing a condition is a computer-implemented method which comprises: (i) receiving, at a host computer, a value representing the genotype for at least 30% of microsatellite loci from a panel of microsatellite loci identified as being informative for the condition, determined by an analysis of nucleic acid obtained from a subject, wherein each informative microsatellite locus is a locus whose distributions of genotypes do not significantly overlap between a population of a plurality of individuals identified as having the condition and a population of a plurality of individuals identified as not having the condition; (ii) comparing, in a host computer, the value to a genotype, for that locus, of a reference population identified as having the condition and/or a genotype or distribution of genotypes of a reference population identified as not having the condition; and (iii) repeating step (ii), in a host computer, for one or more of the remaining genotyped microsatellite loci; wherein, analysis of the genotyped microsatellites identifies an increased risk of developing a condition with a specificity of at least 60% and a sensitivity of at least 60%. It is understood that any one or more of steps may be performed on the same computer or different computers, including across computers interconnected via a network or server or series of servers.

In an eleventh aspect, the disclosure provides a method of identifying condition-associated genotypes in a sample, comprising (i) obtaining a sample comprising nucleic acid from a subject; (ii) analyzing the sample to determine a genotype for at least 30% of the microsatellite loci listed in Table 14; (iii) comparing the genotype of a first microsatellite locus genotyped in (ii) to a genotype or distribution of genotypes, for that locus, of a reference population identified as having breast cancer and/or a reference population identified as not having breast cancer; and (iv) repeating step (iii) for one or more of the remaining genotyped microsatellite loci.

In certain embodiments, a method identifying condition-associated genotypes in a sample is a computer-implemented method which comprises: (i) receiving, at a host computer, a value representing the genotype for at least 30% of the microsatellite loci listed in Table 14, determined by an analysis of nucleic acid obtained from a subject; (ii) comparing, in a host computer, the value to a genotype or distribution of genotypes, for that locus, of a reference population identified as having breast cancer and/or a reference population identified as not having breast cancer; and (iii) repeating step (ii), in a host computer, for one or more of the remaining genotyped microsatellite loci. It is understood that any one or more of steps may be performed on the same computer or different computers, including across computers interconnected via a network or server or series of servers.

In a twelfth aspect, the disclosure provides a method for diagnosing breast cancer in a subject suspected of having breast cancer, comprising: (i) obtaining a sample comprising nucleic acid from a subject; (ii) analyzing the nucleic acid to determine a genotype for at least 30% of the microsatellite loci listed in Table 14; (iii) comparing the genotype of a first microsatellite locus genotyped in (ii) to a genotype or distribution of genotypes, for that locus, of a reference population identified as having breast cancer and/or a genotype of a reference population identified as not having breast cancer; (iv) repeating step (iii) for one or more of the remaining genotyped microsatellite loci; and (v) diagnosing the subject as having breast cancer if at least 70% of the genotyped microsatellites have a genotype that is associated with the reference population identified as having breast cancer.

In certain embodiments, a method diagnosing breast cancer in a subject suspected of having breast cancer is a computer-implemented method which comprises: (i) receiving, at a host computer, a value representing the genotype for at least 30% of the microsatellite loci listed in Table 14, determined by an analysis of nucleic acid obtained from a subject; (ii) comparing, in a host computer, the value to a genotype or distribution of genotypes, for that locus, of a reference population identified as having breast cancer and/or a genotype of a reference population identified as not having breast cancer; (iii) repeating step (ii), in a host computer, for one or more of the remaining genotyped microsatellite loci; and (iv) diagnosing the subject as having breast cancer if at least 70% of the genotyped microsatellites have a genotype that is associated with the reference population identified as having breast cancer. It is understood that any one or more of steps may be performed on the same computer or different computers, including across computers interconnected via a network or server or series of servers.

In a thirteenth aspect, the disclosure provides a method for treating breast cancer, comprising: (i) obtaining a sample comprising nucleic acid from a subject suspected of having breast cancer; (ii) analyzing the sample to determine a genotype for at least one of the microsatellite loci in Table 14 identified as having a relative risk of >1.1; (iii) comparing the genotype of a first microsatellite locus genotyped in (ii) to a genotype or distribution of genotypes, for that locus, of a reference population identified as having breast cancer and/or a reference population identified as not having breast cancer; and (iv) repeating step (iii) for one or more of the remaining genotyped microsatellite loci, if any; (v) diagnosing the subject as having breast cancer if at least one of the genotyped microsatellites having a relative risk of >1.1 has a genotype that is associated with the reference population identified as having breast cancer; and (vi) providing one or more treatment options if the subject is diagnosed as having breast cancer.

In certain embodiments, a method for treating breast cancer is a computer-implemented method which comprises: (i) receiving, at a host computer, a value representing the genotype for at least one of the microsatellite loci in Table 14 identified as having a relative risk of >1.1, determined by an analysis of nucleic acid obtained from a subject; (ii) comparing, in a host computer, the genotype of a first microsatellite locus genotyped in (i) to a genotype or distribution of genotypes, for that locus, of a reference population identified as having breast cancer and/or a reference population identified as not having breast cancer; and (iii) repeating step (ii), in a host computer, for one or more of the remaining genotyped microsatellite loci, if any; (iv) diagnosing the subject as having breast cancer if at least one of the genotyped microsatellites having a relative risk of >1.1 has a genotype that is associated with the reference population identified as having breast cancer; and (v) providing one or more treatment options if the subject is diagnosed as having breast cancer. It is understood that any one or more of steps may be performed on the same computer or different computers, including across computers interconnected via a network or server or series of servers.

In a fourteenth aspect, the disclosure provides a method identifying subjects at increased risk for developing breast cancer, comprising: (i) obtaining a sample comprising nucleic acid from a subject; (ii) analyzing the sample to determine a genotype for at least one high risk breast cancer microsatellite loci, wherein a high risk breast cancer microsatellite loci is one of the microsatellite loci in Table 14 identified as having a relative risk of >1.1; (iii) comparing the genotype of a first microsatellite locus genotyped in (ii) to a genotype or distribution of genotypes, for that locus, of a reference population identified as having breast cancer and/or a reference population identified as not having breast cancer; and (iv) repeating step (iii) for one or more of the remaining genotyped microsatellite loci, if any; wherein, if at least one of the genotyped high risk microsatellites has a genotype that is associated with the reference population identified as having breast cancer, then the subject is identified as being at an increased risk of developing breast cancer.

In certain embodiments, a method identifying subjects at increased risk for developing breast cancer is a computer-implemented method which comprises: (i) receiving, at a host computer, a value representing the genotype for at least one high risk breast cancer microsatellite loci, determined by an analysis of nucleic acid obtained from a subject, wherein a high risk breast cancer microsatellite loci is one of the microsatellite loci in Table 14 identified as having a relative risk of >1.1; (ii) comparing, in a host computer, the genotype of a first microsatellite locus genotyped in (i) to a genotype or distribution of genotypes, for that locus, of a reference population identified as having breast cancer and/or a reference population identified as not having breast cancer; and (iii) repeating step (ii), in a host computer, for one or more of the remaining genotyped microsatellite loci, if any; wherein, if at least one of the genotyped high risk microsatellites has a genotype that is associated with the reference population identified as having breast cancer, then the subject is identified as being at an increased risk of developing breast cancer. It is understood that any one or more of steps may be performed on the same computer or different computers, including across computers interconnected via a network or server or series of servers.

In a fifteenth aspect, the disclosure provides a method of identifying condition-associated genotypes in a sample, comprising (i) obtaining a sample comprising nucleic acid from a subject; (ii) analyzing the sample to determine a genotype for at least 30% of the microsatellite loci listed in Table 17; (iii) comparing the genotype of a first microsatellite locus genotyped in (ii) to a genotype or distribution of genotypes, for that locus, of a reference population identified as having glioblastoma multiforme (GBM) and/or a reference population identified as not having GBM; and (iv) repeating step (iii) for one or more of the remaining genotyped microsatellite loci.

In certain embodiments, a method identifying condition-associated genotypes in a sample is a computer-implemented method which comprises: (i) receiving, at a host computer, a value representing the genotype for at least 30% of the microsatellite loci listed in Table 17, determined by an analysis of nucleic acid obtained from a subject; (ii) comparing, in a host computer, the genotype of a first microsatellite locus genotyped in (i) to a genotype or distribution of genotypes, for that locus, of a reference population identified as having glioblastoma multiforme (GBM) and/or a reference population identified as not having GBM; and (iii) repeating step (ii), in a host computer, for one or more of the remaining genotyped microsatellite loci. It is understood that any one or more of steps may be performed on the same computer or different computers, including across computers interconnected via a network or server or series of servers.

In a sixteenth aspect, the disclosure provides a method of identifying subjects at increased risk for developing glioblastoma multiforme (GBM), comprising: (i) obtaining a sample comprising nucleic acid from a subject; (ii) analyzing the sample to determine a genotype for at least 30% of the microsatellite loci listed in Table 17; (iii) comparing the genotype of a first microsatellite locus genotyped in (ii) to a genotype or distribution of genotypes, for that locus, of a reference population identified as having GBM and/or a reference population identified as not having GBM; and (iv) repeating step (iii) for one or more of the remaining genotyped microsatellite loci; wherein, if at least 50% of the genotyped microsatellites have a genotype that is associated with the reference population identified as having GBM, then the subject is identified as being at an increased risk of developing GBM.

In certain embodiments, a method identifying subjects at increased risk for developing glioblastoma multiforme (GBM) is a computer-implemented method which comprises: (i) receiving, at a host computer, a value representing the genotype for at least 30% of the microsatellite loci listed in Table 17, determined by an analysis of nucleic acid obtained from a subject; (ii) comparing, in a host computer, the genotype of a first microsatellite locus genotyped in (i) to a genotype or distribution of genotypes, for that locus, of a reference population identified as having GBM and/or a reference population identified as not having GBM; and (iii) repeating step (ii), in a host computer, for one or more of the remaining genotyped microsatellite loci; wherein, if at least 50% of the genotyped microsatellites have a genotype that is associated with the reference population identified as having GBM, then the subject is identified as being at an increased risk of developing GBM. It is understood that any one or more of steps may be performed on the same computer or different computers, including across computers interconnected via a network or server or series of servers.

In a seventeenth aspect, the disclosure provides a method for diagnosing glioblastoma multiforme (GBM) in a subject suspected of having GBM, comprising: (i) obtaining a sample comprising nucleic acid from a subject; (ii) analyzing the nucleic acid to determine a genotype for at least 30% of the microsatellite loci listed in Table 17; (iii) comparing the genotype of a first microsatellite locus genotyped in (ii) to a genotype or distribution of genotypes, for that locus, of a reference population identified as having GBM and/or a reference population identified as not having GBM; (iv) repeating step (iii) for one or more of the remaining genotyped microsatellite loci; and (v) diagnosing the subject as having breast cancer if at least 50% of the genotyped microsatellites have a genotype that is associated with the reference population identified as having GBM.

In certain embodiments, a method diagnosing glioblastoma multiforme (GBM) in a subject suspected of having GBM is a computer-implemented method which comprises: (i) receiving, at a host computer, a value representing the genotype for at least 30% of the microsatellite loci listed in Table 17, determined by an analysis of nucleic acid obtained from a subject; (ii) comparing, in a host computer, the genotype of a first microsatellite locus genotyped in (i) to a genotype or distribution of genotypes, for that locus, of a reference population identified as having GBM and/or a reference population identified as not having GBM; (iii) repeating step (ii), in a host computer, for one or more of the remaining genotyped microsatellite loci; and (iv) diagnosing the subject as having breast cancer if at least 50% of the genotyped microsatellites have a genotype that is associated with the reference population identified as having GBM. It is understood that any one or more of steps may be performed on the same computer or different computers, including across computers interconnected via a network or server or series of servers.

In an eighteenth aspect, the disclosure provides a method for treating low-grade glioma (LGG), comprising: (i) obtaining a sample comprising nucleic acid from a subject suspected of LGG; (ii) analyzing the sample to determine a genotype for at least 30% of the microsatellite loci listed in Table 18; (iii) comparing the genotype of a first microsatellite locus genotyped in (ii) to a genotype or genotype distribution, for that locus, of a reference population identified as having LGG and/or a reference population identified as not having LGG; (iv) repeating step (iii) for one or more of the remaining genotyped microsatellite loci; (v) diagnosing the subject as having LGG if at least 30% of the genotyped microsatellites have a genotype that is associated with the reference population identified as having LGG; wherein the method has a sensitivity of at least 85% and a specificity of at least 80% for diagnosing LGG; and (vi) providing one or more treatment options if the subject is diagnosed as having LGG.

In certain embodiments, a method treating low-grade glioma (LGG) is a computer-implemented method which comprises: (i) receiving, at a host computer, a value representing the genotype for at least 30% of the microsatellite loci listed in Table 18, determined by an analysis of nucleic acid obtained from a subject; (ii) comparing, in a host computer, the genotype of a first microsatellite locus genotyped in (i) to a genotype or genotype distribution, for that locus, of a reference population identified as having LGG and/or a reference population identified as not having LGG; (iii) repeating step (ii), in a host computer, for one or more of the remaining genotyped microsatellite loci; (iv) diagnosing the subject as having LGG if at least 30% of the genotyped microsatellites have a genotype that is associated with the reference population identified as having LGG; wherein the method has a sensitivity of at least 85% and a specificity of at least 80% for diagnosing LGG; and (v) providing one or more treatment options if the subject is diagnosed as having LGG. It is understood that any one or more of steps may be performed on the same computer or different computers, including across computers interconnected via a network or server or series of servers.

In a nineteenth aspect, the disclosure provides a method of identifying subjects at increased risk for developing low-grade glioma (LGG), comprising: (i) obtaining a sample comprising nucleic acid from a subject; (ii) analyzing the sample to determine a genotype for at least 30% of the microsatellite loci listed in Table 18; (iii) comparing the genotype of a first microsatellite locus genotyped in (ii) to a genotype or genotype distribution, for that locus, of a reference population identified as having LGG and/or a reference population identified as not having LGG; and (iv) repeating step (iii) for one or more of the remaining genotyped microsatellite loci; wherein, if at least 30% of the genotyped microsatellites have a genotype that is associated with the reference population identified as having LGG, then the subject is identified as being at an increased risk of developing LGG.

In certain embodiments, a method identifying subjects at increased risk for developing low-grade glioma (LGG) is a computer-implemented method which comprises: (i) receiving, at a host computer, a value representing the genotype for at least 30% of the microsatellite loci listed in Table 18, determined by an analysis of nucleic acid obtained from a subject; (ii) comparing, in a host computer, the genotype of a first microsatellite locus genotyped in (i) to a genotype or genotype distribution, for that locus, of a reference population identified as having LGG and/or a reference population identified as not having LGG; and (iii) repeating step (ii), in a host computer, for one or more of the remaining genotyped microsatellite loci; wherein, if at least 30% of the genotyped microsatellites have a genotype that is associated with the reference population identified as having LGG, then the subject is identified as being at an increased risk of developing LGG. It is understood that any one or more of steps may be performed on the same computer or different computers, including across computers interconnected via a network or server or series of servers.

In a twentieth aspect, the disclosure provides a method for diagnosing low-grade glioma (LGG) in a subject suspected of having LGG, comprising: (i) obtaining a sample comprising nucleic acid from a subject; (ii) analyzing the nucleic acid to determine a genotype for at least 30% of the microsatellite loci listed in Table 18; (iii) comparing the genotype of a first microsatellite locus genotyped in (ii) to a genotype or genotype distribution, for that locus, of a reference population identified as having LGG and/or a reference population identified as not having LGG; (iv) repeating step (iii) for one or more of the remaining genotyped microsatellite loci; and (v) diagnosing the subject as having breast cancer if at least 30% of the genotyped microsatellites have a genotype that is associated with the reference population identified as having LGG.

In certain embodiments, a method diagnosing low-grade glioma (LGG) in a subject suspected of having LGG is a computer-implemented method which comprises: (i) receiving, at a host computer, a value representing the genotype for at least 30% of the microsatellite loci listed in Table 18, determined by an analysis of nucleic acid obtained from a subject; (ii) comparing, in a host computer, the genotype of a first microsatellite locus genotyped in (i) to a genotype or genotype distribution, for that locus, of a reference population identified as having LGG and/or a reference population identified as not having LGG; (iii) repeating step (ii), in a host computer, for one or more of the remaining genotyped microsatellite loci; and (iv) diagnosing the subject as having breast cancer if at least 30% of the genotyped microsatellites have a genotype that is associated with the reference population identified as having LGG. It is understood that any one or more of steps may be performed on the same computer or different computers, including across computers interconnected via a network or server or series of servers.

In a twenty-first aspect, the disclosure provides a method of diagnosing whether a subject suspected of having brain cancer has glioblastoma multiforme (GBM) versus low-grade glioma (LGG), comprising: (i) obtaining a sample comprising nucleic acid from a subject; (ii) analyzing the sample to determine a genotype for at least 30% of the microsatellite loci listed in Table 19; (iii) comparing the genotype of a first microsatellite locus genotyped in (ii) to a genotype or genotype distribution, for that locus, of a reference population identified as having LGG and/or a reference population identified as having GBM; (iv) repeating step (iii) for one or more of the remaining genotyped microsatellite loci; and (v) diagnosing the subject as having GBM if at least 75% of the genotyped microsatellites have a genotype that is associated with the reference population identified as having GBM; wherein the method has a sensitivity of at least 70% and a specificity of at least 85% for diagnosing GBM.

In certain embodiments, a method diagnosing whether a subject suspected of having brain cancer has glioblastoma multiforme (GBM) versus low-grade glioma (LGG) is a computer-implemented method which comprises: (i) receiving, at a host computer, a value representing the genotype for at least 30% of the microsatellite loci listed in Table 19, determined by an analysis of nucleic acid obtained from a subject; (ii) comparing, in a host computer, the genotype of a first microsatellite locus genotyped in (i) to a genotype or genotype distribution, for that locus, of a reference population identified as having LGG and/or a reference population identified as having GBM; (iii) repeating step (ii), in a host computer, for one or more of the remaining genotyped microsatellite loci; and (iv) diagnosing the subject as having GBM if at least 75% of the genotyped microsatellites have a genotype that is associated with the reference population identified as having GBM; wherein the method has a sensitivity of at least 70% and a specificity of at least 85% for diagnosing GBM. It is understood that any one or more of steps may be performed on the same computer or different computers, including across computers interconnected via a network or server or series of servers.

In a twenty-second aspect, the disclosure provides a method of diagnosing whether a subject suspected of having brain cancer has glioblastoma multiforme (GBM) versus Grade II low-grade glioma (LGG), comprising: (i) obtaining a sample comprising nucleic acid from a subject; (ii) analyzing the sample to determine a genotype for at least 30% of the microsatellite loci listed in Table 20; (iii) comparing the genotype of a first microsatellite locus genotyped in (ii) to a genotype or genotype distribution, for that locus, of a reference population identified as having Grade II LGG and/or a reference population identified as having GBM; (iv) repeating step (iii) for one or more of the remaining genotyped microsatellite loci; and (v) diagnosing the subject as having GBM if at least 80% of the genotyped microsatellites have a genotype that is associated with the reference population identified as having GBM; wherein the method has a sensitivity of at least 85% and a specificity of at least 65% for diagnosing GBM.

In certain embodiments, a method diagnosing whether a subject suspected of having brain cancer has glioblastoma multiforme (GBM) versus Grade II low-grade glioma (LGG) is a computer-implemented method which comprises: (i) receiving, at a host computer, a value representing the genotype for at least 30% of the microsatellite loci listed in Table 20, determined by an analysis of nucleic acid obtained from a subject; (ii) comparing, in a host computer, the genotype of a first microsatellite locus genotyped in (i) to a genotype or genotype distribution, for that locus, of a reference population identified as having Grade II LGG and/or a reference population identified as having GBM; (iii) repeating step (ii), in a host computer, for one or more of the remaining genotyped microsatellite loci; and (iv) diagnosing the subject as having GBM if at least 80% of the genotyped microsatellites have a genotype that is associated with the reference population identified as having GBM; wherein the method has a sensitivity of at least 85% and a specificity of at least 65% for diagnosing GBM. It is understood that any one or more of steps may be performed on the same computer or different computers, including across computers interconnected via a network or server or series of servers.

In a twenty-third aspect, the disclosure provides a kit comprising: a) one or more solid supports comprising immobilized nucleic acid probes, wherein each nucleic acid probe is hybridizable to a target nucleic acid sequence, wherein the target nucleic acid sequence comprises a microsatellite loci selected from the group consisting of the loci listed in any of tables 14, 17, 18, 19, or 20; and b) one or more reagents for performing hybridizations, washes, and/or elution of target nucleic acid sequences.

In a twenty-fourth aspect, the disclosure provides a kit comprising: a) one or more solid supports comprising immobilized nucleic acid probes hybridizable to a plurality of target nucleic acid sequences, wherein said target nucleic acid sequences comprise at least 2, 5, 10, 15, 25, 30, 35, 40, 45, 50, 55, 60 or all of the microsatellite loci listed in any of tables 14, 17, 18, 19, or 20; and b) one or more reagents for performing hybridizations, washes, and/or elution of target nucleic acid sequences.

In a twenty-fifth aspect, the disclosure provides a computer-implemented method of identifying variant microsatellite loci comprising: (a) receiving, at a computer, a library of sequence reads for subsequences in the nucleic acid from the sample obtained using a Next Generation sequencing platform; (b) aligning a first sequence read from said library to a reference sequence by an alignment method, wherein the alignment method comprises: (i) selecting a microsatellite locus and sequence portion flanking the selected microsatellite locus from said sequence read, wherein the flanking sequence comprises at least 1, 2, 3, 4, 5, 6, 7, 8, 9 or 10 nucleotide bases; and (ii) identifying a similarity between said reference sequence and the selected microsatellite locus and sequence portion flanking the microsatellite locus; (c) determining the sequence and/or length of the microsatellite locus to which a similarity is identified in (ii); (d) repeating (a)-(c) for all the sequence reads in the library of sequence reads; (e) forming a distribution of sequence and/or lengths associated with each microsatellite locus whose length is determined in (c); and (f) assigning a genotype for each microsatellite locus based on its distribution of sequence and/or lengths.

In a twenty-sixth aspect, the disclosure provides a method of identifying informative microsatellite loci comprising: (i) determining a distribution of sequence lengths and/or actual sequences for a plurality of microsatellite loci in nucleic acid obtained from a population of individuals identified as having a condition or a predisposition to a condition; (ii) determining a distribution of sequence lengths and/or actual sequences for a plurality of microsatellite loci in nucleic acid obtained from a population of individuals identified as not having a condition or a predisposition to a condition; (iii) comparing the distribution of sequence lengths and/or actual sequences for a first microsatellite locus in nucleic acid obtained from the population with the condition set forth in (i) to the distribution of sequence lengths for the same first microsatellite locus in nucleic acid obtained from the population without the condition set forth in (ii); (iv) repeating the comparing step (iii) for one or more additional microsatellite loci; and (v) classifying as informative, any microsatellite locus whose distributions of sequence lengths do not significantly overlap between the population of individuals identified as having the condition and the population of individuals identified as not having the condition.

In certain embodiments, a method identifying informative microsatellite loci is a computer-implemented method which comprises: (i) determining, in a host computer, a distribution of sequence lengths and/or actual sequences for a plurality of microsatellite loci in nucleic acid obtained from a population of individuals identified as having a condition or a predisposition to a condition; (ii) determining, in a host computer, a distribution of sequence lengths and/or actual sequences for a plurality of microsatellite loci in nucleic acid obtained from a population of individuals identified as not having a condition or a predisposition to a condition; (iii) comparing, in a host computer, the distribution of sequence lengths and/or actual sequences for a first microsatellite locus in nucleic acid obtained from the population with the condition set forth in (i) to the distribution of sequence lengths for the same first microsatellite locus in nucleic acid obtained from the population without the condition set forth in (ii); (iv) repeating the comparing step (iii), in a host computer, for one or more additional microsatellite loci; and (v) classifying as informative, in a host computer, any microsatellite locus whose distributions of sequence lengths do not significantly overlap between the population of individuals identified as having the condition and the population of individuals identified as not having the condition. It is understood that any one or more of steps may be performed on the same computer or different computers, including across computers interconnected via a network or server or series of servers.

In certain embodiments of any of the foregoing or following aspects and embodiments, the condition is a type of cancer. In certain embodiments of any of the foregoing or following aspects and embodiments, each microsatellite loci has 15× sequence coverage at each microsatellite locus. In certain embodiments of any of the foregoing or following aspects and embodiments, each nucleic acid obtained from a population of individuals has at least 10,000 microsatellite loci called. In certain embodiments of any of the foregoing or following aspects and embodiments, each locus is called in at least 10 samples in each population for inclusion in step (iii). In certain embodiments of any of the foregoing or following aspects and embodiments, step (iv) comprises repeating step (iii) for all of the remaining genotyped microsatellite loci. In certain embodiments of any of the foregoing or following aspects and embodiments, the panel of microsatellite loci identified as being informative comprises a list of at least six, at least seven, at least eight, at least nine, or at least ten microsatellite loci, and the method comprises determining a genotype for at least 30% of the panel of microsatellite loci for any given sample. In certain embodiments of any of the foregoing or following aspects and embodiments, if at least 30% of the genotyped microsatellites have a genotype that is associated with the reference population identified as having the condition, then the subject is identified as being at increased risk of developing the condition.

In certain embodiments of any of the foregoing or following aspects and embodiments, the population of individuals identified as not having the condition have a different condition. In certain embodiments of any of the foregoing or following aspects and embodiments, (iii) comprises comparing the genotype of a first microsatellite locus genotyped in (ii) to the modal genotype from a reference population identified as not having a condition. In certain embodiments of any of the foregoing or following aspects and embodiments, (iii) comprises comparing the genotype of a first microsatellite locus genotyped in (ii) to a distribution of genotypes from a reference population identified as having a condition and/or to a distribution of genotypes from a reference population identified as not having the condition. In certain embodiments of any of the foregoing or following aspects and embodiments, step (iv) comprises, for one or more of the remaining genotyped microsatellite loci, comparing the genotype of the remaining one or more microsatellite loci to the modal genotype from a reference population identified as not having a condition. In certain embodiments of any of the foregoing or following aspects and embodiments, step (iv) comprises, for one or more of the remaining genotyped microsatellite loci, comparing the genotype of a first microsatellite locus genotyped in (ii) to a distribution of genotypes from a reference population identified as having a condition and/or to a distribution of genotypes from a reference population of individuals identified as not having the condition. In certain embodiments of any of the foregoing or following aspects and embodiments, if the relative risk associated with a given genotype for a microsatellite locus is greater than 1.0, then presence of a non-modal genotype in a sample is associated with the condition.

In certain embodiments of any of the foregoing or following aspects and embodiments, the reference population identified as having and/or not having a condition is based on at least 100 members. In certain embodiments of any of the foregoing or following aspects and embodiments, the reference population identified as not having a condition is gender, age, and/or ethnicity matched to the sample. In certain embodiments of any of the foregoing or following aspects and embodiments, the reference population identified as having a condition is gender, age, and/or ethnicity matched to the sample and/or to the reference population identified as not having a condition.

In certain embodiments of any of the foregoing or following aspects and embodiments, analyzing the sample comprises providing a kit comprising reagents for enriching for microsatellite loci in a nucleic acid preparation, prepared from the sample, and contacting nucleic acid from the sample with said reagents to produce an enriched nucleic acid preparation. In certain embodiments of any of the foregoing or following aspects and embodiments, the kit is a kit comprising reagents for enriching, generally, for microsatellite loci. In certain embodiments of any of the foregoing or following aspects and embodiments, analyzing the sample to determine a genotype comprises a computer-implemented method comprising: (a) receiving, at a computer, a library of sequence reads for subsequences in the nucleic acid from the sample obtained using a Next Generation sequencing platform; (b) aligning a first sequence read from said library to a reference sequence by an alignment method, wherein the alignment method comprises: (i) selecting a microsatellite locus and sequence portion flanking the selected microsatellite locus from said sequence read, wherein the flanking sequence comprises at least 1, 2, 3, 4, 5, 6, 7, 8, 9 or 10 nucleotide bases; and (ii) identifying a similarity between said reference sequence and the selected microsatellite locus and sequence portion flanking the microsatellite locus; (c) determining the sequence and/or length of the microsatellite locus to which a similarity is identified in (ii); (d) repeating (a)-(c) for all the sequence reads in the library of sequence reads; (e) forming a distribution of sequence and/or lengths associated with each microsatellite locus whose length is determined in (c); (d) assigning a genotype for each microsatellite locus based on its distribution of sequence and/or lengths.

In certain embodiments of any of the foregoing or following aspects and embodiments, comparing the genotype to a reference population's genotypes for that same locus comprises a computer-implemented method whereby the genotype is compared to a reference population's genotypes or genotype distributions stored in a database or housed on a server. In certain embodiments of any of the foregoing or following aspects and embodiments, analyzing nucleic acid from the subject comprises amplifying the nucleotide sequence of each of said loci by performing polymerase chain reaction (PCR) using primers flanking each of said loci; and

evaluating the amplified fragment by capillary electrophoresis or sequencing. In certain embodiments of any of the foregoing or following aspects and embodiments, analyzing nucleic acid from the sample comprises sequencing the nucleic acids in the sample, such as using a Next Generation sequencing platform.

In certain embodiments of any of the foregoing or following aspects and embodiments, the method has a sensitivity of at least 80% and a specificity of at least 70% for identifying subjects at increased risk of developing breast cancer, the method has a sensitivity of at least 90% and a specificity of at least 70% for diagnosing GBM, the method has a sensitivity of at least 85% and a specificity of at least 80% for identifying subjects at increased risk of developing LGG.

In certain embodiments of any of the foregoing or following aspects and embodiments, at least one of the genotyped microsatellites comprises a microsatellite loci in Table 14 identified as having a relative risk of >1.1. In certain embodiments of any of the foregoing or following aspects and embodiments, at least one of the genotyped microsatellites comprises a microsatellite loci in Table 14 identified as having a relative risk of <0.7. In certain embodiments of any of the foregoing or following aspects and embodiments, the sample comprising nucleic acid is a blood sample or cheek swab, and wherein the sample is not a tumor sample. In certain embodiments of any of the foregoing or following aspects and embodiments, the kit is a kit comprising reagents for enriching for the microsatellite loci listed in Table 14, 17, 18, and/or 20. In certain embodiments of any of the foregoing or following aspects and embodiments, the target nucleic acid sequences comprise, for a particular microsatellite loci, the nucleotide sequence corresponding to one or both alleles of a modal genotype of a reference population identified as healthy.

In certain embodiments of any of the foregoing or following aspects and embodiments, said solid support is a microarray slide. In certain embodiments of any of the foregoing or following aspects and embodiments, said one or more solid supports comprises one or more beads. In certain embodiments of any of the foregoing or following aspects and embodiments, the target nucleic acid sequences comprise the microsatellite loci with at least 5-10 nucleotides of flanking sequence 5′ and/or 3′ to the microsatellite loci. In certain embodiments of any of the foregoing or following aspects and embodiments, the target nucleic acid sequences comprise the microsatellite loci with at least 5-10 nucleotides of flanking sequence 5′ to the microsatellite loci and at least 5-10 nucleotides of flanking sequence 3′ to the microsatellite loci, wherein the number of nucleotides of flanking sequence is independently selected for the 5′ and 3′ flanking sequence. In certain embodiments of any of the foregoing or following aspects and embodiments, the nucleic acid probes are hybridizable to both target nucleic acid sequence corresponding to the microsatellite loci and target nucleic acid sequence corresponding to the flanking sequence. In certain embodiments of any of the foregoing or following aspects and embodiments, the kit comprises a plurality of solid supports, and wherein each solid support comprises probes hybridizable to more than one target nucleic acid sequence. In certain embodiments of any of the foregoing or following aspects and embodiments, the nucleic acid probes are enrichment probes. In certain embodiments of any of the foregoing or following aspects and embodiments, the nucleic acid probes are complementary to the target nucleic acid sequence, without fewer than two mismatches. In certain embodiments of any of the foregoing or following aspects and embodiments, the nucleic acid probes are complementary to the target nucleic acid sequence, without any mismatches.

The disclosure contemplates all combinations of any of the foregoing aspects and embodiments, as well as combinations with any of the embodiments set forth in the detailed description (including tables and figures) and examples.

### BRIEF DESCRIPTION OF THE DRAWINGS

FIG. 1 is a block diagram of a system for microsatellite analysis for diagnosis and predisposition screening of a given physiological condition.

FIG. 2 is a block diagram of a computerized system for microsatellite analysis, according to an illustrative embodiment.

FIG. 3 is a data structure of example allelotype distributions for a set of microsatellite loci, according to an illustrative embodiment.

FIG. 4A is a block diagram of a system for generating genotype data for a given microsatellite data set, according to an illustrative embodiment.

FIG. 4B is a block diagram of a system for aligning short sequence microsatellite data to a reference microsatellite loci dataset, according to an illustrative embodiment.

FIG. 4C is an illustrative example of data manipulation according to the illustrative embodiment shown in FIG. 4B.

FIG. 4D is a block diagram of a system for generating genotype data from a given microsatellite loci data set, according to an illustrative embodiment.

FIG. 5 is an illustrative computing device, which may be used to implement any of the processors and servers described herein.

FIG. 6 is a schematic illustrating a method for the identification of informative microsatellite loci described herein.

FIG. 7 describes the percentage of breast cancer and 1 kGB samples with each allele of 11 informative microsatellite loci identified in the breast cancer analysis. It should be noted that only two different allelotypes were identified. The y-axis describes the percentage of the sample population with each allele and the x-axis describes the 11 signature genes, the prevalence of loci with distinct microsatellite repeats, followed by the microsatellite motif found in each gene, and their transcription factor binding sites. The numbers below the graph represent the percentage of the sample population with each allele.

FIG. 8 describes the percentage of glioblastoma and 1 kGB samples with each allele of 8 informative microsatellites identified in the glioblastoma analysis. Here, four different allotypes were identified. The y-axis describes the percentage of the sample population with each allele and the x-axis describes 8 signature genes and the prevalence of loci with distinct microsatellite repeats. The numbers below the graph represent the percentage of the sample population with each allele.

FIG. 9 shows that it is possible to compute a substantial number of genotypes at microsatellite loci. For example, in approximately 250 samples, up to 9000 loci were successfully sequenced and characterized. Most of the samples displayed are tumor samples.

FIG. 10 shows that a substantial number of loci vary in all the sample types (tumor, non-tumor, unknown), with the mean being approximately six microsatellite loci.

FIG. 11 shows that the level of microsatellite variation (e.g., overall GMI) is significantly greater in genomes from subjects identified as having an ovarian cancer signature (signature of informative microsatellite loci) than in those that were not. Bars indicate the data range. * indicates p≦0.05. This is indicative of experiments that support the use of GMI as a biomarker for cancer risk.

FIG. 12 shows that ovarian cancer-associated intronic microsatellite loci are enriched near exon-intron boundaries. Intronic microsatellites identified as part of the OV-associated loci set are enriched within the 3% of the intron near the exon-intron boundary of the normalized intron as compared to the complete set of introns that are called in at least one of the exome sequenced samples.

FIG. 13 shows the results of an experiment in which microarray-based enrichment was performed to capture specific microsatellite loci in the human genome.

FIG. 14A shows the distributions of exomes based on their genotypes at the 55 BC-associated microsatellite loci set forth in Table 14. In this study, genomes were classified as cancer-like if of the callable microsatellite loci had a cancer associated genotype, as compared to the genotype of a reference population identified as not having breast cancer (“healthy”) and/or a reference population identified as having breast cancer. The comparison may be to the modal genotype of the healthy reference population and/or to the distribution of genotypes of the healthy or the cancer reference population.

FIG. 14B shows the ROC curve of the sensitivity and specificity of the breast cancer signature based on these 55 informative microsatellite loci.

FIG. 15A shows the distributions of exomes based on their genotypes at the 48 GBM-associated microsatellite loci set forth in Table 17. In this study, genomes were classified as cancer-like if ≧57% of the callable microsatellite loci had a non-modal genotype (modal genotype being the most common genotype in a population identified as not having GBM; e.g., a genotype that differed from the most common genotype from a reference population). Genomes were classified as healthy if <57% of callable microsatellite loci have a non-modal genotype.

FIG. 15B shows the ROC curve of the sensitivity and specificity of the GBM signature based on these 48 informative microsatellite loci.

FIG. 16A shows the distributions of exomes based on their genotypes at the 66 LGG-associated microsatellite loci set forth in Table 18. In this study, genomes were classified as cancer-like if ≧35% of the callable microsatellite loci had a non-modal genotype (modal genotype being the most common genotype in a population identified as not having LGG; e.g., a genotype that differed from the most common genotype from a reference population). Genomes were classified as healthy if <35% of callable microsatellite loci have a non-modal genotype.

FIG. 16B shows the ROC curve of the sensitivity and specificity of the LGG signature based on these 66 informative microsatellite loci.

FIG. 17A shows the distributions of exomes based on their genotypes at the 27 microsatellite loci that distinguish GBM from LGG (grades II and III). In this study, genomes were classified as GBM-like if ≧82% of callable microsatellite loci had a non-modal genotype (modal genotype being the most common genotype in a population identified as having LGG). Genomes are classified as LGG if <82% of callable microsatellite loci have a non-modal genotype.

FIG. 17B shows the ROC curve of the sensitivity and specificity of the signature distinguishing GBM from LGG.

FIG. 18 shows that variation at some microsatellite loci correlates with ethnicity. Thus, in certain embodiments, when determining informative microsatellite loci, the reference population may be ethnicity-matched for the intended patient population.

FIG. 19 shows a flow diagram of a microsatellite pipeline. Microsatellite analysis to identify panels of informative microsatellites (PIM) indicative of a state or condition includes the re-building of microsatellite loci in a set of genomes, followed by statistical analysis that includes Type 1 error and False Discovery Rate tests. After which, ancillary data, including ontology, expression and other information that provides independent confidence in the set of informative loci are associated with breast cancer.

FIG. 20 shows the overlap of informative loci distinguishing BC subtypes.

FIG. 21A shows the distributions of exomes based on their genotypes at the 8 microsatellite loci that distinguish GBM from LGG grade II. In this study, genomes were classified as GBM-like if ≧85% of callable microsatellite loci had a non-modal genotype (modal genotype being the most common genotype in a population identified as having LGG Grade II). Genomes were classified as LGG Grade II if <85% of callable microsatellite loci have a non-modal genotype.

FIG. 21B shows the ROC curve of the sensitivity and specificity of the signature distinguishing GBM from LGG Grade II.

FIG. 22A-C depicts the helicase variants DHX36, DICER1, TTF2, DDX20, POLQ and DDX60. These variants represent drug discovery targets.

FIG. 23A-B show the frequency of alleles at STR alleles within exome sequencing data. (A) The majority of all microsatellite alleles are mono- and di-alleleic, even at high read coverage. The peaks ranging from ˜30 reads for loci with three alleles to ˜70 reads for loci determined to have >5 alleles likely demark the minimum read coverage sufficient to call increased numbers of alleles. Error bars represent the SEM. (B) Increasing read coverage did not correlate with an increase in the percentage of loci identified as having multiple (3+) alleles, suggesting that sequencing error does not explain the appearance of multiple alleles.

Table 1 provides information for the initial set of 165 microsatellite loci identified in the breast cancer analysis for which at least one breast cancer (BC) sample was variant from the human genome reference. Such informative microsatellites (e.g., one or more of any such loci) may be used, for example, to predict risk of developing breast cancer in a subject. This list of loci was generated using analysis of allelotype.

Table 2 provides information for the subset of 17 informative microsatellite loci identified in the breast cancer allelotyping analysis. Such informative microsatellites (e.g., one or more any such loci) may be used, for example, to predict risk of developing breast cancer in a subject.

Table 3 reports the percentage of genomes having an ovarian cancer-signature with the indicated minimum variant loci. This signature was identified using allelotyping analysis.

Table 4 provides information for the initial set of 600 microsatellite loci, identified in the ovarian cancer allelotyping analysis, which were conserved in normal females yet had high levels of variation in either ovarian cancer germline nucleic acid, nucleic acid from tumors or both. Such informative microsatellites (e.g., one or more any such loci; including any one or more of loci 1-100) may be used, for example, to predict risk of developing ovarian cancer in a subject.

Table 5 provides information for the initial set of 48 informative microsatellite loci identified in the glioblastoma allelotyping analysis. Of those 48 microsatellite loci, 10 loci (shaded) were identified as being highly informative using “leave-one-out” analysis. Such informative microsatellites (e.g., one or more of any of the 48 loci; or one or more of any of the 10 loci) may be used, for example, to predict risk of developing glioblastoma in a subject.

Table 6 reports the percentage of genomes having a glioblastoma-signature with the indicated minimum variant loci. This signature was identified using allelotyping analysis.

Table 7 provides information for informative microsatellite loci identified in the colon cancer allelotyping analysis. Such informative microsatellites (e.g., one or more of such loci) may be used, for example, to predict colon cancer risk in a subject. The methodologies for identifying informative loci is similar to that described for the breast and ovarian cancer analysis summarized in the above tables.

Table 8 provides information for informative microsatellite loci identified in the lung cancer allelotyping analysis, particularly for lung squamous cell carcinoma. Such informative microsatellites (e.g., one or more of such loci) may be used, for example, to predict lung cancer risk (specifically lung squamous cell carcinoma risk) in a subject. The methodologies for identifying informative loci is similar to that described for the breast and ovarian cancer analysis summarized in the above tables.

Table 9 provides information for informative microsatellite loci identified in the lung cancer allelotyping analysis, particularly for lung adenocarcinoma. Such informative microsatellites (e.g., one or more of such loci) may be used, for example, to predict lung cancer risk (specifically lung adenocarcinoma risk) in a subject. The methodologies for identifying informative loci is similar to that described for the breast and ovarian cancer analysis summarized in the above tables.

Table 10 provides information for informative microsatellite loci identified in the prostate cancer allelotyping analysis. Such informative microsatellites (e.g., one or more such loci) may be used, for example, to predict prostate cancer risk in a subject. The methodologies for identifying informative loci is similar to that described for the breast and ovarian cancer analysis summarized in the above tables.

Table 11 summarizes the changes in protein sequence due to microsatellite variation at 11 informative breast cancer-associated genes. The red amino acids (which are also bolded and underlined) illustrate the alterations in protein sequence caused by variant microsatellites.

Table 12 summarizes data indicating that the overall level of microsatellite variation (global microsatellite instability) was greater in OV patient genomes than in the normal female population. This supports the use of GMI as a biomarker for predicting cancer, such as ovarian cancer, risk.

Table 13 provides the nucleotide sequence for primer pairs suitable for use in amplifying certain informative microsatellite loci.

Table 14 provides information for the 55 BC-associated microsatellite loci identified using genotyping analysis (where genotype, at each locus, was evaluated and used).

Table 15 provides a list of genes with which some of the 55 BC-associated microsatellite loci are associated with or located within and that are known to be associated with cancer generally, specifically with BC, or are involved in other cellular pathways associated with cancer.

Table 16 shows gene expression levels in tumor and germline for genes associated with the 55 BC-associated informative loci from RNASeq. Gray highlighting indicates loci with change in gene expression.

Table 17 provides information for the 48 GBM-associated informative loci identified using genotyping analysis.

Table 18 provides information for the 66 LGG-associated informative loci identified using genotyping analysis.

Table 19 provides information for the loci that can be used to distinguish glioblastoma (GBM) from low grade glioma (LGG), such as to differentially diagnose a subject having a brain lesion.

Table 20 provides information for the loci that can be used to distinguish GBM from LGG grade II, such as to differentially diagnose a subject having a brain lesion.

Table 21 provides examples of variant microsatellites including minor alleles.

Table 22 provides the genotype distribution information for the 55 BC-associated microsatellite loci. The number of times that genotype was observed is in parentheses.

### DETAILED DESCRIPTION OF THE DISCLOSURE

### 1. Overview

Microsatellites, or repetitive DNA, defined as tandem repeats of 1- to 6-mer motifs are pervasive in the human genome. Their analysis and exploitation provide a tremendous opportunity for discovery. However, their analysis is often purposefully excluded from studies, and some would say this is rightfully so. These low complexity elements are difficult to identify and accurately correlate across multiple sequencing reactions. For example microsatellites wreck havoc on certain Next-Generation DNA sequencers (efficacy of Roche 454 drops precipitously for mono-nucleotide runs of 3-4 bases), microarrays (which address individual unique loci in the genome) and especially bioinformatics tools (searching and assembly). Search tools such as BLAST incorporate low complexity filters to mask these sequences, and certain assembly engines perform poorly in these low complexity regions because the read depth is low and because mis-mapped reads can contribute to wrong genotypes and very low accuracy (discussed in further detail below). Target enrichment systems used in the art design their baits to also exclude these low complexity regions, thus exome-sequence sets which dominate current Next-Generation sequencing are depleted for these regions. For these and other reasons the 1-2 million microsatellite loci in the genome are understudied.

It is clear that the study, characterization, and effective use of microsatellite information has been crippled by technological bathers. Moreover, the myths about microsatellites have generally taught away from the use of individual loci and combinations of specific loci as a diagnostic or prognostic indicator. The present disclosure provides methods and systems to permit robust analysis of microsatellites, as well as comparisons of microsatellites between different populations or between an individual patient and a reference population. These tools permit, amongst other things, the identification of informative microsatellite loci that can be used to (i) identify new therapeutic targets (e.g., for drug screening), (ii) assess disease risk, and (iii) prognose disease outcome; as well as to predict likely responsiveness or non-responsive to therapeutic modalities and to definitively diagnose patients non-invasively following an initial test suggestive of a particular disease state. These applications of the technology are described in further detail herein. Moreover, the methods and systems described herein can be used as part of a method of treatment or to initiate a monitoring protocol. Following testing that indicates that an individual is at increased risk for developing, for example, a particular cancer and/or has a particular disease, such as a particular type of cancer, the patient can be monitored, offered prophylactic treatment, and/or offered treatment. Accordingly the present methods can also be used as part of a method of treatment and/or as a diagnostic method.

Before continuing to describe the present disclosure in further detail, it is to be understood that this disclosure is not limited to specific compositions or process steps, as such may vary. It must be noted that, as used in this specification and the appended claims, the singular form “a”, “an” and “the” include plural referents unless the context clearly dictates otherwise.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “about” in the context of a given value or range refers to a value or range that is within 20%, preferably within 10%, and more preferably within 5% of the given value or range.

It is convenient to point out here that “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

When referring to a “population”, such as a reference population, the disclosure contemplates that a characteristic of the population, such as a genotype, is based on information across a plurality of samples, genomes, individuals, or the like. For example, the modal genotype of a reference population refers to the most frequently observed genotype, at a particular microsatellite loci, determined by examination of a plurality of samples, genomes, individuals, or the like. Thus, information about a population is based on information of a plurality of members (e.g., items contributing to the population, such as samples, individuals, genomes, and the like). A population may comprise, for example, at least 2, 5, 8, 10, 15, 20, 25, 30, 35, 40, 45, 50, 60, 70, 75, 80, 85, 90, 100, or greater than 100 members.

When referring to a reference population as “healthy” or “not having a disease or condition”, it is meant that the samples, genomes, individuals, or other members comprising the population were not, at the time, known or suspected of having a significant disease state or pathological condition. Thus, an individual not known to have any type of cancer, at the time of giving a sample for analysis as part of a population, would be consider at “healthy” or as “not having a condition” or “not having cancer”. This, despite the fact that some percentage of healthy people will one day go on to develop cancer. Nevertheless, for the purposes of generating reference populations, evaluation must be made at the time the sample is collected and included as part of the reference population. Throughout the disclosure, when referring to reference populations, the terms “healthy”, or “not having a condition” or “not having cancer” and the like are used.

The present disclosure provides approaches (including methods and systems) for identifying microsatellite loci informative for a particular disease, condition or trait. In these methods, in certain embodiments, information about microsatellite loci is generated for a healthy reference population and for a not-healthy reference population indicative of a particular disease or condition for which informative loci are desired. Microsatellite length and/or sequence are analyzed for the two populations. Distributions of sequence lengths and/or actual sequences at one allele or at two (or more alleles) are assessed in both populations. Whether examining allelotypes (average sequence length; without regard to the genotype at the loci) or genotype (average sequence length or sequence at two or more alleles for each loci; genotype, as a unit comprising two or more alleles), informative loci are identified by comparing the distributions of sequence lengths and/or actual sequences (for allelotypes or genotypes (e.g., genotype units)) between the two populations. Informative microsatellites are those in which the distributions of lengths do not significantly overlap between the reference populations. The identification of informative loci based on comparisons between populations (a plurality of inputs) is, in certain embodiments, a feature of the disclosure.

Moreover, in certain embodiments, once informative loci are identified, these loci can be used to analyze new samples (e.g., a sample from a subject and/or a control sample of known condition used to validate the sensitivity and specificity of the loci). Once again, when looking at the new sample, in certain embodiments, information about the informative loci in the new sample is then compared to the informative loci information of one or more reference populations to categorize the sample as differing from healthy or another condition (e.g., as being modal or not or alternatively as having allelotype or genotype at a microsatellite loci that best fits into the distribution of the reference disease population or the reference healthy population or alternatively comparing to a condition-associated signature). Once information about modal genotype, average sequence length, or allelotype or genotype distribution for a population is determined, that information can then be, for example, stored on a computer or in a database as a value and that value may be used for future comparison. Thus, for example, when analyzing a future test sample, information about the test sample can be compared to a stored value that reflects information obtained from analysis of the populations.

### 2. Genome-Wide Microsatellite-Based Genotyping

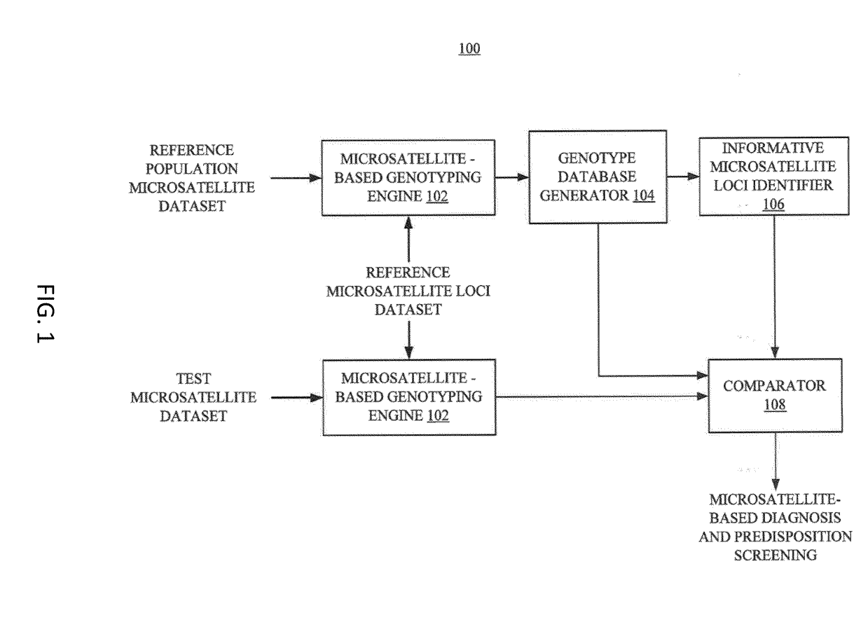

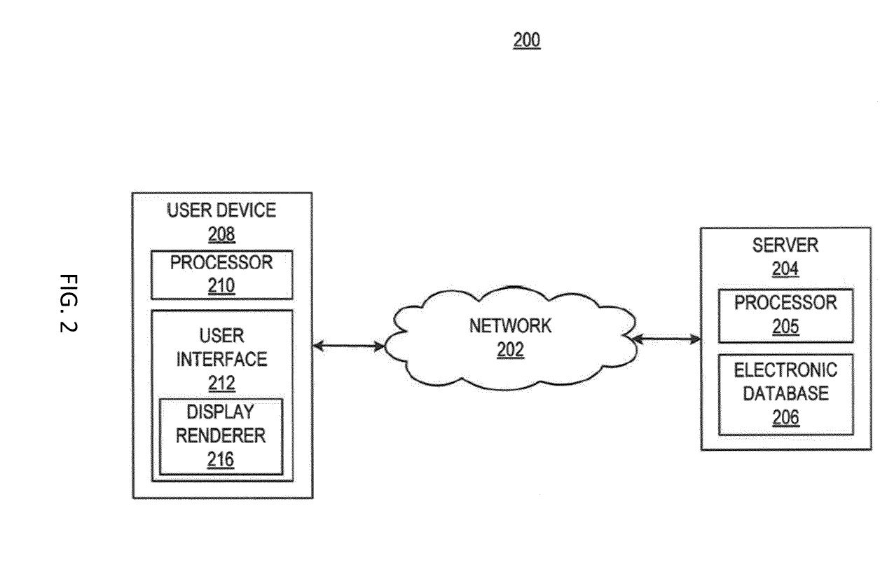

FIG. 1 is a block diagram of a system for global microsatellite instability (GMI) analysis for applications which include, for example, diagnostic, prognostic, and predisposition screening of a given physiological condition based on microsatellite genotyping data from a test subject. The system 100 includes a microsatellite-based genotyping engine 102, which aligns microsatellite data from subjects in a given population, or a test subject, to a reference microsatellite loci dataset. After the alignment is performed, the genotyping engine 102 may aggregate the microsatellites aligned to the same locus and label the aggregate with the loci information, possibly in the form of a loci-specific ID. The genotyping engine 102 then identifies a number associated with each microsatellite loci. For example, the number may correspond to the sequence length of the locus. Since errors may occur during sequencing or alignment, more than two sequence lengths may be identified for each subject whose microsatellite data is used for genotyping. The genotyping engine 102 identifies the genotype of the given subject as a set of loci-specific nucleotide lengths, which can be an identical pair for a homozygous subject. Each loci-specific nucleotide length may be referred to as an “allelotype.” When referring to the sequence length of the microsatellite locus on both alleles, considered together, it may be referred to as determining a “genotype.” Genotype distributions may also be used with the methods and systems of the disclosure. The genotype can also represent more than two alleles given that samples may be composed of heterogeneous cells, thus giving more alleles than just two. These additional alleles are referred to herein as minor alleles. The main genotype, for a particular locus in a sample, is determined by the two most frequent alleles and any remaining alleles that occur in a threshold number of sequence reads, e.g., 3, are minor alleles that may also be considered. Another example of the number or information identified by the genotyping engine 102 is the repetition number. It should be understood that repetition number, sequence length, and nucleotide sequence are exemplary of the parameters that may be considered, and any such parameter may be considered alone or in combination.

In system 100, genotype data obtained from subjects across a reference population, such as that covered by the 1000 Genomes Project, are statistically summarized according to their microsatellite loci information by a genotype database generator 104. For example, distributions may be formed by creating a histogram of, for example, sequence lengths across the reference population at each microsatellite locus. In particular, such distributions may be referred to as “allelotype distributions.” Alternatively, distributions may be formed by creating a histogram of genotypes across the reference population at each microsatellite locus. Such distributions may be referred to as “genotype distribution.” The genotype database generator 104 may require that the number of microsatellites aligned to the same locus exceeds a predetermined threshold value before a distribution may be generated.

Such a database of microsatellite loci based allelotypes or genotypes is useful for the analysis of the complexity of one or more or of a plurality of microsatellite loci on a genome-wide level and for the assessment of a population's or individual's GMI. In addition to allelotype and genotype distributions, other statistics, data characterizations, and measures that can be stored in this database include, but are not limited to, polymorphism rate, quality of sequence reads in repetitive regions, motif lengths and families (AAT, AAAT, AATT, etc.), means and widths for allelotype and genotype distributions, average alignment quality scores (indicative of a quality of the alignment of the microsatellites, for example), average read quality scores (indicative of a confidence value in the reading of the bases that make up the microsatellite data, for example), subject identification data, population data, and physiological states of the subjects being genotyped.

The microsatellite loci based allelotype or genotype database can be made available for study and/or analyzed to extract knowledge as to genome-wide trends, general behavior of microsatellites in a given population sample, and evidence of selection pressure and bias. Moreover, this database can be used as a reference against which future samples (e.g., samples from an individual subject or a plurality of samples from a population of subjects) are evaluated and characterized. An informative microsatellite loci identifier 106 further considers and compares subsets of allelotype or genotype distributions from this database, taking into account other relevant stored data associated with each subset. One example of such relevant data is whether subjects within the subset have been diagnosed with a given disease or condition, such as a type of cancer. A comparator 108 compares the microsatellite-based allelotype or genotype data of a test subject to that from subsets of the database, at informative loci identified by the identifier 106. The result of this comparison can then be used for diagnosis or prognosis purposes. A detailed discussion of how informative microsatellite loci are identified, as well as how identification of informative loci can be used, is set forth below. In certain embodiments, information about two different populations can be compared.

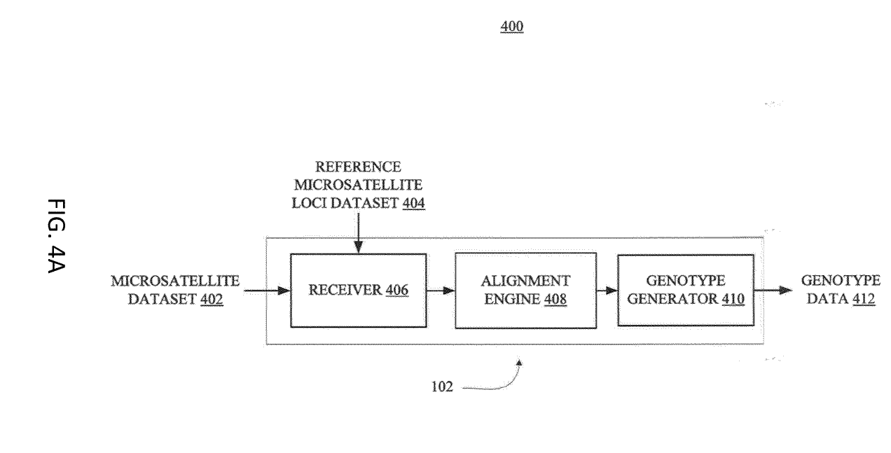

FIG. 3 depicts an example of a microsatellite loci based allelotype or genotype database generated by the database generator 104 to store records of the microsatellite loci that have been identified. A data structure 300 includes four records of microsatellite loci for ease of illustration. Each record in the data structure 300 includes a “microsatellite loci ID” field whose values include identification numbers for microsatellite loci that have been identified. Each record in the data structure 300 also includes a field for allelotype or genotype distribution associated with the microsatellite loci, and other statistics that can be stored in the database.

Many types of allelotype distributions can exist at each locus, each with possible biological consequences. Without being bound by theory, the confinement of allelotypes or genotypes to a narrow distribution may indicate significant selection pressure (and therefore of functional importance), while a wide distribution may indicate a lower selective pressure. Loci in exons and intergenic regions are expected to exhibit differences in the shape of their allelotype or genotype distributions. One exception may exist for microsatellites in intergenic regions that are ultra-conserved or that, for example, involve microRNAs. Bi-modal or multi-modal distributions may also be identified, indicating sub-populations within the sample set that may correlate with any number of factors (measurable phenotypes, disease susceptibility, etc.).

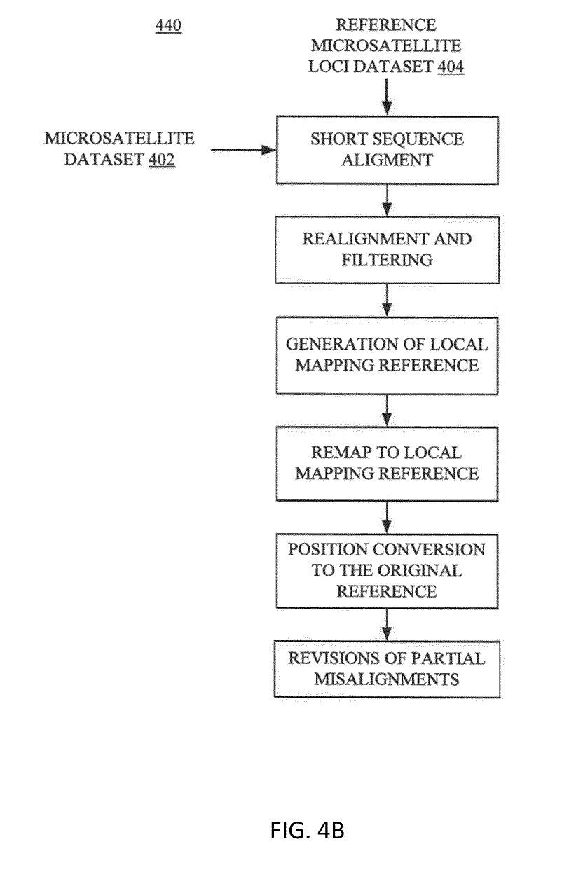

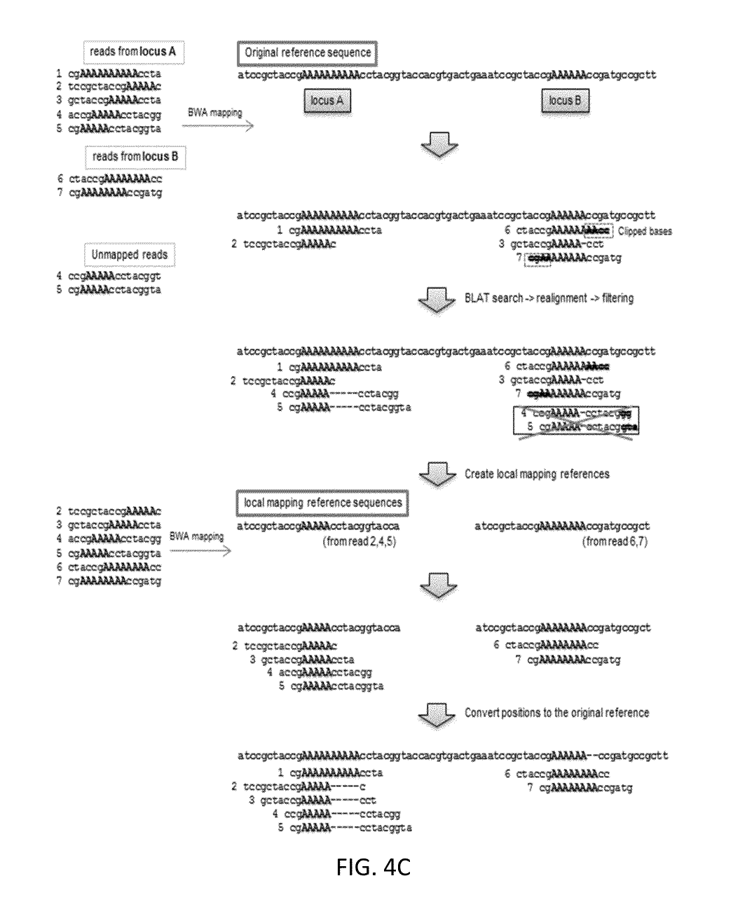

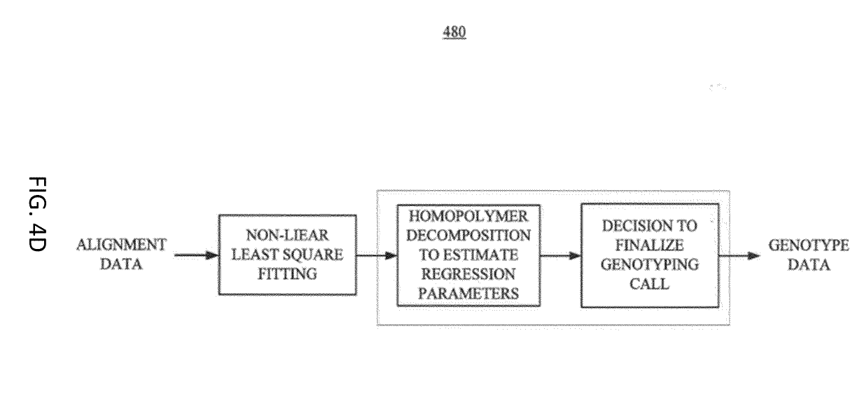

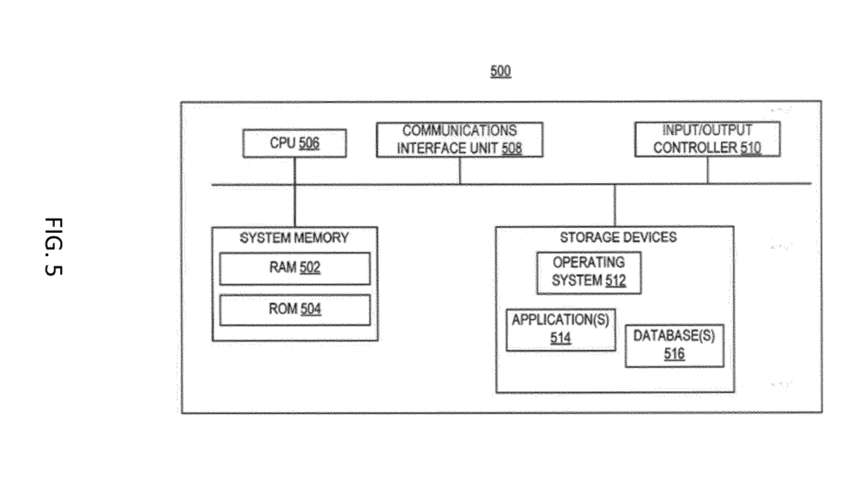

FIG. 4 is a block diagram of the microsatellite-based genotyping engine 102 shown in FIG. 1. The system 400 includes a receiver 406, an alignment engine 408, and a genotype generator 410. The receiver 406 receives a reference microsatellite loci dataset 404, and a microsatellite dataset 402 to be allelotyped or genotyped. The microsatellite dataset 402 may contain microsatellites extracted from general short sequence reads, identified using repetitive sequence identifiers. It may include perfect (contiguous runs of perfectly repeated motifs, without SNPs) or imperfect (including SNPs, indels) microsatellites.

In one embodiment, the reference microsatellite loci dataset 404 is obtained from high quality nucleic acid sequences representative of human genes, such as high quality DNA or RNA; for example, the human reference genome NCBI36/hg18 from the 1000 Genomes Project. The reference microsatellite loci dataset 404 may also be obtained as a consensus among multiple reference subjects. Moreover, filters may be applied to the data set such that microsatellites satisfying one or more criteria are included. For example, the microsatellite data may be limited to include microsatellites of at least 10 base pairs long, with no more than one interruption to the canonical repeat sequence for each ten bases in length (≧90% “pure”), and within 500 base pairs of targeted regions. Such microsatellite data may be found using a repetitive sequence identifier. Examples of such identifiers include Repeatmasker, Tandem Repeats Finder, POMPOUS, JSTRING, TandemSWAN, and many others. The sequence length identifier may search for perfect microsatellites, or microsatellites with imperfections. Depending on the identifier used, different search parameters can be adjusted according to the desired characteristics of the reference microsatellite loci dataset 404. Examples of such parameters include mismatch penalty score, minimum alignment score, and maximum period size to report. Microsatellites within short and long interspersed elements (SLINE/LINE) are optionally removed using known chromosomal locations. Using genomic locations, these microsatellites may be associated with all genes they are in or near. Microsatellites which are located in two gene regions are labeled as belonging to the region in which most of their sequence is contained. Heuristic methods can be further applied to search for microsatellite loci missed from this identification process.

The receiver 406 transmits the microsatellite data 402 and the reference microsatellite loci data 404 to the alignment engine 408, which aligns the microsatellite data 402 to the reference microsatellite loci dataset 404. The alignment engine 408 executes an algorithm to perform this alignment. In particular, the alignment algorithm may also align flanking sequence preceding and following the microsatellite sequence. In some embodiments, the alignment engine 408 is configured to run multiple algorithms on the microsatellite data. For example, if one alignment algorithm is unable to align a particular microsatellite to the reference dataset 404, the alignment engine 408 may be configured to attempt to align the same microsatellite using a different alignment algorithm.

After microsatellites from the given dataset 402 have been aligned to microsatellite loci in the reference dataset 404 by the alignment engine 408, the genotype generator 410 identifies the genotype of the subject that has contributed to the microsatellite dataset 402, in the form of a set of two loci-specific sequence lengths, or allelotypes. Similarly, as described above, genotype may be depicted and analyzed in the form of sequence length and/or nucleotide sequence. For example, the genotype generator 410 may identify a pair of sequence lengths, which can be identical, indicative of a homozygous subject. The genotype generator 410 may also identify more than a pair of allelotypes, each with a quality score indicative of the probability that the particular allelotype is present in the input microsatellite data 402. As an example, in the case of cancer patients, mutations of the gene can be extensive, leading to the presence of more than 2 allelotypes at some loci.

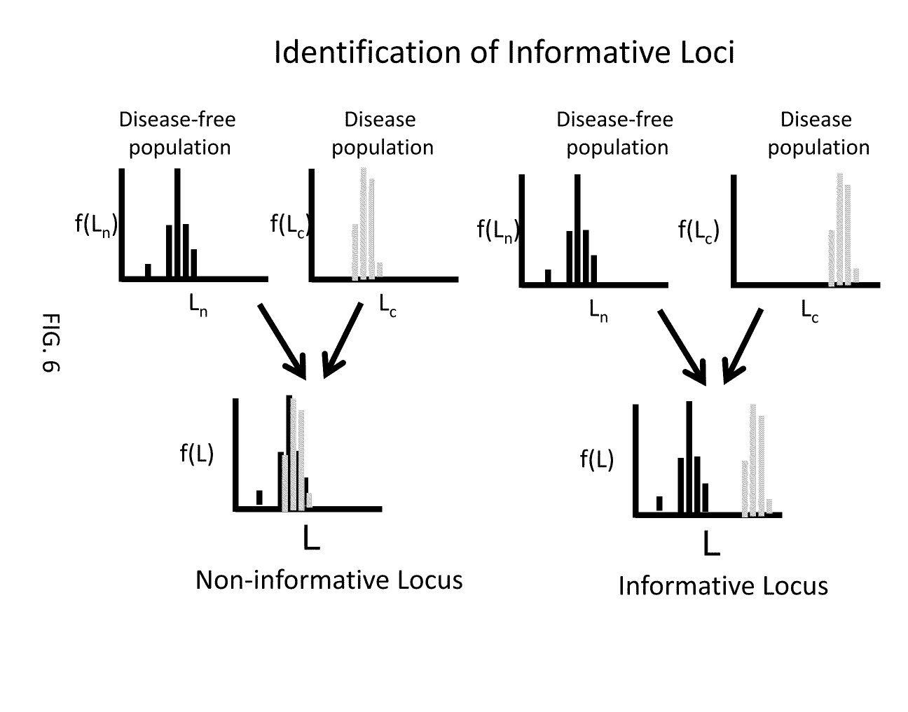

Any of the components in the system 400 may include a processor. As used herein, the term “processor” or “computing device” refers to one or more computers, microprocessors, logic devices, servers, or other devices configured with hardware, firmware, and software to carry out one or more of the computerized techniques described herein. Processors and processing devices may also include one or more memory devices for storing inputs, outputs, and data that are currently being processed. An illustrative computing device 500, which may be used to implement any of the processors and servers described herein, is described in detail with reference to FIG. 5.

The alignment engine 408 may contain a quality evaluator that assesses a quality score for each input microsatellite, or for each alignment provided by the alignment engine 408. For example, the quality score may include a sequence quality score. In another example, the quality score may include an alignment quality score indicative of a degree of match between the aligned microsatellite and the locus in the reference dataset. A sequence quality score may be computed from base-call quality values associated with every read of each base pair. For example, Phred scores representing the probability that a base is miscalled can be used. Depending on the program used to generate this confidence value, the quality score may be based on peak height or area, spacing between peaks, the presence of multiple peaks, or light intensity associated with homopolymers. The quality score may also be a statistic of the miscall probabilities of the bases in each microsatellite, such as a mean, median, mode, or any other suitable statistic. In general, the quality score determined by the data quality evaluator is indicative of a level of confidence in the quality of the data in the microsatellite and/or a quality of the alignment of the microsatellite to the reference dataset. Similar quality score calculation can be performed on flanking sequences used during alignment. The computed quality score may be part of data output from the alignment engine 408.

The alignment engine 408 may also contain a dataset filter that removes any microsatellites that fail to meet one or more criteria. For example, the data set filter may compare the sequencing quality score of a microsatellite to a predetermined threshold, and any microsatellites with quality scores below the predetermined threshold may be discarded. The dataset filter may also remove microsatellites that have alignment scores below a given set of thresholds, corresponding to microsatellite loci in the reference set 404. In general, any criterion may be used to filter the dataset.

In one embodiment of alignment engine 408, microsatellite data 402 can be aligned to the reference set 404 using an existing automatic aligner, optionally with manual heuristical adjustments to the results. Examples of such aligners are BWA, Bowtie2, GATK, SMRA, PINDEL, among others. Non-repetitive flanking sequences preceding and following the microsatellite sequence may also be aligned, using heuristics that are confirmed to obey Mendelian inheritance of informative loci using deep sequencing data of trios under a hereditary relationship. Single base substitutions in tandem repeats may then be identified. Specifically, high quality reads which span the repeat regions plus some unique flanking sequences may be identified. These results may be further filtered using a flanking sequence to enable comparison to common single nucleotide polymorphism (SNP) filtering windows. The flanking sequences may have a pre-defined length, for example, 10 base pairs (bp). Increasing the flanking sequence length would reduce the number of callable loci, but would also increase confidence in the alignments by relying on additional unique sequences.

In one embodiment of the alignment engine 408, reads not aligned by the aligner to the reference along with reads which are aligned to a microsatellite locus by the aligner but do not meet unique flanking sequence criteria may be run through additional computational codes to determine if they should be aligned to another microsatellite locus based on flanking sequences and a short portion of the repeat. This allows the maximal use of reads with repetitive sequences and removes possible restrictions associated with the length of indel calling by the aligner. Using a small portion of the repeat is beneficial as many microsatellites have multiple alignments in the human genome if the flanking sequences are allowed to be separated by a given number of flanking bases, for example, 200 bases.

In another embodiment of the alignment engine 408, single base substitutions can be identified in repeat regions concurrently with microsatellite alignment, with a heuristic applied to account for possible increase in coverage: since a smaller portion of the sequences is being aligned, higher coverage is more likely using the same available data.

FIG. 4B shows another embodiment of the alignment engine 408, for aligning next-generation sequencing (NGS) short sequence microsatellite data to a reference microsatellite loci dataset, i.e., at loci with short tandem repeats (STR). FIG. 4C provides an illustrative example corresponding to the processing steps carried out in the embodiment shown in FIG. 4B.

NGS has enabled investigators to generate a huge amount of sequence data. However, with their inherent sequencing errors and short sequence read lengths, data analysis for several kinds of repeat elements such as transposon elements and tandem repeats still remains limiting and problematic. It can be observed that mapping programs often assign high quality scores to incorrectly mapped reads when two or more tandem repeat loci containing the same motif with different repeat lengths and their flanking sequences show high similarity. This is because mapping program parameters are normally set to minimize the number of mismatch or INDEL (Insertion/Deletions) bases in an alignment. This mismapping leads directly to invalid variant calls in repeat loci because the variation calling programs rely only on the mapping quality scores to filter out false positive variants from incorrectly mapped reads. In the human genome, more than 2/3 of STRs are overlapping or near (within 50 NT) transposon elements. Notably, AT rich STRs are often discovered near the 3′ ends of retrotransposons, which frequently results in the left or right flanking sequence of a STR being highly replicated while the other flanking sequence is unique. The sequence reads mapped to the incorrect STR loci due to length variation of the STRs can be revised if flanking sequences on one side of the STRs are unique and the correct lengths of the STRs in the sequenced sample are known.

Sequence reads are also often partially misaligned to a reference sequence if the reads contain INDEL variants and do not span enough of the flanking sequence of the locus. A few programs such as SMRA and GATK realign sequence reads mapped to the INDEL variant loci to correct misalignment, but their performance is poor for the reads mapped to STR loci containing long INDELs. To realign sequence reads at the INDEL variant loci, the programs require a large number of reads supporting the variants, but the reads containing tandem repeat variation often fail to be mapped to the correct loci and as a result the programs do not obtain sufficient read.

In certain embodiments, the illustrative embodiment 440 of the alignment engine 408 can be described as an automated pipeline using a “local mapping reference reconstruction method” to revise mismapped (mapped to incorrect position) or partially misaligned (mapped to correct position but one of ends misaligned) reads at microsatellite loci. See Tae H, McMahon K W, Settlage R E, Bavarva J H, Garner H R. ReviSTER: an automated pipeline to revise misaligned reads to simple tandem repeats. Bioinformatics. 2013 Jul. 15; 29(14):1734-41, herein incorporated by reference in its entirety. It takes as inputs a reference microsatellite loci dataset 404, containing loci around STRs, and a microsatellite dataset 402. In this implementation, the system 440 performs 6 process steps on the input data, as described below.

First, short sequence alignment is conducted using an existing aligner, such as BWA. The ‘-n’ option which is used for BWA mapping may be taken, to record multiple mapping candidates for reads derived from repeat sequences.

Second, another alignment tool, such as BLAT, can be used to remap unmapped reads to temporary mapping reference sequences which are extracted from the original reference sequence around a given STR loci. Because many false alignments for a read may be generated, system 440 realigns them and chooses the best alignment from several alignment candidates.

Third, system 440 employs a local assembly step using the reads mapped to each microsatellite locus. It generates paths in a graph of reads overlapping at least 30 bases with each other, chooses a given number of paths corresponding to allele candidates, extracts sequences of the allele candidates and creates local mapping reference sequences containing the allele candidates. In this step, sequence reads containing more than one mismatch/INDEL bases or showing abnormally long pair distances may be saved in a separated file along with unmapped reads.

Forth, the reads saved in the separate file are mapped to the local mapping reference sequences by BWA (with the -n option).

Fifth, mapping positions of a read on the local mapping reference sequences are converted to positions on the original reference. Then a mapping position with the most optimal pair distance and the lowest mismatch number is chosen among all mapping candidates identified in the first step and the fifth step.

The final step is to revise reads partially misaligned at microsatellite loci, a process that is independent from the previous steps. Some reads may have been incorrectly aligned to the microsatellite loci containing long INDELs and not revised by the previous steps. The reads are realigned to other reads which have been mapped to the same STR locus and sufficiently span the flanking sequences of the locus.

Alignment data generated by the alignment engine 408 are sent to the genotype generator 410. In one embodiment of the genotype generator 410, aligned microsatellite loci are not allowed to have more than two possible allelotypes, after filtering those alleles supported by less than a pre-defined number of reads, for example, 5 reads. There also may be a pre-defined number of reads supporting each allele. For example, the predefined number of reads could be set at at least 5 and no more than 50, or at least 3 and no more than 50. However, different parameters may also be used. In the case of microsatellites which could possibly be heterozygous, they, in certain embodiments, are only considered to be heterozygous if the reads for each allele are no more than about two times the reads of the second allele. This allows for unequal amplification, which is an issue with whole genome sequencing, and even more of an issue with targeted sequencing. Optionally, data with indels in and near homopolymer regions may be thrown out prior to performing microsatellite-based genotyping.

In another embodiment of the genotype generator 410, a discretized Gaussian mixture model is combined with a rules-based approach to identify allelotype variation of microsatellites from short sequence reads. See Tae H, Kim D Y, McCormick J, Settlage R E, Garner H R. Discretized Gaussian mixture for genotyping of microsatellite loci containing homopolymer runs. Bioinformatics. 2013 Nov. 6, herein incorporated by reference in its entirety. For example, the illustrative embodiment shown in FIG. 4D distinguishes length variants from INDEL errors at homopolymers, or microsatellites containing repetitions of 1-mer motifs. In this case, repetition numbers indicative of allelotypes are the same as microsatellite sequence lengths. Inferring lengths of inherited microsatellite alleles with single base pair resolution from short sequence reads is challenging due to several sources of noise including PCR amplification errors, individual cell mutation, misalignment or mis-mapping caused by the repetitive nature of the microsatellites.

Let l_{L} be the length of a candidate allele L at a target locus and let x be the observed length of the microsatellite sequence with INDEL errors in a read mapped to the locus with an assumption in which the length x is derived from the original length l_{L}. Let F_{L}(t) and f_{L}(t) denote the distribution and the density functions of a Gaussian random variable with mean l_{L} and variance σ_{L}² respectively. Then the probability mass function p_{L}(x) of x is

$${p}_{L} ( x ) = P ( X = x \vert {l}_{L} , {σ}_{L}^{2} ) = \frac{1}{1 - {F}_{L} ( 0.5 )} {\int }_{x - 0.5}^{x + 0.5} {f}_{L} ( t ) d t \qquad ( 1 )$$

where x=0, 1, 2, . . . , and

$$\frac{1}{1 - {F}_{L} ( 0.5 )}$$

is a scale factor.
For the heterozygous loci with allele lengths, l_{L1} and l_{L2}, the mixture distribution of the equation 1 can be used as follows

g(x)=g(x;L₁,L₂,σ_{L1}²,σ_{L2}²,θ)=θ·p_{L}_{₁}(x)+(1−θ)·p_{L}_{₂}(x),0≦θ≦1 (2)

where θ is the unknown mixture proportion parameter for reads derived from one of the two alleles, regardless of the repeat sequence length x. It is also assumed that the associated parameters σ_{L1}² and σ_{L2}² are both unknown. These parameters can be estimated by a nonlinear least squares (NLS) regression function.

If the sequence reads mapped to a same microsatellite locus contain INDEL errors, the number of observed lengths of the microsatellite at the locus would be equal to 2 or more than 2. Because the inherited alleles are unknown, all observed lengths are allele candidates. The g(x) function for each combination of two allele candidates (two same candidates for homozygous genotype) is then applied, calculating the squared error of each combination, and select the allele pair, L₁* and L₂*, that generates the minimum squared error as follows

$$G ( {L}_{1}^{*} , {L}_{2}^{*} ) = {argmin}_{all candidates} \{ {\sum }_{x = a}^{b} {( {o}_{x} - g ( x ; {L}_{1} , {L}_{2} , {\hat{σ}}_{L 2}^{2} , {\hat{σ}}_{L 2}^{2} , \hat{θ} ) )}^{2} \} \qquad ( 3 )$$

where oₓ is an observed proportion of reads containing a length x microsatellite sequence, a is the minimum observed length minus a fixed amount k, and b is the maximum observed length plus k, where k is set to be five as default value. This is necessary because the g(x) function generates output values for all possible sequence lengths, the comparison between observed proportions and expected proportions need to be extended beyond the minimum and maximum observed lengths. Therefore, the boundaries of the calculation are extended by an additional value k.

As an example, suppose that there are 2, 8 and 4 mapped reads containing microsatellite sequences with lengths 14, 15 and 16 bases, respectively, at a locus. The list of possible genotype candidates G(l_{L1},l_{L2}) for the locus are G(14, 14), G(14, 15), G(14, 16), G(15, 15), G(15, 16), and G(16, 16). In the example, the observed minimum and maximum lengths are 14 and 16 respectively, and the observed and expected values from the equation 3 are compared for x ranging from 9 to 21. While the observed ratio of read counts between the highest read frequency allele (l_{L1}=15) and the second highest read frequency allele (l_{L2}=16) is 0.5 (=4/8), the read ratio of those two alleles estimated by the NLS function was 0.163 (=(1−θ)/θ=0.14/0.86). The difference between the two estimated ratios may result in a different decision for the genotype calls, depending on the cutoff ratio to determine if the second highest read frequency allele candidate is noise.

System 480 takes as input microsatellite loci alignment data, possibly with quality scores. For each locus, it then chooses allele candidates which satisfy a given set of conditions. For example, allele candidates can be chosen according to the following three sample conditions: 1) At least 2 reads supporting the same allele candidate overlap at least 3 bases for both flanking sequences and they are not technical duplications (same mapping position and same sequence); 2) Microsatellite sequences of at least 2 reads supporting the same allele candidate have fewer than 10% mismatches in their length; 3) A consensus sequence of the reads span at least 5 bases at both flanking sequences. It is understood that numerical parameters given here can be adjusted according to the characteristics of the input dataset.

In this embodiment of the genotype generator, the genotyping system 480 performs a two-step estimation. In the first step, rough estimates find the candidate genotypes of microsatellite loci using the regression model described previously. In the second step, the regression method requires two additional parameters which are estimated from the results of the first regression step. The first parameter, ω_{L}, represents error bias toward deletion or insertion depending on the homopolymer length in an allele candidate L. Since the Gaussian distribution has a symmetric form, the equation 1 generates symmetric probabilities for deletion and insertion errors for any allele, which does not fit real data. It can be adjusted by adding additional parameters ω_{L1} and ω_{L2} to μ₁ and μ₂ respectively as follows

f_{L1}(t)˜N(μ₁=l_{L1}+ω_{L1},σ₁²=σ_{L1}²),f_{L2}(t)˜N(μ₂=l_{L2}+ω_{L2},σ₂²=σ_{L2}²) (4)

Then, equations 1 and 2 can generate different probabilities for deletion and insertion errors depending on the homopolymer length in L₁ or L₂. To estimate ω_{L} for each allele candidate L, a homopolymer decomposition method can be used, which decomposes a given microsatellite sequence into a set of homopolymers and then estimates parameters from the set.

The second parameter, υ_{L}, represents a variance of the prior probability distribution of read proportions for x derived from an allele candidate L. The NLS regression function to estimate σ_{L1}, σ_{L2} and θ requires as input a data vector containing the observed read proportions for length x microsatellite sequences. These estimated parameters are then used to calculate the probability of each x to be observed in a read at a locus. Recall that, the probability varies depending on the length of the homopolymer in the microsatellite sequence. Since the first regression step uses only the read proportions to estimate σ_{L1}, σ_{L2} and θ, the estimated values of the parameters are always the same regardless of the lengths of homopolymers in alleles, if two or more different loci have different repeat sequences but contain the same proportions of reads. However, it can be observed that the probability of the INDEL error increases with long homopolymer repeats. To apply the homopolymer effect to the NLS regression, different pseudo counts can be used for different repeats. The data vector may be initialized to 0 and pseudo counts (positive fractions) may be estimated from the g(x; l_{L1},l_{L2},υ_{L1},υ_{L2},0.5) function in which the parameters are {σ₁²=υ_{L1}, σ₂²=υ_{L2}, θ=0.5} are added to the vector. And, instead of the numbers of reads, sums of mapping probabilities of reads containing length x microsatellite sequences are added to the vector. If mapping probabilities of reads are high, their sum is near the number of the reads. Then, the values in the vector are converted to the proportions. If υ_{L1} and υ_{L2} are large and the number of total reads is small, the values in the vector get dispersed and the NLS function estimates large σ_{L1} and σ_{L2}. But when the number of total reads is big, the effect of υ_{L1} and υ_{L2} becomes small. The parameter υ_{L} for each allele candidate L is also estimated by the homopolymer decomposition method, described below.

Homopolymer Decomposition:

the homopolymer decomposition method is a process to decompose sequences into a set of homopolymers to estimate parameters ω_{L} and υ_{L}. For example, the ‘TAAACAAATAAA’ sequence is composed of three ‘AAA’, two ‘T’ and one ‘C’ (‘T’ and ‘C’ are monomers but are treated as homopolymers). In one embodiment of the system 480, the following assumption can be made to make the problem tractable:

A1) Insertion and deletion error events in each homopolymer are independent from those in the neighborhood homopolymers.
A2) Each error at a base is independent from the errors at neighborhood bases.
A3) Only one of the insertion or deletion error events in the repeat sequence of a read is considered. This means only the observed event are considered. For example, only 1 base deletion error for {1 base insertion+2 base deletion}, {2 base insertion+3 base deletion} and so on are considered.
A4) All of the insertion errors are derived only from the existing neighborhood nucleotides. If a sequence read has ‘TGAAATAAATAAA’ sequence and the second base ‘G’ is identified as an insertion error, the first homopolymer ‘T’ or the second homopolymer ‘AAA’ are assumed to cause the insertion error.
A5) Probabilities of insertion and deletion errors are affected only by the lengths of homopolymers. The other ignored factors include high error rates at the end bases of sequence reads, GC-content biases during library amplification/sequencing and effects of specific sequences such as ‘GGC’ inducing sequencing errors which are known to occur in the Solexa next generation sequencing platform.

As an example, suppose that 15 and 1 reads containing ‘TAAATAAA’ and ‘TAATAAA’ respectively, have been mapped to a locus A. It would be concluded that the inherited allele is ‘TAAATAAA’ and ‘TAATAAA’ is derived from ‘TAAATAAA’ by a 1-base deletion error. Then an estimated average length of the sequence in a read which is derived from the ‘TAAATAAA’ allele is 7.93 bases (15/16×8+1/16×7). For another example, suppose that 14, 2 and 1 reads containing ‘GTTTGTTT’, ‘GTTGTTT’, and ‘GTTTTCGTTT’ respectively, have been mapped to another locus B. It would be concluded that the inherited allele is ‘GTTTGTTT’, and ‘GTTGTTT’ and ‘GTTTTCGTTT’ have a 1-base deletion error and a 2-base insertion error respectively. Then an estimated average length of the sequence in a read which is derived from the ‘GTTTGTTT’ allele is 7.99 bases (14/17×8+2/17×7+1/17×10). Based on the assumption A5, the alleles of locus A and B can be treated as the same sequence in an abstract form, {1N3N1N3N}, and the average length of the sequence can be calculated together. Then the estimated average length of the sequence in a read derived from {1N3N1N3N} is 7.97 (=29/33×8+3/33×7+1/33×10). By simply subtracting 7.97 from 8, co can be estimated, representing the error bias toward deletion or insertion at the microsatellite sequence in a read derived from the {1N3N1N3N} allele. While the positive result of the subtraction represents bias toward insertion, the negative result represents bias toward deletion in sequence reads derived from the allele.

In certain embodiments, if more reads derived from all loci containing the {1N3N1N3N} alleles are collected, a more accurate average length of repeat sequences can be estimated in reads derived from the alleles. But some alleles (e.g. {40N10N}) may not be covered by enough reads to be used as the training set to estimate the accurate average length, so the homopolymer decomposition method can be applied. The average length of the sequences in the previous example is 7.97 and the abstract form of the allele is {1N3N1N3N}. This form can be decomposed into ‘2·{1N}+2·{3N}’. Since each {iN} can be regarded as an individual variable, they can be defined as {N₁, N₂, N₃, N₄ . . . }, and the example can be described by ‘7.97=2·N₁+2·N₃’. Then an equation can be written to summarize all possible allele sequences as follows

$$Y = {n}_{1} \cdot {N}_{1} + {n}_{2} \cdot {N}_{2} + {n}_{3} \cdot {N}_{3} + … = {\sum }_{i}^{I} {n}_{i} \cdot {N}_{i} \qquad ( 5 )$$

where Y is the average length of repeat sequences in reads derived from a single abstracted allele. Due to the limitation of the current sequencing technology, the maximum length, I, of a sequence, that can be obtained, is not infinite. Y and nᵢ for an allele are simply calculated from the training data, and {N₁, N₂, N₃, N₄ . . . } can be estimated by a linear regression method. Moreover, because of the correlation between Nᵢ and Nᵢ₊₁, Nᵢ is defined with two additional cofactors αₐ and α_{b} as

Nᵢ=i+αₐ·i+α_{b}, (6)

where α_{b} and α_{b} represent a bias gradient and an initial bias respectively. Then equation 2 can be written as

$$Y = {{\sum }_{i}}^{I} {n}_{i} ( i + {α}_{a} \cdot i + {α}_{b} ) \qquad ( 7 )$$

Because the variables i and nᵢ represent the length and the number of each homopolymer at a given abstracted allele respectively, the equation 3 can be simplified as follows

$$Y - ( allele length ) = {\sum }_{i}^{I} {n}_{i} ( {α}_{a} \cdot i + {α}_{b} ) \qquad ( 8 )$$

The cofactors αₐ and α_{b} are estimated by a nonlinear regression method from the genotyping results of the first genotyping regression step and are used to calculate the parameters ω_{L} for a given allele candidate L in the second genotyping regression step from the following function

$${ω}_{L} = get\_mean \_bias ( consensus sequence of allele L , {α}_{a} , {α}_{b} ) = {\sum }_{i}^{I} {n}_{i} ( {α}_{a} \cdot i + {α}_{b} ) \qquad ( 9 )$$

since the number of each length i homopolymer can be simply counted from the consensus sequence of the given allele candidate L.

Based on the assumption A1 and A2, the parameter υ_{L} can be estimated in the same way with ω_{L}. For a given abstracted allele {1N3N1N3N}, the variance is calculated by the NLS regression function. And the abstracted form is decomposed into ‘2·M₁+2·M₃’, where Mᵢ is a corresponding variable to Nᵢ in the previous paragraph. Then an equation can be written to summarize all possible allele sequences as follows

$$Z = {\sum }_{i}^{I} {n}_{i} \cdot {M}_{i} \qquad ( 10 )$$

where Z is an estimated variance of lengths of microsatellite sequences in reads derived from a given abstracted allele. Define Mᵢ with two additional cofactors βₐ and β_{b} as

$$\begin{matrix}{M}_{i} = {i}^{2} \cdot {β}_{a} \cdot {e}^{i \cdot {β}_{b}} & ( 11 ) \\ Z = {β}_{a} \cdot ( {\sum }_{i}^{I} {n}_{i} \cdot {i}^{2} \cdot {e}^{i \cdot {β}_{b}} ) & ( 12 )\end{matrix}$$

which describes rapid change of variances according to the length of homopolymers. They are also estimated by a nonlinear regression, and are used to estimate the parameters υ_{L} for a given allele candidate L in the second genotyping regression step from the following function

$${υ}_{L} = get\_var \_prior ( consensus sequence of allele L , {β}_{a} , {β}_{b} ) = {β}_{b} ( {\sum }_{i}^{I} {n}_{i} \cdot {i}^{2} \cdot {e}^{i \cdot {β}_{b}} ) + ϕ \qquad ( 13 )$$

where φ with default value 0.5, is added to υ_{L} to reduce the probability of allele candidates supported by a small number of reads.

Decision Process to Finalize Genotyping Call:

the most probable genotype for a given set of sequence reads mapped to a locus is decided, in certain embodiments, by the equation 3. But the equation shows a tendency to call heterozygous genotypes, because the Gaussian mixture model is a better fit to the training data when more distributions are mixed. However, since reads supporting one or both predicted alleles may be from noise including individual cell mutation, PCR amplification error, sequencing error and mis-mapping, an evaluation method is necessary.

In this embodiment, a rule-based approach is used to choose alleles and to decide the homozygosity of each locus because the frequencies of INDEL error reads derived from mis-mapping, PCR amplification error and individual cell mutation are more difficult to measure than that from the sequencing error. For this approach, a confidence score is assigned to each allele instead of calculating the probability of a genotype (a two allele set) for a locus. The probability of each allele can be generated by the equation 1 as p_{L1}(l_{L1}) or p_{L2}(l_{L2}) if the read frequencies are assumed from two different alleles at the heterozygotic locus are not correlated. However DNA fragments from two paired chromosomes have the same probability of being sequenced and the read frequencies of two alleles would tend to be similar. If the proportion of reads for an allele candidate L_{low} with lower read frequency is too small compared to that for another allele candidate L_{high} with higher read frequency (e.g. 0.1 vs. 0.9), it may be concluded that the reads for the allele candidate L_{low} are from noise and the locus is homozygous. Considering this condition, ratio of θ_{low} to θ_{high} can be multiplied and the output of p_{Llow} (l_{Llow}), where θ_{low} is the output of MIN {θ, 1−θ} and θ_{high} is the output of MAX {θ, 1−θ}. The confidence scores of two allele candidate are then defined by

$${C}_{high} = {p}_{{L}_{high}} ( {l}_{{L}_{high}} ) , {C}_{low} = \frac{{θ}_{low}}{{θ}_{high}} {p}_{{L}_{low}} ( {L}_{{L}_{low}} ) \qquad ( 14 )$$

In the final tabulation, an allele candidate from the predicted genotype is removed when its confidence score is lower than a given cutoff value (0.35 for L_{high} and 0.25 for L_{low}). When only confidence score of L_{low} is lower than the cutoff value, System 480 generates a partial genotype call for the locus in which only one allele is called while the other allele is reported as unknown. System 480 only reports the genotype of the locus as homozygous when the number of reads supporting the selected allele is more than 4 and its confidence score is ≧0.9. The confidence score of the second allele, L_{high2}, at a homozygous locus is calculated by

C_{high2}=C_{high1}×(1˜0.5^{{read countsupportingL}^{_{high}}^{}}) (15)

where [0.5ⁿ] represents the probability of the other unobserved allele exists when n reads support the selected allele.

### Computer-Implemented Aspects

As understood by those of ordinary skill in the art, the methods and information described herein may be implemented, in whole or in part, as computer executable instructions on known computer readable media. Moreover, any of the methods and processes, including any individual step, may be implemented on a computer, such as by providing information/data to a computer system. For example, the methods described herein may be implemented in hardware. Alternatively, the method may be implemented in software stored in, for example, one or more memories or other computer readable medium and implemented on one or more processors. As is known, the processors may be associated with one or more controllers, calculation units and/or other units of a computer system, or implanted in firmware as desired. If implemented in software, the routines may be stored in any computer readable memory such as in RAM, ROM, flash memory, a magnetic disk, a laser disk, or other storage medium, as is also known. Likewise, this software may be delivered to a computing device via any known delivery method including, for example, over a communication channel such as a telephone line, the Internet, a wireless connection, etc., or via a transportable medium, such as a computer readable disk, flash drive, etc.

More generally, and as understood by those of ordinary skill in the art, the various steps described in this disclosure may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

When implemented in software, the software may be stored in any known computer readable medium such as on a magnetic disk, an optical disk, or other storage medium, in a RAM or ROM or flash memory of a computer, processor, hard disk drive, optical disk drive, tape drive, etc. Likewise, the software may be delivered to a user or a computing system via any known delivery method including, for example, on a computer readable disk or other transportable computer storage mechanism. Thus, in certain embodiments, prior to performing a particular method step, input data is provided to a computer, such as to a processor.

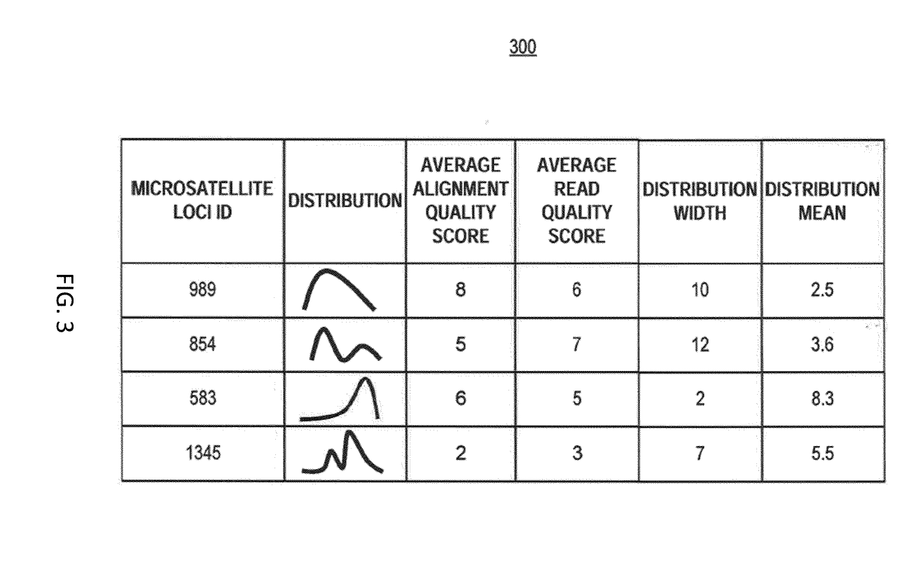

FIG. 2 is a block diagram of a computerized system 200 for implementing the system 100, according to an illustrative implementation. The system 200 includes a server 204 and a user device 208 connected over a network 202 to the server 204. The server 204 includes a processor 205 and an electronic database 206, and the user device 208 includes a processor 210 and a user interface 212. The user interface 212 includes a display render 216 for displaying data and results to a user. As used herein, the term “processor” or “computing device” refers to one or more computers, microprocessors, logic devices, servers, or other devices configured with hardware, firmware, and software to carry out one or more of the computerized techniques described herein. Processors and processing devices may also include one or more memory devices for storing inputs, outputs, and data that are currently being processed. An illustrative computing device 500, which may be used to implement any of the processors and servers described herein, is described in detail below with reference to FIG. 5. As used herein, “user interface” includes, without limitation, any suitable combination of one or more input devices (e.g., keypads, touch screens, trackballs, voice recognition systems, etc.) and/or one or more output devices (e.g., visual displays, speakers, tactile displays, printing devices, etc.). As used herein, “user device” includes, without limitation, any suitable combination of one or more devices configured with hardware, firmware, and software to carry out one or more of the computerized techniques described herein. Examples of user devices include, without limitation, personal computers, laptops, and mobile devices (such as smartphones, blackberries, PDAs, tablet computers, etc.). Only one server and one user device are shown in FIG. 2 to avoid complicating the drawing; the system 200 can support multiple servers and multiple user devices.

A user provides one or more inputs, such as microsatellite data related to one or more individuals, to the system 200 via the user interface 212. The processor 210 may process input or stored data corresponding to the user inputs before transmitting the user inputs, data or the processed data to the server 204 over the network 202. For example, the processor 210 may package the information with a timestamp or encode the information using specific pre-defined codes. The electronic database 206 stores received data and may also store additional data including data that were previously input into the user interface 212 by the user.

The components of the system 200 of FIG. 2 may be arranged, distributed, and combined in any of a number of ways. For example, the system 200 may be implemented as a computerized system that distributes the components of system 200 over multiple processing and storage devices connected via the network 202. Such an implementation may be appropriate for distributed computing over multiple communication systems including wireless and wired communication systems that share access to a common network resource. In some implementations, system 200 is implemented in a cloud computing environment in which one or more of the components are provided by different processing and storage services connected via the Internet or other communications system.

Although FIG. 2 depicts a network-based system for identifying microsatellite data, the functional components of the system 200 may be implemented as one or more components included with or local to the user device 208. For example, a user device 208 may include a processor 210, a user interface 212, and an electronic database. The electronic database may be configured to store any or all of the data stored in database 206. Additionally, the functions performed by each of the components in the system of FIG. 2 may be rearranged. In some implementations, the processor 210 may perform some or all of the functions of the processor 205 as described herein. For ease of discussion, this disclosure describes techniques for GMI analysis with reference to the system 200 of FIG. 2. However, any other type of system may be used, as well as any suitable variations of these systems.

FIG. 5 is a block diagram of a computing device, such as any of the components of the system of FIG. 1, for performing any of the processes described herein. Each of the components of these systems may be implemented on one or more computing devices 500. In certain aspects, a plurality of the components of these systems may be included within one computing device 500. In certain implementations, a component and a storage device may be implemented across several computing devices 500, including across a network.

The steps of the claimed method and system are operational with numerous other general purpose or special purpose computing system environments or configurations. Examples of well known computing systems, environments, and/or configurations that may be suitable for use with the methods or systems of the claims include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The steps of the claimed method and system may be described in the general context of computer-executable instructions, such as program modules, being executed by a computer. Generally, program modules include routines, programs, objects, components, data structures, etc. that perform particular tasks or implement particular abstract data types. The methods and apparatus may also be practiced in distributed computing environments where tasks are performed by remote processing devices that are linked through a communications network. In both integrated and distributed computing environments, program modules may be located in both local and remote computer storage media including memory storage devices.

The computing device 500 comprises at least one communications interface unit, an input/output controller 510, system memory, and one or more data storage devices. The system memory includes at least one random access memory (RAM 502) and at least one read-only memory (ROM 504). All of these elements are in communication with a central processing unit (CPU 506) to facilitate the operation of the computing device 500. The computing device 500 may be configured in many different ways. For example, the computing device 500 may be a conventional standalone computer or alternatively, the functions of computing device 500 may be distributed across multiple computer systems and architectures. In FIG. 5, the computing device 500 is linked, via network or local network, to other servers or systems.

The computing device 500 may be configured in a distributed architecture, wherein databases and processors are housed in separate units or locations. Some units perform primary processing functions and contain at a minimum a general controller or a processor and a system memory. In distributed architecture implementations, each of these units may be attached via the communications interface unit 508 to a communications hub or port (not shown) that serves as a primary communication link with other servers, client or user computers and other related devices. The communications hub or port may have minimal processing capability itself, serving primarily as a communications router. A variety of communications protocols may be part of the system, including, but not limited to: Ethernet, SAP, SAS™, ATP, BLUETOOTH™, GSM and TCP/IP.

The CPU 506 comprises a processor, such as one or more conventional microprocessors and one or more supplementary co-processors such as math co-processors for offloading workload from the CPU 506. The CPU 506 is in communication with the communications interface unit 508 and the input/output controller 510, through which the CPU 506 communicates with other devices such as other servers, user terminals, or devices. The communications interface unit 508 and the input/output controller 510 may include multiple communication channels for simultaneous communication with, for example, other processors, servers or client terminals.

The CPU 506 is also in communication with the data storage device. The data storage device may comprise an appropriate combination of magnetic, optical or semiconductor memory, and may include, for example, RAM 502, ROM 504, flash drive, an optical disc such as a compact disc or a hard disk or drive. The CPU 506 and the data storage device each may be, for example, located entirely within a single computer or other computing device; or connected to each other by a communication medium, such as a USB port, serial port cable, a coaxial cable, an Ethernet cable, a telephone line, a radio frequency transceiver or other similar wireless or wired medium or combination of the foregoing. For example, the CPU 506 may be connected to the data storage device via the communications interface unit 508. The CPU 506 may be configured to perform one or more particular processing functions.

The data storage device may store, for example, (i) an operating system 512 for the computing device 500; (ii) one or more applications 514 (e.g., computer program code or a computer program product) adapted to direct the CPU 506 in accordance with the systems and methods described here, and particularly in accordance with the processes described in detail with regard to the CPU 506; or (iii) database(s) 516 adapted to store information that may be utilized and/or required by the program.

The operating system 512 and applications 514 may be stored, for example, in a compressed, an uncompiled and an encrypted format, and may include computer program code. The instructions of the program may be read into a main memory of the processor from a computer-readable medium other than the data storage device, such as from the ROM 504 or from the RAM 502. While execution of sequences of instructions in the program causes the CPU 506 to perform the process steps described herein, hard-wired circuitry may be used in place of, or in combination with, software instructions for implementation of the processes of the present disclosure. Thus, the systems and methods described are not limited to any specific combination of hardware and software.

Suitable computer program code may be provided for performing one or more functions in relation to validating routing policies for a network as described herein. The program also may include program elements such as an operating system 512, a database management system and “device drivers” that allow the processor to interface with computer peripheral devices (e.g., a video display, a keyboard, a computer mouse, etc.) via the input/output controller 510.

The term “computer-readable medium” as used herein refers to any non-transitory medium that provides or participates in providing instructions to the processor of the computing device 500 (or any other processor of a device described herein) for execution. Such a medium may take many forms, including but not limited to, non-volatile media and volatile media. Non-volatile media include, for example, optical, magnetic, or opto-magnetic disks, or integrated circuit memory, such as flash memory. Volatile media include dynamic random access memory (DRAM), which typically constitutes the main memory. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM or EEPROM (electronically erasable programmable read-only memory), a FLASH-EEPROM, any other memory chip or cartridge, or any other non-transitory medium from which a computer can read.

Various forms of computer readable media may be involved in carrying one or more sequences of one or more instructions to the CPU 506 (or any other processor of a device described herein) for execution. For example, the instructions may initially be borne on a magnetic disk of a remote computer (not shown). The remote computer can load the instructions into its dynamic memory and send the instructions over an Ethernet connection, cable line, or even telephone line using a modem. A communications device local to a computing device 500 (e.g., a server) can receive the data on the respective communications line and place the data on a system bus for the processor. The system bus carries the data to main memory, from which the processor retrieves and executes the instructions. The instructions received by main memory may optionally be stored in memory either before or after execution by the processor. In addition, instructions may be received via a communication port as electrical, electromagnetic or optical signals, which are exemplary forms of wireless communications or data streams that carry various types of information.

Accordingly, the present disclosure also relates to computer-implemented applications of informative microsatellite loci, such as loci described herein to be associated various cancers. Such applications can be useful for storing, manipulating or otherwise analyzing genotype data that is useful in the methods of the invention. One example pertains to storing genotype information derived from an individual on readable media, so as to be able to provide the genotype information to a third party (e.g., the individual, a health care provider or genetic analysis service provider), or for deriving information from the genotype data, e.g., by comparing the genotype data to information about genetic risk factors contributing to increased susceptibility to cancer, and reporting results based on such comparison.

In general terms, computer-readable media has capabilities of storing (i) identifier information for at least one informative microsatellite locus, preferably one or more of those listed in any of Tables 1-10 or 14-22; (ii) an indicator of the frequency of at least one allele of said at least one microsatellite locus, in individuals with cancer; and an indicator of the frequency of at least one allele of said at least microsatellite locus, in a reference population. The reference population can be a disease-free population of individuals. Alternatively, the reference population is a random sample from the general population, and is thus representative of the population at large. The frequency indicator may be a calculated frequency, a count of alleles, or normalized or otherwise manipulated values of the actual frequencies that are suitable for the particular medium. The media may further include genotype data for one or more individuals, in a suitable format, such as genotype identity, genotype counts of particular alleles at particular markers, sequence data that include particular polymorphic positions, etc. Data stored on computer-readable media may thus be used to determine risk of cancer for particular microsatellite loci and particular individuals. The foregoing is merely exemplary, and other specific examples are provided below. Moreover, the same systems and methods are applicable to analyzing microsatellites to identify informative loci associated with increased risk of other diseases or conditions (e.g., diseases and conditions other than cancer), as well as identifying informative loci associated with disease aggressiveness (and thus, life expectancy and/or disease prognosis) and/or likely responsiveness or non-responsiveness to one or more particular therapeutic modalities.

The disclosure contemplates that computer-implemented methods and systems are also applicable and suitable for performing any of the methods of the disclosure. For example, in analyzing a sample from a subject, such as part of a diagnostic or prognostic method, the disclosure contemplates that information from the sample can be obtained, analyzed, and compared to information (including information stored in a database) about the characteristics of one or more microsatellites. Moreover, methods and systems used to align microsatellites across populations to identify informative loci may also be used to analyze sequencing or other microsatellite data obtained from a test subject. In other words, these and other methods may be used not only to identify informative microsatellite loci, but also to analyze microsatellite allelotype or genotype for one or more loci in a test subject and/or to compare that microsatellite information to one or more references (e.g., allelotype or genotype information for a reference population of healthy individuals and/or to some other reference population).

The disclosure provides numerous computer implemented systems that may be applied together or separately. For example, the disclosure provides a computer implemented system that may be used to reliable call microsatellite loci. Reliably called sequence information can be analyzed across a plurality of samples to provide information about microsatellite loci across a reference population. This information includes information about average sequence lengths, considered on an allele-by-allele basis. Additionally or alternatively, this information includes genotype and/or distribution of genotypes, for a given loci, across a plurality of samples. From this distribution, a modal genotype can be determined for that population.

When determining microsatellite loci informative for distinguishing between two states (e.g., between healthy and breast cancer; between aggressive and non-aggressive tumor), information obtained from two populations can be compared. For example, the distribution of sequence lengths and/or genotypes is compared, in a computer system. Using statistical analysis, such as standard statistical analysis known in the art, the distributions, for a particular microsatellite, can be compared to identify loci where the distribution of sequence lengths or genotypes for a first population are separable, in a statistically significant way, from the sequence lengths or genotypes, respectively, of a second population. In other words, the distributions are said to not significantly overlap. In certain embodiments, there may be no overlap in the two distributions (e.g., the distributions are completely separated). However, in other embodiments, the distributions may overlap, to some extent, but they are not identical and, in fact, differ from each other in a statistically significant way. Either of these scenarios are considered examples where the distributions do not significantly overlap.

Once information about informative microsatellite loci is determined, all or a portion of that information may be stored in a data base or host computer or server, and used for future comparison as a reference data set. For example, information about the informative microsatellite loci obtained from analysis of one or both reference populations may be stored as one or more values (e.g., a value of modal genotype; a value of genotype distribution; a value of average sequence length). This value may be use for future comparison when evaluating a new sample, such as in a method of diagnosing a new subject.

The following is a further exemplary method of microsatellite genotyping. DNA samples from the two populations may be optionally exome enriched, or enriched using microsatellite-specific enrichment probes, and sequenced with Next Generation sequencing then aligned to the current human reference.

Creation of microsatellite target set: An initial set of microsatellites may be identified using Tandem Repeats Finder (TRF) (Benson G (1999) Nucleic acids research 27 (2):573-580), with parameters matching weight=2, mismatching penalty=5, indel penalty=5, match probability=80, indel probability=10, minimum alignment score to report=14, maximum period size to report=4, 6, and then 1. Changing the maximum period sizes allows for identifying microsatellites of different canonical repeat lengths, with some uniquely found in each set based on the algorithm used by TRF to identify repeat regions. Those microsatellites which are less than 12 bases in length, except in exons which are allowed to be a minimum of 10 bases in length, may be filtered out. The length of microsatellites may be limited as short microsatellite motifs are less likely to be highly mutable when compared with long microsatellite motifs. Microsatellites which contain single nucleotide polymorphisms (SNPs) and/or insertions and/or deletions (indels) in the human reference which would result in more than 10% differing from an ideal repetition of the canonical repeat may be removed. Microsatellites with embedded SNPs and their associated genotypes can also be reviewed. Microsatellites which overlapped may be removed. Microsats with at least one base overlapping a large repetitive element (SINEs, LINEs, and ALUs) may be removed.

Next, microsatellites may be filtered out which do not have unique flanking sequences. Microsatellites with small repeats in their flanking sequences may be filtered out. Then each pair of flanking sequences may be searched for, individually, in the human genome. Microsatellites which have flanking sequences that occur more than once in the human genome within about 200 bases of each other and have about 5 bases of the repeat in between may be filtered out. Ten base flanking sequences may be used when sequence reads are around 100 bases in length. As the read lengths increase from the next-generation sequencing platforms, flanking sequences having increased lengths may be used in order to filter out fewer microsatellites from the set as the larger flanking sequences will result in a larger set of microsatellites which can be uniquely mapped. The remaining microsatellites may be associated with genes and regions upstream defined as the 1,000 bases preceding the transcription start site.

Calling Repeat Lengths Using Microsatellite-Based Genotyping:

The raw read alignment process begins by mapping the reads to the reference, e.g., by using BWA for short reads or BWA-SW for long LS454 reads (Li H, Durbin R (2009) Bioinformatics 25 (14):1754-1760). This process may not be done as all reads mapped to microsatellites will eventually have their alignments tested and possibly be realigned to the same locus or another locus in the genome. However, this step is useful to speed up future steps. Next, a Perl script plus SAMTOOLS may be used to pull out all of the reads from all of the microsatellite loci in batches to speed up the processing. Using about 5 bases of flanking sequence on either side the reads may be tested to make sure they completely span the microsatellite sequence and also to determine if they are the correct match for the microsatellite locus to which they have been aligned, e.g., by BWA. Once a read is found which is a good match to a microsatellite locus, using the flanking sequences, starting with about 5 bases and increasing to include more flanking sequence and possibly some of the repeat sequence next to the flanking sequence, if needed, we may align this read to the reference. At this point if there are more than two high quality matches for one flanking sequence in the read, this read may be removed from the set as the optimal alignment cannot be determined and so the microsatellite read length cannot be called with confidence. At this step all of the reads which BWA aligned to a microsatellite, but for which we found do not align to that particular microsatellite locus, may be combined with all of the reads which were not found to align with the reference at all, e.g., by BWA, using SAMTOOLS and a custom Perl script to create a fastq file. All of these reads comprise the final batch to process for which we may attempt to align them to any of the microsatellite loci using both 5 base flanking sequences. If it is determined an alignment is possible because there is enough flanking sequence contained on the read and also the flanking sequences match that of a particular locus, another alignment may be performed to find the best mapping of the read to the reference as in some cases there can be more than one possible alignment.

The reads which have been aligned to particular microsatellite loci may then be filtered to determine if at least about 5 bases of their particular repeat are contained within the flanking sequences. If the uniqueness test used about 10 bases of flanking sequence those repeats which do not align to about 10 bases of flanking sequences may be filtered out. The length of the flanking sequences required can be modified in the code to any length from 5 to 10 bases though it may be the same as that which is tested for uniqueness in the initial creation of the microsatellite set to allow for this method to work as accurately as possible. Also the number of SNPs and indels allowed in the uniqueness filtering step may be the same as that allowed here. As the length of reads increases, we will be able to obtain larger flanking sequences from microsatellites and so we can run with larger flanking sequences in our algorithms. This will allow us to accept more variation in the flanking sequences and also cause more microsatellites to have unique flanking sequences because of the increased size.

At this point the set of reads may be significantly reduced from the original set, for they are only reads that map to microsatellite loci. A filter may now be applied to remove those reads which are of low quality, e.g., based on the criteria used by the 1000 Genomes Project. This step may be done at this time for efficiency as few reads at this point need to be filtered out. Next, on a per locus basis, the reads may be binned to group those which have identical repetitive sequences. These bins vary based on repeat length and also SNPs. So for example, two reads supporting a microsatellite of the same length but with different SNPs would be placed in different bins, and thus have different genotypes. If using reads from the LS454, which is known to have issues processing homopolymer sequences, any reads which contain homopolymer indels in the microsatellite or flanking sequence regions may be filtered out. The quality scores from the original fastq files may be used to determine what score is associated with each of the SNPs in the repeat region. Reads with quality scores of less than about 99.9% accuracy for a SNP in a microsatellite may be filtered from the set. The bins with 2 reads or less supporting the allele call may be removed from the set as these reads represent possibly error prone sequences. Reads with 3 times the expected average may be removed as these also indicate an error in this region, or represent highly similar microsatellite loci or genomic regions for which accurate mapping and genotyping may not be possible. Microsats for those loci with at most 2 alleles may be called. Allowing for more than 2 alleles, would only affect ˜0.01% of calls. For some studies, including characterization of sample heterogeneity, for example, more than 2 high quality alleles at a given locus may be called. A heterozygous locus may be called if the 2 alleles do not vary by more than about 2× coverage to allow for unequal amplification. For studies which SNPs are not being examined, all indications of SNPs in the microsatellite calls may be removed so they are only grouped based on repeat length.

Microsatellite Calling Restrictions for Population-Based Statistics:

To increase uniformity of coverage and genotyping rates across samples sequenced at different times with different methods by different studies, at least about 10,000 or about 15,000 microsatellite loci may be required to be called per sample for inclusion in a study. Loci with at least about 15× coverage may be considered “callable” in a given sample. A locus may be called in a minimum of 10 exomes to be included in the genotype distribution comparison analysis to remove loci which may be called at insufficient frequency in one of the two data sets. In certain embodiments, these are rules that are applied for calling alleles and/or genotypes reliably.

With respect to computer-implemented inventions, the disclosures contemplates that software may be written using any of a number of languages, such as PERL, C, C++, Java, and the like.

### 3. Global Microsatellite Patterns as Disease Biomarkers

One of the hallmarks of cancer is increased genomic instability. Microsatellites have extremely high levels of polymorphism and heterozygosity, are ubiquitous, and are over-represented in the human genome. These and other features make microsatellites good candidates as novel informative markers for disease predisposition and disease progression. As detailed above, however, microsatellites are difficult to analyze, and this has thwarted the ability to identify particular microsatellite loci that are informative biomarkers. The present disclosure provides methods and systems to address this deficiency, and thus, allow the effective harnessing of characterizing microsatellites and applying the information to methods of disease predisposition, prognosis, diagnosis, and the like.

The disclosure is based, in part, on the hypothesis that both the germline and tumor genomes of cancer patients have a higher level of global microsatellite variation than is present in the genome of the unaffected population. This hypothesis proved to be true. A comparison of genomes (germline or tumor) from individuals with cancer to individuals identified as not having cancer not only revealed that (1) the genomes of the cancer patients (both germline and tumor) have increased level of microsatellite variation per genome, and (2) the genomes of the cancer patients have specific microsatellite signatures. Of particular note, across the cancer patients, the instability is observed in both the germline and tumor genome, and that instability is very similar. Thus, the level of microsatellite instability is not simply a product of changes that occur in a tumor. Rather, the level of microsatellite instability is present in the non-tumor genome present in a given individual from birth.

The foregoing observations lead to the following themes that apply throughout the disclosure. First, because microsatellite instability and informative microsatellite loci are present in the non-tumor, germline genome, microsatellite instability and informative loci can be used prior to onset of symptoms (and even from birth) to predict risk of developing cancer or other disease. Second, because this predictive information is present in the non-tumor, germline genome, analysis can be performed non-invasively, based on a blood sample, skin sample, cheek swab, and the like.

To do comparative analysis and to evaluate difference that may be informative as a diagnostic or prognostic tool, it was first necessary to determine the normal range of variation of microsatellite in the unaffected population (e.g., population of individuals not diagnosed with or suspected of having a particular disease or condition). This can be done, for example, by analyzing variation within individuals sequenced as part of the 1000 Genomes Project (1 kGP). Methods for computing a microsatellite profile across a plurality of microsatellites, such as across 10,000 loci or genome-wide, on an individual and population scale are described in Section 2 above and in the examples below. The global microsatellite profile among normal individuals then servers as the “baseline” for comparison to the microsatellite profile of individuals diagnosed with a particular condition or disease, such as cancer. Once a baseline profile is obtained, it can be compared to a microsatellite profile obtained from a disease population. The findings of such comparisons provide at least two different ways in which microsatellite information for a particular patient or population can be evaluated to provide information indicative of the risk of developing cancer, and other diseases.

A first is a concept referred to herein as Global Microsatellite Instability or GMI. Global Microsatellite Instability is defined as being a significant increase in the number of variable microsatellite loci across a large number (e.g., 10,000 or even all identifiable microsatellite loci) of identifiable microsatellite loci for a given individual or population, relative to a reference genome or population. In the exemplary comparative analysis outlined above, in which the microsatellite profile of unaffected individuals (e.g., also referred to as healthy—at least with respect to not being suspected of having a particular disease or condition) sequenced as part of the 1000 Genomes Project was compared to that of individuals afflicted with a particular cancer, we found that genomes from cancer patients have a significantly increased level of microsatellite variation per genome. Thus, examining GMI in a subject provides a biomarker for assessing risk of developing cancer. In other words, if the level of variation is similar to or more akin to that observed in the plurality of cancer patients, a subject is characterized as being at risk of developing cancer. On the other hand, if the variation is similar to or more akin to that observed in the plurality of unaffected subjects, a subject is characterized as being at low risk of developing cancer. A level of variability intermittent between the cancer and unaffected populations may indicate that a subject has an intermediate level of risk.

A second is a more specific and thorough analysis of the actual loci that vary between the two populations being examined, which provide an informative novel risk assessment tool for the development, prognosis, diagnosis, and progression of a disease or condition, such as a particular cancer. To identify informative loci, one compares loci among and between two populations, such as an unaffected population and a population having a particular disease or condition (e.g., cancer, such as a particular cancer). Note, as described below, other populations may be compared to identify loci informative in other contexts. The microsatellite loci which vary significantly among the unaffected population (e.g., normal, or cancer-free) generally do not represent loci that are useful for risk assessment, such as cancer risk assessment (e.g., these are not likely to be informative loci for assessing disease risk). Rather, it is the microsatellite loci which are highly conserved among the unaffected population, but highly variable among the afflicted population (in this example, the population previously diagnosed with cancer) which represent likely informative markers useful for assessing risk of developing cancer. Once the informative loci are identified based on these comparisons, the informative loci can than be used to characterize risk or in diagnostics for individual patients (e.g., by examining informative loci and comparing the results to the data generated based on examination of populations of unaffected and/or unaffected individuals). Note, however, that when evaluating distributions of genotypes, as outlined herein, we did not require the genotype for a loci to be invariant, or substantially invariant, or highly conserved within a reference population, such as a reference healthy population. Thus, requiring a high level of conservation at a locus within a reference healthy population is optional when using identifying informative loci based on distributions of genotype.

One of ordinary skill in the art will appreciate that this comparative analysis can be extended to conditions other than cancer. For example, the same type of comparative analysis could be done to determine microsatellite signatures which could serve as potential risk assessment tools for the development of other diseases relating to the following organs, tissues, and metabolic, reproductive and other bodily functions involved in human health, including, but not limited to, cardiovascular, respiratory, kidney and urinary tract; immune system, gastrointestinal, neurological, psychoneurological, and hematological functions and systems. In further aspects, the same analysis could be performed within populations afflicted with a particular disease to determine, for example, microsatellite signatures associated with fast, medium or slow progression of a disease (e.g., aggressiveness) or for determining informative loci indicative of responsiveness to a particular treatment regimen. When making these other comparisons, one must select an appropriate reference population for use as a comparator.

Accordingly, in some aspects, the present disclosure provides methods that can be used to measure a GMI profile in a given population or individual. In a broad sense, a method for measuring GMI in a population comprises (1) determining a distribution of sequence lengths for a plurality of microsatellite loci in nucleic acid obtained from a first population; (2) comparing the distribution of sequence lengths for a first microsatellite locus in nucleic acid obtained from the first population to the sequence length for the same first microsatellite locus in a reference genome; (3) repeating the comparing step (2) for additional microsatellite loci; and calculating the percentage of microsatellite loci whose lengths differ from the lengths of the microsatellite loci of the reference sequence. It will be appreciated that the lengths of the microsatellite loci of the first population can instead be compared to a distribution of sequence lengths for a reference population (e.g., one used to compute a reference genome).

Another method for measuring GMI in a population comprises (1) determining a distribution of genotypes for a plurality of microsatellite loci in nucleic acid obtained from a first population; (2) comparing the distribution of genotypes for a first microsatellite locus in nucleic acid obtained from the first population to the modal genotype for the same first microsatellite locus in a reference population; (3) repeating the comparing step (2) for additional microsatellite loci; and calculating the percentage of microsatellite loci whose genotype differ from the modal genotype of the microsatellite loci of the reference population. It will be appreciated that the genotype of the microsatellite loci of the first population can instead be compared to a distribution of genotypes for a reference population (e.g., one used to compute a reference genome). As used herein, modal genotype is that genotype which is supported by the highest number of samples in a reference population (e.g., the most common genotype). This can similarly be applied to a test sample by determine a genotype for a plurality of microsatellite loci and comparing the genotype data to that from a reference population, e.g., fitting the test data into the distribution data of one or more references or comparing to the reference modal information or a condition-like signature. Moreover, GMI comparisons can be made between a germline sample from a cancer subject and a tumor sample, on an individual or population level, to identify hot spots: microsatellite loci that differ between the germline and tumor subject and are indicative of additional events occurring specifically in the tumor. These hot spots may be in genes that represent targets for drug screening or therapeutic intervention.

In further aspects, the present disclosure provides methods that can be used to identify microsatellite loci useful as markers for assessing presence, potential risk, stage, etc. of various diseases. Such microsatellite loci are referred to herein as “informative microsatellite loci.”

In a broad sense, a method for identifying informative microsatellite loci comprises (1) determining a distribution of genotypes for a plurality of microsatellite loci obtained from a first population (e.g., from nucleic acid or sequence information obtained from a first population); (2) determining a distribution of genotypes for a plurality of microsatellite loci obtained from a second population (e.g., from nucleic acid or sequence information obtained from a first population); (3) comparing the distribution of genotypes for a first microsatellite locus obtained from the first population to the distribution of genotypes for the same first microsatellite locus obtained from the second population; (4) repeating the comparing step (3) for additional microsatellite loci; and classifying as informative any microsatellite locus whose distributions of genotypes do not significantly overlap between the two populations.

An alternative method for identifying informative microsatellite loci comprises (1) determining a distribution of sequence lengths for a plurality of microsatellite loci obtained from a first population (e.g., from nucleic acid or sequence information obtained from a first population); (2) determining a distribution of sequence lengths for a plurality of microsatellite loci obtained from a second population (e.g., from nucleic acid or sequence information obtained from a first population); (3) comparing the distribution of sequence lengths for a first microsatellite locus obtained from the first population to the distribution of sequence lengths for the same first microsatellite locus obtained from the second population; (4) repeating the comparing step (3) for additional microsatellite loci; and classifying as informative any microsatellite locus whose distributions of sequence lengths do not significantly overlap between the two populations. In certain embodiments, analysis of sequence lengths permits analysis of both length (e.g., number of repeats), as well as sequence, thus allowing analysis of polymorphisms within a microsatellite or flanking a microsatellite. Similarly, when analyzing genotype, length and sequence may be analyzed, thus allowing analysis of polymorphisms within a microsatellite or flanking a microsatellite. On an individual sample basis, determining a genotype for a locus comprises determining the sequence length and/or sequence for both alleles and then assigning a genotype based on information from both alleles (e.g., a genotype unit).

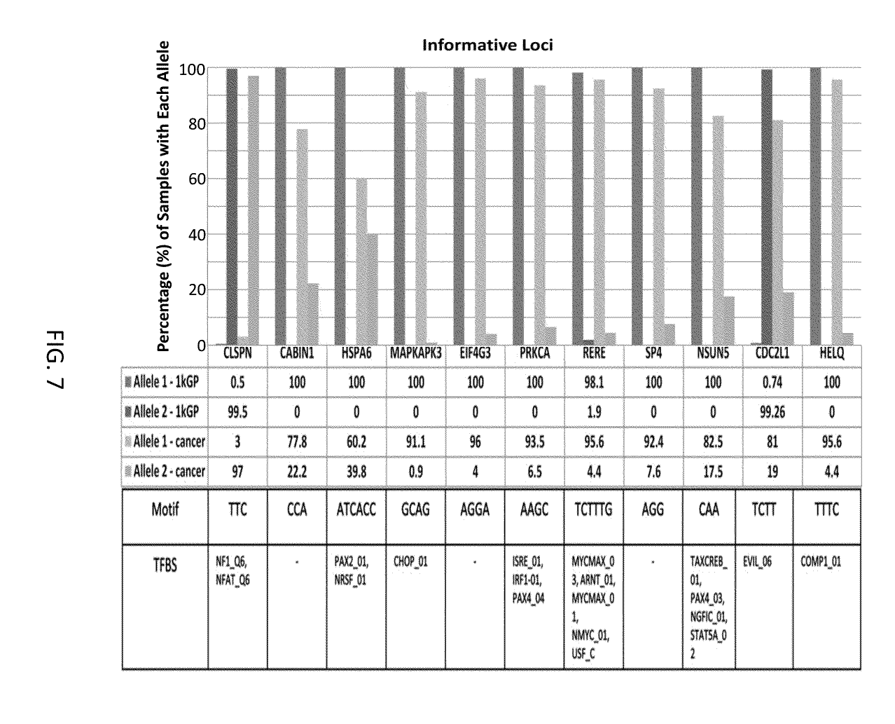

FIG. 6 provides a schematic illustrating such a method for identifying informative microsatellite loci, as described herein. As will be readily appreciated the selection of the first and second populations is selected based on the goal (e.g., for what characteristics are you looking for informative loci). Thus, in certain embodiments, one of the populations is affected with a particular disease or condition and the other population is not affected with that same disease or condition. As detailed above, the disclosure recognizes that, for specific members of a population, there may be members who ultimately will be diagnosed with a particular disease but are thought to be healthy at the time. This, however, is expected when generating reference populations and does not detract for the use of populations including these samples as an appropriate healthy reference. This permits identification of loci informative for that particular disease or condition. In other embodiments, one of the populations responded well to a particular therapeutic regimen for a particular condition and the other population did not respond to that regimen. This permits identification of loci informative for selecting a treatment plan and/or predicting responsiveness to a treatment plan. In other embodiments, one of the populations had an aggressive form of a particular disease or condition and the other population had a less aggressive or non-aggressive form of that same disease or condition. This permits identification of loci informative for predicting disease course and outcome. Although what is considered to be aggressive or non-aggressive when referring to the etiology and progression of a disease will varying depending on the disease and other factors. In certain embodiments, “aggressive” refers to one or more of the following: (i) having a life expectancy lower than the average life expectancy for that disease or condition (e.g., at least 10%, 20%, 25%, or even 50% less than the average life expectancy), (ii) having a life expectancy of less than three months from diagnosis, (iii) having a disease progression at least 25% greater than the average disease progression for that disease or condition, or (iv) characterized as aggressive by the treating physician in their professional judgment. In certain embodiments, “non-aggressive” refers to one or more of the following: (i) having a life expectancy equal to or greater than the average life expectancy for that disease or condition, (ii) having a disease progression equal to or slower than the average disease progression for that disease or condition, or (iii) characterized as non-aggressive by the treating physician in their professional judgment.

Rules for the identification of a microsatellite locus whose distributions of sequence lengths and/or actual sequence do not significantly overlap between the two populations may vary in accordance to certain embodiments of the present disclosure. Similarly, in certain other aspects, actual sequence and/or sequence lengths for both alleles are determined and examined (e.g., determining a genotype; analysis based on that determined genotype rather than allelotype). The same or differing rules can be used to evaluate distribution of allelotype or genotype. In certain embodiments, the lack of significant or substantial overlap is a statistically significant lack of overlap between a distribution from populations. In certain embodiments, the lack of significant or substantial overlap does not mean that there is no overlap between the distribution of two populations, but rather means there is a statistically significant difference between the distributions of the populations.

In some embodiments, a baseline for variation is established by analyzing genotype variation at a plurality of microsatellite loci in a control population. The samples may be age, sex and/or ethnically matched. The analysis may be restricted to those loci that are callable with sufficient coverage (about 15×) in at least about 10 exomes from both the condition and control populations. In certain embodiments, sufficient coverage may be about 10×, 11×, 12×, 13×, 14×, 15×, 16×, 17×, 18×, 19×, 20× or greater. In certain embodiments, sufficient coverage may be represented in about 5, 6, 7, 8, 9, 10, 11, 12, 13, 14, 15 or more exomes from both the condition and control populations. A profile or distribution of genotypes for the condition and control cohorts is then generated for each locus. An allele is defined by a genomic locus with a specific microsatellite repeat and nucleotide sequence length and/or actual sequence. In each sample a pair of loci is identified and each allelic pair is then defined as a genotype. The genotype most prevalent from a distribution of genotypes identified (called) in the control population is defined as the modal genotype. If more than a pair of alleles is identified for a locus that sample may be taken out of the analysis. A comparison of the profiles is done to identify loci that individually show a statistically significant difference in a genotype distribution between the condition and control populations. In certain embodiments, the statistically significant difference is determined using a two-sided Fisher's p and/or Benjamini-Hochberg analysis.

In certain embodiments of any of the methods described herein, a reference population is generated from members that are matched based on one or more traits, such as age, gender, and ethnicity. In certain embodiments, when comparing two populations the two populations may be selected so that they are each generated from members that are matched based on the same one or more traits. In other words, when comparing a population of healthy members to a population of members having breast cancers, the two populations can each be comprised of members having certain traits, and these shared traits can be the same in the two populations to which you are making the comparison. Moreover, the traits of the population may be selected based on the anticipated traits of ultimate test subjects. Thus, for identifying informative loci for breast cancer, where the ultimate test subjects will be predominantly female, the one population or two populations used to identify loci and/or to compare test data may be comprised of female members.

In some embodiments, the rules include the following parameters: (1) locus is called in at least 25 individuals in the reference population with less than 2% variation, (2) at least 3% of locus-specific alleles in the target population vary relative to the most common allele in the reference population, and (3) ≧3 locus-specific alleles in the target population are different from the most common allele in the reference population. These and other rules may be used. As discussed herein, the rules may be used in any of the contemplated contexts, including to identify informative loci for risk of a particular cancer, loci for evaluating tumor aggressiveness, or loci for predicting responsiveness of a therapy.

In some embodiments, the more stringent rules may be employed such as, for example, the use of cross-validation analysis. In some embodiments, loci that have passed the initial test, e.g., those whose distributions of sequence lengths do not significantly overlap between the two populations, are cross-validated using methods such as Random Subsampling, K-Fold Cross-Validation, and Leave-one-out Cross-Validation. These methods are well known in the art, and commonly used in the bioinformatics industry. Such further analysis may be useful for selecting from amongst an initial set of informative loci, a subset of informative loci for further use. However, the disclosure contemplates that informative loci for use in methods of, for example, (i) evaluating predisposition to a disease or condition, (ii) prognosing aggressiveness or therapeutic responsiveness of a disease or condition, or (iii) providing a confirming diagnosis of a disease or condition may be based on examination of one or more informative loci selected from an initial, larger data set based on a first set of selection criteria and/or may be based on examination of one or more informative loci selected from a subset of such informative loci based on a second set of selection criteria. In certain embodiments, this is applied to informative loci selected based on allelotype distribution and in other embodiments, this is applied to informative loci selected based on genotype distribution.

Rules for the identification of a microsatellite locus whose distributions of genotypes do not significantly overlap between the two populations may also vary in accordance to certain embodiments of the present disclosure.

Thus, the disclosure contemplates methods of evaluating the presence or predisposition to a condition comprising determining a genotype for at least 5%, 10%, 20%, 30%, 40%, 50%, 60%, 70%, or 80% of informative microsatellite loci from a panel. In some embodiments, the panel of microsatellite loci identified as being informative comprises a list of at least six, at least seven, at least eight, at least nine, or at least ten or more microsatellite loci. In some embodiments, each sample is sequenced to a depth of at least 15× at each microsatellite locus. In some embodiments, the lack of significant or substantial overlap does not mean that there is no overlap between the distribution of two populations, but rather means there is a statistically significant difference between the distributions of the populations. In some embodiments, the subject is identified as having or having a predisposition to a condition if at least 30%, 35%, 40%, 45%, 50%, 55%, 60%, 65%, 70%, 75%, 80%, 85%, 90%, 95% of the genotyped loci show a condition-like genotype or a genotype that has a larger association with the reference population identified as having the condition than the with the reference population identified as not having the condition or having a different condition, e.g., the genotypes best fit into the distribution of the reference population with the condition. In some embodiments, the number of loci that are associated with the condition for diagnosis or prognosis is determined by a threshold that maximally differentiates the two populations via the distributions of the panel of informative loci that resemble the genotypes of the two populations. In a preferred embodiment, the method comprising determining a genotype at least one of the loci having a relative risk of >1.3 or <0.6. Variation at any one or more of the loci having a relative risk of >1.1, 1.2 or 1.3 may be indicative of the presence or predisposition to a condition. Variation at one any one or more of the loci having a relative risk of <0.9, 0.8, 0.7 or 0.6 may be indicative of a lowered risk of the presence or predisposition to a condition (a protective loci). In some embodiments, the relative risks are weighted in the analysis. In some embodiments, the depth coverage of each loci is weighted in the analysis. In some embodiments, the presence of minor alleles is weighted in the analysis. In some embodiments, the analysis of the genotyped microsatellites identifies a condition-associated genotype in a sample with a specificity of at least 60%, 70%, 80%, 90%, 95%, 99% or greater and a sensitivity of at least 60%, 70%, 80%, 90%, 95%, 99% or greater. In some embodiments, the reference populations are based on at least 100 members. In some embodiments, the reference populations are gender, age, and/or ethnicity matched to the sample. In some embodiments, the methods are implemented on a computer. In some embodiments, each reference population has at least 10,000 microsatellite loci called. These embodiments may be applicable to any of the disclosed methods, e.g., identifying an increased risk for cancer or for analyzing other conditions, characteristics or traits.

By way of example, we have used these methodologies to successfully identify informative microsatellite loci associated with breast cancer, ovarian cancer, glioblastoma, prostate cancer, colon cancer and lung cancer. Moreover, as described herein, we have identified informative loci based on analysis of allelotypes, as well as based on determining a genotypes. As explained above, one of skill in the art will appreciate that these methodologies can be used to identify informative microsatellite loci that correlate with a wide range of conditions including, but not limited to, other cancers (e.g., liver cancer, kidney cancer, pancreatic cancer, leukemias, lymphomas, pediatric cancers, melanoma, and the like). Identification of informative loci associated with other cancers requires analyzing a plurality of microsatellites from a plurality of patient samples already diagnosed with the particular cancer of interest. This population can be evaluated and compared to a healthy reference population or to another reference population. Then the same types of comparisons can be made between the microsatellite signature for the cancer samples and that of healthy genomes. In addition, identification of informative loci associated with aggressiveness and/or responsiveness to particular therapeutic modalities is also contemplated. In such embodiments, the two populations of samples are selected so that a comparison reveals informative loci associated with aggressiveness or responsiveness to treatment. For example, to identify informative loci associated with aggressiveness of a particular cancer, a signature of a plurality of microsatellite loci examined for a plurality of subjects in which a particular cancer was very aggressive (e.g., survival from date of diagnosis was at least 50% shorter than average survival time for that cancer) is compared to a signature of a plurality of microsatellite loci examined for a plurality of subjects in which that same type of cancer was not aggressive (e.g., survival from date of diagnosis was equal to or exceeded average survival time). Also contemplated and described herein, is the use of informative loci to distinguish between two types of cancers of a particular tissue, such as between different types of brain cancers or different types of lung cancers. By way of example, in the case of brain cancers, the ability to distinguish, non-invasively, between an aggressive cancer requiring immediate and significant intervention versus a low grade cancer provides significant benefits and enhances patient safety.

Similarly, identification of informative microsatellite loci can be applied to other diseases or conditions, such as neurological diseases and conditions, neurodegenerative disorders, autoimmune diseases and conditions, inflammatory disorders, cardiovascular diseases, and the like. Identification of informative loci associated with other conditions requires analyzing a plurality of micro satellites from a plurality of patient samples already diagnosed with the particular disease or condition of interest. Then comparisons can be made between the microsatellite signature for the afflicted samples and that of healthy genomes. Because this approach is not biased to focus on particular types of genes, it is amenable to use with complex, multigenic conditions.

Once informative microsatellite loci are identified, these informative loci may be used to evaluate subjects (e.g., patients), such as patients suspected of having a disease state or subjects for whom it is advantageous to evaluate disease-risk. When evaluating a new test subject, the same methodologies can be applied (e.g., determining allelotypes or genotype at one or more informative loci and comparing to that of one or more reference populations, such as a healthy reference population and/or a reference population of individuals having the condition). This comparison can be performed by determining if the patient's genotype for one or more informative loci better fits into the distribution for the healthy population or the diseased population. Alternatively, the patient's genotype can be compared to the modal genotype of the healthy population at one or more informative loci or a condition-like signature or compared to the non-modal genotypes.

Breast Cancer

Breast cancer is a serious public health problem. Aside from skin cancer, breast cancer is the most common form of cancer in women, with a lifetime incidence rate of about 12% among women in the United States population. Breast cancer also remains one of the top ten causes of death for women in the US, and the second leading cause of cancer deaths in this population.

According to the invasive breast cancer estimates from the American Cancer Society, there will be 226,870 new cases in 2012 and females have a 1 in 8 chance for developing this cancer within their lifetime. Men have a 1 in 1000 chance of developing breast cancer in their lifetime. Breast cancers, like many other cancers, have significant known inherited or spontaneous components for which only a fraction has been explained by genetic variation to date. For example, less than 25 variants in the BRCA1 and BRCA2 genes account for 5 and 10% of inherited breast cancer susceptibility. Breast cancer is highly responsive to treatment when diagnosed early. Women (and men) afflicted with breast cancer would benefit significantly if more informative, actionable genetic markers were identified, thereby facilitating early and effective diagnosis.

Identification of Informative Microsatellite Loci Using Allelotyping

A baseline variation was first established by analyzing allelotype variation at a plurality of microsatellite loci in individuals from next-generation sequencing data from four different populations in the 1,000 Genome Project (1 kGP) data set, as well as next-generation sequencing data from transcriptomes of cancer-free individuals in the The Cancer Genome Atlas (TCGA). These individuals had not been diagnosed with cancer at the time of sequencing, and thus are considered to be representative of the normal or “unaffected” population.

Next-generation sequencing data from transcriptomes of women with invasive breast carcinoma were obtained from The Cancer Genome Atlas (TCGA). A profile or distribution of alleles was then computed for each microsatellite locus. A comparison of profiles from cancer and cancer-free samples revealed 165 loci for which at least one breast cancer (BC) sample was variant from the human genome reference (hg18) (Table 1). Thus, Table 1 provides a first set of informative microsatellite loci associated with increased risk of breast cancer.

GMI analysis revealed that the average level of GMI in the breast cancer population is 1.7 times greater than the normal population at coding loci. Thus GMI level is an independent indicator of risk for breast cancer. However, because the range of variation within both populations was broad, leading to overlap in the standard deviations, samples were assigned into three GMI classes—with low (non-cancer-like) as less than 0.04% variation, intermediate as 0.04% to 0.06% variation, and high (cancer-like) as variation of 0.06% and greater. Thus, in some embodiments, a person with a GMI of less than 0.04% has a low risk of developing breast cancer; a person with a GMI of 0.04%-0.06% has an intermediate risk of developing breast cancer; and a person with a GMI of more than 0.06% has a high risk of developing breast cancer. Thus, in certain embodiments, analysis of GMI permits predicting risk in either or both of an absolute sense (e.g., a subject has an increased risk) and in terms of the degree of risk (e.g., low, intermediate, or high risk).

Further analysis revealed that 50.4% of the 1 kGP normal samples would be considered low GMI, 30.4% would be intermediate, and 19.2% would be GMI high. For the BC samples, 17.3% were low GMI, 22.1% intermediate and 60.7% high GMI. This difference would likely be even more pronounced if comparing variation levels at non-coding microsatellite loci as the frequency of variation for all genomic regions in the 1 kGP data was 36 times that found in coding regions, consistent with previous measurements and the fact that these loci lie in a variety of genomic locations (introns, exons, intergenic spaces) which exhibit differing pressures.

A further analysis of the variant microsatellite loci revealed a set of 13 microsatellite loci which were highly conserved in cancer-free genomes (0.4% varying) but were highly variable in cancer transcriptomes (over 87% had differing alleles) (Table 2). Thus, Table 2 provides a subset of informative microsatellite loci associated with increased risk of breast cancer and selected based on a more stringent selection criteria.

The disclosure contemplates methods of evaluating breast cancer predisposition, as well as prognostic and diagnostic methods in which any one or more (e.g., 1, 2, 3, 4, 5, 6, 7, 8, 9, 10, 11, 12, 13, or greater than 13) of the microsatellite loci set forth in Table 1 and/or Table 2 are examined in a patient (e.g., in a particular patient in need of evaluation). Moreover, the disclosure contemplates that analysis of any one or more (e.g., 1, 2, 3, 4, 5, 6, 7, 8, 9, 10, 11, 12, 13) of the loci set forth in Table 2 may be combined with any one or more (e.g., 1, 2, 3, 4, 5, 6, 7, 8, 9, 10, 11, 12, 13, 14, 15, more than 15) of the loci set forth in Table 1. In certain embodiments, the disclosure contemplates that all of the 13 informative microsatellite loci set forth in Table 2 are evaluated as part of a method. In certain embodiments, the disclosure contemplates that all of the 165 informative loci set forth in Table 1 are evaluated. In either case, it should be appreciated that one or more additional loci (in addition to the 13 or 165 informative loci identified herein) can also be included for evaluation.

Using the 13 informative microsatellite loci set forth in Table 2, we were able to distinguish between breast cancer genomes as inferred from RNA sequence data and normal genomes at a sensitivity of 87.2% (breast cancer tumor; nucleic acid from tumors of breast cancer data set) and 100% (breast cancer somatic; germline nucleic acid of breast cancer data set) with a minimum specificity of 96.2%. Note, the difference observed when assessing sensitivity in the BC data sets (e.g., tumor nucleic acid versus germline nucleic acid) is a function of the difference in the number of samples and is not thought to reflect a statistically relevant difference in sensitivity between the two data sets.

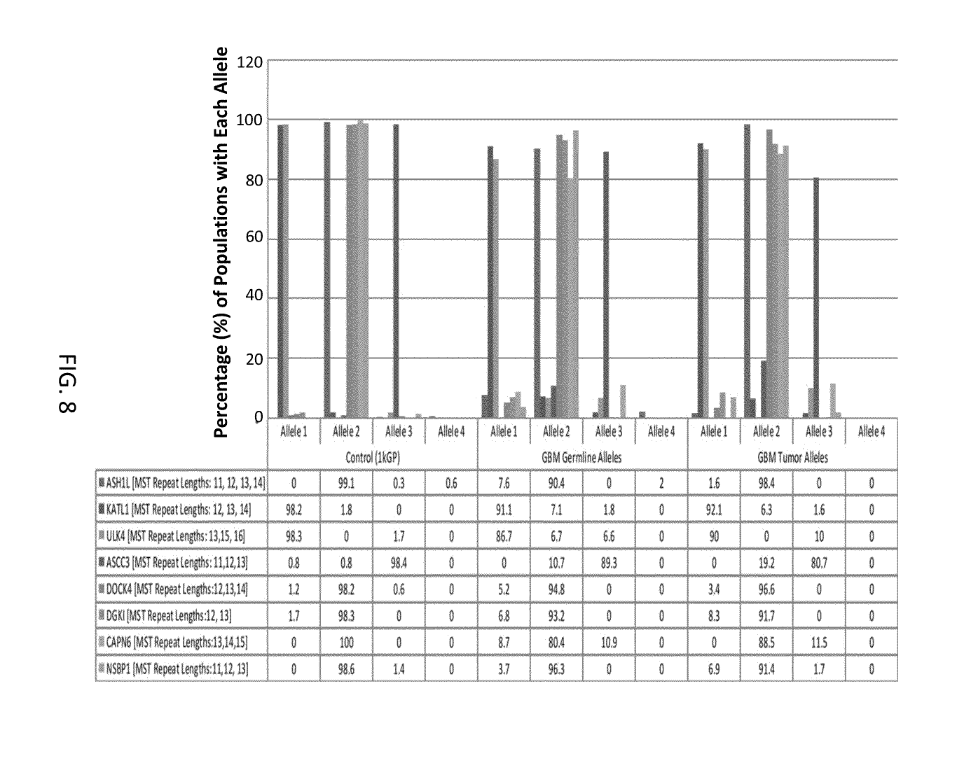

Importantly, it should also be noted that these loci are highly conserved in the cancer-free population, which consists of females from four different ethnic groups; therefore these loci are conserved across ethnic groups and the variations seen in the breast cancer samples are unlikely to be attributed to ethnicity. Of the 13 informative loci, 5 were called with higher frequency in the breast cancer data and are therefore considered highly informative. Using these 5 loci, samples were classified as breast cancer or healthy (unaffected) with a sensitivity of 86.1% (breast cancer tumor) and 100% (breast cancer somatic) and with a specificity of 99.2%. These loci reside in the MAPKAPK3, CABIN1, HSPA6, NSUN5 and CDC2L1 genes and had a variation frequency of 54.5%, 51.4%, 74.2%, 72.8% and 99.5% respectively (FIG. 7). The disclosure contemplates, in certain embodiments, methods of evaluating breast cancer predisposition, as well as prognostic and diagnostic methods in which any one or more of the microsatellite loci set forth in FIG. 7 (e.g., 1, 2, 3, 4, or 5) are examined in a patient (e.g., in a particular patient in need of evaluation). Moreover, the disclosure contemplates that analysis of 1, 2, 3, 4, or 5 of the loci set forth in FIG. 7 can be combined with analysis of any one or more (e.g., 1, 2, 3, 4, 5, 6, 7, 8, 9, 10, 11, 12, 13) of the loci set forth in Table 1 or 2.

The high frequency of variation at the 5 highly informative breast cancer-associated loci, and particularly at CDC2L1, can be explained by either (1) these markers are pre-existing in people who develop cancer and as such can be used as a novel risk assessment tool for breast cancer or (2) these variations arise at a high frequency in tumors implying that they likely provide an advantage to the tumor and are potential markers or targets. To determine if these variants are found within the germline (e.g., in nucleic acid from non-tumor, somatic tissue) of people who develop breast cancer, the inventors analyzed their variation within 10 somatic/germline transcriptomes from breast cancer patients. The variant in the CDC2L1 gene was identified in all 6 samples in which the locus could be identified. The HSPA6 variant was identified in 8 out of 9 samples, and the NSUN5 variant was identified in 2 out of the 4 samples for which the locus was called. The high frequency of these three variants in germline transcriptomes indicates that they are exemplary of the identified, informative microsatellite loci useful as novel risk-assessment markers for breast cancer.

Identification of Informative Microsatellite Loci for BC Using Microsatellite Genotyping

For this analysis, we established a baseline for variation by analyzing genotype variation at a plurality of microsatellite loci in healthy females from European ancestral populations in the 1,000 Genome Project data set (1 kGP-EUF). These individuals had not been diagnosed with cancer at the time of sequencing, and thus are considered to be representative of the normal or “healthy” population (e.g., population of people not diagnosed with or suspected of having cancer at the time).

Next-generation sequencing data from germline exomes from breast cancer female patients were obtained from The Cancer Genome Atlas (TCGA) Importantly, in this example, the healthy females from 1 kGP data set and the females from the TCGA data set were ethnically matched. Furthermore, we restricted our analysis to those loci that were callable with sufficient coverage (15×) in at least 10 exomes from both the 1 kGP-EUF and breast cancer populations.

A profile or distribution of genotypes for the affected (TCGA) and unaffected (1 kGP) cohorts was then generated for each locus. An allele is defined by a genomic locus with a specific microsatellite repeat and nucleotide sequence length. In each sample a pair of loci was identified and each allelic pair was then defined as a genotype. The genotype most prevalent from a distribution of genotypes was identified (called) in 1 kGP samples; this genotype was defined as the modal genotype (if more than a pair of alleles was identified for a locus that sample was not used).

A comparison of the profiles revealed 55 loci that each individually showed a statistically significant difference in a genotype distribution between 1 kGP-EUF and breast cancer germline (p≦0.01, two-sided Fisher's p and Benjamini-Hochberg) (Table 14). 25.1%±13.1% and 31.3%±9.4% of the 55 loci were genotyped in the 1 kGP-EUF and BC germline exomes, respectively, which is not surprising given that we used very stringent conditions for coverage and alignment, and because Lander-Waterman distributions in random fragment sequencing limits the number of callable loci in each sample.

The genotypic differences at these 55 informative loci appear to have two effects on the likelihood of breast cancer. At 30 of the 55 informative loci, the presence of a non-modal genotype is potentially protective against breast cancer (relative risk of <0.6; Table 14), whereas at 25 of the loci a non-modal genotype appears to promote breast cancer (relative risk >1.3; Table 14). Thus, the disclosure contemplates methods of evaluating breast cancer predisposition, as well as prognostic and diagnostic methods in which any one or more of the loci having a relative risk of >1.3 are evaluated. Variation at any one or more of the loci having a relative risk of >1.3 is indicative of an increased risk of developing cancer.

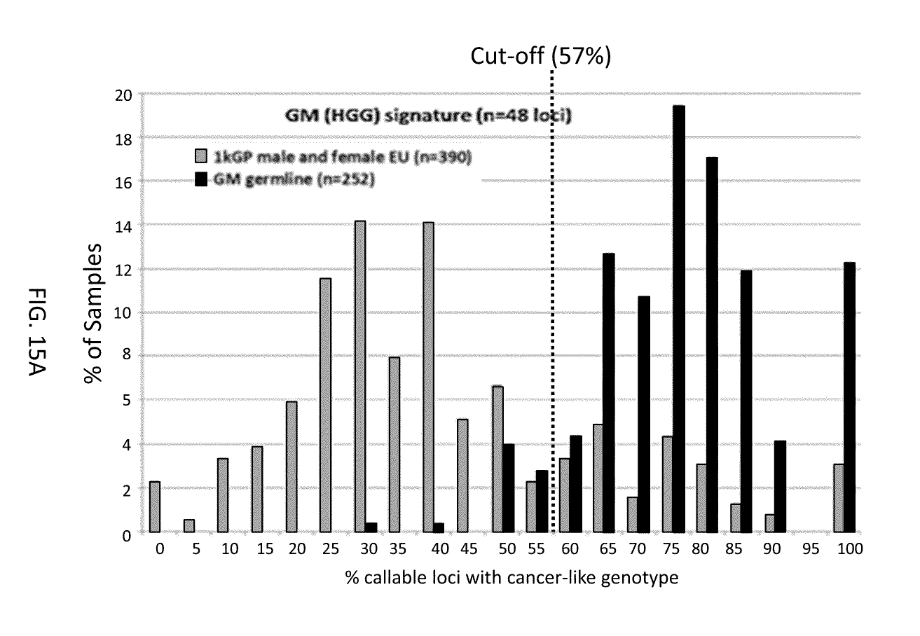

We used the frequency of modal or non-modal genotypes at each of the 55 informative loci, which we refer to as the BC-PIM (breast cancer panel of informative microsatellites) within the breast cancer population relative to the 1 kGP-EUF population to create a breast cancer genotype profile. FIG. 14 shows the distribution of exomes based on the number of genotypes at the 55 signature loci that match the cancer profile. Using the false positive and false negative rates within the training set, we were able to determine the receiver operating characteristic (ROC) for the 55 BC loci. Through maximizing the area under the ROC curve, we determined the optimal cut-off for a classifier as having 76% of the 55 BC loci matching the cancer-like profile (FIG. 14). We were then able to classify the BC germline exomes as cancer (≧76%) or healthy (<76%) with a sensitivity of 88.4%, and a specificity of 77.1% (FIG. 14).

Thus, the disclosure contemplates methods of evaluating breast cancer predisposition, as well as prognostic and diagnostic methods, comprising genotyping at least 5%, 10%, 20%, 30%, 40%, 50%, 60%, 70%, or 80% of the 55 BC loci from Table 14. Alternatively, the method may comprise genotyping at least 2, 5, 10, 15, 20, 25, 30, or 35 BC loci from Table 14. In some embodiments, the patient is identified as having an increased risk of developing cancer if at least 76% of the genotyped BC loci have a cancer-like genotype (e.g., if at least 76% of the genotyped loci have a genotype that differs from the modal genotype of a healthy, reference population or the sample data best fits the cancer-like distribution). In some embodiments, the patient is identified as having an increased risk of developing cancer if at least 50%, 55%, 60%, 65%, 70%, 75%, 80%, 85%, 90%, 95% of the genotyped BC loci from Table 14 have a cancer-like genotype.

As detailed herein, GMI instability and/or informative microsatellite loci can be used in a variety of prognostic and diagnostic methods. The disclosure contemplates that, for example, any one or more of the informative loci discussed herein or set forth in the figures and tables can be used in diagnostic and prognostic methods.

Ovarian Cancer

Ovarian cancer is the fifth most common cause of cancer death in women in the US. Five-year relative survival rate is less than 45% with the stage at diagnosis being the major prognostic factor. Only 19% of ovarian cancer cases are diagnosed while the cancer is still localized and chances of cure are over 90%. A striking 68% are diagnosed after the cancer has already metastasized.

In the absence of effective treatment for advanced ovarian cancer, the major emphasis is on developing screening programs that will detect the disease at an early stage, thereby drastically improving the opportunity for cure and/or meaningful five year survival rates. Ovarian cancer screening with transvaginal ultrasound (TVU) and CA-125 screening was evaluated in the Prostate, Lung, Colorectal and Ovarian (PLCO) Trial, and included almost 40,000 women. Screening identified both early- and late-stage neoplasms; however, the predictive value of both tests was relatively low and the effect of screening on ovarian cancer mortality will require longer-term follow-up to evaluate.

Given that approximately 1 in 72 women will be diagnosed with cancer of the ovary during their lifetime, repeated screening of the whole population with costly and invasive procedures like ultrasound is not a feasible strategy. This is particularly true considering the large number of false positive cases that need follow-up by surgical procedures with the associated risks of side effects. Management strategies that aim to identify those individuals at highest risk of the disease could be used to focus screening efforts on women who will benefit the most from them while minimizing unnecessary interventions and anxiety amongst those at lower risk.

Identification of Informative Microsatellite Loci for OV Using Microsatellite Allelotyping

For this analysis, a baseline variation was established by analyzing variation at a plurality of microsatellite locus in females from four different populations in the 1,000 Genome Project (1 kGP) data set. These individuals had not been diagnosed with cancer at the time of sequencing, and thus, were considered representative of the normal (non-ovarian cancer) population.

After establishing the ‘expected’ percentage of variant microsatellite alleles within the normal population, we asked whether there was an increase in the overall frequency of microsatellite variation in ovarian cancer. Next-generation sequencing data from germline and tumor samples from females diagnosed with epithelial ovarian carcinoma were obtained from The Cancer Genome Atlas. A distribution of allelotypes was then computed for each microsatellite locus for the ovarian cancer population.

Microsatellite variation was significantly higher in ovarian cancer patients relative to the exome equivalent in healthy females (1.4% in germline and tumor vs. 1.0% in 1 kGP females, p≦0.005). The WGS samples showed an even more distinct increase in microsatellite instability with ≧4% variation in ovarian cancer genomes vs. 1.5% in the normal females. A subset of 600 microsatellite loci was conserved in normal females yet had high levels of variation in either ovarian cancer germline DNA, tumors or both. These 600 loci constitute the initial set of informative loci (see loci 101-600 of Table 4). This subset was narrowed down to a set of 100 ‘ovarian cancer-associated loci’ using leave-one-out cross-validation (see loci 1-100 of Table 4).

Variations within the ovarian cancer-associated subset of loci were used to classify genomes as ‘normal’ or having an ‘ovarian cancer-signature’. It was determined that, in certain embodiments, a minimum of 4 variant loci in the ovarian cancer microsatellite subset could successfully classify genomes as having an ‘ovarian cancer signature’ with a specificity of 99.2% and a sensitivity of 46%. Accordingly, the disclosure contemplates methods in which at least 3, preferably at least 4, of the informative microsatellite loci set forth in Table 4 are evaluated. In certain embodiments, the at least 4 loci are selected from loci 1-100 in Table 4. In certain embodiments, the at least 4 loci are selected from loci 101-600 in Table 4.

The rate of ovarian cancer in a normal population is approximately 1/58 (1.7%), and we identified ˜50% of known ovarian cancer-patients as having an OV signature. Combined, these two factors make the expected detectable frequency of ovarian cancer within the normal population 0.8%, which is consistent with what was observed when requiring a minimum of 4 variant alleles within the OV-associated loci set.

The disclosure contemplates, in certain embodiments, methods of evaluating ovarian cancer predisposition, as well as prognostic and diagnostic methods, in which any one or more of the 100 informative ovarian cancer microsatellite loci set forth in Table 4 (e.g., 1, 2, 3, 4, 5, 6, 7, 8, 9, 10, more than 10, or even 100) are examined in a patient (e.g., in a particular patient in need of evaluation). In certain embodiments, 3, 4, 5, or 6 loci are analyzed. In certain embodiments, 4 loci are evaluated. In certain embodiments, in addition to analyzing one or more of the 100 informative ovarian cancer microsatellite loci set forth in Table 3, one or more (e.g., 1, 2, 3, 4, 5, 6, 7, 8, 9, 10, more than 10, or even 500) additional loci selected from the remaining 500 loci initially identified as informative using less stringent selection criteria are analyzed.

As detailed herein, GMI instability and/or informative microsatellite loci can be used in a variety of prognostic and diagnostic methods. The disclosure contemplates that, for example, any one or more of the informative loci discussed herein or set forth in the figures and tables can be used in diagnostic and prognostic methods.

Glioblastoma Multiforme

Glioblastoma Multiforme (GBM) is a rapidly growing, malignant brain tumor that is the most common brain tumor in adults. In 2010, more than 22,000 Americans were estimated to have been diagnosed and 13,140 were estimated to have died from brain and other nervous system cancers. GBM accounts for about 15 percent of all brain tumors and occurs in adults between the ages of 45 to 70 years. Patients with GBM have a poor prognosis and usually survive less than 15 months following diagnosis. Currently there are no effective long-term treatments for this disease. The lifetime risk of developing a brain cancer is 0.65% in men and 0.5% in women.

The most common and aggressive brain tumors are glioblastoma multiforme (GBM; astrocytoma IV). There are three main groups of adult gliomas which can become GBM: astrocytoma (A); oligodendroglioma (OD) which are slower-growing but rarely progress to GBM; and mixed glioma such as oligoastrocytomas (OA), a mix of A and OD.

Astrocytoma is graded from I to IV according to the World Health Organization's classification criteria and OD and OA come primarily in grades II and III. Lower grade adult astrocytomas can progress into higher grade tumors, upon reoccurrence. Treatment for Grade III and IV gliomas are similar; reoccurrence after therapy is common with A, OA, and some OD and is generally associated with progressively more aggressive and infiltrative tumors, with most neoplasms appearing at the original site of lesion. Grade II tumors are treated differently, with resection (if operable) and regular MRIs. Treatment for adult gliomas is largely ineffective, leading to 10,000 deaths annually, prompting The National Cancer Institute (NCI) to propose an initiative to increase 5-year GBM patient survival. A better understanding of glioma genomics is anticipated to lead to improved diagnostic and prognostic markers, as well as new therapeutic targets which could contribute to this goal. High-throughput sequencing studies of tumor genomes have produced new molecular markers that have enhanced classification of GBM and highlighted genes and molecular pathways that propagate GBM pathogenesis and disease progression. Clinical markers which could differentiate and confirm Grade II and IV gliomas prior to biopsy or surgery could vastly benefit therapy decisions, patient quality of life, and expand upon observations necessary to individualize treatment based on patient-specific risk assessment.

Identification of Informative Microsatellite Loci for GBM Using Allelotyping

For this analysis, a baseline variation was established by analyzing variation at a plurality of microsatellite locus normal brain tissue samples from the 1,000 Genome Project (1 kGP) dataset. After computing a distribution of allelotypes in the normal population, we asked whether there was an increase in the overall frequency of microsatellite variation in GBM samples. Next-generation sequencing data from GBM tumor and GBM non-tumor samples were obtained. A distribution of allelotypes was then computed for each microsatellite locus for the GMB samples. A comparison of the allelolype distribution obtained with the normal population to that obtained with the GMB samples identified 48 loci that varied between the two populations (Table 5; a first set of informative loci). Using the ‘leave-one-out’ statistical analysis method to determine which loci are most informative for properly assigning genomes to the correct cancer and non-cancer populations, 10 signature loci that contribute significantly (P≦0.05) to specificity and sensitivity in calling GBM positive samples were identified (e.g., highly informative loci).

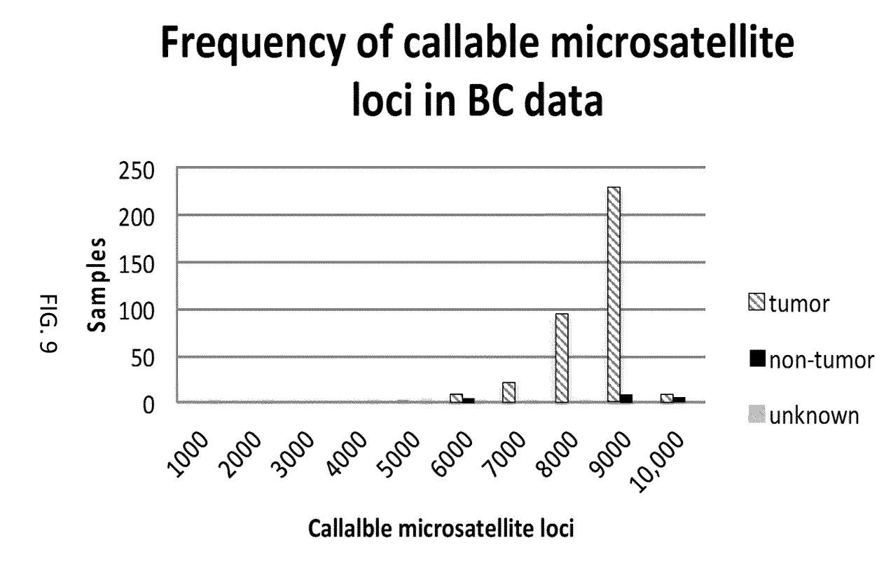

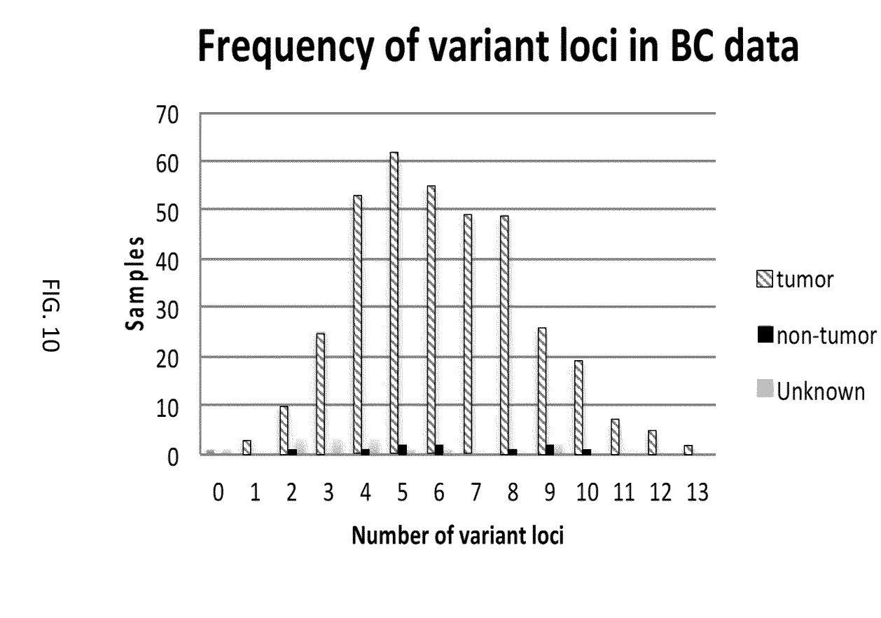

Through this unique analysis method, we determined that if 4 of the 48 informative loci with microsatellite variants were used to randomly identify GBM, 0% of normal samples would test positive while 29.4% of GBM tumors and 33.3% of germline, non-tumor GBM samples would test positive. Note, as above, the difference observed when assessing sensitivity in the GBM data sets (e.g., tumor nucleic acid versus germline nucleic acid) is a function of the difference in the number of samples and is not thought to reflect a statistically relevant difference in sensitivity between the two data sets. With just 3 of the informative loci, 1.6% of normal samples would test positive (false positive); however, 39.5% of tumor tissue and 69.7% of GBM non-tumor blood samples tested positive for these markers (Table 6). This demonstrates that microsatellite repeats are a predicative marker of GBM. Additionally, this demonstrates that microsatellite repeats could serve as a biomarker for GBM/cancer/disease in individuals before disease develops, since the signature microsatellite loci are present in germline samples and are not exclusive to tumors. These findings are discussed in more detail in FIG. 8.

Thus, the disclosure contemplates, in certain embodiments, methods of evaluating GBM predisposition, as well as prognostic and diagnostic methods in which any one or more of the microsatellite loci set forth in FIG. 8 (e.g., 1, 2, 3, 4, or 5) are examined in a patient (e.g., in a particular patient in need of evaluation). Moreover, the disclosure contemplates that analysis of 1, 2, 3, 4, or 5 of the loci set forth in FIG. 8 can be combined with analysis of any one or more (e.g., 1, 2, 3, 4, 5, 6, 7, 8, 9, 10, 11, 12, 13) of the loci set forth in Table 5.

Identification of Informative Microsatellite Loci for GBM and Lower-Grade Gliomas (LGG) Using Microsatellite Genotyping

For this analysis, Exome sequencing data, from Illumina HiSeq sequencing machines (an example of a Next Generation sequence platform) were obtained from The Cancer Genome Atlas (TCGA) and the 1000 Genomes Project (1 kGP). Only loci with sequencing reads with 15× or greater depth of coverage were used to identify possible informative loci. A profile or distribution of genotypes for the affected (TCGA) and unaffected (1 kGP) cohorts was then generated for each microsatellite locus. An allele is defined by a genomic locus with a specific microsatellite repeat and nucleotide sequence length, in each sample a pair of loci was identified and each allelic pair was then defined as a genotype. The genotype most prevalent from a distribution of genotypes was identified (called) in 1 kGP samples; this genotype was defined as the consensus or modal genotype (if more than a pair of alleles was identified for a locus that sample was not used).

Similar to the 1 kGP samples, LGG and GBM samples were analyzed for genotypes from the same genomic loci. Loci different from the consensus or between LGG and GBM and with differing frequency-of-occurrence were then called. The statistically significant genotypes were determined from data adjusted for false discovery rate (FDR), using a two-sided Fisher's p-test and Benjamini-Hochberg correction; relative risk (RR) was calculated for each locus and loci with a P≦0.01 were considered significant. Those genotypes, although individually informative, were also assembled into a ‘signature’ or ‘cancer-associated’ informative loci which together increase the statistical significance across all samples. This signature provides a PIM (panel of informative microsatellites) for each of these cancer types.

The number of informative loci that passed the statistical tests that differentiated cancer-associated from “healthy” included 48 loci for GBM (Table 17) and 66 loci for LGG (Table 18); of these, 10 of the signature loci in GBM overlapped with those in the LGG signature.

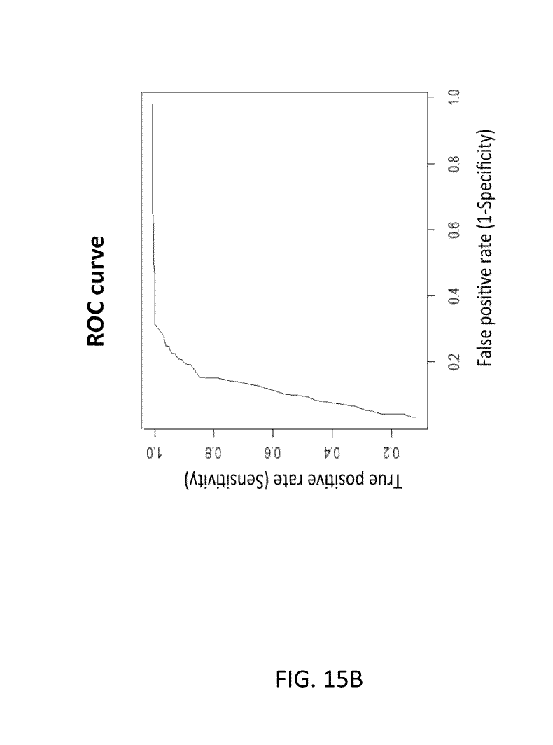

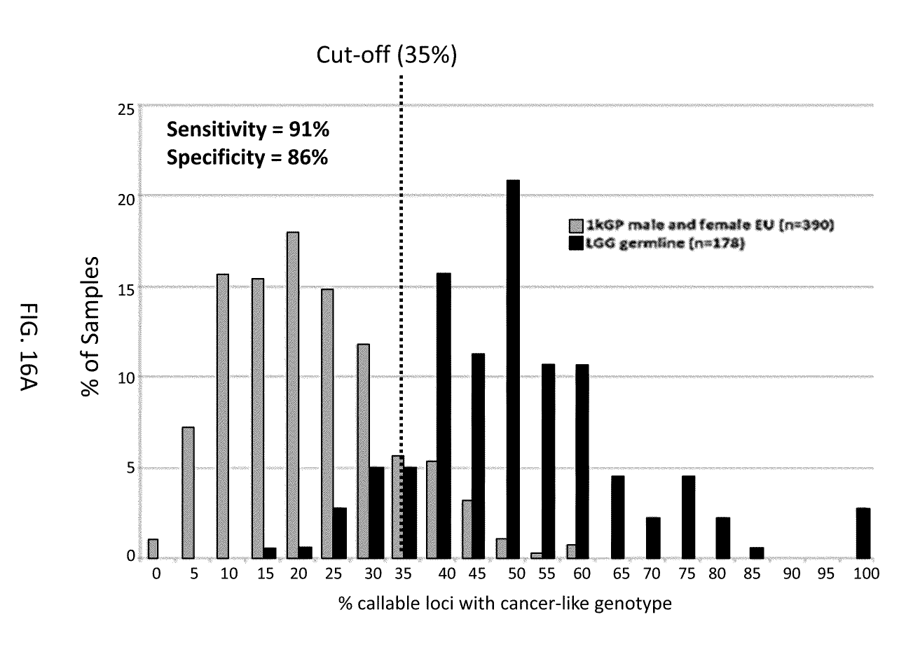

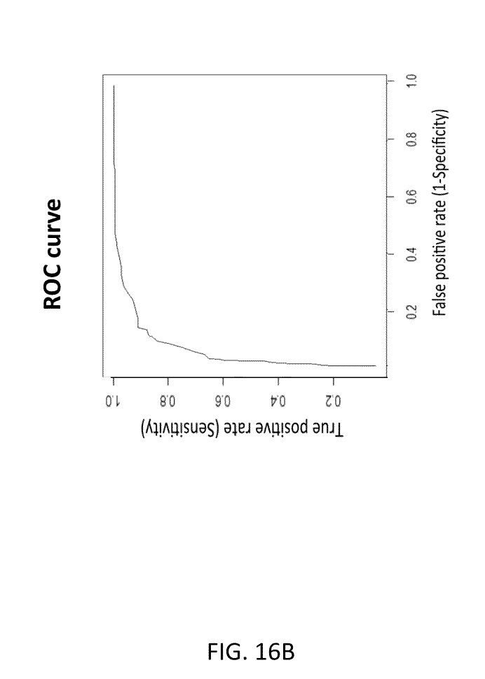

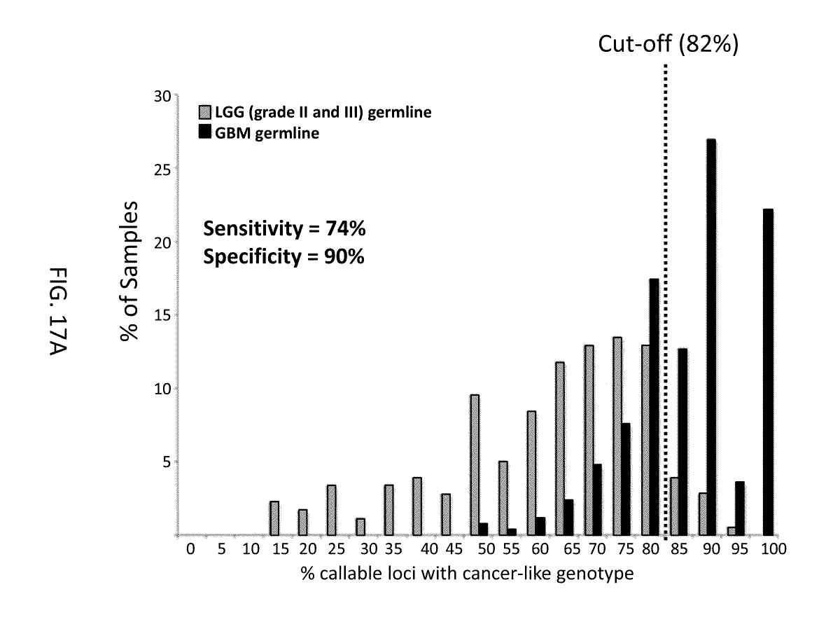

Using the false positive and false negative rates within the training set, we were able to determine the receiver operating characteristic (ROC) for the 66 LGG and 48 GBM loci. Through maximizing the area under the ROC curve, we determined that the optimal cut-off classifier for GBM was 57%, that is, at least 57% of the callable 48 GBM loci matching the GBM-like profile (FIG. 15) (e.g., 57% of callable loci having a genotype that differs from the reference, healthy modal genotype or the sample data best fits the cancer-like distribution). We were then able to classify the GBM samples as GBM-like (≧57%) or healthy (<57%) with a sensitivity of 94%, and a specificity of 77% (FIG. 15). As to LGG, we determined that the cut-off was 35%, that is, at least 35% of the callable 66 LGG loci matching the LGG-like profile (FIG. 16) (e.g., 35% of callable loci having a genotype that differs from the reference, healthy modal genotype or the sample data best fits the cancer-like distribution). We were then able to classify the LGG samples as LGG-like (≧35%) or healthy (<35%) with a sensitivity of 91%, and a specificity of 86% (FIG. 16). The number of callable genotypes will depend on many factors, such as the quality of reads, the number of reads required for inclusion, and the quality of alignment tools for evaluating the sequencing data. Examples of the percentages of callable loci contemplated are provided below.

Thus, the disclosure contemplates methods of evaluating GBM predisposition, as well as prognostic and diagnostic methods, comprising genotyping at least 5%, 10%, 20%, 30%, 40%, 50%, 60%, 70%, 80%, 80% or 100% of the 48 GBM informative loci from Table 17. Alternatively, the method may comprise genotyping at least 2, 5, 10, 15, 20, 25, 30, 35, 40, 45 or all of the GBM loci from Table 17. In some embodiments, the patient is identified as having an increased risk of developing cancer if at least 57% of the genotyped GBM loci from Table 17 have a GBM-like genotype (e.g., have a genotype that differs from the modal genotype of a healthy, reference population or the sample data best fits the cancer-like distribution). In some embodiments, the patient is identified as having an increased risk of developing GBM if at least 10%, 15%, 20%, 25%, 30%, 35%, 40%, 45%, 50%, 55%, or 60% of the genotyped GBM loci (the callable loci) from Table 17 have a GBM-like genotype.

The disclosure also contemplates methods of evaluating LGG predisposition, as well as prognostic and diagnostic methods, comprising genotyping at least 5%, 10%, 20%, 30%, 40%, 50%, 60%, 70%, 80%, 80% or 100% of the 66 LGG informative loci from Table 18. Alternatively, the method may comprise genotyping at least 2, 5, 10, 15, 20, 25, 30, 35, 40, 45, 50, 55, 60 or all of the LGG loci from Table 18. In some embodiments, the patient is identified as having an increased risk of developing cancer if at least 35% of the genotyped LGG loci from Table 18 have a LGG-like genotype (e.g., have a genotype that differs from the modal genotype of a healthy, reference population or the sample data best fits the cancer-like distribution). In some embodiments, the patient is identified as having an increased risk of developing LGG if at least 5%, 10%, 15%, 20%, 25%, 30%, 35%, or 40% of the genotyped LGG loci (the callable loci) from Table 18 have a LGG-like genotype.

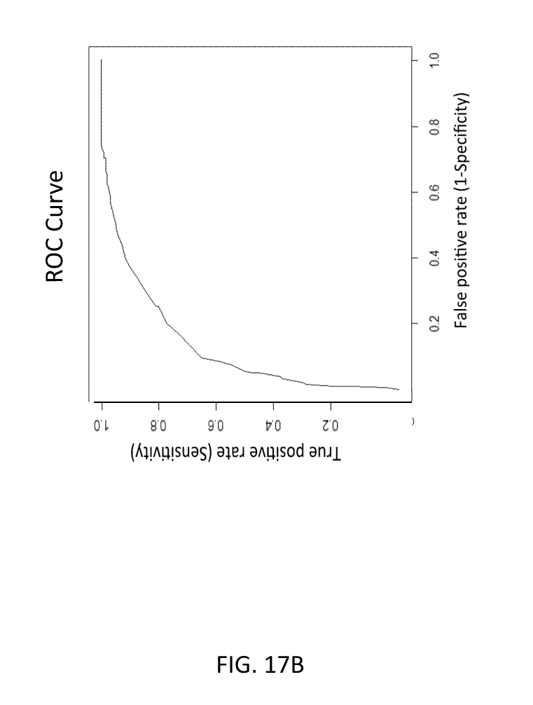

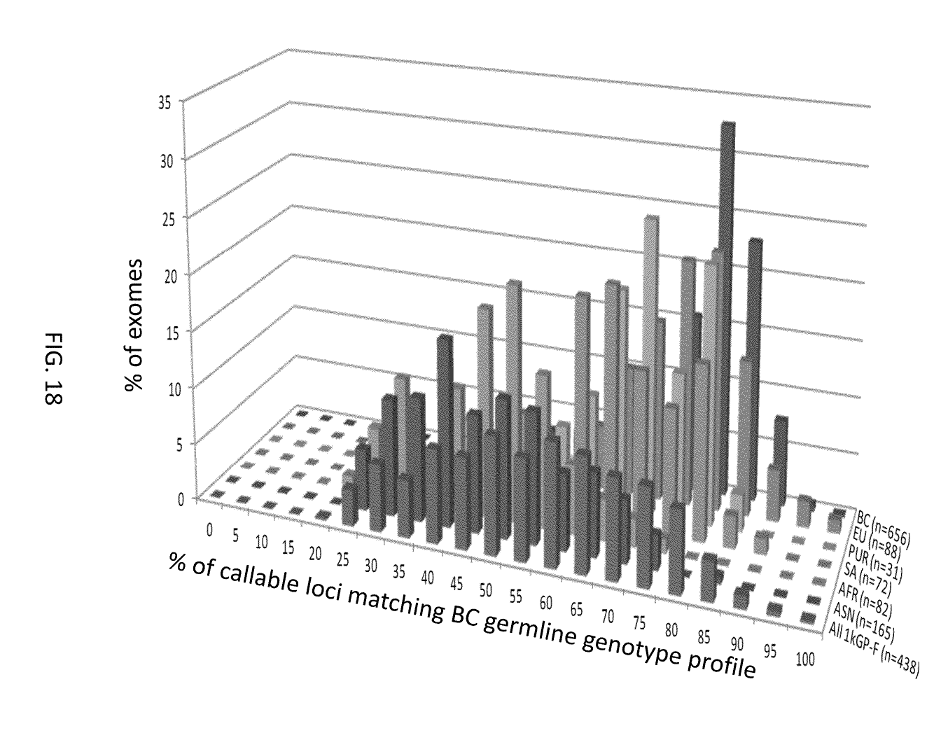

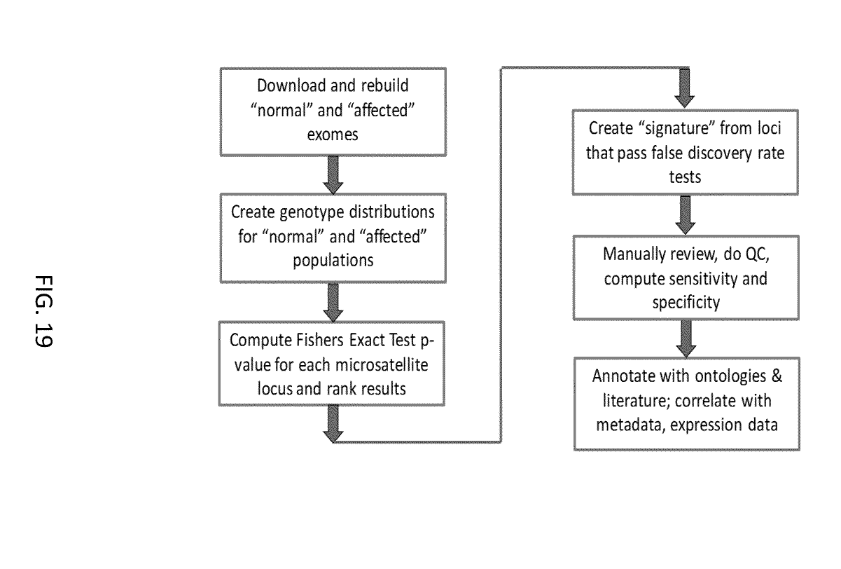

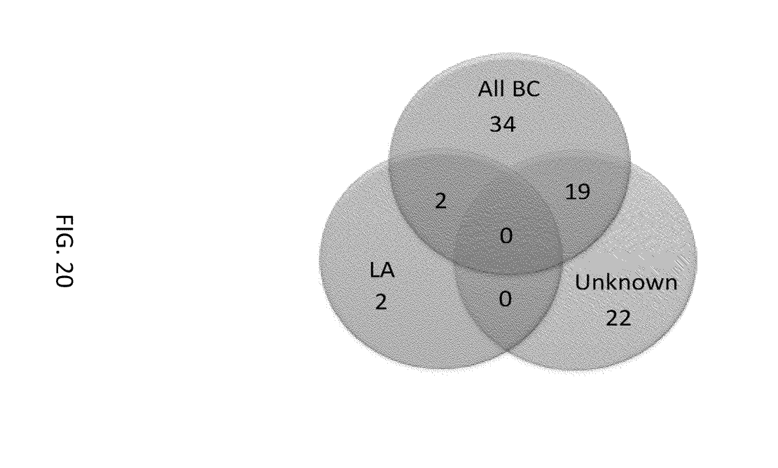

Additionally, we compared LGG and GBM germlines and discovered 26 signature loci that were unique to GBM as compared to LGG (Table 19). Specifically, these loci were determined by computing modal genotypes at microsatellite loci in the LGG population and comparing the genotypes for the same loci in the GBM population (e.g., the LGG population was used as the reference population). We then measured the percentage of samples (GBM and LGG) with these genotypes. We were able to classify the GBM samples (≧82% of callable microsatellite loci have non-modal genotype) or LGG samples (<82% of callable microsatellite loci have non-modal genotype) with a sensitivity of 74%, and a specificity of 90% (FIG. 17). These markers are thus selective biomarkers able to differentiate LGG from GBM.

The disclosure thus contemplates methods of distinguishing LGG from GBM, such as in a subject suspected of having a brain lesion, comprising genotyping at least 5%, 10%, 20%, 30%, 40%, 50%, 60%, 70%, 80%, 80% or 100% of the 27 GBM informative loci from Table 19 in the subject. Alternatively, the method may comprise genotyping at least 2, 5, 10, 15, 20, 25 or all GBM loci from Table 19 in the subject. In some embodiments, a patient is identified as having GBM if at least 82% of the callable, genotyped loci from Table 19 have a GBM-like genotype (e.g., have a genotype that differs from the modal genotype of a LGG reference population). In some embodiments, the patient is identified as having GBM if at least 50%, 55%, 60%, 65%, 70%, 75%, 80%, 85%, 90%, 95% or 100% of the genotyped loci (callable genotyped loci) from Table 19 have a GBM-like genotype (e.g., have a genotype that differs from the modal genotype of a LGG reference population).

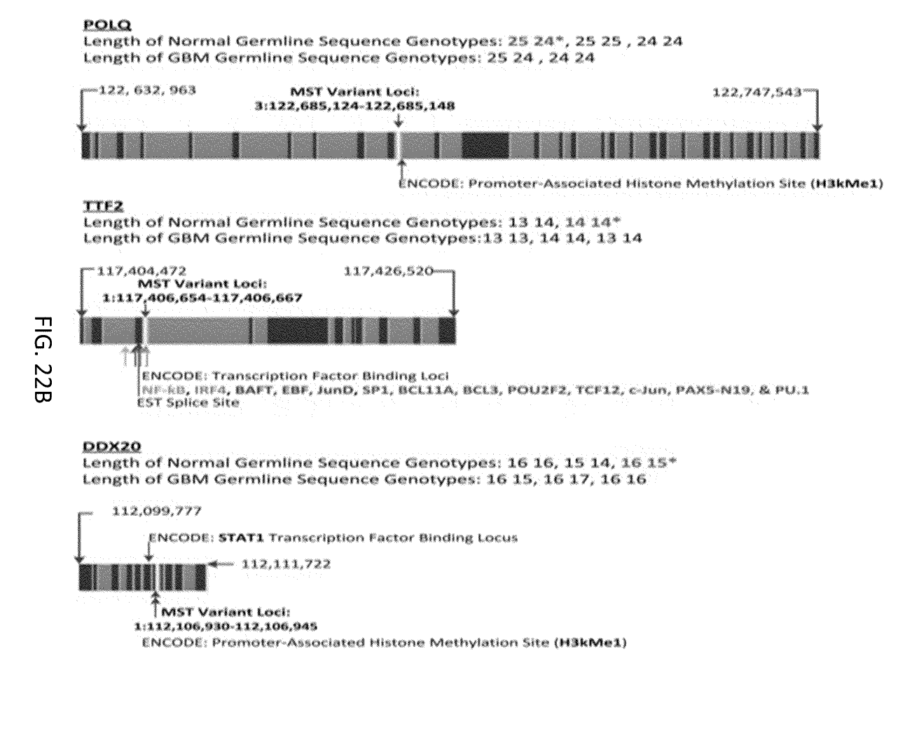

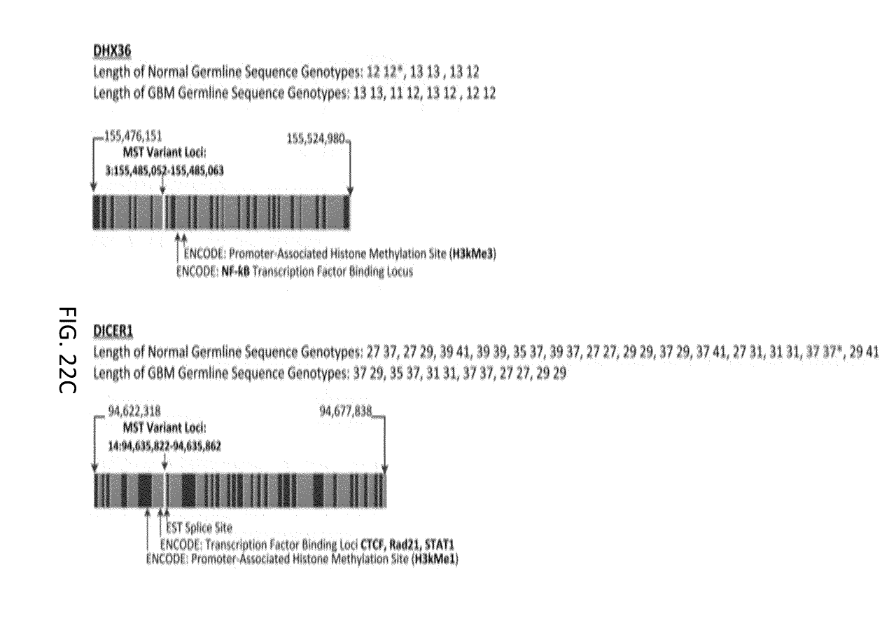

Additionally, we compared LGG Grade II and GBM germlines. Our results identified 8 signature loci that were unique to GBM as compared to LGG Grade II (Table 20). Specifically, these loci were determined by computing modal genotypes at microsatellite loci in the LGG grade II population and comparing the genotypes for the same loci in the GBM population. We were able to classify the GBM (≧85% of callable microsatellite loci have non-modal genotype—where the reference population is the LGG Grade II modal genotype) samples or LGG samples (<85% of callable microsatellite loci have non-modal genotype) with a sensitivity of 90%, and a specificity of 70% (FIG. 21). These markers are thus selective biomarkers able to distinguish LGG Grade II from GBM.

Thus, the disclosure contemplates methods of distinguishing LGG grade II from GBM, in a patient suspected of having a brain lesion, comprising genotyping at least 5%, 10%, 20%, 30%, 40%, 50%, 60%, 70%, 80%, 80% or 100% of the 8 loci from Table 19. Alternatively, the method may comprise genotyping at least 1, 2, 3, 4, 5, 6, 7, or 8 of the loci from Table 19. In some embodiments, the patient is identified as having GBM if at least 85% of the genotyped loci from Table 19 have a GBM-like genotype (e.g., have a genotype that differs from the modal genotype of a LGG reference population). In some embodiments, the patient is identified as having GBM if at least 50%, 55%, 60%, 65%, 70%, 75%, 80%, 85%, 90%, 95% or 100% of the genotyped loci from Table 19 have a GBM-like genotype (e.g., have a genotype that differs from the modal genotype of a LGG reference population).

The foregoing microsatellites are particularly useful for distinguishing between GBM and low grade glioma. Evaluating genotype for these microsatellite loci may be used to help distinguish, without the need for an invasive brain biopsy, whether a patient suspected of having a brain lesion is likely to have GBM or is likely to have a much less aggressive cancer. This provides a mechanism for evaluating risk that the patient has GBM before initiating highly invasive and dangerous diagnostic and therapeutic interventions.

Comparing adult gliomas we identified distinct populations of variant DNA microsatellite loci unique to LGG and GBM. Several loci identified are associated with genes important to early neuronal development, progenitor cell development, and neuronal cell differentiation—which are often exploited in cancer cell proliferation (including, FRMD7, FUBP3, NEO1, DIP2B, LNX2, OFD1, SRC (which interacts with ESR1, CBL (a signature loci), EGFR, BCAR1, STAT3 and several other transcription regulators), NBPF1, MYCBP2, KIF1B, KLAQ1, and BEND2 (BEND domains are found in proteins which interact with DNA, including chromatin restructuring and transcription, including alternative splicing) from GBM or LGG. The heterogeneity of glioma types that compose the LGG samples may contribute to the broader spectrum of cancer-associated loci in LGG, relative to GBM samples. This suggests that for GBM, or disease progression to GBM, microsatellite genotypes that are cancer-associated may be more conservative.

The aberrant alteration of six helicases (DICER1, DDX20, DDX60, DHX36, POLQ, and TTF) in GBM suggests that genes important to microsatellite identification and removal (POLQ), along with transcription and RNA synthesis (TTF2, DHX36, DDX20, and DICER1 from GBM; SSX, YTHDC2, and DDX20 from LGG) are themselves modified with MST variants. As such, one mechanism may be that GBM tumors produce atypical RNA in-part due to these variants which otherwise promote RNA degradation. This is further supported by the enrichment of MST variant loci in helicase genes activated through interferon (DDX60, TRIM25, TTF2, and DICER1); interferon can initiate helicases and ubiquitin ligases to degrade viral RNAs and other dsRNAs. However, if these genes are themselves modified, recognition of alternative RNAs may be altered. A second cancer promoting modification (including those in DDX20, NSUN5, DICER1, or NUFIP1 from GBM; RBM5 from LGG), prompted by these variants may introduce changes to gene-products that compose spliceosome complexes (snRNA, snRNP, or snoRNP); through these modifications, alternatively spliced RNA could support spliceosome-associated proteins differently, which may further modify mature RNAs. A third system is modifications to ubiquitin proteasome system proteins (ligases and ubiquitin complex proteins) which could alter protein degradation or signal transduction (including, ATG3, PSME3, and especially E3 ligases-TRIM25, TRIML1, DDX60, and CBL in GBM; MYCBP2, UBXN7, KLHL3, NCAPD3, CDC16, and C8orf38 in LGG). Exploiting these inherent cell-signaling mechanisms could promote tumorogenesis by changes in methylation of DNA and RNA, histone proteins, and tyrosine kinase activity. A supplementary mechanism may be that genes with repeat sequences are more susceptible to repeat modifications in introns or ‘fragile-sites’, in addition to exon sequences—as evidenced in DIP2B and BRWD2. Previous studies on repeats within FMR1 demonstrate that different repeat lengths can produce diverse disease phenotypes. We repeatedly see the same genes in differing diseases and with MST-specific genetic perturbations which contribute to disease differently. This further supports the possibility of stem cells with aberrant genetic modifications that produce disease relative to the combination, type, and abundance of effected microsatellite loci.

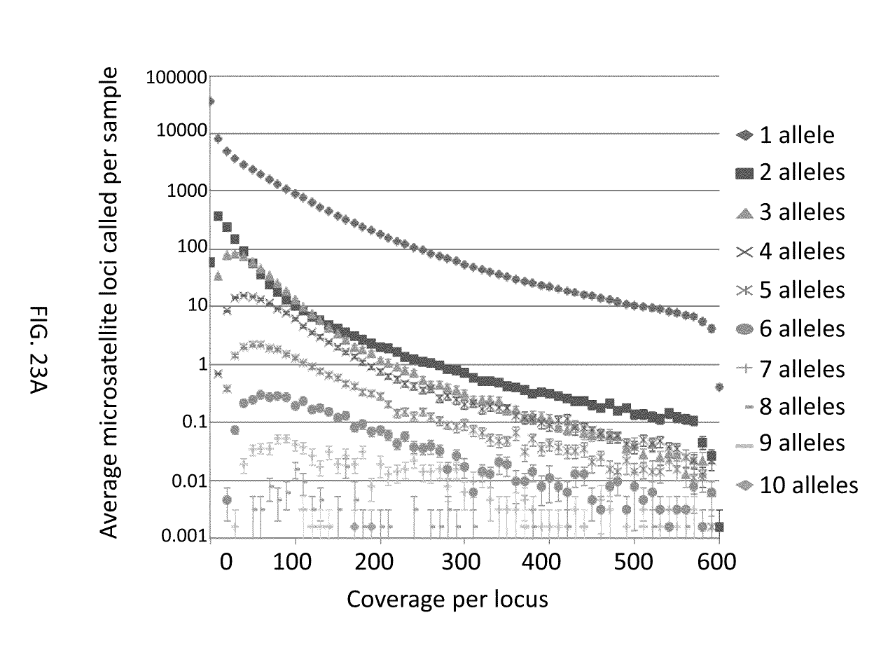

FIG. 22A-C is a depiction of the helicase variants DHX36, DICER1, TTF2, DDX20, POLQ and DDX60. At the location of each variant we have described significant genomic elements, including: histone methylation markers described through ENCODE (H3kMe3 or H3kMel), transcription factor binding loci or exon splice sites (ESTs). The total length of the gene and the microsatellite loci are described with exons; also provided are the lengths of those microsatellite allelic pairs (genotypes) from normal and GBM germlines, with the consensus denoted (denoted by *). The location of these microsatellite variants could change gene/exon transcription or expression due to their location near histone methylation markers, transcription factors, and splice sites. These changes could modify the abundance of these proteins or introduce phenotypic changes that may modify their function (although non-coding, if the MST are near splice sites); these changes will be relative to (1) the location of the variant (2) the genomic regulatory elements linked with the variant loci (3) the importance of the gene-region at which the variant is located.

Given that these cancer-associated microsatellites are identifiable in somatic DNA and the loci are conserved in tumors lends to the hypothesis that glioma stem-cell populations would exist and are inherent to the individual and their disease. Microsatellite loci are different in GBM, LGG, and normal germline samples. Thus, modification to gene sequences by MST variants could be an inherent mechanism exploited by cancer cells that contributes to their survival via alternative signaling mechanisms associated to ubiquitin conjugated pathways, changes to spliceosome complexes, helicases, cell cycle, signaling, mobility, and metabolism; collectively, a monumental set of cellular modifications. Variation at these loci are predictable therefore, it is less likely the result of “random” events and could potentially be viewed as a purposefully exploited mechanism where defects in synonymous replication or transcription machinery are used by cancer cells to evolve and establish a tissue specific community. If so, we could predict that global microsatellite instability contributes to cancer-specific genomics and occurs during embryogenesis which has also been predicted in other MST associated diseases including Huntington's disease and Fragile X syndrome.

We have observed microsatellite instability in or near genes associated with DNA replication, transcription, mRNA splice variants- and more so genes with protective functions, such as helicases, tumor suppressors, or ubiquitin proteasome system—this would suggest that microsatellites contribute to the acceleration of glioma cell adaptability versus a mechanism that causes normal cell function to run awry. Therefore, we further hypothesize that DNA microsatellite variability are a mechanism for adaptability that is conserved in all cancers—by which we should be able to identify and measure the frequency of (1) those genes that are essential for cancer cell survival (and conserved across a cancer type) (2) contribute intermittently—to cancer cell phenotypes like metastasis, heterogeneity, or aggressiveness, and (3) tissue-specificity, those genes associated with only one type of tumor or tissue origin. Additionally, we predict that with such a mechanism at play—stem cells are the source of these cancer-associated microsatellite loci, as evidence by germline-specific biomarkers for LGG and GBM.

Colon Cancer

To identify informative biomarkers for colon cancer, the GMI profiles of normal individuals from the 1000 Genome Project were compared to the GMI profiles of individuals with colon cancer. Table 7 provides information about the informative microsatellite loci identified in this analysis.

The disclosure contemplates, in certain embodiments, methods of evaluating colon cancer predisposition, as well as prognostic and diagnostic methods, in which any one or more of the informative colon cancer microsatellite loci set forth in Table 7 (e.g., 1, 2, 3, 4, 5, 6, 7, 8, 9, 10, more than 10, etc.) are examined in a patient (e.g., in a particular patient in need of evaluation).

Lung Cancer

To identify informative biomarkers for colon cancer, the GMI profiles of normal individuals from the 1000 Genome Project were compared to the GMI profiles of individuals with lung cancer. Tables 8 and 9 provide information about the informative microsatellite loci identified in this analysis.

The disclosure contemplates, in certain embodiments, methods of evaluating lung cancer predisposition, as well as prognostic and diagnostic methods, in which any one or more of the informative lung cancer microsatellite loci set forth in Table 8 or Table 9 (e.g., 1, 2, 3, 4, 5, 6, 7, 8, 9, 10, more than 10, etc.) are examined in a patient (e.g., in a particular patient in need of evaluation).

Prostate Cancer

To identify informative biomarkers for colon cancer, the GMI profiles of normal individuals from the 1000 Genome Project were compared to the GMI profiles of individuals with prostate cancer. Table 10 provides information about the informative microsatellite loci identified in this analysis.

The disclosure contemplates, in certain embodiments, methods of evaluating prostate cancer predisposition, as well as prognostic and diagnostic methods, in which any one or more of the informative prostate cancer microsatellite loci set forth in Table 10 (e.g., 1, 2, 3, 4, 5, 6, 7, 8, 9, 10, more than 10, etc.) are examined in a patient (e.g., in a particular patient in need of evaluation).

### 4. Disease Diagnosis and Predisposition Screening

The present disclosure provides methods and systems by which one can effectively identify informative microsatellite loci which correlate with specific conditions. The identification of informative microsatellite loci can be exploited in several ways. For example, in the case of a highly statistically significant association between one or more informative microsatellite loci with predisposition to a disease for which treatment is available, detection of one or more informative microsatellite loci in an individual may justify immediate administration of treatment or at least the institution of regular monitoring of the individual which exceeds the level of routine monitoring typically recommended for a subject of similar age and gender. Detection of the informative microsatellite loci associated with serious disease in a couple contemplating having children may also be valuable to the couple in their reproductive decisions. In the case of a weaker but still statistically significant association between an informative microsatellite loci and a human disease, immediate therapeutic intervention or monitoring may not be justified after detecting the informative microsatellite loci. Nevertheless, the subject can be motivated to begin simple life-style changes (e.g., diet, exercise) that can be accomplished at little or no cost to the individual but would confer potential benefits in reducing the risk of developing conditions for which that individual may have an increased risk by virtue of having the informative microsatellite allele(s). Moreover, even for individuals in which analysis of microsatellite profile indicates a relatively low risk, increased monitoring may be instituted.

The informative microsatellite loci of the present disclosure may contribute to disease in an individual in different ways. Some microsatellite polymorphisms occur within a protein coding sequence and contribute to disease phenotype by affecting protein structure. Other polymorphisms occur in noncoding regions but may exert phenotypic effects indirectly via influence on, for example, replication, transcription, translation, splicing and post-transcriptional modification. A single microsatellite variation may affect more than one phenotypic trait. Likewise, a single phenotypic trait may be affected by multiple microsatellite variations in different genes.

As used herein, the terms “diagnose”, “diagnosis”, and “diagnostics” include, but are not limited to any of the following: detection of disease that an individual may presently have, predisposition/susceptibility screening (i.e., determining the increased risk of an individual in developing the disease in the future, or determining whether an individual has a decreased risk of developing the disease in the future, determining a particular type or subclass of disease in an individual known to have the disease, confirming or reinforcing a previously made diagnosis of the disease, pharmacogenomic evaluation of an individual to determine which therapeutic strategy that individual is most likely to positively respond to or to predict whether a patient is likely to respond to a particular treatment, predicting whether a patient is likely to experience toxic effects from a particular treatment or therapeutic compound, and evaluating the future prognosis of an individual having the disease. Such diagnostic uses are based on the microsatellite profile of the individual.

“Risk evaluation,” or “evaluation of risk” in the context of the present disclosure encompasses making a prediction of the probability, odds, or likelihood that an event or disease state may occur, the rate of occurrence of the event or conversion from one disease state to another, i.e., from a primary tumor to a metastatic tumor or to one at risk of developing a metastatic, or from at risk of a primary metastatic event to a secondary metastatic event or from at risk of a developing a primary tumor of one type to developing a one or more primary tumors of a different type. Risk evaluation can also comprise prediction of future clinical parameters, traditional laboratory risk factor values, or other indices of cancer, either in absolute or relative terms in reference to a previously measured population.

It will, of course, be understood by practitioners skilled in the treatment or diagnosis of a disease that, in certain embodiments, the present disclosure does not provide an absolute identification of individuals who are at risk (or less at risk) of developing cancer, and/or pathologies related to cancer, but rather to indicate a certain increased (or decreased) degree or likelihood of developing the disease based on statistically significant association results. However, this information is extremely valuable as it can be used to, for example, initiate preventive treatments or to allow an individual carrying one or more significant informative microsatellite loci combinations to foresee warning signs such as minor clinical symptoms, or to have regularly scheduled physical exams to monitor for appearance of a condition in order to identify and begin treatment of the condition at an early stage. Particularly with types of cancers that are fatal if not treated on time, the knowledge of a potential predisposition, even if this predisposition is not absolute, would likely contribute in a very significant manner to treatment efficacy. In certain embodiments, an individual is already suspected of having a disease or condition, and examination of microsatellite loci can be used as a further diagnostic measure. The diagnostic value of the instant methods is particularly useful because the informative microsatellite loci can be evaluated in simple blood or cheek-swab samples. In the case of cancer, this permits analysis before a tumor or other lesion is detectable or present and, even when a lesion is present, permits evaluation non-invasively or minimally invasively. This is a significant advantage, particularly where obtaining a tumor sample itself involves significant risk to the patient.

As described herein, a diagnostic method may be based on the detection of single informative microsatellite locus or a group of informative microsatellite loci. Combined detection of a plurality of microsatellite loci (for example, 2, 3, 4, 5, 6, 7, 8, 9, 10, 11, 12, 13, 14, 15, 16, 17, 18, 19, 20, 24, 25, 30, 32, 48, 50, 64, 96, 100, or any other number in-between, or more, of the microsatellite loci provided in Tables 1-10, 14, 17-22 may increase accuracy. In certain embodiments, the method comprises evaluating at least 25%, at least 30%, at least 35%, at least 40%, or at least 50% of a set of informative microsatellite loci.

However, a person of reasonable skill in the art will recognize that depending on the loci combination, the sensitivity and/or specificity of the method may vary. Sensitivity refers to the ability of a method of the present disclosure to correctly identify an individual at increased risk of developing the disease and/or diagnosing an individual of the disease. More precisely, sensitivity is defined as True Positives/(True Positives+False Negatives). A test with high sensitivity has few false negative results, while a test with low sensitivity has many false negative results. In particular embodiments, the combination of microsatellite loci has a sensitivity of least about: 40, 50, 60, 70, 80, 90, 91, 92, 93, 94, 95, 96, 97, 98, 99, or 100%, or a sensitivity falling in a range with any of these values as endpoints.

Specificity, on the other hand, refers to the ability of a method of the present disclosure to give a negative result when risk and/or disease is not present. More precisely, specificity is defined as True Negatives/(True Negatives+False Positives). A test with high specificity has few false positive results, while a test with a low specificity has many false positive results. In certain embodiments, the combination microsatellite loci has a specificity of at about: 40, 50, 60, 70, 80, 90, 91, 92, 93, 94, 95, 96, 97, 98, 99, or 100%, or a specificity falling in a range with any of these values as endpoints. The disclosure contemplates methods in which the number and choice of microsatellite loci evaluated is selected to achieve a particular level of sensitivity and specificity, including any combination of any of the foregoing levels of sensitivity and specificity.

In general, microsatellite loci combinations with the highest combined sensitivity and specificity to correctly identify an individual at increased risk of developing a disease and/or diagnosing an individual of cancer are preferred. In exemplary embodiments the combination of microsatellite loci has a sensitivity and specificity of at least about: 40% and 90%, 45% and 90%, 50% and 90%, 60% and 90%, 70% and 90%, 80% and 90%, 90% and 90%, 95% and 95%, 99% and 99%, 100% and 100% respectively, or any combination of sensitivity and specificity based on the values given above for each of these parameters.

There is no limit to the number of informative microsatellite loci that can be employed in a combination. For example, 2 informative microsatellite loci selected from the microsatellite loci in Tables 1-10, 14, 17-22 can be combined. Alternatively, at least 3, 5, 6, 7, 8, 9, 10, 20, 30, 40, 50 informative microsatellite loci selected from the microsatellite loci in Tables 1-10, 14, 17-22 can be combined. It will be understood that the particular loci selected from analysis are based on, for example, the condition for which predisposition or diagnosis is being performed. Thus, if breast cancer predisposition is being performed, the informative microsatellite loci are selected from the loci set forth in Table 1 and/or 2. Of course, one or more of such loci can be combined with other loci or even combined with GMI analysis. However, at least one of the analyzed loci is selected from the loci set forth in Table 1 or 2. Similarly, if ovarian cancer predisposition is being performed, the informative microsatellite loci are selected from the loci set forth in Table 4. Of course, one or more of such loci can be combined with other loci or even combined with GMI analysis. However, at least one of the analyzed loci is selected from the loci set forth in Table 4.

Generally, the sensitivity of an assay increases as the number of informative microsatellite loci in a set increases. However, increasing the number of microsatellite loci in a combination may decrease the specificity of the method. Accordingly, a microsatellite loci combination for use in the methods of the present disclosure typically includes two, three, or four informative microsatellite loci, as necessary to provide optimal balance between sensitivity and specificity.

In some embodiments, a diagnostic method comprises detecting variations at microsatellite loci selected from the group consisting of microsatellite loci 1-100 set forth in Table 4. The disclosure contemplates, in certain embodiments, methods of evaluating ovarian cancer predisposition, as well as prognostic and diagnostic methods, in which any one or more of the 100 informative ovarian cancer microsatellite loci set forth in Table 3 (e.g., 1, 2, 3, 4, 5, 6, 7, 8, 9, 10, more than 10, or even 100) are examined in a patient (e.g., in a particular patient in need of evaluation). In certain embodiments, 3, 4, 5, or 6 loci are analyzed. In certain embodiments, 4 loci are evaluated. In certain embodiments, in addition to analyzing one or more of the 100 informative ovarian cancer microsatellite loci set forth in Table 3, one or more (e.g., 1, 2, 3, 4, 5, 6, 7, 8, 9, 10, more than 10, or even 500) additional loci selected from the remaining 500 loci initially identified as informative using less stringent selection criteria are analyzed.

In some embodiments, the method comprises detecting variations at microsatellite loci selected from the group consisting of the microsatellite loci set forth in Table 2. The disclosure contemplates, in certain embodiments, methods of evaluating breast cancer predisposition, as well as prognostic and diagnostic methods in which any one or more of the microsatellite loci set forth in FIG. 7 (e.g., 1, 2, 3, 4, or 5) are examined in a patient (e.g., in a particular patient in need of evaluation). Moreover, the disclosure contemplates that analysis of 1, 2, 3, 4, or 5 of the loci set forth in FIG. 7 can be combined with analysis of any one or more (e.g., 1, 2, 3, 4, 5, 6, 7, 8, 9, 10, 11, 12, 13) of the loci set forth in Table 2 and/or any one or more (e.g., 1, 2, 3, 4, 5, 6, 7, 8, 9, 10, 11, 12, 13, 14, 15, more than 15) of the loci set forth in Table 1.

In some embodiments, the method comprises detecting variations at microsatellite loci selected from the group consisting of the microsatellite loci set forth in Table 5. The disclosure contemplates, in certain embodiments, methods of evaluating glioblastoma predisposition, as well as prognostic and diagnostic methods in which any one or more of the microsatellite loci set forth in FIG. 8 (e.g., 1, 2, 3, 4, or 5) are examined in a patient (e.g., in a particular patient in need of evaluation). Moreover, the disclosure contemplates that analysis of 1, 2, 3, 4, or 5 of the loci set forth in FIG. 8 can be combined with analysis of any one or more (e.g., 1, 2, 3, 4, 5, 6, 7, 8, 9, 10, 11, 12, 13) of the loci set forth in Table 5.

In some embodiments, the method comprises detecting variations at microsatellite loci selected from the group consisting of the microsatellite loci set forth in Table 7. The disclosure contemplates, in certain embodiments, methods of evaluating colon cancer predisposition, as well as prognostic and diagnostic methods, in which any one or more of the informative colon cancer microsatellite loci set forth in Table 7 (e.g., 1, 2, 3, 4, 5, 6, 7, 8, 9, 10, more than 10, etc.) are examined in a patient (e.g., in a particular patient in need of evaluation).

In some embodiments, the method comprises detecting variations at microsatellite loci selected from the group consisting of the microsatellite loci set forth in Table 8 or 9. The disclosure contemplates, in certain embodiments, methods of evaluating lung cancer predisposition, as well as prognostic and diagnostic methods, in which any one or more of the informative lung cancer microsatellite loci set forth in Table 8 or Table 9 (e.g., 1, 2, 3, 4, 5, 6, 7, 8, 9, 10, more than 10, etc.) are examined in a patient (e.g., in a particular patient in need of evaluation).

In some embodiments, the method comprises detecting variations at microsatellite loci selected from the group consisting of the microsatellite loci set forth in Table 10. The disclosure contemplates, in certain embodiments, methods of evaluating prostate cancer predisposition, as well as prognostic and diagnostic methods, in which any one or more of the informative prostate cancer microsatellite loci set forth in Table 10 (e.g., 1, 2, 3, 4, 5, 6, 7, 8, 9, 10, more than 10, etc.) are examined in a patient (e.g., in a particular patient in need of evaluation).

The disclosure also contemplates, in certain embodiments, methods of evaluating breast cancer predisposition, as well as prognostic and diagnostic methods in which any one or more of microsatellite loci set forth in Tables 14 and 15 are evaluated. In a preferred embodiment, the method is one that evaluates breast cancer predisposition, comprising genotyping at least one of the loci in Table 14 having a relative risk of >1.3 or <0.6. Relative risk is calculated as the percent of individuals with the non-modal genotype from the cancer population divided by the percent of individuals with the non-modal genotype in the non-cancer population. Variation at any one or more of the loci having a relative risk of >1.1, 1.2 or 1.3 may be indicative of an increased risk of developing cancer. Variation at one any one or more of the loci having a relative risk of <0.9, 0.8, 0.7 or 0.6 may be indicative of a lowered risk of developing cancer (a protective loci). In some embodiments, the relative risks are weighted in the analysis. In some embodiments, the depth coverage of each loci is weighted in the analysis. In some embodiments, the presence of minor alleles is weighted in the analysis. In another preferred embodiment, the method is one that evaluates breast cancer predisposition, comprising genotyping at least 5%, 10%, 20%, 30%, 40%, 50%, 60%, 70%, or 80% of the loci listed in Table 14 in a subject. Alternatively, the method may comprise genotyping at least 2, 4, 5, 6, 7, 8, 9, 10, 11, 12, 13, 14, 15, 20, 25, 30, or 35 of the loci listed Table 14. In some embodiments, a patient is identified as having an increased risk of developing breast cancer if at least 76% of the genotyped BC loci (callable, genotyped loci) have a cancer-like genotype (e.g., have a genotype that differs from the modal genotype determined for a reference population, such as a healthy population or the sample data best fits the cancer-like distribution). In some embodiments, the patient is identified as having an increased risk of developing cancer if at least 50%, 55%, 60%, 65%, 70%, 75%, 80%, 85%, 90%, 95% of the genotyped BC loci from Table 14 have a cancer-like genotype (e.g., have a genotype that differs from the modal genotype determined for a reference population, such as a healthy population or the sample data best fits the cancer-like distribution). The disclosure also contemplates diagnostic methods, wherein the patient is identified as having breast cancer if at least 50%, 55%, 60%, 65%, 70%, 75%, 80%, 85%, 90%, 95% of the genotyped BC loci (callable, genotyped loci) from Table 14 have a cancer-like genotype (e.g., have a genotype that differs from the modal genotype determined for a reference population, such as a healthy population or the sample data best fits the cancer-like distribution). The disclosure also contemplates prognostic methods, wherein the patient is identified as having a poor cancer prognosis if at least 50%, 55%, 60%, 65%, 70%, 75%, 80%, 85%, 90%, 95% of the genotyped BC loci from Table 14 have a cancer-like genotype (e.g., have a genotype that differs from the modal genotype determined for a reference population, such as a healthy population or the sample data best fits the cancer-like distribution). For any of the foregoing, in certain embodiments, the method is based on genotyping at least 30%, at least 40%, or at least 50% of the informative loci set forth in Table 14, and evaluating likelihood of developing breast cancer if at least 75%, at least 76%, or at least 77% of the genotyped loci are indicative of a cancer-associated state (e.g., data for the test sample, for a particular informative locus, is non-modal in comparison to a healthy reference population and/or the genotype or distribution of genotypes is more like that of the breast cancer population and less like that of the healthy population). In certain embodiments, the method is a computed implemented method where information about the modal genotype and/or genotype distribution for one or more reference populations are stored in a database, server, or host computer, optionally as a value or values), and new sequence information obtained for a test subject is obtained by reliably calling the genotypes for the informative microsatellite loci, providing that sequence information to a host computer, and comparing, in the host computer or between host computers or servers, the information from the test sample to the stored information about one or more reference populations (e.g., the stored value or values).

The disclosure also contemplates, in certain embodiments, methods of evaluating GBM predisposition, as well as prognostic and diagnostic methods in which any one or more of the loci in Table 17 are evaluated in a subject. In a preferred embodiment, the method is one that evaluates GBM predisposition, comprising genotyping at least 5%, 10%, 20%, 30%, 40%, 50%, 60%, 70%, 80%, 80% or 100% of the loci from Table 17 in a subject. Alternatively, the method may comprise genotyping at least 2, 3, 4, 5, 6, 7, 8, 9, 10, 11, 12, 13, 14, 15, 20, 25, 30, 35, 40, 45 or all of the loci from Table 17. In some embodiments, the patient is identified as having an increased risk of developing GBM if at least 57% of the genotyped loci from Table 17 (callable, genotyped loci) have a GBM-like genotype (e.g., have a genotype that differs from the modal genotype determined for a reference population, such as a healthy population or the sample data best fits the cancer-like distribution). In some embodiments, the patient is identified as having an increased risk of developing GBM if at least 10%, 15%, 20%, 25%, 30%, 35%, 40%, 45%, 50%, 55%, or 60% of the genotyped loci from Table 17 have a GBM-like genotype (e.g., have a genotype that differs from the modal genotype determined for a reference population, such as a healthy population or the sample data best fits the cancer-like distribution). The disclosure also contemplates diagnostic methods, wherein the patient is identified as having GBM if at least 10%, 15%, 20%, 25%, 30%, 35%, 40%, 45%, 50%, 55%, or 60% of the genotyped loci from Table 17 have a GBM-like genotype (e.g., have a genotype that differs from the modal genotype determined for a reference population, such as a healthy population or the sample data best fits the cancer-like distribution). The disclosure also contemplates prognostic methods, wherein the patient is identified as having a poor GBM prognosis if at least 10%, 15%, 20%, 25%, 30%, 35%, 40%, 45%, 50%, 55%, or 60% of the genotyped loci from Table 17 have a GBM-like genotype (e.g., have a genotype that differs from the modal genotype determined for a reference population, such as a healthy population or the sample data best fits the cancer-like distribution). For any of the foregoing, in certain embodiments, the method is based on genotyping at least 30%, at least 40%, or at least 50% of the informative loci set forth in Table 17, and evaluating likelihood of developing GBM if at least 50%, at least 55%, or at least 57% of the genotyped loci are indicative of a cancer-associated state (e.g., data for the test sample, for a particular informative locus, is non-modal in comparison to a healthy reference population and/or the genotype or distribution of genotypes is more like that of the GBM population and less like that of the healthy population). In certain embodiments, the method is a computed implemented method where information about the modal genotype and/or genotype distribution for one or more reference populations are stored in a database, server, or host computer, optionally as a value or values), and new sequence information obtained for a test subject is obtained by reliably calling the genotypes for the informative microsatellite loci, providing that sequence information to a host computer, and comparing, in the host computer or between host computers or servers, the information from the test sample to the stored information about one or more reference populations (e.g., the stored value or values).

The disclosure also contemplates, in certain embodiments, methods of evaluating GBM predisposition, as well as prognostic and diagnostic methods in which any one or more of the microsatellite loci, such as the specific loci set forth in Table 17, located in genes DHX36, DICER1, TTF2, DDX20, POLQ and DDX60 are evaluated. A GBM-like genotype (e.g., a genotype that differs from the modal genotype determined for a reference population, such as a healthy population or the sample data best fits the cancer-like distribution) at one or more of the six loci is indicative of an increased predisposition to GBM. Alternatively, a GBM-like genotype (e.g., have a genotype that differs from the modal genotype determined for a reference population, such as a healthy population or the sample data best fits the cancer-like distribution) at one or more of these six loci may be indicative of having GBM or of having a poor GBM prognosis.

The disclosure also contemplates, in certain embodiments, methods of evaluating LGG predisposition, as well as prognostic and diagnostic methods in which any one or more of microsatellite loci set forth in Table 18 are evaluated. In a preferred embodiment, the method is one that evaluates LGG predisposition, comprising genotyping at least 5%, 10%, 20%, 30%, 40%, 50%, 60%, 70%, 80%, 80% or 100% of the loci from Table 18. Alternatively, the method may comprise genotyping at least 2, 3, 4, 5, 6, 7, 8, 9, 10, 11, 12, 13, 14, 15, 20, 25, 30, 35, 40, 45, 50, 55, 60 or all of the loci from Table 18. In some embodiments, the patient is identified as having an increased risk of developing cancer if at least 35% of the genotyped LGG loci from Table 18 have a LGG-like genotype (e.g., have a genotype that differs from the modal genotype determined for a reference population, such as a healthy population or the sample data best fits the cancer-like distribution). In some embodiments, the patient is identified as having an increased risk of developing LGG if at least 5%, 10%, 15%, 20%, 25%, 30%, 35%, or 40% of the genotyped LGG loci from Table 18 have a LGG-like genotype (e.g., have a genotype that differs from the modal genotype determined for a reference population, such as a healthy population or the sample data best fits the cancer-like distribution). The disclosure also contemplates diagnostic methods, wherein the patient is identified as having LGG if at least 5%, 10%, 15%, 20%, 25%, 30%, 35%, or 40% of the loci from Table 18 have a LGG-like genotype (e.g., have a genotype that differs from the modal genotype determined for a reference population, such as a healthy population or the sample data best fits the cancer-like distribution). The disclosure also contemplates prognostic methods, wherein the patient is identified as having a poor LGG prognosis if at least 5%, 10%, 15%, 20%, 25%, 30%, 35%, or 40% of the loci from Table 18 have a LGG-like genotype (e.g., have a genotype that differs from the modal genotype determined for a reference population, such as a healthy population or the sample data best fits the cancer-like distribution). For any of the foregoing, in certain embodiments, the method is based on genotyping at least 30%, at least 40%, or at least 50% of the informative loci set forth in Table 18, and evaluating likelihood of developing LGG if at least 30%, at least 33%, or at least 35% of the genotyped loci are indicative of a cancer-associated state (e.g., data for the test sample, for a particular informative locus, is non-modal in comparison to a healthy reference population and/or the genotype or distribution of genotypes is more like that of the LGG population and less like that of the healthy population). In certain embodiments, the method is a computed implemented method where information about the modal genotype and/or genotype distribution for one or more reference populations are stored in a database, server, or host computer, optionally as a value or values), and new sequence information obtained for a test subject is obtained by reliably calling the genotypes for the informative microsatellite loci, providing that sequence information to a host computer, and comparing, in the host computer or between host computers or servers, the information from the test sample to the stored information about one or more reference populations (e.g., the stored value or values).

The disclosure also contemplates, in certain embodiments, methods of differentiating LGG from GBM in which any one or more of microsatellite loci set forth in Table 19 are evaluated. In a preferred embodiment, method is one that differentiates LGG from GBM, comprising genotyping at least 5%, 10%, 20%, 30%, 40%, 50%, 60%, 70%, 80%, 80% or 100% of the loci from Table 19. Alternatively, the method may comprise genotyping at least 2, 3, 4, 5, 6, 7, 8, 9, 10, 11, 12, 13, 14, 15, 20, 25 or all GBM loci from Table 19. In some embodiments, the patient is identified as having GBM over LGG if at least 82% of the genotyped loci from Table 19 have a GBM-like genotype (e.g., have a genotype that differs from the modal genotype determined for a reference population, where the reference population is patients with LGG). In some embodiments, the patient is identified as having GBM over LGG if at least 50%, 55%, 60%, 65%, 70%, 75%, 80%, 85%, 90%, 95% or 100% of the genotyped loci from Table 19 have a GBM-like genotype (e.g., have a genotype that differs from the modal genotype determined for a reference population, where the reference population is patients with LGG). The foregoing is indicative of the use of the disclosure to differentiate between disease-affected populations, such as to distinguish between individuals with an aggressive GBM brain tumor and those with a less aggressive tumor. Here, the selection of the reference populations is chosen to distinguish between those two states. Similarly, when making other types of comparisons based on likelihood that a tumor is aggressive or that a patient will respond to a particular treatment, the reference populations may be similarly selected. For any of the foregoing, in certain embodiments, the method is based on genotyping at least 30%, at least 40%, or at least 50% of the informative loci set forth in Table 19, and evaluating likelihood of developing GBM if at least 80%, at least 81%, or at least 82% of the genotyped loci are indicative of GBM (e.g., data for the test sample, for a particular informative locus, is non-modal in comparison to the GBM population and/or the genotype or distribution of genotypes is more like that of the GBM population and less like that of the LGG population). In certain embodiments, the method is a computed implemented method where information about the modal genotype and/or genotype distribution for one or more reference populations are stored in a database, server, or host computer, optionally as a value or values), and new sequence information obtained for a test subject is obtained by reliably calling the genotypes for the informative microsatellite loci, providing that sequence information to a host computer, and comparing, in the host computer or between host computers or servers, the information from the test sample to the stored information about one or more reference populations (e.g., the stored value or values).

The disclosure also contemplates, in certain embodiments, methods of differentiating LGG grade II from GBM in which any one or more of microsatellite loci set forth in Table 20 are evaluated. In a preferred embodiment, method is one that differentiates LGG grade II from GBM, comprising genotyping at least 5%, 10%, 20%, 30%, 40%, 50%, 60%, 70%, 80%, 80% or 100% of the loci from Table 19. Alternatively, the method may comprise genotyping at least 1, 2, 3, 4, 5, 6, 7, or 8 of the loci from Table 19. In some embodiments, the patient is identified as having GBM over LGG grade II if at least 85% of the genotyped loci from Table 19 have a GBM-like genotype (e.g., have a genotype that differs from the modal genotype determined for a reference population, where the reference population is patients with LGG). In some embodiments, the patient is identified as having GBM over LGG if at least 50%, 55%, 60%, 65%, 70%, 75%, 80%, 85%, 90%, 95% or 100% of the genotyped loci from Table 19 have a GBM-like genotype (e.g., have a genotype that differs from the modal genotype determined for a reference population, where the reference population is patients with LGG). For any of the foregoing, in certain embodiments, the method is based on genotyping at least 30%, at least 40%, or at least 50% of the informative loci set forth in Table 20, and evaluating likelihood of having GBM over LGG Type II if at least 80%, at least 81%, or at least 82% of the genotyped loci are indicative of GBM (e.g., data for the test sample, for a particular informative locus, is non-modal in comparison to the GBM population and/or the genotype or distribution of genotypes is more like that of the GBM population and less like that of the LGG type II population). In certain embodiments, the method is a computed implemented method where information about the modal genotype and/or genotype distribution for one or more reference populations are stored in a database, server, or host computer, optionally as a value or values), and new sequence information obtained for a test subject is obtained by reliably calling the genotypes for the informative microsatellite loci, providing that sequence information to a host computer, and comparing, in the host computer or between host computers or servers, the information from the test sample to the stored information about one or more reference populations (e.g., the stored value or values).

In certain embodiments of any of the foregoing, when using informative microsatellite loci as part of a diagnostic, prognostic, or risk assessment method for a patient, one or more microsatellite loci are evaluated, such as by determining length and/or nucleotide sequence at one or both alleles. Allelotype and/or genotype for each loci can then be compared to distribution data from one or more references, such as a modal genotype obtained from a reference population (e.g., a modal genotype from a references population of healthy subjects, such as subjects not diagnosed with cancer). In certain embodiments, information for comparison is a value stored on a computer to allow a yes/no comparison of test data to the stored value.

The foregoing is exemplary of using comparisons genotypes between two populations to identify informative microsatellite loci. The two populations are selected based on the desired application (e.g., distinguishing healthy from breast cancer; distinguishing an aggressive tumor from a non-aggressive tumor; distinguishing good responders of a therapy from poor responders; distinguishing healthy from a neurological condition; distinguishing healthy from a cardiovascular condition; etc.). Once the informative loci are identified, these loci may be used to prognose or diagnose future, test subjects. In certain embodiments, the method is used to determine whether a subject is at increased risk of developing a disease or condition. In such methods, having a disease associated genotype at informative microsatellite loci indicates increased risk of developing that disease or condition. In other embodiments, the method is used to diagnose a disease or condition, in a subject already suspected at having the disease or condition. In other embodiments, the method is used to distinguish between two conditions, such as an aggressive versus a non-aggressive tumor or a tumor that is likely to respond versus not respond to a therapy.

In certain embodiments, a detection, preventative and/or treatment regimen is specifically prescribed and/or administered to individuals who have been identified as having an increased risk of developing a condition, such as breast cancer, assessed by the methods described herein.

In certain embodiments, if a subject is identified as having an increased risk of or predisposition for breast cancer, a monitoring regimen is initiated that exceeds the standard level of monitoring typically recommended for a patient of the same gender and similar age. A detection regimen for individuals identified as having an increased risk of developing breast cancer may include, for example, more frequent mammography regimen (e.g., once a year, or once every six, four, three or two months); an early mammography regimen (e.g., mammography tests are performed beginning at age 25, 30, or 35); one or more biopsy procedures (e.g., a regular biopsy regimen beginning at age 40); breast biopsy and biopsy from other tissue; breast ultrasound and optionally ultrasound analysis of another tissue; breast magnetic resonance imaging (MRI) and optionally MRI analysis of another tissue; electrical impedance (T-scan) analysis of breast and optionally another tissue; ductal lavage; nuclear medicine analysis (e.g., scintimammography); BRCA1 and/or BRCA2 sequence analysis results; and/or thermal imaging of the breast and optionally another tissue.

In certain embodiments, if a subject is identified as having an increased risk of or predisposition for ovarian cancer, a monitoring regimen is initiated that exceeds the standard level of monitoring typically recommended for a patient of the same gender and similar age. A detection regimen for individuals identified as having an increased risk of developing ovarian cancer may include more frequent or regular pelvic examinations (e.g., once a year, or once every six, four, three or two months), transvaginal ultrasounds (e.g., once a year, or once every six, four, three or two months), CT scans, MRIs, laparotomies, laparoscopies, and even biopsies, or BRCA1 and/or BRCA2 sequence analysis.

Treatments sometimes are preventative (e.g., is prescribed or administered to reduce the probability that a breast cancer associated condition arises or progresses), sometimes are therapeutic, and sometimes delay, alleviate or halt the progression of ovarian and/or another cancer or condition. Any known preventative or therapeutic treatment may, in certain embodiments, be prophylactically initiated following indication that a subject is at increased risk for developing the disease. The decision to initiate prophylactic treatment, such as a prophylactic mastectomy, prophylactic ovarectomy, or prophylactic hysterectomy may be influenced by prior family history of cancer, when considered in combination with microsatellite analysis.

Additional examples of prophylactic treatments that may be initiated based on predisposition, even without a diagnosis of cancer, include administration of agents that are the standard of care for treating the particular cancer or disease. Further possible agents include selective hormone receptor modulators (e.g., selective estrogen receptor modulators (SERMs) such as tamoxifen, reloxifene, and toremifene); compositions that prevent production of hormones (e.g., aramotase inhibitors that prevent the production of estrogen in the adrenal gland, such as exemestane, letrozole, anastrozol, groserelin, and megestrol); other hormonal treatments (e.g., goserelin acetate and fulvestrant); biologic response modifiers such as antibodies (e.g., trastuzumab (herceptin/HER2)); or surgery (e.g., lumpectomy, mastectomy, or oophorectomy).

Any female patient or patient population may be assessed using the screening and diagnostic methods of the disclosure. For example, the methods disclosed herein may be performed on the general female patient population, as well as on the narrower population of post-menopausal women. The term “post-menopausal” is understood by those of skill in the art. In particular embodiments, post-menopausal generally refers to, for example, women over the age of 55. In particular embodiments, the screening methods are performed routinely (e.g., annually, every two years, etc.) on the general female population. Regular screening of patients may begin, for example, at the onset of menses, at age 30, or at the beginning of menopause. Screening of the high-risk patient population, will typically be performed on a routine basis independent of patient age. Patients who are both asymptomatic and symptomatic can be assessed for an increased likelihood of having ovarian using the screening and diagnostic methods of the disclosure. Women that are at a low-risk of developing ovarian and/or breast and those that are considered high-risk based on clinical and family history risk factors may also be assessed using the present methods. Patients considered “high-risk” based on such clinical and family history risk factors include but are not limited to patients living with breast cancer, colon cancer, or breast/ovarian syndrome, women with a first-degree relative with ovarian cancer (e.g., mother, daughter, or sister), patients positive for at least one breast cancer gene (BRCA 1 or 2), and women suffering from HNPCC (i.e., Hereditary non-polyposis colorectal cancer).

As breast and/or ovarian cancer preventative and treatment information can be specifically targeted to subjects in need thereof (e.g., those at risk of developing breast and/or ovarian cancer or those that have early signs of breast and/or ovarian cancer), provided herein is a method for preventing and/or reducing the risk of developing breast and/or ovarian cancer in a subject, which comprises: (a) detecting the presence or absence of a variation in an informative microsatellite loci identified by the methods of the disclosure in a nucleic acid sample from a subject; (b) identifying a subject at risk of breast cancer, whereby the presence of a variation in an informative microsatellite loci is indicative of a risk of breast cancer in the subject; and (c) if such a risk is identified, providing the subject with information about methods or products to prevent or reduce breast and/or ovarian cancer or to delay the onset of breast and/or ovarian cancer.

Pharmacogenomics

The present disclosure also provides methods for assessing the pharmacogenomics of a subject harboring particular microsatellite alleles to a particular therapeutic agent or pharmaceutical compound, or to a class of such compounds. Pharmacogenomics deals with the roles which clinically significant hereditary variations (e.g., microsatellite loci variations) play in the response to drugs due to altered drug disposition and/or abnormal action in affected persons. The clinical outcomes of these variations can result in severe toxicity of therapeutic drugs in certain individuals or therapeutic failure of drugs in certain individuals as a result of individual variation in metabolism. Thus, the global microsatellite profile of an individual can determine the way a therapeutic compound acts on the body or the way the body metabolizes the compound. For example, variations in microsatellite loci located the genes of drug metabolizing enzymes can alter the amino acid sequence, and thus activity of these enzymes, which in turn can affect both the intensity and duration of drug action, as well as drug metabolism and clearance.

The discovery of microsatellite variations in loci located in the genes of drug metabolizing enzymes, drug transporters, and other drug targets may explain why some patients do not obtain the expected drug effects, show an exaggerated drug effect, or experience serious toxicity from standard drug dosages. Accordingly, an alteration in global microsatellite profile may lead to allelic variants of a protein in which one or more of the protein functions in one population are different from those in another population. An assessment of an individual's global microsatellite profile thus provides a way to ascertain a genetic predisposition that can affect treatment modality. The disclosure provides methods and kits for use as companion diagnostics for such treatments.

For example, in a ligand-based treatment, a microsatellite variation in a gene coding for the target of the ligand may give rise to amino terminal extracellular domains and/or other ligand-binding regions that are more or less active in ligand binding, thereby affecting subsequent protein activation. Accordingly, ligand dosage would necessarily be modified to maximize the therapeutic effect within a given population containing particular microsatellite alleles. Thus, characterization of an individual's global microsatellite profile may permit the selection of effective compounds and effective dosages of such compounds for prophylactic or therapeutic uses based on the individual's global microsatellite profile, thereby enhancing and optimizing the effectiveness of the therapy. Furthermore, the production of recombinant cells and transgenic animals containing particular microsatellite variations may allow effective clinical design and testing of treatment compounds and dosage regimens. For example, transgenic animals can be produced that differ only in specific microsatellite alleles in a gene that is orthologous to a human disease susceptibility gene.

Accordingly, a method of the disclosure may include comparing the global microsatellite profile of a group of individuals known to respond positively to a particular treatment to the global microsatellite profile of a group known to respond poorly to the same treatment. Those microsatellite loci whose sequence lengths distributions differ significantly between populations may be used as informative microsatellite loci in optimizing the effectiveness of treatment in a particular individual.

Moreover, informative microsatellite loci may be identified, based on analysis of genotypes of allelotypes, to predict responsiveness to a therapy. This may be particularly useful in the design of clinical trials, such as to identify a microsatellite signature indicative of likelihood to respond to a therapy. This information may be harnessed for developing a companion diagnostic useful for determining, prior to initiating treatment, patients likely to respond to treatment.

Therapeutics/Drug Development

The informative microsatellite loci identified using the methods of the present disclosure also can be used to identify novel therapeutic targets, such as for cancer. For example, genes (and/or their products) containing the informative microsatellite loci, as well as genes (and/or their products) that are directly or indirectly regulated by or interacting with these variant genes or their products, can be targeted for the development of therapeutics that, for example, treat the cancer or prevent or delay cancer onset. The therapeutics may be composed of, for example, small molecules, proteins, protein fragments or peptides, antibodies, nucleic acids, or their derivatives or mimetics which modulate the functions or levels of the target genes or gene products.

The informative microsatellite loci identified using the methods of the present disclosure are also useful for designing RNA interference reagents that specifically target nucleic acid molecules comprising particular informative microsatellite loci. RNA interference (RNAi), also referred to as gene silencing, is based on using double-stranded RNA (dsRNA) molecules to turn genes off. When introduced into a cell, dsRNAs are processed by the cell into short fragments (generally about 21, 22, or 23 nucleotides in length) known as small interfering RNAs (siRNAs) which the cell uses in a sequence-specific manner to recognize and destroy complementary RNAs (Thompson, Drug Discovery Today, 7 (17): 912-917 (2002)). Accordingly, an aspect of the present disclosure specifically contemplates isolated nucleic acid molecules that are about 18-26 nucleotides in length, preferably 19-25 nucleotides in length, and more preferably 20, 21, 22, or 23 nucleotides in length, and the use of these nucleic acid molecules for RNAi. Because RNAi molecules, including siRNAs, act in a sequence-specific manner, the informative microsatellite of the present disclosure can be used to design RNAi reagents that recognize and destroy nucleic acid molecules having specific microsatellite alleles, while not affecting nucleic acid molecules having alternative microsatellite alleles. As with antisense reagents, RNAi reagents may be directly useful as therapeutic agents (e.g., for turning off defective, disease-causing genes), and are also useful for characterizing and validating gene function (e.g., in gene knock-out or knock-down experiments).

In cases in which a microsatellite locus variation results in a variant protein that is ascribed to be the cause of, or a contributing factor to, a pathological condition, a method of treating such a condition can include administering to a subject experiencing the pathology the wild-type/normal cognate of the variant protein. Once administered in an effective dosing regimen, the wild-type cognate provides complementation or remediation of the pathological condition. A method of treating such a condition may also include administering to a subject experiencing the pathology an agent or compound that inhibits the variant protein (e.g., that restores wildtype function to the variant protein).

The disclosure further provides a method for identifying a compound or agent that can be used to treat cancer. The informative microsatellite loci identified by the methods disclosed herein are useful as targets for the identification and/or development of therapeutic agents. A method for identifying a therapeutic agent or compound typically includes assaying the ability of the agent or compound to modulate the activity and/or expression of a variant microsatellite locus-containing nucleic acid or the encoded product and thus identifying an agent or a compound that can be used to treat a disorder characterized by undesired activity or expression of the variant microsatellite locus-containing nucleic acid or the encoded product. The assays can be performed in cell-based and cell-free systems. Cell-based assays can include cells naturally expressing the nucleic acid molecules of interest or recombinant cells genetically engineered to express certain nucleic acid molecules.

In a specific example, an assay includes screening for agents or molecules that bind to and/or inhibit and/or restore wildtype function to the variant MAPKAPK3 disclosed herein. This variant protein results from the microsatellite variation associated with increased breast cancer risk, described herein. As discussed in more detail in the Examples, one of the informative microsatellite locus variants identified herein creates a putative frame-shift mutation in MAPKAPK3, producing a mutant protein with an extended C-terminus, 17 amino acids longer than the wild-type Importantly, these changes are located in the p38 MAPK-binding site (a.a. 345-369) and bipartite nuclear localization signal 2 (a.a. 364-368) regions. This suggests breast cancer patients with this variation may have an alternative MAPKAPK3 protein that is unable to localize to the nucleus for transcription regulation and/or has altered affinity to the p38 MAPK-binding site. Accordingly, in some aspects, the present disclosure provides a method for identifying an agent, such as a protein, peptide, or small molecule, which binds to the extended C-terminal portion of the variant MAPKAPK3 disclosed herein. In further aspects, the method is used to identify an agent, such as a protein, peptide, or small molecule, which inhibits the variant MAPKAPK3 disclosed herein. By way of example, such a screening assay may be performed in a cell free system where the variant protein is provided and contacted with test agents to identify those agents that bind the C-terminal portion. Controls may include wildtype MAPKAPK3 protein (e.g., lacking the C-terminal portion). This permits selection of test agents that specifically bind the C-terminal portion but do not otherwise bind MAPKAPK3. Such test agents can be further analyzed in functional assays to evaluate whether they rescue native function in the variant protein.

In another specific example, an assay includes screening for agents or molecules that bind to and/or inhibit and/or restore native function of the variant HSPA6 disclosed herein. This variant protein results from the microsatellite variation associated with increased breast cancer risk, described herein. As discussed in more detail in the Examples, one of the informative microsatellite locus variants identified herein create a putative two amino acid deletion in HSPA6. These changes occur in residues 502-505 where Lys (a.a. 502) is a modification site. Lysine modifications in macromolecular proteins such as HSPA6 are associated with chromatin remodeling, cell cycle, splicing, nuclear transport, and actin nucleation. Thus, modifications introduced through microsatellite variants may alter HSPA6 acetylation leading to changes in normal cellular processes. Accordingly, in some aspects, the present disclosure provides a method for identifying an agent, such as a protein, peptide, or small molecule, which binds to the variant HSPA6 disclosed herein. In further aspects, the method is used to identify an agent which inhibits the variant HSPA6 disclosed herein and/or restores normal function to the variant protein (e.g., restores the function typically seen with the wildtype protein).

In another specific example, an assay includes screening for agents or molecules that bind to and/or inhibit and/or restore native function of any one of the proteins encoded by variant DHX36, DICER1, TTF2, DDX20, POLQ and DDX60 disclosed herein. These variants result from the microsatellite variation associated with increased GBM risk, described herein. For example, an agent or molecule may reduce alternative splicing associated with the variant.

DHX36 is known to deadenylate and degrade mRNA. Thus, modifications introduced through microsatellite variants may alter DHX36 activity leading to changes in normal cellular processes. Accordingly, in some aspects, the present disclosure provides a method for identifying an agent, such as a protein, peptide, or small molecule, which binds to the variant DHX36 disclosed herein. In further aspects, the method is used to identify an agent which inhibits the variant DHX36 disclosed herein and/or restores normal function to the variant protein (e.g., restores the function typically seen with the wildtype protein).

DICER1 has been implicated in cancer and neuroskeletal disease Importantly, it cleaves dsRNA to siRNA and is essential to processing miRNA into mature miRNA. Accordingly, in some aspects, the present disclosure provides a method for identifying an agent, such as a protein, peptide, or small molecule, which binds to the variant DICER1 disclosed herein. In further aspects, the method is used to identify an agent which inhibits the variant DICER1 disclosed herein and/or restores normal function to the variant protein (e.g., restores the function typically seen with the wildtype protein).

TTF2 represses mitotic transcription and pre-mRNA-splicing and therefore would be especially important to cell-division. Accordingly, in some aspects, the present disclosure provides a method for identifying an agent, such as a protein, peptide, or small molecule, which binds to the variant TTF2 disclosed herein. In further aspects, the method is used to identify an agent which inhibits the variant TTF2 disclosed herein and/or restores normal function to the variant protein (e.g., restores the function typically seen with the wildtype protein).

DDX20 contributes to miRNA containing RNP complexes which suppress NF-\hat{k}B via modulation of miRNA-140 (potential tumor suppressor). Accordingly, in some aspects, the present disclosure provides a method for identifying an agent, such as a protein, peptide, or small molecule, which binds to the variant DDX20 disclosed herein. In further aspects, the method is used to identify an agent which inhibits the variant DDX20 disclosed herein and/or restores normal function to the variant protein (e.g., restores the function typically seen with the wildtype protein).

POLQ is a DNA polymerase activity on nicked double-stranded DNA and on a singly primed DNA template. It may be involved in the repair of inter-strand cross-links. Accordingly, in some aspects, the present disclosure provides a method for identifying an agent, such as a protein, peptide, or small molecule, which binds to the variant POLQ disclosed herein. In further aspects, the method is used to identify an agent which inhibits the variant POLQ disclosed herein and/or restores normal function to the variant protein (e.g., restores the function typically seen with the wildtype protein).

DDX60 is an RNA helicase that possess the activity to bind to viral RNA and DNA. In some aspects, the present disclosure provides a method for identifying an agent, such as a protein, peptide, or small molecule, which binds to the variant DDX60 disclosed herein. In further aspects, the method is used to identify an agent which inhibits the variant DDX60 disclosed herein and/or restores normal function to the variant protein (e.g., restores the function typically seen with the wildtype protein). In another specific example, an assay includes screening for agents or molecules that bind to and/or inhibit and/or restore native function of the any one of the proteins encoded by variant COQ10B, NUFIP1, KDM1A, SPHK2, STC1, CRNKL1, PIAS2, MLL, SAR1B, DNAH3, ATXN2L, WWC3, TLN2, MT1X, DHX40, CUL1, POP4, PDGFRA, OFD1, PTPN22, MICALL1, NUP54, ADAM2, and TRG disclosed herein. These variant proteins result from the microsatellite variation associated with increased breast cancer risk, described herein.

Expression of mRNA transcripts and encoded proteins may be altered in individuals with a particular microsatellite allele in a regulatory/control element, such as a promoter or transcription factor binding domain, that regulates expression. In this situation, methods of treatment and compounds can be identified, that regulate or overcome the variant regulatory/control element, thereby generating normal, or healthy, expression levels.

In cases in which a microsatellite locus variation results aberrant expression of a gene product (overexpression or reduced expression), modulators of gene expression can be identified in a method wherein, for example, a cell is contacted with a candidate compound/agent and the expression of target mRNA determined. The level of expression of mRNA in the presence of the candidate compound is compared to the level of expression of mRNA in the absence of the candidate compound. The candidate compound can then be identified as a modulator of variant gene expression based on this comparison and be used to treat a disorder such as cancer that is characterized by variant gene expression. When expression of mRNA is statistically significantly greater in the presence of the candidate compound than in its absence, the candidate compound is identified as a stimulator of nucleic acid expression. When nucleic acid expression is statistically significantly less in the presence of the candidate compound than in its absence, the candidate compound is identified as an inhibitor of nucleic acid expression.

Definitive Diagnosis

In certain embodiments, the methods of the disclosure are used for definitive diagnosis. In such cases, prior to microsatellite analysis, a patient is already suspected of having a particular cancer (or other disease or condition). For example, the patient is suspected of having a particular cancer because the patient (i) has already has one or more tests consistent with the cancer, (ii) has one or more symptoms consistent with the cancer, (iii) has a family history of the cancer, or (iv) any combination of the foregoing.

In this context, analysis of informative microsatellites can be used to confirm the suspected diagnosis of the cancer (or other disease or condition). This is of particular use because it provides a non-invasive method to confirm the diagnosis before initiating more invasive measures. So, for example, if a patient is already suspected of having breast cancer because of a suspicious lump on a mammogram, and analysis of one or more informative microsatellite loci indicates a high risk for developing breast cancer, these data taken together support a diagnosis of breast cancer. At that point, further more invasive testing may be performed. Alternatively, the patient may begin treatment immediately, such as surgery or a therapeutic regimen.

Tumor Microsatellite Instability

In certain embodiments, the methods of the disclosure are used to compare the microsatellite loci of germline and tumor of a particular type, e.g., breast cancer or a subtype of breast cancer. The germline and tumor samples may be matched patient samples or unmatched. The methods of the disclosure may be used to compare within a population the germline and tumor genotype distribution to identify loci that differentiate a patient's germline genome from the tumor. These comparisons may be used to identify individual loci that are tumor hot spots (frequently mutated) or causative of disease as identified by a change in the tumor. Alternatively, a panel may be used to assay GMI or microsatellite instability as a whole.

The disclosure provides methods of identifying microsatellite instability in a tumor, comprising: (i) obtaining a tumor sample and a germline sample comprising nucleic acid from a subject; (ii) analyzing the nucleic acid to determine a genotype for at least 30% of microsatellite loci from a panel of microsatellite loci identified as being variant within a population; (iii) comparing the genotypes of the two samples of a first microsatellite locus genotyped in (ii); and (iv) repeating step (iii) for the remaining genotyped microsatellite loci; wherein, differences in length or sequence of the loci indicate microsatellite instability at those loci. The disclosure provides methods of identifying microsatellite instability in a tumor type, comprising: (i) obtaining a population of tumor samples of a specific type and a population of germline samples comprising nucleic acid from a subject; (ii) analyzing the nucleic acid to determine a genotype for at least 30% of microsatellite loci from a panel of microsatellite loci identified as being variant within a population; (iii) comparing the distribution of genotypes of the tumor samples of a specific type and a population of germline samples of a first microsatellite locus genotyped in (ii); and (iv) repeating step (iii) for the remaining genotyped microsatellite loci; wherein, differences in genotype distribution indicate microsatellite instability at those loci.

### 5. Kits

The disclosure also provides various kits. They kits may be used, for example, in a method of diagnosis or prognosis or treatment, as described herein, as well as to methods for identifying other informative microsatellite loci. Moreover, these kits are applicable to identifying informative microsatellite loci and diagnostic/prognostic/treatment methods based on either analysis of allelotype of microsatellite loci or based on analysis of genotype of microsatellite loci.

A microsatellite detection kit/system of the present disclosure may include components that are used to prepare nucleic acids from a test sample for the subsequent amplification and/or detection of a microsatellite locus-containing nucleic acid molecule. Such sample preparation components can be used to produce nucleic acid extracts (including DNA and/or RNA), proteins or membrane extracts from any bodily fluids (such as blood, serum, plasma, urine, saliva, phlegm, gastric juices, semen, tears, sweat, etc.), skin, hair, cells (especially nucleated cells), biopsies, buccal swabs or tissue specimens. Although the instant methods are suitable for use on non-tumor sample, in certain embodiments the sample is a tumor sample. Nucleic acid may be prepared, for example, from fresh biopsy tissue, frozen tissue, or formalin-fixed tissue. The test samples used in the above-described methods will vary based on such factors as the assay format, nature of the detection method, and the specific tissues, cells or extracts used as the test sample to be assayed. Methods of preparing nucleic acids, proteins, and cell extracts are well known in the art and can be readily adapted to obtain a sample that is compatible with the system utilized. Automated sample preparation systems for extracting nucleic acids from a test sample are commercially available, and examples are Qiagen's BioRobot 9600, Applied Biosystems' PRISM™ 6700 sample preparation system, and Roche Molecular Systems' COBAS AmpliPrep System.

A person skilled in the art will recognize that, based on the microsatellite loci and flanking sequence information disclosed herein, detection reagents can be developed and used to assay any microsatellite locus of the present disclosure individually or in combination, and such detection reagents can be readily incorporated into one of the established kit formats which are well known in the art.

The terms “kits”, as used herein in the context of microsatellite detection reagents, are intended to refer to such things as combinations of multiple microsatellite detection reagents, or one or more microsatellite detection reagents in combination with one or more other types of elements or components (e.g., other types of biochemical reagents, containers, packages such as packaging intended for commercial sale, substrates to which microsatellite detection reagents are attached, electronic hardware components, etc.). Accordingly, the present disclosure further provides microsatellite detection kits, including but not limited to, packaged probe and primer sets (e.g., TaqMan probe/primer sets), arrays/microarrays of nucleic acid molecules, and beads that contain one or more probes, primers, or other detection reagents for detecting one or more microsatellites of the present disclosure. The kits can optionally include various electronic hardware components; for example, arrays (“DNA chips”) and microfluidic systems (“lab-on-a-chip” systems) provided by various manufacturers typically comprise hardware components. Other kits/systems (e.g., probe/primer sets) may not include electronic hardware components, but may be comprised of, for example, one or more microsatellite detection reagents (along with, optionally, other biochemical reagents) packaged in one or more containers.

Microsatellite detection kits may contain, for example, one or more probes, or pairs of probes, that hybridize to a nucleic acid molecule at or near each target microsatellite locus. Multiple pairs of allele-specific probes may be included in the kit to simultaneously assay large numbers of microsatellite loci, at least one of which is a microsatellite of the present disclosure. In some kits, the allele-specific probes are immobilized to a substrate such as an array or bead. For example, the same substrate can comprise allele-specific probes for detecting at least 1; 10; 100; 1000; 10,000; 100,000 (or any other number in-between) or substantially all of the microsatellites shown in Tables 1-10. In certain embodiments, the kits of the disclosure comprise appropriate controls to ensure the kit is working as intended.

The terms “arrays”, “microarrays”, and “DNA chips” are used herein interchangeably to refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate. In one embodiment, the microarray is prepared and used according to the methods described in U.S. Pat. No. 5,837,832, Chee et al., PCT application WO95/11995 (Chee et al.), Lockhart, D. J. et al. (1996; Nat. Biotech. 14: 1675-1680) and Schena, M. et al. (1996; Proc. Natl. Acad. Sci. 93: 10614-10619), all of which are incorporated herein in their entirety by reference. In other embodiments, such arrays are produced by the methods described by Brown et al., U.S. Pat. No. 5,807,522.

A microarray can be composed of a large number of unique, single-stranded polynucleotides, fixed to a solid support. Typical polynucleotides are preferably about 6-60 nucleotides in length, more preferably about 15-30 nucleotides in length, and most preferably about 18-25 nucleotides in length. For certain types of microarrays or other detection kits/systems, it may be preferable to use oligonucleotides that are only about 7-20 nucleotides in length.

In certain embodiments, the kits comprise a bait set of polynucleotides described above for Next-Gen sequencing. Features of enrichment probes suitable for enriching prior to Next-Gen sequencing are described in U.S. 2012/0208706, herein incorporated by reference in its entirety.

In certain embodiments, the kits may be companion diagnostics for treatments described above.

Global Microsatellite Content Array

An array used in the kits and systems of the present disclosure can be a Global Microsatellite Content Array. This array is described in US 2010/0317534, which is incorporated herewith in its entirety. Briefly, the array probe design is based on computationally-derived simple repeat DNA sequences (i.e. all possible 1- to 6-mer microsatellite motif combinations, including every cyclic permutation and corresponding complement sequence), not on unique sequences derived from any specific genome. Unlike a CGH array recorded hybridization intensities that are used to estimate copy variations at specific positions within the genome, the global microsatellite array is used to directly compare intensity values that represent the sum across all individual microsatellite motif-containing loci. For example, the intensity recorded on the probe for the AATT motif (and probes for its cyclic permutations, ATTT, TTTA, and TTAA) measures the contributions from the 886 AATT motif specific microsatellite loci spread throughout the reference human genome. The global microsatellite array can therefore be used to specifically and accurately measure significant motif-specific variations (polymorphisms), whether they are in the germ line or arise as somatic mutations, in any nucleic acid sample.

Target Enrichment for Microsatellite Using Loci-Specific Probes

Given that next-generation sequencing reads are statistically distributed according the Lander-Waterman equation, each genome sequence set may have sufficient depth of coverage to measure only a fraction, typically 50% of the microsatellite loci for typical moderate coverage data sets. In addition, as described herein, only the reads that span the repetitive region and have sufficient high complexity flanking sequence aid in the calling of the genotype at a given locus. Therefore, the many reads that terminate in the repetitive region do not contribute, thus overall the effective depth of coverage is lower than for a given single base. Accordingly, the kits and methods of the disclosure may comprise an array including probes containing, in addition to microsatellite repeat sequences, flanking sequence so that only the reads comprising flanking sequences are captured. The captured nucleic acid sequences can then be released for sequencing.

Given that next-generation sequencing reads are statistically distributed according the Lander-Waterman equation, each genome sequence set may have sufficient depth of coverage to measure only a fraction, typically 50% of the microsatellite loci for typical moderate coverage data sets. In addition, as described herein, only the reads that span the repetitive region and have sufficient high complexity flanking sequence aid in the calling of the genotype at a given locus. Therefore, the many reads that terminate in the repetitive region do not contribute, thus overall the effective depth of coverage is lower than for a given single base. Accordingly the methods and kits of the disclosure may include means to enrich for particular microsatellite loci of interest, prior to performing sequencing of the nucleic acid sample. Such methods may be used to enrich for informative read when constructing a database of information based on comparing two populations. Additionally or alternatively, such methods and kits may be used when analyzing a particular sample from a subject. The enrichment methods and compositions are useful, for example, for increasing the relative abundance of nucleic acid sequence prior to deep sequencing (such as NextGen sequencing). Other uses include discovering new genomic regions of value, finding companion diagnostics, and measuring quantitatively the amount of repetitive elements in a genome.

The term “enrichment” or “enrich” refers to the process of increasing the relative abundance of particular nucleic acid sequences in a sample relative to the level of nucleic acid sequences as a whole initially present in said sample before treatment. Thus the enrichment step provides a percentage or fractional increase rather than directly increasing for example, the copy number of the nucleic acid sequences of interest as amplification methods, such as PCR, would. The enrichment step described herein may be used to remove DNA strands that it is not desired to sequence, rather than to specifically amplify only the sequences of interest.

The enrichment step may be performed using a high density DNA-array for specific capturing of the gene regions of interest, e.g., the microsatellite loci of interest. Thus a kit of the present disclosure may comprise such an array, along with instructions for using such an array. Optionally, the kit may include, in separate containers, reagents needed to use the array (e.g., buffers, etc.). An array for the specific capturing of the microsatellite loci of interest may bear more than 1 million different capture sequences or probes. Thus, in the context of the present disclosure, the term “plurality of oligonucleotide probes” is understood as comprising more than 100 and preferably more than 1000 oligonucleotides.

The capture probes are preferably nucleic acids, such as oligonucleotides, capable of binding to a target nucleic acid sequence through one or more types of chemical bonds, usually through complementary base pairing, usually through hydrogen bond formation. Such probes may include natural or modified bases and may be RNA or DNA. In addition the bases in probes may be joined by a linkage other than a phosphodiester bond so long as it does not interfere with hybridization. Thus probes may also be peptide nucleic acids (PNA) in which the constituent bases are joined by peptide bonds rather than phosphodiester linkages.

Capture probes are populations of nucleic acid sequences. These have been selected such that said probes relate to, by way of non-limiting examples, particular microsatellite loci of interest Importantly, to permit the capture of whole, rather than partial microsatellite loci, such capture probes preferentially contain, in addition to microsatellite repeat sequences, the unique sequences flanking the microsatellite repeat. Furthermore, the population of capture probes may comprise 1-mers to 6-mers of: perfect repeats, single mismatches, double mismatches and single nucleotide deletions of particular microsatellite loci of interest.

Capture probes can be obtained from a commercial source, such as NimbleGen (Roche) or Integrated DNA Technologies (IDT) for DNA oligos. Oligos can also be obtained from Agilent Technologies. Protocols for enrichment are publicly available, e.g., SureSelect Target Enrichment System or ILLUMINA Target Enrichment System.

The terms “target” or “target sequence” refer to nucleic acid sequences of interest that is, those which hybridize to the capture probes. Thus the term includes those larger nucleic acid sequences, a sub-sequence of which binds to the probe and/or to the overall bound sequence. Since the target sequences are for use in sequencing methods, said target sequences do not need to have been previously defined to any extent, other than the bases complementary to the capture probes.

Capture probes hybridize to target sequences in the complex nucleic acid sample. It will be apparent to one skilled in the art that prior to hybridization said complex nucleic acid sample will preferably comprise single stranded nucleic acid sequences. This can be achieved by a number of well-known methods in the art such as, for example using heat to denature or separate complementary strands of double stranded nucleic acids, which on cooling can hybridize to the capture probes.

To provide enrichment, the capture probes are preferably immobilized onto a support, either before or after hybridization, such that sequences that do not hybridize to said capture probes can be removed for example, by washing.

In one embodiment the target sequences can be removed from the probe-target complex prior to sequencing for example by elution. Removal by denaturation of the selected targets from the immobilized capture probes will generally give a solution of single stranded targets.

The solid support may be any of the conventional supports used in arrays or “DNA chips”, beads, including magnetic beads or polystyrene latex microspheres, arrays of beads, or substrates such as membranes, slides and wafers made from cellulose, nitrocellulose, glass, plastics, silicon and the like.

Preferably the solid support is a flat planar surface or an array of beads. Still more preferably said solid support is an array and most preferably said array is a “high density array” such as a micro-array.

In a specific embodiment, the capture probes are designed to contain the repetitive microsatellite repeats (oligos consist of many copies of the different 1-6 mer repeat motifs) so that it concentrates (enriches) for all the microsatellite loci in a genome. In certain embodiments, the oligos are about 20, 30, 30, 50, 60, 70, 80, 90, 100, 110, 120, 130, 140, 150, 160, 170, 180, 190, or 200 nucleotides. In certain preferred embodiments, the oligos are about 120 nucleotides. In some embodiments, each oligo is composed of about four 30 nucleotide regions each of which targets a different motif sequence. In certain embodiments, the oligos have approximately a 40% G/C content along the full length of the oligo. In certain embodiments, motifs for each oligo are selected to have a lower probability of internal hairpin formation.

In another specific embodiment, the capture probes are designed for specific microsatellite containing loci, for example, the informative loci from all the different cancer types or for a subset of cancer type (e.g., a kit for enriching for BC informative microsatellites), and this is done by using the unique flanking sequence adjacent to the microsatellite of interest.

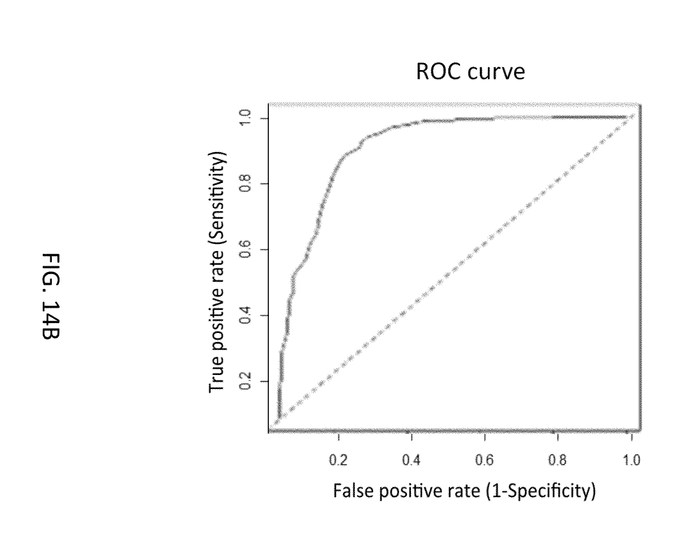

FIG. 13 show the results of an experiment in which enrichment was performed to capture specific microsatellite loci in the human genome.

In some embodiments, a kit of the disclosure includes capture probes specific for any of the cancer types disclosed herein. For example, a kit may include a set of capture probes specific for the informative microsatellite loci listed in any one or more of Tables 1-22. It is also contemplated that a kit may contain probes for enriching for a subset of loci (e.g., it is not necessary that a kit contain probes specific for all of a particular set of informative loci). In a specific embodiment, a kit includes a set of capture probes specific for informative microsatellite loci associated with breast cancer. In another specific embodiment, a kit includes a set of capture probes specific for informative microsatellite loci associated with GBM. In another specific embodiment, a kit includes a set of capture probes specific for informative microsatellite loci associated with LGG. In another embodiment, a kit includes a set of capture probes specific for at least 5%, 10%, 20%, 30%, 40%, 50%, 60%, 70%, 80%, 90% or 100% of the loci listed in Table 14. In another embodiment, a kit includes a set of capture probes specific for at least 2, 5, 10, 15, 20, 25, 30, 35, 40, 45, 50 or all of the loci listed in Table 14. In another embodiment, a kit includes a set of capture probes specific for at least 5%, 10%, 20%, 30%, 40%, 50%, 60%, 70%, 80%, 90% or 100% of the loci listed in Table 17. In another embodiment, a kit includes a set of capture probes specific for at least 2, 5, 10, 15, 20, 25, 30, 35, 40, 45 or all of the loci listed in Table 17. In another embodiment, a kit includes a set of capture probes specific for at least 5%, 10%, 20%, 30%, 40%, 50%, 60%, 70%, 80%, 90% or 100% of the loci listed in Table 18. In another embodiment, a kit includes a set of capture probes specific for at least 2, 5, 10, 15, 20, 25, 30, 35, 40, 45, 50, 55, 60, 65 or all of the loci listed in Table 18. In another embodiment, a kit includes a set of capture probes specific for at least 5%, 10%, 20%, 30%, 40%, 50%, 60%, 70%, 80%, 90% or 100% of the loci listed in Table 19. In another embodiment, a kit includes a set of capture probes specific for at least 2, 5, 10, 15, 20, 25, 30, 35, 40, 45, 50, 55, 60, 65 or all of the loci listed in Table 19. In another embodiment, a kit includes a set of capture probes specific for at least 5%, 10%, 20%, 30%, 40%, 50%, 60%, 70%, 80%, 90% or 100% of the loci listed in Table 20. In another embodiment, a kit includes a set of capture probes specific for at least 2, 5, 10, 15, 20, 25, 30, 35, 40, 45, 50, 55, 60, 65 or all of the loci listed in Table 20.

In certain embodiments, samples may be multiplexed when using the target enrichment kits in order to increase efficiency for calling loci and to decrease costs. In certain embodiments, at least 2, 4, 6, 8, 10 or more samples are used in a reaction.

Amplification Methods

Primers for one or more microsatellite loci are provided in each embodiment of the method of the present disclosure. At least one primer is provided for each locus, more preferably at least two primers for each locus, with at least two primers being in the form of a primer pair which flanks the locus. When the primers are to be used in a multiplex amplification reaction it is preferable to select primers and amplification conditions which generate amplified alleles from multiple co-amplified loci which do not overlap in size or, if they do overlap in size, are labeled in a way which enables one to differentiate between the overlapping alleles.

Exemplary primers suitable for the amplification of individual loci according to the methods of the present disclosure are provided in Table 13. It is contemplated that other primers suitable for amplifying the same loci or other sets of loci falling within the scope of the present invention could be determined based on the present disclosure of informative loci and their position in the genome.

In certain embodiments, suitable primer pairs are selected to amplify the entire microsatellite loci of interest, as well as at least 5, at least 6, at least 7, at least 8, at least 9, or at least 10 flanking nucleotides 5′ and/or 3′ to the microsatellite loci. In certain embodiments, suitable primer pairs are selected to amplify the entire microsatellite loci of interest, as well as flanking nucleotides, but the flanking nucleotides amplified are less than 50, less than 40, less than 30, or less than 25 nucleotides on one or both sides of the microsatellite loci.

Amplification methods that are optionally utilized to amplify microsatellite DNA from the samples of biological material include, e.g., various polymerase, ligase, or reverse-transcriptase mediated amplification methods, such as the polymerase chain reaction (PCR), the ligase chain reaction (LCR), reverse-transcription PCR (RT-PCR), and/or the like. Details regarding the use of these and other amplification methods can be found in any of a variety of standard texts, including, e.g., Berger, Sambrook, Ausubel 1 and 2, and Innis, which are referred to above. Many available biology texts also have extended discussions regarding PCR and related amplification methods. Nucleic acid amplification is also described in, e.g., Mullis et al., (1987) U.S. Pat. No. 4,683,202 and Sooknanan and Malek (1995) Biotechnology 13:563, which are both incorporated by reference Improved methods of amplifying large nucleic acids by PCR are summarized in Cheng et al. (1994) Nature 369:684, which is incorporated by reference. In certain embodiments, duplex PCR is utilized to amplify target nucleic acids. Duplex PCR amplification is described further in, e.g., Gabriel et al. (2003) “Identification of human remains by immobilized sequence-specific oligonucleotide probe analysis of mtDNA hypervariable regions I and II,” Croat. Med. J. 44(3)293 and La et al. (2003) “Development of a duplex PCR assay for detection of Brachyspira hyodysenteriae and Brachyspira pilosicoli in pig feces,” J. Clin. Microbiol. 41(7):3372, which are both incorporated by reference.

In some embodiments, the informative microsatellite loci of the disclosure are amplified using primer pairs listed in Table 13. In an exemplary embodiment, an informative microsatellite locus located in the C5orf41 gene is amplified using forward primer TGCAGTAAAGAAGTCACGGAGA and reverse primer CCTGGAAGCCAGCTTATTTTT. In another exemplary embodiment, an informative microsatellite locus located in the PRKCA is amplified using forward primer ACGCCATTCTGACGTCTCTT and reverse primer ATTTAGTGTGGAGCGGATGG. In another exemplary embodiment, an informative microsatellite locus located in the MAPKAPK3 is amplified using forward primer CTTAGTGCCCACCATCCTGT and reverse primer CCCCATGAGCTACTGGTTGT. In another exemplary embodiment, an informative microsatellite locus located in the NSUN5 gene is amplified using forward primer TTCCAACAGGTCCTCATTCC and reverse primer GCTTCATGCTTAGGGCATTT. In another exemplary embodiment, an informative microsatellite locus located in the EIF4G3 gene is amplified using forward primer GGAGGAGAAGCTGGAGGAGT and reverse primer ACGGAGAGCATTGTGGAAAT. In another exemplary embodiment, an informative microsatellite locus located in the CABIN1 gene is amplified using forward primer GGAGGAGCTGAGCATCAGTG and reverse primer ACGGTAGGCATCCAACAGAA. In another exemplary embodiment, an informative microsatellite locus located in the CDC2L1 gene is amplified using forward primer CAGCCCACTCACCTTTCTCT and reverse primer GGCCTCGTGAAATTTTTGAA. In another exemplary embodiment, an informative microsatellite locus located in the RPL14 gene is amplified using forward primer CCTGAAAGCTTCTCCCAAAA and reverse primer TGCCACTTATGCTTTCTTGC. In another exemplary embodiment, an informative microsatellite locus located in the gene HSPA6 is amplified using forward primer GGGGTCTTCATCCAGGTGTA and reverse primer AACCATCCTCTCCACCTCCT.

The disclosure contemplates methods of amplifying an informative microsatellite locus using, for example, the primer pairs set forth above or other primer pairs that flank the microsatellite. The disclosure also contemplates compositions of these useful primer pairs. Such compositions comprise a set of primers (e.g., a primer pair). In certain embodiments, each primer of the pair is less than 100 nucleotides, such as less than 90, 85, 80, 75, 70, 65, 60, 55, or less than or equal to 50 nucleotides. Each such primer pair comprises a nucleotide sequence, such as the sequences set forth in Table 13.

A kit of the disclosure may, in certain embodiments, comprise a set of primers (a primer pair) suitable for amplifying an informative microsatellite loci. The kit may optionally include other reagents, such as in separate containers, for (i) performing the amplification reaction and/or for extracting nucleic acid from a sample. Such other reagents include buffers, polymerase, nucleotides, and the like. The kit may further include instructions for use.

In certain embodiments, the disclosure provides a composition comprising a set of primers (a primer pair) suitable for amplifying an informative microsatellite locus from a sample. The composition comprises a first nucleic acid comprising a first nucleotide sequence (a forward primer) and a second nucleic acid comprises a second nucleotide sequence (a reverse primer). Exemplary primer pairs for amplifying informative breast cancer loci are provided in Table 13. In certain embodiments, the composition comprises any of the set of nucleic acids provided in Table 13. As noted above, the primers are of less than or equal to 100 nucleotides in length (e.g., less than or equal to 100, 90, 80, 75, 70, 65, 60, 55, 50, 45, 40, 35, 30, 25, or 20) and comprise a nucleotide sequence suitable for amplifying an informative loci. In other words, the primer comprises a sequence that is complementary to and/or hybridizes under stringent conditions to human nucleic acid flanking an informative microsatellite loci.

In certain embodiments, the informative microsatellite loci are identified using the computer implemented methods described herein.

In certain embodiments, a sample from a subject (or samples from a plurality of subjects) is analyzed using a Next-Generation sequencing platform. In certain embodiments, sample preparation and/or enrichment for microsatellites is performed using reagents compatible with a Next-Generation sequencing platform. In other words, exemplary kits, including amplification and enrichment kits, include reagents compatible with Next-Generation sequencing platforms.

In certain embodiments, allelotypes or genotypes are determined using a Next-Generation sequencing platform, including using methods for generating a library of sequencing data, aligning sequences, and ultimately determining high quality reads.

Any method of sequencing known in the art can be used. Sequencing of nucleic acids isolated by selection methods are typically carried out using next-generation sequencing (NGS). Next-generation sequencing includes any sequencing method that determines the nucleotide sequence of either individual nucleic acid molecules or clonally expanded proxies for individual nucleic acid molecules in a highly parallel fashion (e.g., greater than 10⁵ molecules are sequenced simultaneously). Next generation sequencing methods are known in the art, and are described, e.g., in Metzker, M. (2010) Nature Biotechnology Reviews 11:31-46. Platforms for next-generation sequencing include, but are not limited to, Roche/454's Genome Sequencer (GS) FLX System, Illumina/Solexa's Genome Analyzer (GA), Life/APG's Support Oligonucleotide Ligation Detection (SOLiD) system, Polonator's G.007 system, Helicos BioSciences' HeliScope Gene Sequencing system, and Pacific Biosciences' PacBio RS system.

In certain embodiments, the disclosure provides kits comprising reagents suitable for enriching samples prior to sequencing using a Next-Generation sequencing platform. Such kits are described herein.

Samples

A “sample” may be any source from which nucleic acid may be obtained. Suitable nucleic acid that may be obtained is DNA and RNA. Exemplary samples include, but are not limited to, for example, a sample may be a buccal swab, a saliva sample, a blood sample, or other suitable samples containing genomic DNA or RNA, as described herein. In certain embodiments, the sample is obtained by non-invasive means (e.g., for obtaining a buccal sample, saliva sample, hair sample or skin sample). In certain embodiments, the sample is obtained by non-surgical means, i.e. in the absence of a surgical intervention on the individual that puts the individual at substantial health risk. Such embodiments may, in addition to non-invasive means also include obtaining sample by extracting a blood sample (e.g., a venous blood sample).

In other embodiments, the sample is a tumor sample. In other embodiments, the sample is taken from tissue adjacent to the tumor (the margin).

Regardless of tissue source, the nucleic acid examined may be DNA or RNA. In certain embodiments, the DNA is genomic DNA. The nucleic acid may be tumor specific, and tumor specific nucleic acid is analyzed by analyzing tumor samples. Additionally or alternatively, the nucleic acid may be germline. In the context of the present application, the term “germline” does not indicate that the sample is taken from, for example, germline tissues. Rather, the term indicates that the sample is such that the nucleic acid is indicative of the nucleic acid existing in the non-tumor somatic cells of the body from birth. Nucleic acid of tumor cells may differ from germline nucleic acid content due to tumor-specific mutations. One of the surprising discoveries described in the instant disclosure is that analysis of germline nucleic acid reveals variability in microsatellites indicative of increased risk of disease. In other words, increased risk can be evaluated proactively, prior to onset of detectable disease, by assessment of germline nucleic acid. Further, informative microsatellite loci can be determined by assessment of germline nucleic acid. In certain embodiments, risk assessment for an individual subject is performed at birth or early childhood based on analysis of a sample taken at birth, soon after birth, or in early childhood.

The disclosure contemplates that a sample may be a fresh or frozen sample, and nucleic acid may be isolated from that sample. Once nucleic acid is obtained, it may be processed to obtain sequence information, such as processed for analysis using a Next Generation sequence platform. Alternatively, nucleic acid information for a particular sample or for members of the population may be previously obtained, such as information from the 1000 genomes project. If nucleic acid sequence information was previously obtained, that information may be provided for further analysis, such as provided to a host computer as sequence information.

### 5. Reports, Programmed Computers, Business Methods, and Systems

The results of a test (e.g., an individual's risk for cancer, or an individual's predicted drug responsiveness, based on determining a variation at one or more informative microsatellite loci disclosed herein), and/or any other information pertaining to a test, may be referred to herein as a “report”. A tangible report can optionally be generated as part of a testing process (which may be interchangeably referred to herein as “reporting”, or as “providing” a report, “producing” a report, or “generating” a report).

Examples of tangible reports may include, but are not limited to, reports in paper (such as computer-generated printouts of test results) or equivalent formats and reports stored on computer readable medium (such as a CD, USB flash drive or other removable storage device, computer hard drive, or computer network server, etc.). Reports, particularly those stored on computer readable medium, can be part of a database, which may optionally be accessible via the internet (such as a database of patient records or genetic information stored on a computer network server, which may be a “secure database” that has security features that limit access to the report, such as to allow only the patient and/or the patient's medical practitioners to view the report while preventing other unauthorized individuals from viewing the report, for example). Additionally or alternatively, reports can be displayed on a computer screen (or the display of another electronic device or instrument), and such displays are also examples of tangible reports.

A report can include, for example, an individual's risk for a disease or condition, such as cancer. The report may indicate a general risk, such as a general risk of cancer based on GMI analysis. Additionally or alternatively, a report may indicate risk of developing a particular cancer, such as breast or ovarian cancer. The report of risk may be in the form of, for example, a graphical distribution, a binary conclusion (e.g., “yes” the subject is at increased risk or “no” the subject is not), or a qualitative or quantitative risk conclusion (e.g., the subject's risk is low, intermediate, or high). Additionally or alternatively, the report may provide information regarding the allele(s)/genotype that an individual carries at one or more informative microsatellite loci, such as the loci disclosed herein, which may optionally be linked to information regarding the significance of having the allele(s)/genotype at the microsatellite (for example, a report on computer readable medium such as a network server may include hyperlink(s) to one or more journal publications or websites that describe the medical/biological implications, such as increased or decreased disease risk, for individuals having a certain allele/genotype). Thus, for example, the report can include disease risk or other medical/biological significance (e.g., drug responsiveness, etc.) as well as optionally also including the allele/genotype information, or the report may just include allele/genotype information without including disease risk or other medical/biological significance (such that an individual viewing the report can use the allele/genotype information to determine the associated disease risk or other medical/biological significance from a source outside of the report itself, such as from a medical practitioner, publication, website, etc., which may optionally be linked to the report such as by a hyperlink).

A report can further be “transmitted” or “communicated” (these terms may be used herein interchangeably), such as to the individual who was tested, a medical practitioner (e.g., a doctor, nurse, clinical laboratory practitioner, genetic counselor, etc.), a healthcare organization, a clinical laboratory, and/or any other party or requester intended to view or possess the report. The act of “transmitting” or “communicating” a report can be by any means known in the art, based on the format of the report. Furthermore, “transmitting” or “communicating” a report can include delivering a report (“pushing”) and/or retrieving (“pulling”) a report. For example, reports can be transmitted/communicated by various means, including being physically transferred between parties (such as for reports in paper format) such as by being physically delivered from one party to another, or by being transmitted electronically or in signal form (e.g., via e-mail or over the internet, by facsimile, and/or by any wired or wireless communication methods known in the art) such as by being retrieved from a database stored on a computer network server, etc.

In certain exemplary embodiments, the disclosure provides computers (or other apparatus/devices such as biomedical devices or laboratory instrumentation) programmed to carry out the methods described herein. For example, in certain embodiments, the disclosure provides a computer programmed to receive (i.e., as input) the identity (e.g., the allele(s) or genotype at an informative microsatellite loci) of one or more informative microsatellite loci disclosed herein and provide (i.e., as output) the disease risk (e.g., an individual's risk for cancer) or other result (e.g., disease diagnosis or prognosis, drug responsiveness, etc.) based on the identity of the one or more informative microsatellite loci. Such output (e.g., communication of disease risk, disease diagnosis or prognosis, drug responsiveness, etc.) may be, for example, in the form of a report on computer readable medium, printed in paper form, and/or displayed on a computer screen or other display.

In various exemplary embodiments, the disclosure further provides methods of doing business (with respect to methods of doing business, the terms “individual” and “customer” are used herein interchangeably). For example, exemplary methods of doing business can comprise assaying one or more informative microsatellite loci disclosed herein and providing a report that includes, for example, a customer's risk for a disease (based on which allele(s)/genotype is present at the one of more assayed informative microsatellite loci) and/or that includes the allele(s)/genotype at the one or more assayed informative microsatellite loci which may optionally be linked to information (e.g., journal publications, websites, etc.) pertaining to disease risk or other biological/medical significance such as by means of a hyperlink (the report may be provided, for example, on a computer network server or other computer readable medium that is internet-accessible, and the report may be included in a secure database that allows the customer to access their report while preventing other unauthorized individuals from viewing the report), and optionally transmitting the report. Customers (or another party who is associated with the customer, such as the customer's doctor, for example) can request/order (e.g., purchase) the test online via the internet (or by phone, mail order, at an outlet/store, etc.), for example, and a kit can be sent/delivered (or otherwise provided) to the customer (or another party on behalf of the customer, such as the customer's doctor, for example) for collection of a biological sample from the customer (e.g., a buccal swab for collecting buccal cells), and the customer (or a party who collects the customer's biological sample) can submit their biological samples for assaying (e.g., to a laboratory or party associated with the laboratory such as a party that accepts the customer samples on behalf of the laboratory, a party for whom the laboratory is under the control of (e.g., the laboratory carries out the assays by request of the party or under a contract with the party, for example), and/or a party that receives at least a portion of the customer's payment for the test). The report (e.g., results of the assay including, for example, the customer's disease risk and/or allele(s)/genotype at the one or more assayed informative microsatellite loci) may be provided to the customer by, for example, the laboratory that assays the one or more assayed informative microsatellite loci or a party associated with the laboratory (e.g., a party that receives at least a portion of the customer's payment for the assay, or a party that requests the laboratory to carry out the assays or that contracts with the laboratory for the assays to be carried out) or a doctor or other medical practitioner who is associated with (e.g., employed by or having a consulting or contracting arrangement with) the laboratory or with a party associated with the laboratory, or the report may be provided to a third party (e.g., a doctor, genetic counselor, hospital, etc.) which optionally provides the report to the customer. In further embodiments, the customer may be a doctor or other medical practitioner, or a hospital, laboratory, medical insurance organization, or other medical organization that requests/orders (e.g., purchases) tests for the purposes of having other individuals (e.g., their patients or customers) assayed for one or more informative microsatellite loci disclosed herein and optionally obtaining a report of the assay results.

In certain exemplary methods of doing business, kits for collecting a biological sample from a customer (e.g., a swab for collecting cells from the inside of the cheek) are provided (e.g., for sale), such as at an outlet (e.g., a drug store, pharmacy, general merchandise store, or any other desirable outlet), online via the internet, by mail order, etc., whereby customers can obtain (e.g., purchase) the kits, collect their own biological samples, and submit (e.g., send/deliver via mail) their samples to a laboratory which assays the samples for one or more informative microsatellite loci disclosed herein (such as to determine the customer's risk for a disease) and optionally provides a report to the customer (of the customer's disease risk based on their informative microsatellite profile, for example) or provides the results of the assay to another party (e.g., a doctor, genetic counselor, hospital, etc.) which optionally provides a report to the customer (of the customer's disease risk based on their informative microsatellite profile, for example).

Certain further embodiments of the disclosure provide a system for determining an individual's risk for a particular disease, or whether an individual will benefit from a drug treatment (or other therapy) in reducing disease risk. Certain exemplary systems comprise an integrated “loop” in which an individual (or their medical practitioner) requests a determination of such individual's risk for a particular disease (or drug response, etc.), this determination is carried out by testing a sample from the individual, and then the results of this determination are provided back to the requester. For example, in certain systems, a sample (e.g., blood or buccal cells) is obtained from an individual for testing (the sample may be obtained by the individual or, for example, by a medical practitioner), the sample is submitted to a laboratory (or other facility) for testing (e.g., determining the genotype of one or more informative microsatellite loci disclosed herein), and then the results of the testing are sent to the patient (which optionally can be done by first sending the results to an intermediary, such as a medical practitioner, who then provides or otherwise conveys the results to the individual and/or acts on the results), thereby forming an integrated loop system for determining an individual's risk for a particular disease (or drug response, etc.). The portions of the system in which the results are transmitted (e.g., between any of a testing facility, a medical practitioner, and/or the individual) can be carried out by way of electronic or signal transmission (e.g., by computer such as via e-mail or the internet, by providing the results on a website or computer network server which may optionally be a secure database, by phone or fax, or by any other wired or wireless transmission methods known in the art). Optionally, the system can further include a risk reduction component (i.e., a disease management system) as part of the integrated loop. For example, the results of the test can be used to reduce the risk of the disease in the individual who was tested, such as by implementing a preventive therapy regimen (e.g., administration of a drug regimen such as an anticoagulant and/or antiplatelet agent for reducing risk for a particular disease), modifying the individual's diet, increasing exercise, reducing stress, and/or implementing any other physiological or behavioral modifications in the individual with the goal of reducing disease risk. For reducing disease risk, this may include any means used in the art for improving cardiovascular health. Thus, in exemplary embodiments, the system is controlled by the individual and/or their medical practitioner in that the individual and/or their medical practitioner requests the test, receives the test results back, and (optionally) acts on the test results to reduce the individual's disease risk, such as by implementing a disease management component.

The disclosure contemplates all operable combinations of any of the foregoing or following aspects and embodiments of the disclosure. Moreover, the various method steps described herein may be computer-implemented, such as by providing suitable information to a processor. Moreover, providing risk assessment, prognostic, and/or diagnostic information to, for example, a patient or medical professional can be computer implemented and done via a computer interface such as a web-based user interface.

These and other aspects of the present disclosure will be further appreciated upon consideration of the following Examples, which are intended to illustrate certain particular embodiments of the disclosure but are not intended to limit its scope, as defined by the claims.

### EXAMPLES

### Example 1

### Global Microsatellite Instability and Identification of Informative Microsatellite Loci: Breast Cancer

### Methods

Identifying Microsatellites.

Using Tandem Repeats Finder (Benson, G. Tandem repeats finder: a program to analyze DNA sequences. Nucleic acids research 27, 573-580 (1999)), over a million microsatellites in the human genome (NCBI36/hg18) were identified with the following parameters: matching weight=2, mismatching penalty=5, indel penalty=5, match probability=80, indel probability=10, minimum alignment score to report=14, maximum period size to report=4 and 6. All monomers, microsatellite loci in or near large repetitive elements, as found using RepeatMasker (Smit A F A, H. R., Green P. RepeatMasker Open-3.0, <http://www.repeatmasker.org> (1996-2012)), and microsatellites with non-unique flanking sequences were removed from this set, resulting in a subset of 744,618 microsatellite loci. Microsatellites were associated with their corresponding location in or near Refseq genes using the UCSC Genome Browser (Rhead, B. et al. The UCSC Genome Browser database: update 2010. Nucleic acids research 38, D613-D619 (2010)).

RNA-Seq Equivalent Microsatellite Subset.

To allow for comparisons between samples that were RNA and exome sequenced, a set of microsatellites which were captured at least one of the 380 RNA-seq BC tumor samples were selected. This set totaled 13,739 exonic microsatellites.

Genotyping Microsatellites.

All reads were filtered to remove low quality reads using the same methods applied to the 1,000 Genomes Project data. These reads were then aligned to the human reference genome (NCBI36/hg18) using BWA (Li, H. et al. The Sequence Alignment/Map format and SAMtools. Bioinformatics (Oxford, England) 25, 2078-2079 (2009); and Li, H. & Durbin, R. Fast and accurate short read alignment with Burrows-Wheeler transform. Bioinformatics (Oxford, England) 25, 1754-1760 (2009)). Microsatellite loci were called with high accuracy using software that considers only reads which completely span the microsatellite and contain at least 5 bp of unique flanking sequence on both sides (McIver, L. J., Fondon, J. W., 3rd, Skinner, M. A. & Garner, H. R. Evaluation of microsatellite variation in the 1000 Genomes Project pilot studies is indicative of the quality and utility of the raw data and alignments. Genomics 97, 193-199 (2011) and McIver L J, McCormick J F, Martin A, Fondon J W 3rd, Garner H R. Population-scale analysis of human microsatellites reveals novel sources of exonic variation. Gene. 10; 516(2):328-34 (2013), incorporated by reference in their entireties herein). Allele lengths that are not confirmed by a minimum of 3 reads are not considered reliable and are removed from the analysis. Microsatellites are considered to be heterozygous if the reads for each allele are no more than two times the reads of the second allele. This allows for unequal amplification, which is an issue with next-generation sequencing, with only 17-40% of microsatellite alleles sequencing equally. Wells, D., Sherlock, J. K., Handyside, A. H. & Delhanty, J. D. Detailed chromosomal and molecular genetic analysis of single cells by whole genome amplification and comparative genomic hybridisation. Nucleic acids research 27, 1214-1218 (1999); and Sherlock, J., Cirigliano, V., Petrou, M., Tutschek, B. & Adinolfi, M. Assessment of diagnostic quantitative fluorescent multiplex polymerase chain reaction assays performed on single cells. Ann Hum Genet 62, 9-23 (1998).

Consensus Microsatellite Lengths.

Consensus microsatellite lengths were developed from the set of 131 female normal samples. They are the most common allele called in these samples.

Identifying Novel Microsatellite Variants.

Using data from dbSNP v128 build to correspond to hg18 we were able to computationally determine which variants were known (Sherry, S. T. et al. dbSNP: the NCBI database of genetic variation. Nucleic acids research 29, 308-311 (2001)). Additionally some exonic variants were manually checked using the latest version of dbSNP v137, to ensure these variants had not been recently documented.

Validation of Microsatellite Variants.

Select microsatellite loci in 28 normal bloodline samples (also referred to as germline samples—in other words, samples from non-tumor tissue such that the nucleic acid is indicative of germline nucleic acid), 66 breast cancer bloodline samples and 6 ovarian cancer bloodline samples obtained from UTSR were analyzed. PCR amplification of loci contained in the following genes was performed using primers described in Table 13: CABIN1, NSUN5, CDC2L1, PRKCA and MAPKAPK3. All of the PCR amplifications were then run on the QIAGEN QIAxcel system using the DNA High Resolution Cartridge. The results were analyzed using the QIAxcel Screengel Software and compiled using Microsoft Excel. The loci located in MAPKAPK3 and CDC2L1 were examined in greater detail by the Genomics Research Laboratory at Virginia Bioinformatics Institute.

Determining GMI.

GMI was calculated as the # of microsatellite loci containing at least one non-consensus microsatellite allele length/total callable microsatellite loci for a given sample. To allow for comparisons between samples that were RNA and exome sequenced, only RNA-seq equivalent microsatellite subset were considered in this calculation.

Prediction of Transcription Factor Binding Sites.

Data from Transfac that predicted transcription factor binding sites based on conserved locations from the human/mouse/rat alignment were used to computationally find if microsatellites were located in or near these sites (Matys, V. et al. TRANSFAC and its module TRANSCompel: transcriptional gene regulation in eukaryotes. Nucleic acids research 34, D108-D110 (2006)).

Identifying Relationships Between Genes Containing BC-Associated Microsatellites.

Molecular, cellular, and biological processes involving genes with significant BC-associated microsatellite variants were determined from the analysis of Genome Ontology (GO) terms using the Panther Classification System (Thomas, P. D. et al. PANTHER: a browsable database of gene products organized by biological function, using curated protein family and subfamily classification. Nucleic acids research 31, 334-341 (2003)). GO terms over-represented (P≦0.1) in comparison to a reference Homo sapiens gene list provided through Panther were analyzed. All of the signature loci represented in Table 2 were manually inspected using the UCSC Genome Browser to determine if they had any associations with other data sets of interest included the data provided by ENCODE (Rhead, B. et al. The UCSC Genome Browser database: update 2010. Nucleic acids research 38, D613-D619 (2010); Bernstein, B. E. et al. Genomic maps and comparative analysis of histone modifications in human and mouse. Cell 120, 169-181 (2005); Bernstein, B. E. et al. A bivalent chromatin structure marks key developmental genes in embryonic stem cells. Cell 125, 315-326 (2006); and Mikkelsen, T. S. et al. Genome-wide maps of chromatin state in pluripotent and lineage-committed cells. Nature 448, 553-560 (2007)).

Protein Threading.

For each informative locus, the reference amino acid sequence and variant-associated amino acid sequence was determined. The position of each mapped gene was located using Ensembl, in NCBI36 (Ensembl release 54) and data were exported as FASTA files with 100 bp upstream and 300 bp downstream from the location of the gene. FASTA sequences were exported to ExPASy and DNA sequences were translated to protein sequence output. Manually, changes introduced to exonic DNA by MSI were introduced to FASTA sequences and translated with ExPASy. The reference protein sequence was identified using UniProtKB— these included the following queries: MAPKAPK3 (Q16644; MAPK3_Human); HSPA6 (P17066; HSP76_Human); CABIN1 (Q9Y6J; CABIN_HUMAN); NSUN5 (Q96P11; NSUN5_Human); and CDC2L1 (P21127; CD11B_Human). Both the reference and mutant amino acid sequences were threaded using RaptorX (Kallberg, M. et al. Template-based protein structure modeling using the RaptorX web server. Nature protocols 7, 1511-1522, doi:10.1038/nprot.2012.085 (2012)); from RaptorX, pdb files for the aligned sequences were used in other modeling methods—ligand binding sites were predicted using the protein modeling software Phyre 2 (Kelley, L. A. & Sternberg, M. J. Protein structure prediction on the Web: a case study using the Phyre server. Nature protocols 4, 363-371, doi:10.1038/nprot.2009.2 (2009)) and the individual amino acids altered in the protein structure pdb files were highlighted using Swis-PDB Viewer (Version 4.1.0). Phyre2 was also used to determine the percent confidence and identity for each model.

### Results

GMI in Breast Cancer and Normal Samples

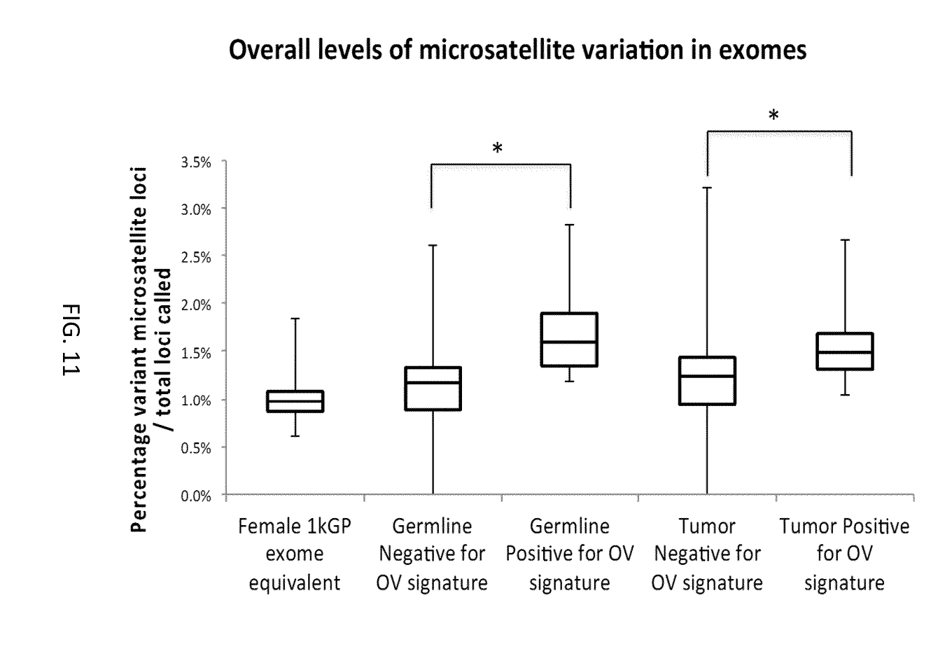

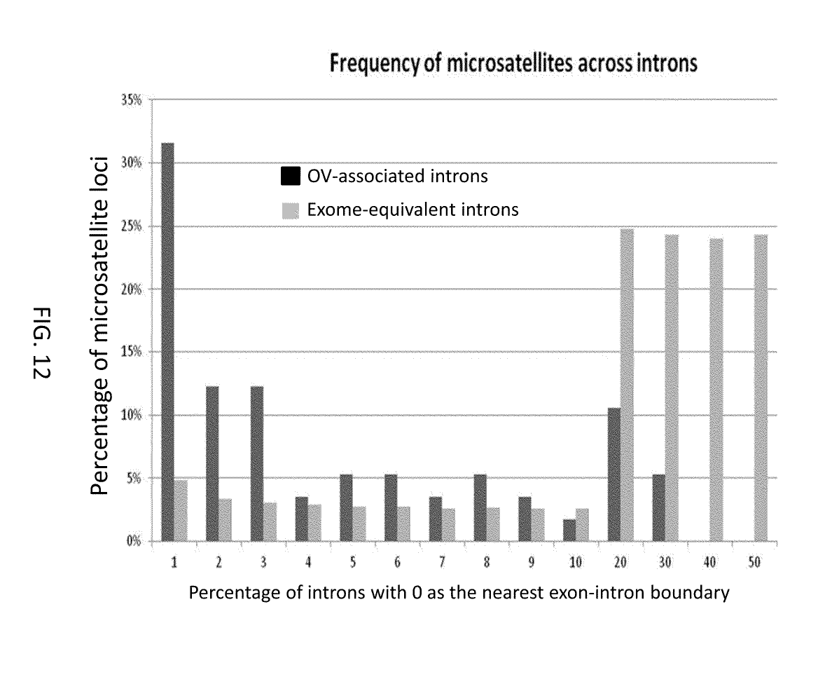

GMI was analyzed in 399 transcriptomes of women with invasive breast carcinoma (Newman, B. et al. Frequency of breast cancer attributable to BRCA1 in a population-based series of American women. Jama 279, 915-921 (1998)), and 100 germline and 100 tumor exome-enriched genomic samples and compared with 118 transcriptomes of cancer-free individuals and exon-matched genomic microsatellite loci from 131 cancer-free women (and 119 men), from The Cancer Genome Atlas (TCGA) and 1,000 Genomes Projects (Durbin, R. M. et al. A map of human genome variation from population-scale sequencing. Nature 467, 1061-1073), respectively. The TCGA invasive breast carcinoma dataset (BC) contained RNA-seq data from 375 samples from tumor, 10 samples from non-tumor of which 5 are matched, and 14 samples of whose tumor/non-tumor status was “unknown”. In addition 100 BC germline and 100 BC tumor genomes that were exome sequenced (WXS) were analyzed. Unless otherwise specified, for the most accurate comparisons between all the data types (RNA-seq, exome, and whole-genome sequencing), the analysis was restricted to the 13,739 microsatellite loci that were identifiable in at least one sample from the BC RNA-seq data. Previous studies have shown that accurate allele calls can be inferred from RNA-seq data (Levin, J. Z. et al. Targeted next-generation sequencing of a cancer transcriptome enhances detection of sequence variants and novel fusion transcripts. Genome biology 10, R115, doi:gb-2009-10-10-r115). 9 of the 375 BC RNA tumor samples were removed from the subsequent analysis because the inability of obtaining any reliable microsatellite loci in those genomes. For the remaining 366 samples, genotypes were called at an average of 7,976 loci per sample with only 6 samples having less than 5,000 reliable microsatellite calls (FIG. 9). Approximately, 75% of the BC samples had between 4 and 8 variant microsatellite loci (FIG. 10), with an average of 6 variant loci per sample. In addition, 82% of the BC RNA samples had at least one variant microsatellite locus that is projected to result in a transcript with a frame shift.

The total GMI variation frequency was not significantly different between tumor and non-tumor samples of cancer patients, 0.071% and 0.069%, respectively. This indicates that there is an increase in GMI in the germline of people at risk for BC rather than exclusively in BC tumors. In this case there should be a significant increase in GMI between BC and the normal population. To test this hypothesis, basal level of GMI in the ‘normal’ population was determined using the sequencing data of individuals whose genomes and/or transcriptomes were sequenced as part of The 1,000 Genomes Project (1 kGP). The female 1 kGP genomic samples had a mean GMI of 0.041%±0.020% while the transcriptomes had a mean GMI of 0.036%±0.106%. The 118 normal transcriptomes were highly similar to the total 1 kGP population with variation frequency of 0.036%±0.106%.

A comparison of normal samples to BC demonstrates the average level of GMI in the BC population is 1.7 times greater than the normal population at coding loci, supporting the hypothesis that GMI level may be an indicator of risk for BC. However the range of variation within both populations was broad, leading to overlap in the standard deviations. Therefore, three GMI classes were assigned—with low (non-cancer-like) as less than 0.04%, intermediate as 0.04% to 0.06%, and high (cancer-like) as 0.06% and greater. A closer analysis revealed that 50.4% of the 250 1 kGP normal samples would be considered low GMI, 30.4% would be intermediate, and 19.2% would be GMI high. For the BC samples, 17.3% were low GMI, 22.1% intermediate and 60.7% high GMI. This difference would likely be even more pronounced if comparing variation levels at non-coding microsatellite loci as the frequency of variation for all genomic regions in the 1 kGP data was 36 times that found in coding regions, consistent with previous measurements and the fact that these loci lie in a variety of genomic locations (introns, exons, intergenic spaces) which exhibit differing selective pressures.

BC Associated Microsatellite Loci.

Each of the 13,739 microsatellite loci included in this analysis was called in an average of 251 of the RNA BC samples. There were 165 loci for which at least one BC RNA sample was variant from the human genome reference (hg18) (Table 1). A leave-one-out statistical approach was employed to identify those loci that are most informative for properly assigning the genomes to the correct cancer and non-cancer populations. In addition, it was found that 1 kGP genomes had (<4% variation) and the 100 BC germline exome data had >4.5% variation.

BC RNA Signature.

Short read length limited the number of microsatellites that could be successfully genotyped in the normal RNA data set (few reads contained the complete microsatellite and sufficient flanking sequence for accurate microsatellite length detection). Therefore, the variations within 1 kGP normal genomes was used in the comparative analysis to identify ‘BC-associated’ loci (Table 2) which had significantly greater variation within the BC RNA samples over that seen in the 1 kGP females. Using these loci, BC transcriptomes as carrying a ‘BC signature’ were identified with a sensitivity of 87.2% (BC tumor) and 100% (BC somatic) and a minimum specificity of 96.2% Importantly, it should also be noted that the majority of these loci are highly conserved in the cancer-free population, which consists of females from four different ethnic groups; therefore these loci are conserved across ethnic groups and the variations seen in the BC samples are unlikely to be attributed to ethnicity. These loci are also conserved independent of sex as they are also conserved in a set of 119 normal males. Of the informative loci, 5 had variant transcripts in over 50% of both the BC tumor and germline RNA samples. Using these 5 loci to classify samples as having a BC signature, it was possible to distinguish between BC and normal with a sensitivity of 86.1% (BC tumor) and 100% (BC somatic) with a specificity of 99.2%. These loci reside in the MAPKAPK3, CABIN1, HSPA6, NSUN5 and CDC2L1 genes and had a variation frequency of 54.5%, 51.4%, 74.2%, 72.8% and 99.5% respectively (Table 2 and FIG. 7). The high frequency of variation at the 5 highly variable BC-associated loci, and particularly at CDC2L1, can be explained by either (1) these markers are pre-existing in people who develop cancer and as such can be used as a novel risk assessment tool for BC or (2) these variations arise at a high frequency in tumors implying that they likely provide an advantage to the tumor and are potential markers or targets. Although it was not possible to accurately genotype most loci from the normal RNA samples with sufficient population depth and read depth to determine their normal variation frequency, NSUN5 was genotyped in 41 normal samples with only 2.4% variation, confirming that there was a significant increase in genomes carrying the NSUN5 variation in the RNA from BC vs normal individuals.

Altered Protein Sequences.

To predict if the 5 highly-variable BC-associated microsatellites variants potentially introduce alterations in protein sequence or structure, RaptorX was used to model the protein structures with and without the variants (Table 11). The variant in MAPKAPK3 resulted in a putative frame-shift mutation producing a mutant protein with an extended C-terminus, 17 amino acids longer than the wild-type Importantly, these changes are located in the p38 MAPK-binding site (a.a. 345-369) and bipartite nuclear localization signal 2 (a.a. 364-368) regions. This suggests breast cancer patients with this variation may have an alternative MAPKAPK3 protein that is unable to localize to the nucleus for transcription regulation and has altered affinity to the p38 MAPK-binding site. In HSPA6, the microsatellite variation is predicted to result in a two amino acid deletion but not a frame-shift; importantly, these changes occur in residues 502-505 where Lys (a.a. 502) is a modification site. Lysine modifications in macromolecular proteins such as HSPA6 are associated with chromatin remodeling, cell cycle, splicing, nuclear transport, and actin nucleation as described by Choudhary et al (Choudhary, C. et al. Lysine acetylation targets protein complexes and co-regulates major cellular functions. Science 325, 834-840, doi:10.1126/science.1175371 (2009)). Thus, modifications introduced through microsatellite variants may alter HSPA6 acetylation leading to changes in normal cellular processes. The variations in CABIN1, NSUN5, and CDC2L1 were in non-conserved domains and were not predicted to create frameshifts (Table 11), however modifications to the amino acid sequence may introduce conformational changes and alternative binding affinities that permit ligands—otherwise not associated with these proteins (or regions of the same protein) to bind more freely in the altered structures. The microsatellite variations in both CABIN1 and CDC2L1 are predicted to alter ligand binding. Additionally, changes in regions associated with post-translational modification could result in changes to normal protein activities that regulate key cellular functions.

### Example 2

### Global Microsatellite Instability and Identification of Informative Loci: Ovarian Cancer

### Methods

Data Sets.

The set of 250 genomes used to develop a set of normal microsatellite distributions were sequenced by the 1000 Genomes Project (R. M. Durbin et al., Nature 467, 1061 (Oct. 28, 2010)). These individuals were whole genome sequenced at low coverage and exome sequenced at high coverage. Samples from individuals with ovarian cancer were sequenced by The Cancer Genome Atlas for study phs000178.v5.p5 (Nature 474, 609 (Jun. 30, 2011)). The majority of the samples were exome sequenced. The raw sequencing reads obtained for this study through NCBI SRA were downloaded, decrypted, and decompressed using software by NCBI SRA. Then they were filtered based on the quality score requirements set forth by the 1000 Genomes Project (R. M. Durbin et al., Nature 467, 1061 (Oct. 28, 2010)).

Identifying Microsatellites.

Microsatellites at least 10 base pairs long, with no more than one interruption to the canonical repeat sequence per ten bases in length were identified within the human reference genome (NCBI36/hg18) using Tandem Repeat Finder with parameters 2, 5, 5, 80, 10, 14, 6 to create a set of 1 to 6-mers (G. Benson, Nucleic acids research 27, 573 (Jan. 15, 1999)). Microsatellites within or adjacent to other repetitive elements identified using RepeatMasker were removed. The USCS Genome Browser provided information as to the chromosomal location of Refseq genes with this study (T. R. Dreszer et al., Nucleic acids research 40, D918 (January, 2012)).

Identifying Variations at Microsatellite Loci Using Microsatellite-Based Genotyping.

Quality filtered reads from The Cancer Genome Atlas (Nature 474, 609 (Jun. 30, 2011)), were aligned to the human reference genome (NCBI36/hg18) using BWA (H. Li, R. Durbin, Bioinformatics (Oxford, England) 25, 1754 (Jul. 15, 2009)). The microsatellite-based genotyping used herein uses non-repetitive flanking sequences to ensure reliable mapping and alignment at microsatellite loci by filtering out all microsatellite-containing reads that do not completely span the repeat as well as provide some additional unique flanking sequence on both sides (L. J. McIver, J. W. Fondon, 3rd, M. A. Skinner, H. R. Garner, Genomics 97, 193 (April, 2011)). The unique flanking sequence, along with a small portion of the repeat is then used for local alignment of the read to the correct genomic locus. The same local alignment procedure is used to align reads which were not aligned to the reference by BWA, obtaining additional coverage at some loci.

For each of the ˜850,000 loci, reads were grouped based on the repeat length variations or SNPs they contained. Allelic variations supported by less than three reads were filtered. A locus was considered to be heterozygous only when the number of reads for the major allele was less than twice the reads of the second most abundant allele. This method is conservative in estimations of heterozygosity yet allows for unequal amplification of alleles during the library preparation prior to sequencing. All microsatellites whose reads did not meet the criteria for calling two alleles were considered to be homozygous and only the most abundant allele was reported.

Consensus Vs Reference.

Reads from 250 genomes, from four different ethnic backgrounds, sequenced by the 1000 Genomes Project were aligned to the human reference genome (NCBI36/hg18) using BWA. Microsatellite-based genotyping, identical to that used with the matched ovarian samples, was run on these samples to obtain a distribution of variations for ˜850,000 loci. The consensus microsatellite length for each of the 850,000 loci was the allele which was called in the majority of the samples. 3.2% (23,934/742,562) of the microsatellites at high-credibility loci were identified in which the major allele from the 1 kGP did not agree with the hg18 human reference length, indicating that the hg18 reference genome does not always have the most common allele, and emphasizing the need to use the distribution of alleles within the normal population as a baseline for variant calling. For all comparisons to these loci, the consensus allele length from the 1 kGP was used instead of the human reference.

Rule Set for Identification of Ovarian Cancer-Variant Loci.

The rules used for identification of informative microsatellite loci were (1) conserved within the 1 kGP females (called in at least 25 females with less than 2% variation), (2) at least 3% of ovarian cancer alleles varied from the female consensus, and (3) ≧3 ovarian cancer alleles were different from the consensus. These loci are listed in Table 4.

Microsatellites Located Near Splice Sites and Transcription Factor Binding Sites in Normal and Cancer Data.

The locations of splice cites for all Refseq genes was obtained from the UCSC Genome Browser and then stored in a MySQL database for quick retrieval. A perl script was written to determine the location of each microsatellite with respect to the nearest splice site. The same process was done using those transcription factor binding sites (TFBS) that were conserved in the human/mouse/rat alignments. The script reported all TFBS/splice cites that were near each microsatellite including their distances.

Identifying Associations with Cancer.

Evaluation of the ovarian cancer-associated loci set for genes associated with cancer was done using Gene Ontology terms from OMIM and using the set distiller from GeneDecks, part of the GeneCards suite (A. Hamosh, A. F. Scott, J. S. Amberger, C. A. Bocchini, V. A. McKusick, Nucleic acids research 33, D514 (Jan. 1, 2005); G. Stelzer et al., OMICS 13, 477 (December, 2009)).

High-Credibility Loci.

Loci that are called in at least 25 of the 1 kGP samples are referred to as high-credibility loci. This was determined as the minimum number of genomes required for the absence of variant loci to be considered credible using a bayesian upper boundary.

### Results

Establishment of ‘Baseline’ GMI for Comparative Analysis To establish a baseline for variation, variation at each microsatellite locus in 250 individuals from four different populations in the 1 kGP data set was determined. These individuals had not been diagnosed with cancer at the time of sequencing therefore they should be representative of the normal population and should not be enriched for cancer-associated variants. It was possible to determine the microsatellite lengths in 86.7% of the possible 856,384 mono- to hexamer microsatellites in the hg18 human reference genome, in a minimum of 25 genomes. Only those loci called in at least 25 genomes were considered as having ‘high-credibility’ or sufficient coverage at the population level to reliably establish the normal allelic distribution. Of the 742,562 high credibility loci, only 11.9% had a variant allele in one or more of the 250 1 kGP samples. 670,090 microsatellite loci were ‘conserved’ within the 1 kGP population, defined as having less than 2% variant alleles at a high-credibility locus. The majority of exonic microsatellites (97.5%) were conserved in the 1 kGP population. Surprisingly, 84.1% of intronic and 85.0% of intergenic loci were also conserved, indicating potential conservation constraints for these microsatellite loci.

Comparison of GMI in Ovarian Cancer and Normal Samples

After establishing the ‘expected’ percentage of variant microsatellite alleles within the normal population, it was asked whether there was an increase in the overall frequency of microsatellite variation in ovarian cancer. For comparisons to the ovarian cancer data set, only data from the 131 1 kGP females was used to determine baseline variation. Ninety four percent of the microsatellite loci that were conserved in the 1 kGP population were also conserved within the female-only subset. Next-generation sequencing data from 78 germline samples, 60 of which also had matched tumors, and an additional 15 tumor samples from females diagnosed with epithelial ovarian carcinoma, were obtained from The Cancer Genome Atlas (Nature 474, 609 (Jun. 30, 2011)).

Microsatellite variation was significantly higher in ovarian cancer patients relative to the exome equivalent in healthy females (1.4% in germline and tumor vs. 1.0% in 1 kGP females, p≦0.005; Table 12). The WGS samples showed an even more distinct increase in microsatellite instability with ≧4% variation in OV genomes vs. 1.5% in the normal females (Table 12). Ovarian cancer individuals also had higher variation at conserved microsatellite loci. A subset of 600 microsatellite loci that were conserved in normal females yet had high levels of variation in either ovarian cancer germline DNA, tumors or both was identified. We narrowed this down to a set of 100 ‘ovarian cancer-associated loci’ using leave-one-out cross-validation (Table 4; the first 100 microsatellites represent the narrowed down set of informative microsatellite loci). Allele calls from the matched germline and tumor genomes at the 100 ovarian cancer-associated microsatellite loci were examined in order to get an overview of the frequency at which the ovarian cancer germline and tumor were consistent in their variation from the normal consensus. Twenty one loci had a higher level of coverage across exome-sequenced genomes. Several of these lie within known cancer-associated genes therefore the higher calling is likely due to higher probe coverage near these loci during exome enrichment. Overall, there were 1039 instances where a genotype was determined for both the germline and matched tumor. In 51/1039 cases (5.0%) both the germline and tumor had matched genotypes (either homozygous or heterozygous) that were different from the normal consensus, suggesting that germline microsatellite variation within our loci set could be a valuable novel risk assessment tool for ovarian cancer.

The ovarian cancer-associated subset of loci (e.g., informative microsatellite loci for ovarian cancer) was used to classify genomes as ‘normal’ or having an ‘OV signature’. It was found that requiring a minimum of 4 variant loci in the OV microsatellite subset was sufficient to classify genomes as having an ‘ovarian cancer signature’ with a specificity of 99.2% and a sensitivity of 46% (Table 3). Of the 49 matched tumor/germline genomes, 13 had both the germline and tumor samples identified as carrying an ovarian cancer signature including all four WGS genomes. The rate of ovarian cancer in a normal population is approximately 1/58 (1.7%), and ˜50% of known OV-patients were identified as having an ovarian cancer signature. Combined, these two factors make the expected detectable frequency of ovarian cancer within the normal population 0.8%, which is consistent with what was observed when requiring a minimum of 4 variant alleles within the OV-associated loci set (Table 4). Similar analyses with a set of 100 random loci and the 500 microsatellite loci that were dropped from the informative loci set were unable to distinguish between OV signature and normal with the same high sensitivity and specificity as our OV-associated loci, indicating that the informative microsatellite locus set (microsatellites 1-100 in Table 4) is powerful in its ability to detect an OV signature with a low false discovery rate.

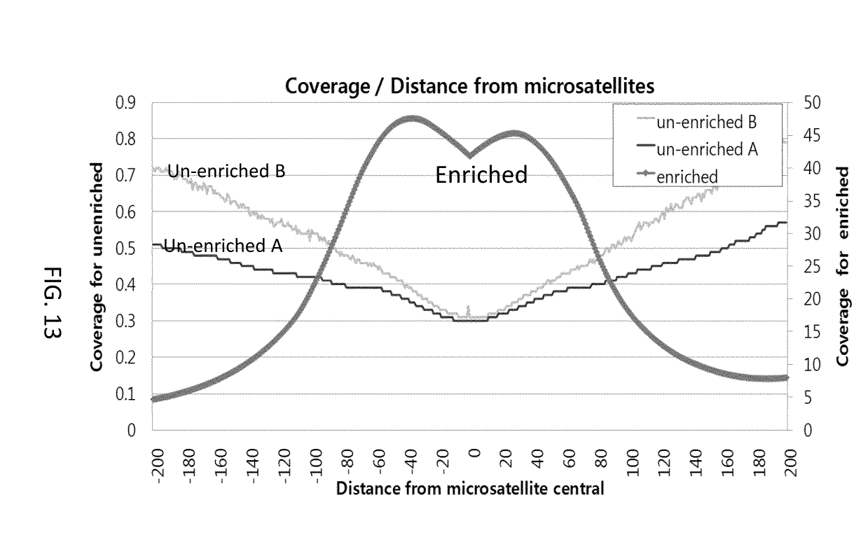

Analysis of the overall level microsatellite variation at all callable loci in the exome data revealed that germline and tumor exomes carrying an ovarian cancer signature have significantly higher level of variation than those that were not classified as having an ovarian cancer signature (FIG. 11). This indicates that the overall level of microsatellite instability is fairly represented by the 100-informative microsatellite subset, and suggests that there is a general microsatellite destabilization mechanism driving enhanced variation in individuals at risk for ovarian cancer.

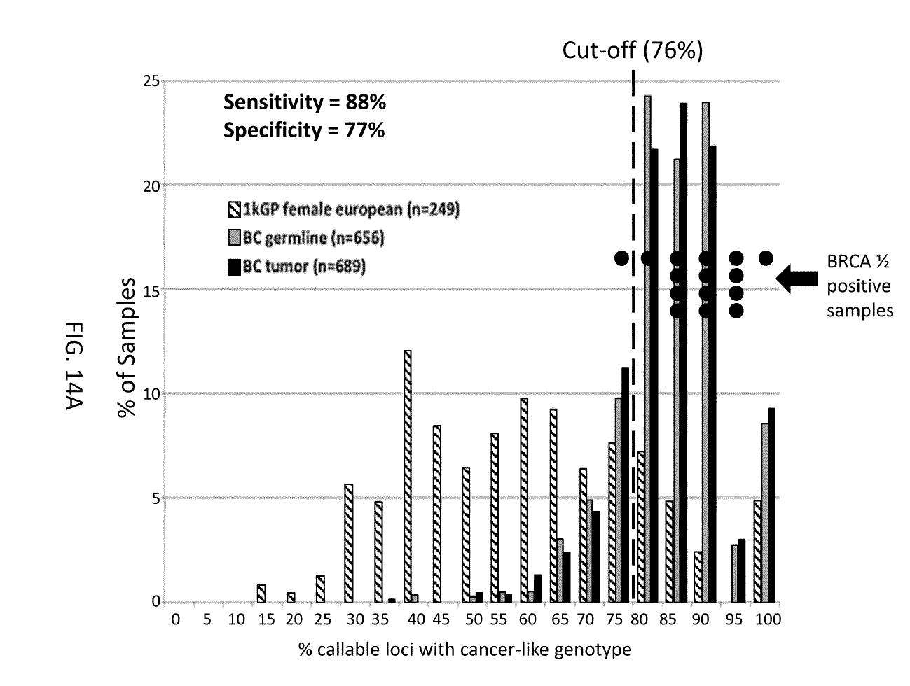

Furthermore, many of the conserved loci in the 1 kGP lie in introns, and 57% of the loci included in the ovarian cancer-associated subset are intronic. Splice sites are important regulatory elements that, if altered, can have dramatic effects on proteins and subsequent cellular function. Microsatellites that fall near exon-intron junctions have the potential to affect splicing (Y. Lian, H. R. Garner, Bioinformatics (Oxford, England) 21, 1358 (Apr. 15, 2005)). In general, microsatellite loci were evenly distributed across the introns, however those that were identified as being ovarian cancer-associated (e.g., microsatellites 1-100 in Table 4) are enriched near exon-intron boundaries (FIG. 12). Indeed, while only 3% of total intronic microsatellites fall within 50 nt of an exon-intron junction, 46% of the intronic loci that are included in the ovarian cancer-associated subset were identified as falling within this region. This suggests that variations at the ovarian cancer-associated loci may represent direct effectors of cellular function as well as risk-assessment markers.

### Example 3

### Global Microsatellite Instability and Identification of Informative Loci: Glioblastoma

Glioblastoma sequencing data was downloaded from The Cancer Genome Atlas and used to identify loci near and/or in genes that show changes in microsatellite length when compared with the consensus from the 1000 Genomes Project (1 kGP). A microsatellite genotype was reliably called at every repeat-containing locus in each sample which had sufficient depth and quality at 1000-10,000 of these loci to establish a basal level of GMI. A profile or distribution of alleles was then computed at each locus. Profiles generated for cancer and cancer-free samples at each locus were compared to identify those loci which exhibited significant levels of variation in cancer samples yet were conserved in cancer-free samples. These loci and the genes containing them were further analyzed to better understand their possible role in cancer etiology and to evaluate their potential as risk measures, possible therapeutic diagnostics and new therapy targets for glioblastoma.

Specifically, 250 (n=131 female; n=119 male) normal brain tissue samples from the 1 kGP was compared to GBM tumor (n=34) and GBM non-tumor samples (n=33) through a microsatellite identification software system ((McIver, L. J., Fondon, J. W., 3rd, Skinner, M. A. & Garner, H. R. Evaluation of microsatellite variation in the 1000 Genomes Project pilot studies is indicative of the quality and utility of the raw data and alignments. Genomics 97, 193-199 (2011)). 48 loci that are associated to glioblastoma were identified (Table 5). ‘Leave-one-out’ statistical analysis method was then used to determine which loci are most informative for properly assigning genomes to the correct cancer and non-cancer populations. Through this method we were able to identify 8 signature loci that contribute significantly (P≦0.05) to specificity and sensitivity in calling GBM positive samples (shaded in Table 5). It was determined that 4 of the 48 informative loci could be used to randomly identify GBM; 0% of normal samples tested positive while 29.4% of GBM tumors and 33.3% of germline, non-tumor glioblastoma samples tested positive (Table 6). With just 3 of the informative loci, 1.6% of normal tested positive (false positive); however, 39.5% of tumor tissue and 69.7% of glioblastoma non-tumor blood samples tested positive for these markers (Table 6). This demonstrates that the informative microsatellite loci identified in this study are a predicative marker of glioblastoma. Additionally, this demonstrates that these informative microsatellite loci could serve as a biomarker for glioblastoma in individuals before disease develops, since the informative microsatellite loci are present in bloodline samples and are not exclusive to tumors. These findings are depicted further in FIG. 8.

### Example 4

### Microsatellite Genotyping Reveals a Signature in Breast Cancer Exomes

### Methods

Data Sets and Selection of Background Samples:

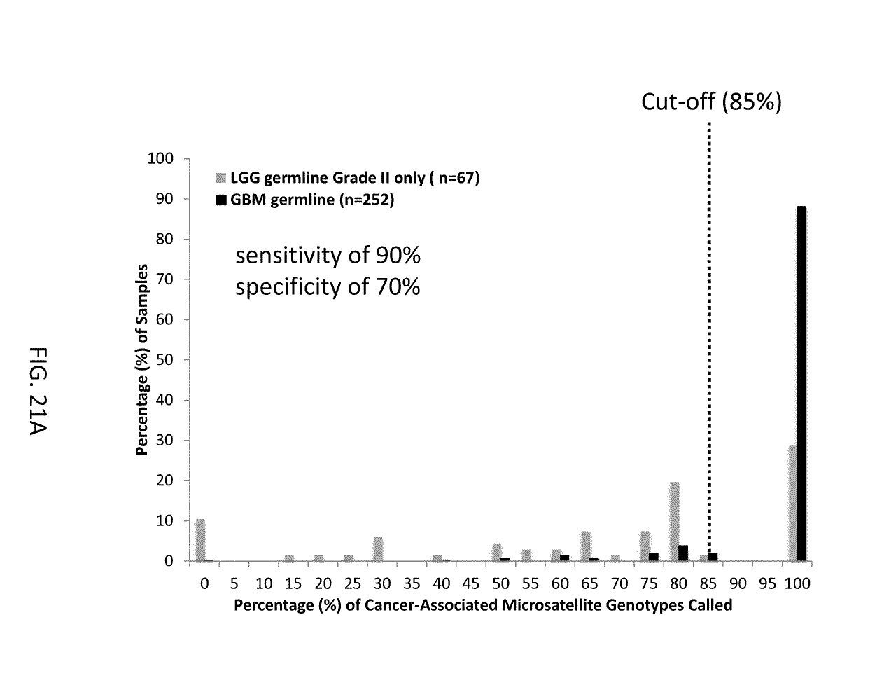

For the normal/healthy population, we downloaded all available exome samples from the phase 1 publication (n=886) of the 1000 Genomes Project (1 kGP) plus additional female samples (n=132) which were of the populations that best matched the cancer samples (FIG. 18). Germline (n=656) and tumor (n=689) samples from patients with BC, collected prior to any treatment, were obtained from The Cancer Genome Atlas (TCGA) (dbGAP Study Accession: phs000178.v8.p7). All available samples were downloaded including a set of 60 samples that were waiting for QC processing. These samples, like all others run through our pipeline, were processed to remove any reads that did not meet the QC thresholds as required in the 1000 Genomes Project, and then used as an independent set for validation. Additionally, we downloaded 104 RNAseq BC germline samples and 842 RNAseq BC tumor samples.

Microsatellite Genotyping:

All DNA samples from the 1 kGP and TCGA were exome enriched and sequenced on the Illumina platform then aligned to the current human reference, hg19, using BWA by their respective projects. We performed re-alignment and genotyping of microsatellites using our software and methods outlined below.

Creation of Microsatellite Target Set:

We produced a set of over 850,000 microsatellites which have flanking sequences unique in the human genome. Initially a set of over a million microsatellites was first found in the human genome (NCBI36/hg18) using Tandem Repeats Finder (TRF) (Benson G (1999) Nucleic acids research 27 (2):573-580), with parameters matching weight=2, mismatching penalty=5, indel penalty=5, match probability=80, indel probability=10, minimum alignment score to report=14, maximum period size to report=4, 6, and then 1. Changing the maximum period sizes allows us to identify microsatellites of different canonical repeat lengths, with some uniquely found in each set based on the algorithm used by TRF to identify repeat regions. We filter out those microsats which are less than 12 bases in length, except in exons which are allowed to be a minimum of 10 bases in length. We limit the length of microsatellites as short microsatellite motifs are less likely to be highly mutable when compared with long microsatellite motifs. We also filter out those microsatellites which contain single nucleotide polymorphisms (SNPs) and insertions and/or deletions (indels) in the human reference which would result in more than 10% differing from an ideal repetition of the canonical repeat. We perform this step as microsatellite purity also affects mutability with those microsatellites containing more replicates of the canonical repeat more likely to vary in part due to replication slippage. Microsatellites with embedded SNPs and their associated genotypes can also be reviewed. Microsatellites which overlapped were also removed as were microsats with at least one base overlapping a large repetitive element (SINEs, LINEs, and ALUs) as identified with RepeatMasker.

Next, multiple steps were performed to filter out microsatellites from the set which did not have unique flanking sequences. This is essential for the local alignment and re-alignment steps that are part of our microsatellite calling process. First, a Perl script filters out those microsatellites with small repeats in their flanking sequences found using TRF (parameters: 2, 5, 5, 80, 10, 14, 6). Then each pair of flanking sequences is searched for, individually, in the human genome using a Perl string search function, as BLAST will not run properly with short search queries. A Perl script was written to filter out those microsatellites which have flanking sequences that occur more than once in the human genome within 200 bases of each other and have 5 bases of the repeat in between. Ten base flanking sequences are used as the majority of our reads are from the Illumina platform and are around 100 bases in length. The length of the reads is also why the 200 base search range was chosen for the flanking uniqueness search. As the read lengths increase from the next-generation sequencing platforms, flanking sequences having increased lengths may be used. This will allow us to filter out fewer microsatellites from our set as the larger flanking sequences will result in a larger set of microsatellites which can be uniquely mapped. The remaining microsatellites are associated with genes and regions using the RefSeq data provided by the UCSC Genome Browser, with upstream defined as the 1,000 bases preceding the transcription start site.

Calling Repeat Lengths Using Microsatellite-Based Genotyping:

The raw read alignment process begins by mapping the reads to the reference using BWA for short reads or BWA-SW for long LS454 reads (Li H, Durbin R (2009) Bioinformatics 25 (14):1754-1760). This process is not essential as all reads mapped to microsatellites will eventually have their alignments tested and possibly be realigned to the same locus or another locus in the genome. However, this step is useful to speed up future steps. Next, a Perl script plus SAMTOOLS pulls out all of the reads from all of the microsatellite loci in batches to speed up the processing. Using 5 bases of flanking sequence on either side the reads are tested to make sure they completely span the microsatellite sequence and also to determine if they are the correct match for the microsatellite locus to which they have been aligned by BWA. BWA has issues aligning repeats which contain mostly the repetitive sequence and little unique flanking sequences as BWA relies on the repetitive sequence for mapping. Therefore, BWA can align two different microsatellites with the same canonical repeat to the same microsatellite locus if not enough unique flanking sequence is present on each read. Once we find a read which is a good match to a microsatellite locus, using the flanking sequences, starting with 5 bases and increasing to include more flanking sequence and possibly some of the repeat sequence next to the flanking sequence, if needed, we align this read to the reference. At this point if there are more than two high quality matches for one flanking sequence in the read, this read is removed from the set as the optimal alignment cannot be determined and so the microsatellite read length cannot be called with confidence. This realignment is an important step as for some microsatellite loci there are multiple alignments possible. Using these rules, our code will find the optimal alignment which might not always be found by BWA. At this step all of the reads which BWA aligned to a microsatellite, but for which we found do not align to that particular microsatellite locus, are combined with all of the reads which were not found to align with the reference at all, by BWA, using SAMTOOLS and a custom Perl script to create a fastq file. All of these reads comprise the final batch to process for which we attempt to align them to any of the microsatellite loci using both 5 base flanking sequences. If we determine an alignment is possible because there is enough flanking sequence contained on the read and also the flanking sequences match that of a particular locus, we then perform our alignment to find the best mapping of the read to the reference as in some cases there can be more than one possible alignment.

The reads which have been aligned to particular microsatellite loci using our software are then filtered to determine if at least 5 bases of their particular repeat are contained within the flanking sequences. This step is essential as when we determined if the flanking sequences uniquely captured a specific microsatellite locus, our test included 5 bases of the repeat in between the flanking sequences. Since our uniqueness test used 10 bases of flanking sequence we also filter out those repeats which do not align to 10 bases of flanking sequences using a Perl string function. Using a Perl function is faster than using BLAST and allows us to check for shorter flanking sequences, as BLAST does not perform well with queries of less than 50 bases. The length of the flanking sequences required can be modified in the code to any length from 5 to 10 bases though it must be the same as that which is tested for uniqueness in the initial creation of the microsatellite set to allow for this method to work as accurately as possible. Also the number of SNPs and indels allowed in the uniqueness filtering step would be the same as that allowed here. As the length of reads increases, we will be able to obtain larger flanking sequences from microsatellites and so we can run with larger flanking sequences in our algorithms. This will allow us to accept more variation in the flanking sequences and also cause more microsatellites to have unique flanking sequences because of the increased size.

At this point we have a set of reads which is significantly reduced from the original set, for they are only reads that map to microsatellite loci. We now apply a filter to remove those reads which are of low quality based on the criteria used by the 1000 Genomes Project. This step is done at this time for efficiency as few reads at this point need to be filtered out. Next, on a per locus basis, the reads are binned to group those which have identical repetitive sequences. These bins vary based on repeat length and also SNPs. So for example, two reads supporting a microsatellite of the same length but with different SNPs would be placed in different bins, and thus have different genotypes. If we are using reads from the LS454, which is known to have issues processing homopolymer sequences, we will filter out any reads which contain homopolymer indels in the microsatellite or flanking sequence regions. We now use the quality scores from the original fastq files to determine what score is associated with each of the SNPs in the repeat region. Reads with quality scores of less than 99.9% accuracy for a SNP in a microsatellite are filtered from the set. The bins with 2 reads or less supporting the allele call are now removed from the set as these reads represent possibly error prone sequences. Also all of those with reads 3 times the expected average are removed as these also indicate an error in this region, or represent highly similar microsatellite loci or genomic regions for which accurate mapping and genotyping is not possible. We now call microsats for those loci with at most 2 alleles. If we allow for more than 2 alleles, we estimate it would only affect ˜0.01% of our calls, which total over 138 million, from testing 250 normal samples with low WGS and targeted exome sequencing provided by the 1000 Genomes Project. For some studies, including characterization of sample heterogeneity, for example, we allow for more than 2 high quality alleles at a given locus. A heterozygous locus is called if the 2 alleles do not vary by more than 2× coverage to allow for unequal amplification. For studies which we are not interested in examining the SNPs, the final step is to remove all indications of SNPs in the microsatellite calls so they are only grouped based on repeat length.

Accuracy Validation of Our Microsatellite-Based Genotyping Method:

We used microsatellite-genotyping to identify novel variations in 551 individuals whose genomes were targeted exome sequenced by the 1000 Genomes Project. We found over 68% of the exonic repeat length variations microsatellite-based genotyping identified were novel. Only 5.8% of the exonic repeat length variations we identified were also identified with indel-based (standard) genotyping. Using Sanger sequencing and data from HapMap, we were able to validate 96.5% of a subset of 85 non-synonymous variations composed of repeat length variations and SNPs contained in microsatellites. The novel variants we validated using Sanger sequencing were submitted under the lab handle SGARNER and are available on-line in the latest release of NCBI, NIH dbSNP. In a second accuracy study, we estimated the accuracy of our original software by computing the number of microsatellites which do not conform with Mendelian inheritance for a trio (mother, father, and daughter) sequenced at high depth by the 1000 Genomes Project. The accuracy of our microsatellite-based genotyping method for those 1,095 microsatellite loci which differed between the samples was estimated at 94.4%. Based on this computation, this study estimated that with low coverage only 21% of microsatellite loci are accurately called by the standard indel-based genotyping.

Recent Updates to Our Software to Reduce Runtime:

The software was updated to accept hg19 alignments by converting the prior microsatellite coordinates using the UCSC Genome Lift-Over tool (Hinrichs A S et al. (2006) Nucleic acids research 34 (Database issue):D590-598). This conversion is not required to be accurate to a single nucleotide granularity as our microsatellite software only needs to know the general region in which a microsatellite is located to assign a call as the flanking sequences and not the chromosomal coordinates are used for local alignment. The software was also updated to speed up the sub-functions allowing us to run an exome-sequenced sample in under 3 hours on a single core of an Intel Xeon 5500/5600 processor. We performed tests between our original hg18 software and the new, faster hg19 version to determine if any microsatellites calls differ. We identified 530 microsatellites out of 850,000 for which different genotypes were obtained. These microsatellites were removed from our analysis set.

Microsatellite Calling Restrictions for Population-Based Statistics:

To increase uniformity of coverage and genotyping rates across samples sequenced at different times with different methods by different studies, we required at least 15,000 microsatellite loci to be called per sample for inclusion in this study. This filtered out one 1 kGP-F sample and 235 1 kGP-M samples (the first 1000 Genomes Project samples released were male, and were of significantly lower quality and depth). Only those loci with at least 15× coverage are considered “callable” in a given sample (healthy or cancer genomes). This is an increase in the coverage from our prior work (McIver L J et al., (2011) Genomics 97 (4):193-199; McIver L J et al., (2013) Gene 516 (2):328-334) with the goal of increasing accuracy as it was now possible with the sequencing depth of these samples to call a large set of microsatellites while requiring this increase in our coverage requirement. Using this process, 184,839 microsatellite loci were genotyped with sufficient coverage in at least one BC germline exome, and 68,164 microsatellite loci were genotyped from at least one 1 kGP-EUF exome. A locus had to be called in a minimum of 10 exomes to be included in the genotype distribution comparison analysis to remove loci which may be called at insufficient frequency in one of the two data sets.

Validation that No Informative Loci Will be Found when Sample Sets are Artificially Divided and Tested (Female Vs. Female):

The 1 kGP-F samples, representing all different ethnicities, were divided into two groups. Group 1 had 223 samples and group 2 had 215 samples. Following our procedures to obtain informative loci, using group 1 as the healthy set and group 2 as the test set, and using a False Discovery Rate (FDR) of 0.01%, we were not able to identify any informative loci. All FDR adjusted p-values for these two sets were 1.0.

Determining the Possible Ethnicity of the BC Samples:

We compiled a list of modal genotypes for all loci called in the 439 1 kGP-F samples that represented 18 different ethnicities. We then identified informative loci differentiating this set from the BC germline set. Graphing each ethnicity and the BC germline samples based on the percent of loci that match the cancer-like set, we were able to identify a sub-set of ethnicities (CEU, FIN, GBR, IBS, and TSI and PUR) that very closely matched the cancer set (FIG. 18). As the majority of these individuals are of European ancestry, we have referred to them together as EU.

Subsequently, after this analysis was completed, the race of the BC samples was released in the clinical data set downloadable from TCGA Data Portal. Considering the 656 BC germline samples, 489 (74.5%) were labeled as “White” implying European ancestry, 6.6% were labeled as “Asian”, and 6.1% were labeled as “Black or African American”. For the remaining 9.6% of the samples the race was labeled as “Not Available.” This supports our initial analysis identifying the BC samples as well represented by mostly individuals of European ancestry.

Modal Genotype Determination:

We compiled the genotypes from all the 1 kGP-EUF samples for each microsatellite locus. The genotype supported by the highest number of samples was determined to be the modal genotype. In cases where more than one genotype was equally represented, the genotype listed first in our compiled set was used consistently as the modal genotype. In a diagnostic or prognostic method, such a modal genotype for a locus determined across a reference population can be used as the reference for evaluating a subject.

Hardy-Weinberg Equilibrium Computation:

The polynomial expansion of the Hardy Weinberg equation for the presence of multiple alleles was used to derive the expected genotype distribution for each of the 55 loci for the 1 kGP-EUF and BC populations. A chi-square statistic was then employed to identify those loci in Hardy-Weinberg equilibrium.

Computing Statistics for Each Microsatellite Locus:

2×2 tables were created for each locus for the 1 kGP-F normals and the BC germline samples that were called in at least 10 samples in each set: 1 kGP-EUF with modal/non-modal genotypes by BC germline with modal/non-modal genotypes. An R script computed the p-value for each locus using the two sided fisher.test function. The Benjamini-Hochberg cut-off was selected as 0.01% (FDR<1/3750 (total number of loci with p-value <1)) to make it unlikely that any locus is a false positive from our data set. 55 loci passed the FDR test and were considered to be informative in distinguishing the healthy EUF from the cancer samples. Relative risk for each locus was computed as the percent of individuals with the non-modal genotype from the cancer set divided by the percent of individuals with the non-modal genotype in the normal set.

Calculating Sensitivity and Specificity:

Using the 55 loci which differentiate breast cancer germline genomes from healthy genomes, we computed the sensitivity and specificity at each point in the spectrum of the percent of loci matching the cancer-like signature. The area under the curve of 0.88 was determined for this ROC curve of 1—specificity vs sensitivity (data not shown) with the ROC Bioconductor package in R (Carey V, Henning R ROC: utilities for ROC, with uarray focus, vol R package version 1.28.0). An additional R script was written to compute the sensitivity and specificity based on maximizing the area under the curve. The optimal cut-off was found to be 76% of callable, genotyped loci matching the cancer-like signature. In other words, when a sample is compared to a reference (e.g., a modal genotype in a non-cancer/healthy population), the optimal cut-off for distinguishing whether the sample is likely to be a cancer sample or have an increased risk of cancer versus being a healthy sample is when 76% of the callable, genotyped loci have a non-modal genotype when compared to the reference.

Microsatellite Genotypes for Matched Samples (Germline—Tumor—RNASeq):

We grouped microsatellite calls by matched samples to identify those that varied between the exome sequence and matched RNAseq data for the BC samples. There was no matched RNAseq data for the 1 kGP-EUF samples with 15× coverage. There are 5,078 instances (0.29% of all matched loci) where the tumor had a different genotype than the germline. For the exome vs RNAseq datasets, only 5% of the loci in the germline samples were both callable in the exome and contained in a characterized transcript in the RNAseq data. This number was larger for the tumor RNAseq samples with 29% of the loci analyzable as there were more RNAseq tumor samples available (n=813).

Associating Microsatellite Loci with the Genes Containing them:

We used the RefSeq genes downloaded from the UCSC Genome Browser to associate microsatellite loci with genes and identify their genomic region. Upstream and downstream boundaries were defined as 1000 bases from the transcription start and end points. Microsatellite loci were associated with the gene region the majority of their sequences were contained in if they overlapped two regions. Manual investigation of our 55 loci using UCSC revealed that two loci initially indicated as intergenic are associated with genes (potentially an update since our download of refseq). These loci were modified to indicate their associated genes.

Alternative Splicing:

We processed the 917 RNAseq data sets with Cufflinks by using the CuffCompare function to identify possibly alternatively spliced transcripts (Trapnell C et al. (2010) Nature biotechnology 28 (5):511-515). For each transcript for each sample, we determined it was possibly alternatively spliced if one of the transcripts called by CuffCompare was not a complete match of the intron chain. We did not use any transcripts which CuffCompare indicated an intron matches one on the opposite strand as these were likely due to read mapping errors as stated in the Cufflinks documentation. Each gene symbol was then given a value of “normal” or “alternative splicing” based on the splicing values for all of its transcripts. A gene symbol was labeled as “normal” only if all transcripts associated with that gene symbol exhibited “normal” splicing. These were then matched up with the microsatellite genotypes called for each informative gene for each sample. Overall, we analyzed splicing at 20,387 transcripts in the BC germline samples and 23,503 transcripts in the tumor samples with 85.9% and 84.5% of transcripts indicated as alternative splicing events, respectively. Within our 55 loci, we were able to analyze 48 transcripts in the BC tumor samples and 41 in the BC germlines, 80.1% and 80.5% of which were indicated as possible alternative splicing events respectively.

RNA Analysis:

We processed the 917 RNAseq data sets using Cufflinks. We were only able to analyze a small portion of all possible data points as only 5% of the loci were both callable in a sample and contained in a characterized transcript for the germline samples, possibly due to the limited number of RNAseq germline samples (n=104). This number was larger for the tumor RNAseq samples with 29% of the loci analyzable as there were more RNAseq tumor samples provided (n=813). 740 matched with exomes.

Ontology:

GO enrichment analysis of genes associated with the 55 signature loci was performed using DAVID (Huang da W et al., (2009) Nature protocols 4 (1):44-57; Huang da W et al., (2009) Nucleic acids research 37 (1):1-13) functional annotation tools (P<0.1), Genedecks (Safran M et al. (2010) GeneCards Version 3) and GSEA (Subramanian A et al. (2005) PNAS 102 (43):15545-15550). Pathway enrichment was performed using Panther (Mi H et al. (2005) Nucleic acids research 33 (Database issue):D284-D288).

Expression of Genes in Breast Tissue:

Each gene was manually researched in GeneCards (Safran M et al. (2010) GeneCards Version 3), which contains expression data from BioGPS (Su A I et al. (2004) PNAS 101 (16):6062-6067; Su A I, Cooke M P, Ching K A, Hakak Y, Walker J R, Wiltshire T, Orth A P, Vega R G et al. (2002) PNAS 99 (7):4465-4470), Body Map 2.0 (provided by Gary Schroth at Illumina and accessible from ArrayExpress accession no. E-MTAB-513), and SAGE (Velculescu V E et al., (1995) Science 270 (5235):484-487) to obtain data on possible expression levels in breast tissue. All values are included in eTable 2. We were able to find expression data on all genes except for two (TRG and FAM157A) that were not included in the AgilentG4502A expression kit.

FAM157A Protein Modeling:

The protein structure for FAM157A was determined using the gene sequence identified in hg18 (3:199364528-199364569) from the UCSC genome browser, and the cDNA sequence was used as the reference. FASTA files were exported to ExPASy (Artimo P et al. (2012) Nucleic acids research 40 (Web Server issue):W597-W603) and DNA sequences were translated to protein sequences. Manually, modifications introduced to exonic DNA by microsatellite repeats were introduced to FASTA sequences and translated with ExPASy. The reference and DNA sequences with microsatellite variants were threaded using RaptorX (Peng J, Xu J (2011) Proteins 79 Suppl 10:161-171); from RaptorX, pdb files for the aligned protein sequences were used for protein modeling. Using Phyre2 3-D structures were assembled using a one-to-one threading procedure with the amino acid sequence for each protein and corresponding pdb file.

Drug Targets:

All of the genes containing informative loci were run through CancerResource (Ahmed J et al. (2011) Nucleic acids research 39 (Database issue):D960-D967) to identify any possible drugs which target these genes. Each of the 37 results, corresponding to 13 genes (24.1% of the 54 genes of interest), were manually researched to filter out those which were not recognized as pharmaceuticals by MedlinePlus, DrugBank or the National Cancer Institute Cancer Drug List (either FDA approved or experimental), resulting in a final list of 22 drugs targeting 11 genes.

### Results

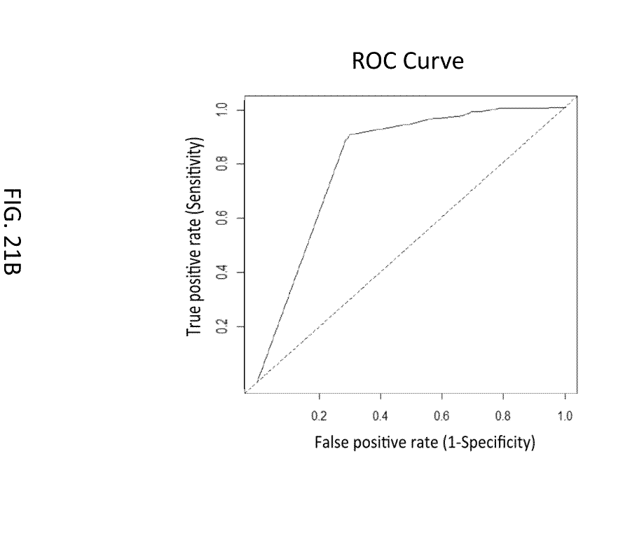

Many studies attempt to link the presence or absence of specific mutations to a disease state. This has been a successful strategy for discovering novel disease-associated genes; however, complex disease states may not be due to a single mutation, but to additive effects of multiple common variants, as seen, for example, in the multiple SNPs associated with telomere maintenance and BC risk. To uncover this type of interaction, we must employ a methodology that examines the frequency at which alleles are seen across multiple loci in an affected population. However, focusing solely on the frequency at which an allele is represented, such as the studies described in Examples 1-3 above, may result in missing a significant shift in the frequency at which an allele is heterozygous, as opposed to homozygous. Therefore, we have performed our analysis on the frequency of genotypes rather than alleles within the examined populations, using the algorithm described above. We employed this methodology to determine the genotype of all microsatellite loci in exome sequences from apparently healthy females from the 1000 Genomes Project and in 656 germline exomes from BC patients sequenced as part of TCGA (FIG. 19). Comparison of healthy females from different ethnic backgrounds revealed that variation at some microsatellite loci was correlated with ethnicity; thus we selected only the 249 individuals from European ancestral populations (1 kGP-EUF) because the microsatellite profile of the BC germline samples was the closest to these exomes (FIG. 18). We restricted our analysis to those 49,297 loci that were genotyped with sufficient coverage (15×) in at least 10 exomes from both the 1 kGP-EUF and BC populations. The most frequent genotype in the 1 kGP-EUF population was then considered as the modal genotype for that locus and the frequency of alternative genotypes present within both populations was calculated. On average, 29,809±4,688 and 34,849±4,371 microsatellite loci were genotyped per 1 kGP-EUF and BC germline sample, with 283±134 and 426±124 non-modal genotypes, respectively. We identified 55 loci that each individually showed a statistically significant difference in genotype distribution between 1 kGP-EUF and BC germline (p≦0.01, two-sided Fisher's p and Benjamini-Hochberg). A comparison of females from the 1 kGP randomly divided into two sub-groups did not identify any significant loci using this FDR cut-off, showing that normal variations at loci in two similar populations are not significant using our methods. 25.1%±13.1% and 31.3%±9.4% of the 55 loci were genotyped in the 1 kGP-EUF and BC germline exomes respectively which is not surprising given that we use very stringent conditions for coverage and alignment, and because Lander-Waterman distributions in random fragment sequencing limits the number of callable loci in each sample. Notably, for the 1 kGP-EUF, the most frequent genotype of 24% of the 55 loci is heterozygous while 36.4% of the loci are heterozygous for the BC germline exomes. This confirms that we are able to identify loci where the modal genotype is different between the BC and healthy populations. Analysis of the genotype distributions at the 55 loci revealed that 80% (44/55) of the loci are in Hardy-Weinberg equilibrium in the 1 kGP-EUF samples while only 40% (22/55) are in Hardy-Weinberg equilibrium for the BC germline (Table 14), raising the possibility that there is a reduction in selective pressure in BC germline genomes that may result in increased susceptibility to BC.

Thirty-two of the genes associated with the 55 microsatellite loci have previously been shown to have some association with cancer, and eighteen have been specifically linked to breast cancer (Table 15). Forty-nine of the 55 informative loci are located in introns, 24 of which are located within 50 nt of an exon/intron boundary; three additional loci are intergenic. Notably, four are in the 3′UTRs of known genes (PIAS2, WWC3, MT1X, and TBP), and one is exonic (a CAG triplet repeat in the FAM157A gene; data not shown).

The genotypic differences at these 55 informative loci appear to have two effects on the likelihood of BC. At 30 of the 55 informative loci, the presence of a non-modal genotype is potentially protective against BC (relative risk of <0.6; Table 14), whereas at 25 of the loci a non-modal genotype appears to promote BC (relative risk >1.3; Table 14). Gene ontology enrichment analysis showed that genes involved in notch signaling were enriched among those potential BC-promoting loci while the set that potentially protects against BC includes proteins known to be involved in maintaining genomic stability (e.g. WRN, FANCI, HSP90) and programmed cell death (e.g. PDCD6IP). Some of the genes involved in signaling pathways that are associated with the 55 signature loci, include p53, integrin, and MAPKK pathways.

Risk Classifier

We used the frequency of modal or non-modal genotypes at each of the 55 informative loci within the BC population relative to the 1 kGP-EUF population to create a BC genotype profile. FIG. 14 shows the distribution of exomes based on the number of genotypes at the 55 signature loci that match the cancer profile. Using the false positive and false negative rates within the training set, we were able determine the receiver operating characteristic (ROC) for the 55 BC loci. Through maximizing the area under the ROC curve, we determined the optimal cut-off for a classifier as having 76% of the callable 55 BC loci matching the cancer-like profile. (FIG. 14). We were then able to classify the BC germline exomes as cancer (≧76%) or healthy (<76%) with a sensitivity of 88.4%, and a specificity of 77.1% (FIG. 14). Using this same analysis on a set of BC tumor samples, we identified 88.1% of the BC tumor exomes as cancer-like, a difference that was not statistically significant from the number of germline BC samples that were cancer-like (FIG. 14). This is in contrast to the 1 kGP-EUF samples, of which 77.1% were normal and only 22.9% were cancer-like (FIG. 14). In addition, an independent set of 60 BC germline samples (IND) showed a similar high frequency of exomes being classified as cancer-like with 85.0% as cancer-like and 15% as normal, whereas other healthy individuals, including males and non-European females are more similar to the 1 kGP-EUF exomes.

Table 22 provides the repeat motif, its coordinate in the human genome reference, its modal genotype in the healthy populations, the genotype distributions, the gene in which it is found (if it is not intergenic), and if that gene is expressed in breast tissue (>0), and the ontologies associated with the gene that confirms it potential to contribute to cancer. The number of times that genotype was observed is in parentheses. These informative loci are mostly invariant in tumors. Therefore, it is possible to use germline or tumor tissue to make these measurements.

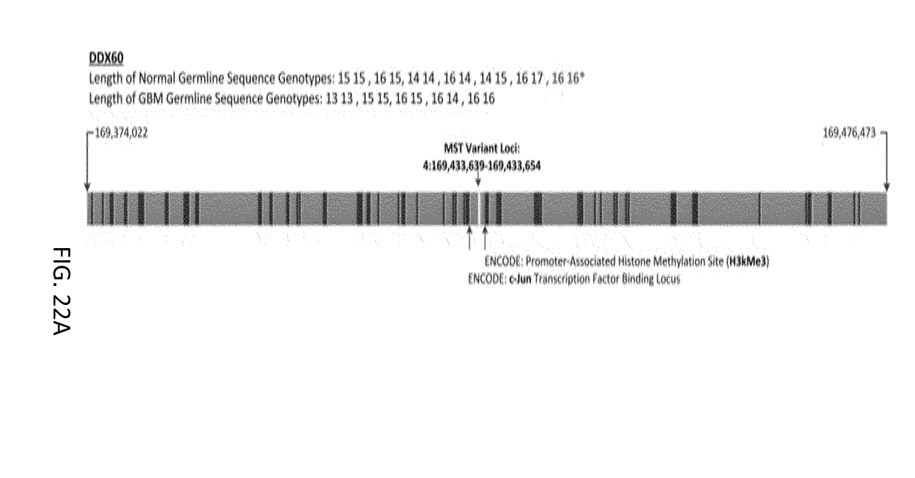

The 55 signature loci were derived from analysis of BC germline exomes regardless of BC subtype. To show that we are able to classify individuals with different subtypes of BC using our germline measure, we divided the BC samples into their subtypes, and show that we are able to classify exomes associated with each of the known BC subtypes, and a set of samples where a subtype was not specified (unknown), to a similar extent. Surprisingly, the BC exome samples for which no subtype was assigned (unknown) appeared to have a distinct profile within the 55 informative loci, distinguishing them from those exomes classified with established BC subtypes. An independent set of 60 BC germline samples had a similar genotype profile as those BC germlines for which there was a subtype specified as opposed to the 1 kGP-EUF samples or the unknown BC germline samples. In addition, we re-analyzed the genotype distribution of all 49,297 microsatellites for each subtype individually with respect to the 1 kGP-EUF to identify those loci that are significantly associated with each or multiple subtypes. There were four loci associated with the luminal A (LA) subtype (FIG. 20). No loci passed our rigorous statistical requirements for the luminal B (LB), ERBB2/HER2+(HER2), or basal-like/triple negative (BL) subtypes, likely because of the smaller number of exomes that were available for these BC subtypes. As can be seen in the Venn diagram, there are informative loci that distinguish the LA and ‘unknown’ subtypes in addition to the 55 that distinguish all BC from healthy genomes (FIG. 20). There were 19 loci that were unique to the ‘unknown’ subset, including loci in genes involved in cell cycle control, chromatin remodeling and programmed cell death. There were also 21 loci that overlapped with the 55 loci identified when all the BC samples were considered together. Surprisingly, there were no loci shared between the LA and Unknown subtypes indicating that our method of genotype analysis at microsatellite loci may be useful for distinguishing between BC subtypes.

Breast Cancer Tumor Vs. Germline Exomes

595 of the BC germline exome samples had matched tumor/germline exome data available. For the 496 matched samples where we could genotype at least 10 of the 55 loci in both the germline and tumor, 75.2% were cases where both the tumor and germline were cancer-like, 8.9% the tumor was cancer-like while the germline was not, and 12.1% the germline was cancer-like while the tumor was not. There were only 3.8% of cases where neither the germline nor the matched tumor was cancer-like. It is important to note that no exome was sequenced with >15× coverage at all 55 loci, so in instances where only one of the matched germline and tumor exomes was classified as cancer-like, the difference may be due to differences in which loci could be genotyped for a given sample. Comparing the tumor and matched germline exomes with our analytical pipeline did not reveal any additional loci that were statistically different. This is not unexpected given that microsatellite instability associated with tumors could re-distribute genotypes non-uniformly across a population or even within a single individual. Importantly, this analysis highlights the strength of our methodology for identifying cancer-like exomes from germline sequencing data without requiring tumor analysis.

Thirty-three germline exome sequenced samples had known mutations in TP53; of these, 28 were identified by our method as cancer-like. Additionally, fifteen samples were identified as having a potential mutation in BRCA1 or BRCA2 of which fourteen are identified by our method as cancer-like (FIG. 14). That the majority of exomes with BRCA/TP53 mutations are also classified by our method as cancer-like is not surprising given that these genes are known to be important for maintaining genomic stability. However, our measure is not restricted to identifying only those individuals carrying these known high-risk markers as we were able to identify 541 individuals who did not carry any of these known disease predisposing mutations as having a cancer-like signature at the 55 microsatellite loci.

In addition to exome sequencing data, the TCGA had RNAseq data available for 813 BC tumors and 104 BC germline samples, of which 636 and 87 had available DNA sequence data, respectively. We performed genotype prediction from the RNAseq data for 18,148 exonic microsatellite loci that were potentially callable in the matched RNAseq genotypes and the respective genotypes in the germline and tumor samples. At 99.98% of those loci that were called in both DNA and RNA sequencing, the predicted genotype from RNAseq was consistent with the genotype determined from the matched exome sequencing. Those loci that were genotyped differently between the matched exome and RNASeq data were located at 72 loci, none of which are in genes associated with our 55 loci. However, genes associated with loci that differ between BC germline and RNAseq data are enriched for the VEGF signaling pathway, which influences vascular growth and angiogenesis. These loci may be additional biomarkers for alternatively spliced transcripts that may contribute to BC.

Gene set enrichment analysis (GSEA) indicated that the 55 informative loci and those loci that were identified in the individual subtypes were enriched for association with genes whose expression positively correlates with BRCA1. We analyzed the RNAseq data to identify additional potential shifts in gene expression that might correlate with BC. We were able to analyze the expression level for 52 of the genes in the BC tumor exomes but only 46 genes in the BC germline samples because gene expression data were provided for 304 tumor samples but only 39 germline samples from the TCGA. No expression information was available for FAM157A or TRG, for which no bait was included in the AgilentG4502A expression kit. Of the signature loci, 48 had previously been shown to have some level of expression in breast tissue (Table 14). Comparing all germline and tumor samples, analysis of the expression levels of the genes associated with the 55 informative microsatellite loci revealed that seven of these showed >2× increased expression in tumors, while four showed decreased expression (Table 16). One gene in the germline set (CRISP1) and one gene in the tumor set (ABHD12B) showed >2× difference in expression between individuals who had a genotype matching the cancer profile and those who did not. In both cases, the individuals with a genotype that matched the cancer profile showed a higher expression level than those who did not.

Microsatellite variation at intronic loci may result in alternatively spliced transcripts that have the potential to contribute to oncogenesis, with estimates that ˜95% of multi-exon genes exhibit alternative splicing. Additionally, 49.0% of the intronic loci were within 50 nt of an exon/intron junction, a higher frequency than expected given that only 3.4% of all intronic microsatellites that were genotyped in at least one exome sample were within this boundary. This led us to hypothesize that they may be affecting splicing of transcripts. We used Cufflinks to identify possible alternative splicing events in transcripts containing the signature loci. If we consider only those loci for which we can capture both the transcript splicing and signature loci, we find that samples which have cancer-like genotypes are more likely to exhibit possible alternative splicing in their respective transcripts. For the germline set, 84.9% of the transcripts with cancer-like loci show possible alternative splicing compared with 77.4% of those transcripts which contained non-cancer like genotypes. These numbers were similar for the tumor set, with 81.5% of the alternative spliced transcripts also having cancer-like genotypes compared with 79.8% with non-cancer-like genotypes.

Ten of the genes associated with the 55 loci are targets of, or affected by, pharmaceuticals several of which are prescribed or in clinical trials for BC (Genes: MLL, HSP90AA1, MT1X, PDGFRA, PTPN22, STC1, NCOR1, PCYT1A, MME, RDX). This is ˜1.2× greater than expected given the drug target interactions within the CancerResource database and emphasizes that the genes associated with the loci identified by our method are already candidates for drug targets for BC therapy. Thus, our analysis may provide novel drug targets or drug re-positioning opportunities for additional or combinatorial BC treatment plans.

### Example 5

### Somatic Microsatellite Loci Differentiate Glioblastoma Multiforme from Lower-Grade Gliomas

Genomic studies of brain cancer sub-types have amassed new disease specific mutations, yet only partially explain how these mutations are linked to predisposition or progression. Significant clinical benefits from new informative biomarkers, whether germline or from somatic tumors could improve diagnostics and treatment. We hypothesized that microsatellite instability and individual microsatellite-based loci could be a new source to further understand the etiology of brain cancers. Using the same genotyping method outlined in Example 4 above, we compared “healthy” germline DNA sequences from the 1000 Genomes Project (n=390) with lower-grade glioma (LGG, n=178) and Glioblastoma multiforme (GBM, n=252) germline sequences from The Cancer Genome Atlas to identify cancer-associated microsatellite loci.

Exome sequencing data, from Illumina HiSeq sequencing machines were obtained from The Cancer Genome Atlas (TCGA) and the 1000 Genomes Project (1 kGP). Only loci with sequencing reads with 15× or greater depth of coverage were used to identify possible informative loci. A profile or distribution of alleles for the affected (TCGA) and unaffected (1 kGP) cohorts was then generated for each locus. An allele is defined by a genomic locus with a specific microsatellite repeat and nucleotide sequence length, in each sample a pair of loci was identified and each allelic pair was then defined as a genotype. The genotype most prevalent from a distribution of genotypes was identified (called) in 1 kGP samples; this genotype was defined as the consensus sequence (the modal genotype; if more than a pair of alleles was identified for a locus that sample was not used). Similar to the 1 kGP samples, LGG and GBM samples were analyzed for genotypes from the same genomic loci, loci different from the consensus or between LGG and GBM and with differing frequency-of-occurrence were then called. The statistically significant genotypes were determined from data adjusted for false discovery rate (FDR), using a two-sided Fisher's p-test and Benjamini-Hochberg correction; relative risk (RR) was calculated for each locus and loci with a P≦0.01 were considered significant. Those genotypes, although individually informative, were also assembled into a ‘signature’ or ‘cancer-associated’ informative loci which together increase the statistical significance across all samples. Samples included 390 (n=249 female; n=141 male) normal samples from the 1 kGP, GBM germline (n=252), and LGG germline (n=178) sequencing samples.

The number of informative loci that passed all statistical tests that differentiated cancer-associated from “healthy” included 66 LGG and 48 GBM loci (Tables 17 and 18, respectively); of these, 10 of the signature loci in GBM overlapped with those in the LGG signature. Callable loci included 26,427.46 (SD±2,333.70) from LGG Grade II, and 27,021.47 (SD±4,859.31) for GBM. From these we identified 179 significant loci (P≦0.01) in LGG and corrected for false discovery rate for a final set of 66 signature LGG loci (average callable loci in LGG samples 20.0 (±8.2 loci); in “healthy” sample 21.6 (±7.7 loci). In GBM sequences, we identified 179 significant loci (P≦0.01) and 48 that passed FDR correction (average callable loci in GBM samples were 13.1 (±6.6 loci; in “healthy” samples 14.3 (±7.4 loci). From these signatures, a percentage of the callable loci that either had the “healthy” consensus or were not—‘cancer-associated’—in 1 kGP, GBM and LGG samples were identified. Between 75-80% of callable GBM cancer-associated loci (e.g., genotype differs from the modal genotype ascertained from the reference population of non-cancer samples) could be identified in 19% and 17% of GBM germlines versus 4% and 3% of normal samples; a similar population of GBM tumors (16%) had 75-80% of cancer-associated loci (e.g., genotype differs from the modal genotype ascertained from the reference population of non-cancer samples). Twelve-percent of GBM germline or tumor samples had 100% of the cancer-associated loci (e.g., genotype differs from the modal genotype ascertained from the reference population of non-cancer samples), while 3% of “healthy” samples showed similar results; this suggests that there may be individuals in the 1 kGP cohort who are predisposed to GBM but due to age and other disease specific variables, the illness has not manifested itself. Between 10-30% of the LGG loci could be identified in 76% of the normal germlines (ranging between 11-17%) while 69% (15, 11, 20, and 11%) of LGG germline samples had 40-60% of the cancer-associated loci (e.g., genotype differs from the modal genotype ascertained from the reference population of non-cancer samples), the largest population of LGG (20%) had 50% of the identifiable cancer-associated loci (e.g., genotype differs from the modal genotype ascertained from the reference population of non-cancer samples).

To determine the sensitivity and specificity of the GBM and LGG informative microsatellite loci identified above, we generated an ROC (receiver operating characteristic) curves. We determined that for LGG, an analysis using the 66 LGG informative microsatellite loci give a sensitivity of 91% and a specificity of 86%, with a cut-off of 35% (FIG. 16) (LGG tumor sensitivity was 84% and specificity is 86%). With regards to GBM, we determined that an analysis using the 48 GBM informative microsatellite loci give a sensitivity of 94% and specificity of 77%, with a cut-off of 57% (FIG. 15) (GBM tumor sensitivity is 96% and specificity is 75%).

Additionally, we compared LGG and GBM germlines and discovered 26 informative microsatellite loci that distinguish LGG from GBM. Specifically, these loci were determined by computing modal genotypes at microsatellite loci in the LGG population and comparing the genotypes for the same loci in the GBM population. Nineteen of the 26 signature loci were found in the LGG signature, and 11 are significant (P≦0.01) to the LGG cancer-associated genotypes. Two loci were found in the GBM signature (in 9:42626-42640 and SSX2) but only one locus (in 9:42626-42640) is in the GBM cancer-associated signature. We then measured the percentage of samples (GBM and LGG) with these genotypes. GBM germline sequences shared an abridged population of LGG genotypes; upwards of 82% of callable germline genotypes were identified in GBM samples. Between, 85-100% of LGG loci could be identified in 13, 27, 4, and 22% (66% total) of GBM samples. Below 82%, the percentage of genotypes in LGG samples were more enriched (FIG. 17). Using an ROC curve, we determined that an analysis with these loci gives a sensitivity at 74% and a specificity at 90%, with a cut-off of 82% (FIG. 17) (tumor analysis shows sensitivity at 76% and specificity at 72%).

We also compared Grade II LGG and GBM germline sequences and discovered eight informative microsatellite loci that distinguish GBM from LGG grade II. Specifically, these loci were determined by computing modal genotypes at microsatellite loci in the LGG grade II population and comparing the genotypes for the same loci in the GBM population. In Grade II LGG samples, 75-80% of loci could be called in 7-19% of samples whereas, 1-3% could be called in the GBM samples. The 80% of genotypes identified in 19% of the samples were located within the following genes (in order of significance): KIAA1219 (13 samples), SNX17 (12 samples), SACMIL (9 samples), MYCBP2 (8 samples), GFM1 (7 samples), COPS4 (6 samples), and CDC16 (1 sample). All eight signature loci were identifiable in the majority of Grade II LGG, GBM, and the general population (1 kGP; data not shown) suggesting that these markers would not be used to screen the general public for gliomas but are instead selective biomarkers able to differentiate LGG Grade II from GBM. Furthermore, using an ROC curve, we determined that an analysis with these loci gives a sensitivity of 90% and specificity of 70%, with a cut-off of 85% (FIG. 21).

Thus, these markers are valuable to screen risk of occurrence in families with a history of cancer or gliomas, and other neurological diseases with increased incidence of gliomas (e.g., epilepsy, Li-Fraumeni syndrome), or the likelihood of GBM in LGG patients.

Molecular, cellular, and biological processes associated to microsatellite signature loci were analyzed using DAVID annotation tools. GO terms over-represented (P≦0.1) in comparison to a reference Homo sapiens gene list are reported. From our GBM data, terms associated with key functions included helicase activity (6 loci); neurogenesis (3 loci), alternative splicing (22 loci), ubiquitin conjugation pathways (4 loci), and polymorphism (29 loci) were identified. Of these, ‘helicase’ was highly significant (P≦0.05; 9.13×10⁻⁴ with Bonferroni correction). Biological processes that complemented these functions were also identified, and included: ribonucleoprotein complex assembly (3 loci), transmembrane receptor protein tyrosine kinase signaling pathway (3 loci), autophagy (2 loci), RNA processing (4 loci), and proteolysis/cellular protein catabolic processes (4 loci). Additionally, 15 loci (STRC, CBL, LAMP1, FGFR2, ENAH, TNIK, POLQ, BRWD2, SEMA3E, PSME3, NSUN5, DICER1, NRP1, BRMS1L, SPOPL) were identified as previously associated with cancer and three with GBM-BRWD2 (WD repeat domain 11), NRP1 (neuropilinl), and FGFR2. From these annotations, we further analyzed individual genes and their potential in GBM biology, as described below.

Helicases & RNA Processing:

Helicases are important to RNA decay, remodeling and nuclear export among several other functions that contribute to RNA processing. Those helicases with cancer-associated microsatellite loci function in splicesome complexes (DHX36, DICER1, and TTF2) and ribonucleoprotein complexes (RNPs, snRNPs, or snoRNPs) including DDX20, DHX36, and DDX60. Several of these helicases function with other genes identified in our GBM signature list and respond to interferon activation. Specifically, DDX60 regulates DDX58 (also known as RIG-I) and MDA5 complex RIG-I and MDA5 are RNA helicases and sensors for viral RNA. RIG-I is activated upon viral RNA detection, and is ubiquinated by TRIM25 (which also has GBM signature loci); both are interferon dependent methylation and ubiquitination complexes. Other genes with functional associations included, DDX20 and NSUN5, Nop1/2 family (NSUN) proteins modify RNA methylation and snRNP or snoRNP (small nucleolar RNPs). NSUN5 has tri-nucleotide repeat (CAA) in the exon and functions as a methyl-transferase protein which can contribute to unequal crossing-over in low-repeat sequences flanking deleted regions of a gene; NSUN family members are especially contributive to neural morphogenesis. Other genes which respond to interferon included TTF2 and DICER1; TTF2 represses mitotic transcription and pre-mRNA-splicing and therefore would be especially important to cell-division, DICER1—has been implicated in cancer and neuroskeletal disease—importantly, it cleaves dsRNA to siRNA and is essential to processing miRNA into mature miRNA. miRNA synthesis, and specifically tumor suppressing miRNA, are linked to multiple genes with GBM signature loci—among helicases, DDX20 and DICER1 are notable. DDX20 contributes to miRNA containing RNP complexes which suppress NF-\hat{k}B via modulation of miRNA-140 (potential tumor suppressor). miRNA are non-coding small RNAs that can regulate DNA expression post-transcriptionally; these sequences can bind to the 3′ UTRs of mRNA and degrade or inhibit translation. Thus, DDX20 and DICER1 may be important to controlling cancer-propagating inflammation, in gliomas. Other genomic modifications—including epigenetic changes in mRNA and miRNA are controlled through DHX36 and DDX20. DHX36 is known to deadenylate and degrade mRNA. DNA methytransferase (DNMT) is regulated by miRNA-140, previously described. Where DDX20 expression is deficient, hypermethylation at metallothionein genes by DNMT leads to decreased expression of miRNA-140 and increases NF-\hat{k}B activity. Thus, methylation status in gliomas, via MGMT may also be complemented by DNMT, if DDX20 expression is modified.

Ubiquitin Proteasome System:

Protein modification at ubiquitin binding loci can change the destiny of a given protein, altering its status from degradation, especially in the case of cancer. PSME3 is a proteasome regulator which facilitates Mdm2-p53/TP53 interaction by promoting ubiquination and degradation of p53 (limiting p53 accumulation promotes apoptosis); therefore MST loci in PSME3 may contribute to the misregulation of p53 via Mdm2 (also an E3 ligase). Others included: ATG3, which contains an E2 catalytic domain and is essential to autophagy; TRIM25 (also known as estrogen-responsive finger protein; EFP) is activated through interferon and ubiquinates DDX58 (a signature helicase described above). Additionally, TRIM25 interacts directly with RNA and is an RNA binding protein which is preferentially expressed in embryonic stem cells (ESCs) and is down-regulated in embryoid bodies. A second TRIM gene, in the same subfamily as TRIM25, TRIML1 is produced during pre-implantation in ESCs to blastocysts and is otherwise only detected in adult testis. Much like the helicases previously described, TRIM25 and TRIML1 are associated with miRNA and RNA synthesis. TRIM25 and TRIML1 were identified in LGG but were not statistically significant loci; this could be due to sample heterogeneity and population size as compared to GBM.

We identified several E3 ligases with variant MST loci, important to GBM and LGG. SPOPL is a part of the E3-ubiquitin ligase complex and mediates glioma-associated oncogenes (Gli), Gli2 and Gli3 both zinc-finger associated transcription factors which mediate Sonic hedgehog signaling pathway (Shh). Shh arbitrates metastasis and invasion through expression of BCL-2, c-MYC, and VEGF among many others. Also, SPOPL functions with SPOP, SPOP mediates BRMS1L (also a gene in the GBM signature loci) with Cul3 domains; BRMS1L is a tumor suppressor that regulates the expression of metastasis suppressive miRNA (mi-146a and miR-146b) which decreases EGFR expression.

Angiogenesis & Cell Signaling:

Glioma-promoting inflammatory responses are pervasive in the microenvironment of the tumor which is perpetuated through tyrosine kinase receptors. Another well-known E3 ubiquitin ligase, CBL, was identified with cancer-associated MST loci, CBL recognizes activated tyrosine kinases (including FGFR, PDGFR, EGFR, FLT1, KIT and others, which are over-expressed or mutated in GBM). Thus, MST modified near CBL may contribute to the mis-regulation of angiogenic receptors. We identified several other key genes associated to tyrosine kinase receptor pathways, many of which have previously been identified with cancer, including: FGFR2, TNIK, and NRP1. SEMA3E (contains a GBM signature locus) may down-regulate emergent angiogenesis, a balance between SEMA3s and VEGF-165 binding to KDR are regulated through NRP1 (which also contains a GBM MST variant); therefore NRP1 and SEMA3E could be therapeutic targets and loci that require further study. Supportive of this idea, SEMA3E RNA expression was significantly (P≦0.01) decreased in GBM tumors compared to “healthy” germline samples (Figure S2).

Several GBM signature loci were connected with genes essential to Wnt signaling (OFD1 and TNIK), Notch (CORIN), and Hh signaling pathways (ARL13B and EVC; ARL13B may interact with OFD1, also a GBM/LGG signature loci); these pathways are notably up-regulated in GBM and are contributive to glioma stem cell proliferation.

Cell Cycle & Development:

Six loci associated with genes important to cell-cycle were discovered. NCOR1 is a component of a repressor complex that is recruited to methylated CpG dinucleotide islands; which are prognostic indicators for gliomas. Additionally, NCOR1 contributes to transcriptional repression by regulating nuclear receptors and promotes histone deacetylation to form repressive chromatic structures to prevent basal transcription. Thus, genes central to transcriptional repression are modified by MST loci. Interestingly, cancer-associated microsatellite genotypes in ATM were identified in more than half of all LGG primary gliomas (53%); genomic aberrations in ATM increase mutations produced during mitosis that contribute to cancer.

Signature loci associated with developmental or cell differentiation genes, included: DIP2B, NEO1, FRMD7 KCTD20, and FUBP3 (FUBP3 modifies gene expression and interacts with ssDNA; similarly, mutations in FUBP1 along with IDH1 have previously been linked to OD). DIP2B has signature loci in GBM and LGG. DIP2B functions with FRA12A a folate sensitive gene linked with Fragile X syndrome. Repeats sequences have previously been identified at the 5′ UTR of DIP2B (CGG repeat) and has a functional locus for DNA methylation; elongation of this repeat sequence reduces mRNA expression by half in individuals with ‘fragile sites’ in FRA12A. A second group of genes associated with Fragile X syndrome, includes NUFIP1 which binds a RNA binding protein coded by FMR1, FMRP. FMR1 has previously been identified with microsatellite repeats. NUFIP1 has a nuclear localization signal (NLS) and co-localizes in the nucleus with FMRP; FMRP also has NLS and a nuclear export signal allowing it to shuttle between the nucleus and cytoplasm, suggesting that NUFIP1 with FMRP may be associated with snRNPs or snoRNPs and also mRNA stabilization and export for translation. Additional studies have demonstrated NUFIP1 to interact with BRCA1 to stimulate ‘activator-independent’ RNA polymerase II and are associated with multiple complexes that instigate transcription and elongation.

Microsatellites and other repeat elements are associated with DNA ‘fragile sites’, locations within chromatin susceptible to constrictions or break-points that are linked to cancers and mental retardation diseases. DIP2B appears to be an important gene in neurocognitive development and also susceptible to repeat modifications which further advocates its potential in gliomagenesis. Similarly, BRWD2 is located at a break-point on chromosome 10 and allelic deletions within 10q 25-26 and 19q 13.3-13.4 are the most common alterations in glial tumors. Given the location of the break, BRWD2 is considered a candidate tumor suppressor. Clinical markers for GBM include loss or deletions in chromosome 10. Loss of 10p is found in 47% and 10q in 70% of primary GBMs and 10q loss is observed in 63% of secondary GBMs. In our GBM signature we identified 4 loci (and in total 8) in Ch10 at FGFR2, BRWD2 (WDR11), GLUD1, and NRP1; none were identified in the LGG signature though variant loci were found from genes in chromosome 10 (including those in NRP1 and COL17A1).

Disease-Associated Genes & Links to Male-Associated Biology:

Several genes highlighted are linked to other diseases or conditions with neurological or cognitive functions, including: STR, ARL13B and OFD1 (Joubert syndrome), NBPF1, and ICA1L (a contributor to amyotrophic lateral sclerosis).

A number of studies have highlighted a bias in gliomas in males compared to females. In this analysis, within the signature loci we observed loci associated with eight genes contributive to male specific biological processes, including the following: OFD1, STRC (with exonic repeat CAG), FRMD7, BRWD2, DICER1, HYDIN (may interact with neuroblastoma breakpoint family genes 1, 9, 10, and 12; a duplicate copy is found on Chromosome 1), DHX36, and DPY19L2P2. DDX20 is well known for its regulation and suppression of steroidogenic factor 1 (SF-1) which is expressed in gonadal tissues. These genes have brain and testis specific expression, including spermatogenesis, and some with testis only expression. Microsatellite loci with genotypes specific for cancer may be important to GBM in males.

Gene Ontologies & Cell Functions Important in Lower Grade Gliomas:

Here we analyzed a population of Grade II and III OD, OA, and A from a collective population of 178 samples, referenced as LGG. The LGG cancer-associated signature loci included 66; nine of these were also identified in the GBM signature (PSME, LAMP1, FUBP3, ATG3, EVC, SLC44A4, NEO1 and DDX20) and 2 loci in intergenic regions. From 16 of the 66 loci, are linked to genes previously identified with cancer, including: PSME, DEC1 (a tumor suppressor that deacetylates HDAC1/2-deacetylation of core histones is important to epigenetic repression and transcriptional regulation), ATM, LAMP1, GPR125, ACOXL, RAB2B, REL (interacts with multiple NFKB binding partners that regulate inflammation, immunity, differentiation, cell growth, tumorigenesis and apoptosis, HAVCR2 (mediates immunotolerance), XAGE3, CT45-1, RBM5 (regulates alternative splicing of mRNA and is a part of the splicesome A complex), SSX2 (transcription modulator), SNX25 (may interact with KIF1B), KIF1B and NPAT. Nine genes were associated with male biology, including: DEC1, ATM, XAGE3, CT45-1 (may interact with multiple XAGE family proteins), SSX, WNK1, TTLL5 (interacts with TP53 and TP73), CHODL, and CRISP1. C1orf77 interacts with several pre-mRNA modifying proteins; RNA polymerase II associated protein (RRAP2) and snRNA.

Ubiquitin Proteasome System:

Mutations in two known oncogenes that regulate cell signaling and cell cycle—ATM and REL—were both identified with signature microsatellite loci in LGG germline sequences. Both genes had monomeric microsatellite loci in the introns and were significantly different compared to “healthy” germline sequences. Similar to GBM our results for LGG demonstrate genes involved in ubiquitin proteasome system—including UBXN7 (function with HIF1-α and transcription activators FAF2, RBX1, DLX1/6, TCEB1 and several others), MYCBP2 (important to proteasomal degradation and also a key regulator of transcription by MYC), ATG3, and KLHL3 (a protein ligase that interact with multiple other KLHL proteins and possibly TNFAIP1). KLHL3 interacts with SLC12A3, which is regulated by WNK4, and WNK4 activity is inhibited by WNK1 (a GBM signature loci)). Some loci were identified with genes that interact with ubiquinone-NCAPD3 (donates electrons to ubiquinone and contributes to chromosomal rigidity) and C8orf38 (assembly of NADH: ubiquinone oxidoreductase complex [complex I]). Thus, similar to GBM, LGG microsatellite variant loci populate genes important to ubiquitin signaling, strengthening the importance of ubiquitin pathways in gliomas.

Cell Cycle & Development:

Cell cycle genes with cancer-associated genotypes included CDC16 (apart of the APC complex and an E3 ubiquitin ligase that regulates G1/M phase transition) and NPAT (G1 to S phase transition). Also, NPAT positively regulates ATM (a transcription repressor that binds RB 1 promoters), MIZF (a transcriptional activator that promotes H4, and is also a CpG island methylator), and PRKDC (which promotes and activates transcription of several histones with MIZF). This suggests that NPAT could be vital to DNA damage repair and cell proliferation and therefore a good therapeutic target. Additionally, we again see sets of genes (ATM and NPAT) with functional associations and both with LGG cancer-associated microsatellite genotypes. Several transcriptional regulatory genes were also identified, including: RBM5 (a component of the spliceosome A complex), SSX, YTHDC2 and DDX20.

Within the LGG signature loci, several are connected to genes that function in neural development, cell differentiation and proliferation; in total 11. More specifically, LNX2 interacts with the phosphotyrosine domain of NUMB in neurogenesis but also maintains progenitor cells (specifically, radial glial cells). MYCBP2 with FBXO45 are a part of an ubiquitin ligase complex that is necessary for neuronal development and possibly synaptogenesis, expression of both these genes are mostly in the brain and thymus. FBXO45 also interacts with TP73 (increase in ANp73 is associated with tumor progression and poor prognosis in human cancers and are also associated with neurological defects). CDRT1 and KIF1B are associated with Charcot-Marie-Tooth disease Type 1, a type of neuropathy. The top-ranked loci from the LGG signature was associated to KLAQ1, which works with PPP1CA (a protein phosphatase) that is associated with over 200 regulatory proteins, and contributes to neural tube and optic tissue closure; suggesting an important regulatory role in protein accessibility, early neuronal cell development and therefore a potentially important target in glioma cell development.

Ca²⁺ Regulation, Transport, & Metabolism:

Two signature loci were identified in SLC25A13 which is a Ca2+ dependent transporter exchanging glutamate for aspartate, as previously described glutamate metabolism can contribute to glioma phenotypes, dependent on IDH1 mutation; this protein also interacts with BRE (brain and reproductive organs) and is a modulator of TNFRSF1A and is also a component of the BRCA1-A complex and multiple TRIMMs (translocase of inner mitochondrial membrane proteins). Suggesting, metabolic genes may be important in LGGs.

### Example 6

### Microsatellites in the Exome are Predominantly Single-Allelic and Invariant

Re-analysis of microsatellites was performed on NextGen sequencing data from 651 healthy individuals (212 males and 439 females) exome sequenced as part of the 1000 genomes project. Microsatellite lengths were determined using the Garner Lab microsatellite pipeline. This pipeline determines lengths for all 1 to 6 mer microsatellites at least 10 nt long in exons and 12 nt outside of exons that can be uniquely mapped to the human reference genome (hg19). Sequencing reads used to call microsatellite lengths span the microsatellite with additional flanking sequence, which is used to map the read. We identified at least of 856,104 microsatellite loci genome-wide, of which 18,915 fall within exons. Although exome enrichment increases the number of reads targeting genomic exons, there are still non-exon reads present in exome sequencing data, therefore we were able to analyze an average of 70,518 (±34,793) microsatellite loci callable from exome sequencing data per individual. All individuals included in our analysis had at least 15,000 callable microsatellite loci.

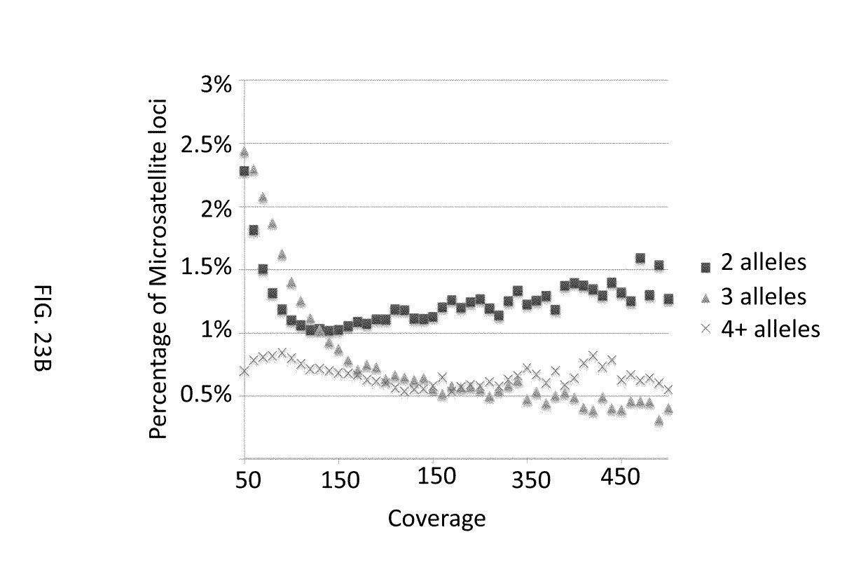

For this analysis the assumption that there are two alleles per individual at any given locus was removed to allow multiple alleles, or somatic variability to be identified. At every locus, an allele was determined when it was supported by a minimum of three unique sequencing reads. Therefore, a minimum of only 3 microsatellite-spanning reads was needed to identify a single allele at a locus while a minimum of 30 reads, if evenly divided would be sufficient to identify 10 alleles at that locus. We found that 95% of all microsatellite loci within the average individual exome were monoallelic. The combined mono- and di-allelic loci, the presumed homo- and heterozygotic loci, make up over 98% of all loci analyzed. This was true even at sequencing depths of >100× (FIG. 23A). From these results we conclude two things: first, that sequencing and bioinformatic errors are not overly abundant within microsatellite loci. This conclusion is supported by the overall decrease in the number of loci that are multi-allelic (used here to discuss those loci having 4 alleles) even at high sequencing coverage (FIG. 23A), and that there was no increase in the relative percentage of multi-allelic loci with increasing coverage (FIG. 23B). In addition, an error model for random sequencing error confirms that as the error rate increases, there are fewer loci that are mono-allelic at higher coverages (data not shown). The slope of the mono-allelic line for the linear portion of the 1 kGP data indicates that the error rate is less that 1% (data not shown), which is consistent with reported error rates for contemporary sequencers, but is contraindicative for the hypothesis that there is significantly more error in repeat regions. Second, we conclude that the majority of the microsatellites captured in exome sequencing are actually stable within an individual to the level detectable by NextGen exome sequencing of whole blood. This implies that only a small subset of microsatellites within an individual's exome is variable, i.e. have multiple alleles.

To determine if somatic variability is associated with ethnic background, we divided the exomes into four groups based on ethnicity (Asian: ASN, African: AFR, South American: SA, and European: EU). We found no difference between the ethnic backgrounds in the average numbers of multi-allelic loci that are present (data not shown).

To determine if specific loci are variable in multiple individuals, representing a possible unstable subset of microsatellites, we identified loci that were repeatedly multi-allelic. We chose a multi-allelic cut-off of four alleles based on the assumption that having one or two alleles at a locus is expected due to the two chromosomal copies of each locus, but it is unlikely that four or more alleles would be repeatedly present at an otherwise stable locus. Of the 55,870 loci that were called in at least 10 individuals with at least 15× coverage (sufficient to call multiple alleles if they are present), 1,584 loci were repeatedly multi-allelic (≧4 alleles were called in a minimum of 10 individuals), or ‘variable’, while 50,968 loci are invariant alleles were present in >99% of individuals at which the locus was called). The remaining 3,362 loci are intermediate, and include those loci with 3 alleles. We examined these classes of loci in more detail to try to identify properties that can influence variability of microsatellite loci.

We examined whether the genomic position of microsatellites might affect their variability. We found that loci that are intronic or located in the 3′UTR have a higher percentage of variation than loci in other genomic regions, including those loci that are intergenic (data not shown). Of the variable loci, 1,257 were intronic, monomeric repeats, all but one of which had an A/T motif (Table 21). The single variable C/G repeat was not unexpected given that we are only able to call an average of 26 C/G monomer repeats per exome whereas we are able to call an average of 3,975 A/T repeats. That monomeric A/T microsatellites are ‘unstable’ is consistent with their use as markers for instability in colorectal cancer.

To determine if microsatellite motif length affected variability within individuals we separated the microsatellites according to their motif-length (mono-, di-, tri- etc.). We found that a higher percentage of monomers are repeatedly multi-allelic (variable) or intermediate than any other motif (data not shown). Consistent with this, monomers, but not other motif lengths, had 3 or more alleles present in the average exome at sequencing read depths of >100 (data not shown). However, it should be noted that over 70% of monomeric microsatellites are invariant or intermediate (data not shown), showing that even in this class of microsatellites those that are variable are in the minority.

The microsatellites we were able to examine in this study were limited in length by sequencing read length, but we examined those that we can call to see if they are more frequently variant with increased length. We find that a higher percentage of the longer microsatellites (>40 nt) are considered intermediate (56%) or variant (11%) within the population (data not shown), whereas only 6% and 3% of loci <40 nt are considered intermediate or variant respectively. In contrast, variable loci <20 nt in length had 4 or more alleles present in a higher fraction of individuals in which they were called (data not shown). Importantly, the majority of all the loci identified as variant, including all of those loci >40 nt, were called in over 200 individuals (data not shown). From this we conclude that the number of alleles present in sequencing data at a microsatellite does not necessarily increase with increasing length of the microsatellite.

### Methods

We downloaded all available exome samples from the phase 1 publication (n=886) of the 1000 Genomes Project (1 kGP). All DNA samples from the 1 kGP were exome enriched and sequenced on the Illumina platform then quality filtered and aligned to hg19 using BWA. We performed re-alignment and allele identification at microsatellites using the pipeline described with minor modifications. The accuracy of our pipeline has been reported to be between 94.4% and 96.5% (3-4). This software was recently updated to accept hg19 alignments by converting the prior microsatellite coordinates using the UCSC Genome Lift-Over tool. The software was also updated to speed up the sub-functions allowing us to run an exome-sequenced sample in under 3 hours on a single core of an Intel Xeon 5500/5600 processor. We performed tests between our original hg18 software and the new, faster hg19 version to determine if any microsatellites calls differ. We identified 530 microsatellites for which different genotypes were obtained. These microsatellites were removed from our analysis set. We required a minimum of 15,000 microsatellite loci to be called per sample for inclusion in this study. This filtered out one female exome and 235 male exomes. A locus had to be called in a minimum of 10 exomes with at least 15× coverage to be included in our invariant/variant analysis.

Ethnic backgrounds: For evaluation of the effect of ethnicity on microsatellite variation, the exomes from the 1000 Genomes Project were divided into four broader ethnic categories: Asian or ASN (CDX, CHB, CHS, GIH and KHV populations); African or AFR (ACB, ASW, LWK and YRI populations); South American or SA (CLM, MXL and PEL); and European EU (CEU, FIN, GBR, IBS, PUR and TSI).

Genomic Regions: We used the refseq genes downloaded from the UCSC Genome Browser to associate microsatellite loci with genes and identify their genomic region. Upstream and downstream boundaries were defined as 1000 bases from the transcription start and end points. Microsatellite loci were associated with the gene region the majority of their sequences were contained in if they overlapped two regions.

### INCORPORATION BY REFERENCE

All publications and patents mentioned herein are hereby incorporated by reference in their entirety as if each individual publication or patent was specifically and individually indicated to be incorporated by reference.

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

### TABLES

TABLE 1
Breast Cancer

| | | | | | | | | | BC | |
|---|---|---|---|---|---|---|---|---|---|---|
| Microsatellite | | | | | 1kGP | | | BC | RNA_Seq | |
| Location | motif | refer- | | | total | 1kGP | 1kGP | RNA_seq | total | BC RNA_Seq |
| (Chromosome: nt | family | ence | re- | | sam- | total | alleles | total | samples | alleles |
| position) | cyclic | length | gion | gene symbol | ples | diffs | (calls) | samples | diff | (calls) |
| 1: 215860189-215860199 | ATT | 11 | exon | GPATCH2 | 128 | 0 | 11 (256) | 359 | 1 | 11 (717), 12 (1) |
| 11: 82321789-82321798 | AATG | 10 | exon | C11orf82 | 125 | 0 | 10 (250) | 289 | 1 | 8 (2), 10 (576) |
| 1: 112107101-112107110 | ATG | 10 | exon | DDX20 | 124 | 0 | 10 (248) | 382 | 1 | 7 (2), 10 (762) |
| 10: 102673750-102673761 | AAAAAG | 12 | exon | FAM178A | 123 | 0 | 12 (246) | 294 | 1 | 13 (1), 12 (587) |
| 1: 78731629-78731639 | TTTTC | 11 | exon | PTGFR | 122 | 0 | 11 (244) | 23 | 1 | 11 (45), 12 (1) |
| 6: 49533421-49533430 | ATGT | 10 | exon | MUT | 121 | 0 | 10 (242) | 380 | 1 | 11 (1), 10 (759) |
| 12: 21535856-21535869 | AATTTG | 14 | exon | RECQL | 121 | 0 | 14 (242) | 376 | 1 | 13 (1), 14 (751) |
| 1: 75002330-75002346 | ATG | 17 | exon | TYW3 | 121 | 0 | 17 (242) | 375 | 2 | 17 (746), 14 (4) |
| 5: 168950721-168950731 | AAC | 11 | exon | CCDC99 | 121 | 0 | 11 (242) | 367 | 1 | 11 (732), 12 (2) |
| 10: 119034325-119034334 | TTGC | 10 | exon | PDZD8 | 121 | 0 | 10 (242) | 361 | 5 | 11 (5), 10 (717) |
| 11: 107708788-107708800 | ATATT | 13 | exon | ATM | 121 | 0 | 13 (242) | 313 | 1 | 8 (2), 13 (624) |
| 1: 113437654-113437663 | AATAT | 10 | exon | LRIG2 | 121 | 0 | 10 (242) | 261 | 1 | 8 (2), 10 (520) |
| 10: 34689085-34689096 | ACACTG | 12 | exon | PARD3 | 120 | 0 | 12 (240) | 381 | 1 | 6 (2), 12 (760) |
| 11: 58676193-58676205 | AAAAGT | 13 | exon | FAM111A | 120 | 0 | 13 (240) | 373 | 1 | 9 (1), 13 (745) |
| 10: 17775294-17775306 | AAG | 13 | exon | STAM | 120 | 0 | 13 (240) | 367 | 6 | 11 (1), 13 (727), 14 |
| | | | | | | | | | | (6) |
| 13: 47779490-47779499 | AG | 10 | exon | RB1 | 120 | 0 | 10 (240) | 359 | 1 | 10 (716), 12 (2) |
| 10: 115653292-115653303 | AAAAAC | 12 | exon | NHLRC2 | 120 | 0 | 12 (240) | 354 | 4 | 13 (6), 12 (702) |
| 6: 144917570-144917579 | AGC | 10 | exon | UTRN | 120 | 0 | 10 (240) | 353 | 1 | 7 (1), 10 (705) |
| 5: 172470291-172470300 | AAGG | 10 | exon | C5orf41 | 120 | 0 | 10 (240) | 343 | 14 | 11 (17), 10 (669) |
| 1: 61326530-61326543 | AAG | 14 | exon | NFIA | 120 | 0 | 14 (240) | 307 | 1 | 15 (2), 14 (612) |
| 14: 54499444-54499466 | TTC | 23 | exon | WDHD1 | 120 | 0 | 23 (240) | 187 | 1 | 23 (372), 20 (2) |
| 13: 51905818-51905830 | TTTTC | 13 | exon | VPS36 | 119 | 0 | 13 (238) | 369 | 4 | 13 (734), 14 (4) |
| 11: 77072476-77072487 | TTTTC | 12 | exon | RSF1 | 119 | 0 | 12 (238) | 358 | 2 | 13 (2), 12 (714) |
| 12: 32025985-32025999 | TCC | 15 | exon | C12orf35 | 119 | 0 | 15 (238) | 356 | 2 | 12 (3), 15 (709) |
| 10: 76272683-76272697 | AAAAGC | 15 | exon | MYST4 | 119 | 0 | 15 (238) | 316 | 3 | 16 (6), 15 (626) |
| 4: 40505181-40505193 | AAG | 13 | exon | NSUN7 | 119 | 0 | 13 (238) | 135 | 6 | 13 (262), 14 (8) |
| 17: 62113782-62113791 | AAGC | 10 | exon | PRKCA | 119 | 0 | 10 (238) | 123 | 10 | 11 (16), 10 (230) |
| 11: 27328529-27328541 | TTTTC | 13 | exon | CCDC34 | 118 | 0 | 13 (236) | 365 | 5 | 13 (724), 14 (6) |
| 5: 154285777-154285786 | AAGG | 10 | exon | GEMIN5 | 118 | 0 | 10 (236) | 314 | 1 | 11 (1), 10 (627) |
| 20: 29694946-29694956 | TTC | 11 | exon | COX4I2 | 118 | 0 | 11 (236) | 270 | 1 | 8 (1), 11 (539) |
| 1: 195375584-195375594 | TTTG | 11 | exon | ASPM | 118 | 0 | 11 (236) | 198 | 1 | 11 (395), 10 (1) |
| 1: 158071599-158071611 | AAAAAG | 13 | exon | SLAMF8 | 118 | 0 | 13 (236) | 192 | 1 | 13 (383), 14 (1) |
| 11: 27335559-27335570 | TTTTTC | 12 | exon | CCDC34 | 117 | 0 | 12 (234) | 388 | 1 | 9 (1), 12 (775) |
| 9: 72157030-72157039 | CGG | 10 | exon | SMC5 | 117 | 0 | 10 (234) | 377 | 1 | 11 (2), 10 (752) |
| 11: 116138518-116138527 | TTGC | 10 | exon | BUD13 | 117 | 0 | 10 (234) | 365 | 1 | 11 (1), 10 (729) |
| 1: 11225884-11225896 | TTCTCC | 13 | exon | FRAP1 | 117 | 0 | 13 (234) | 335 | 1 | 13 (669), 12 (1) |
| 1: 232623159-232623170 | ACTTGG | 12 | exon | TARBP1 | 116 | 0 | 12 (232) | 371 | 4 | 13 (5), 12 (737) |
| 1: 159762579-159762591 | ATCACC | 13 | exon | HSPA6 | 116 | 0 | 13 (232) | 315 | 192 | 7 (251), 13 (379) |
| 13: 27795047-27795059 | TTTC | 13 | exon | FLT1 | 116 | 0 | 13 (232) | 262 | 3 | 13 (521), 14 (3) |
| 4: 84589090-84589102 | TTTC | 13 | exon | HELQ | 116 | 0 | 13 (232) | 91 | 4 | 13 (174), 14 (8) |
| 12: 47584393-47584405 | AAAG | 13 | exon | CCDC65 | 116 | 0 | 13 (232) | 67 | 1 | 13 (133), 14 (1) |
| 10: 94229068-94229079 | ATATGC | 12 | exon | IDE | 115 | 0 | 12 (230) | 381 | 1 | 13 (1), 12 (761) |
| 10: 105150196-105150207 | AAAAAC | 12 | exon | PDCD11 | 115 | 0 | 12 (230) | 343 | 5 | 13 (5), 12 (681) |
| 11: 35414083-35414092 | TGC | 10 | exon | DKFZP586H2123 | 115 | 0 | 10 (230) | 189 | 1 | 8 (1), 10 (377) |
| 3: 50660436-50660447 | AGGC | 12 | exon | MAPKAPK3 | 114 | 0 | 12 (228) | 370 | 64 | 13 (66), 12 (674) |
| 2: 237909603-237909616 | AGC | 14 | exon | COL6A3 | 114 | 25 | 11 (29), | 289 | 2 | 11 (2), 14 (576) |
| | | | | | | | 14 (199) | | | |
| 17: 63252843-63252858 | ACG | 16 | exon | BPTF | 114 | 3 | 13 (3), | 280 | 5 | 13 (9), 16 (551) |
| | | | | | | | 16 (225) | | | |
| 10: 127658854-127658864 | AAG | 11 | exon | FANK1 | 114 | 0 | 11 (228) | 274 | 6 | 8 (8), 11 (540) |
| 18: 75576176-75576196 | AGG | 21 | exon | CTDP1 | 113 | 12 | 21 | 343 | 9 | 21 (672), 24 (14) |
| | | | | | | | (211), | | | |
| | | | | | | | 24 (15) | | | |
| 5: 140999345-140999354 | AAGG | 10 | exon | RELL2 | 113 | 0 | 10 (226) | 288 | 1 | 11 (1), 10 (575) |
| 12: 70519831-70519841 | CGG | 11 | exon | TBC1D15 | 113 | 0 | 11 (226) | 152 | 1 | 11 (302), 12 (2) |
| 6: 33763867-33763879 | AGG | 13 | exon | ITPR3 | 112 | 1 | 10 (1), | 385 | 2 | 10 (3), 13 (767) |
| | | | | | | | 13 (223) | | | |
| 10: 57788416-57788438 | AGCCTC | 23 | exon | ZWINT | 112 | 0 | 23 (224) | 369 | 1 | 23 (737), 29 (1) |
| 5: 6808013-6808026 | AC | 14 | exon | POLS | 112 | 0 | 14 (224) | 340 | 1 | 15 (2), 14 (678) |
| 15: 62760043-62760065 | ACC | 23 | exon | ZNF609 | 112 | 0 | 23 (224) | 256 | 1 | 23 (511), 20 (1) |
| 19: 50966936-50966946 | TCC | 11 | exon | DMPK | 111 | 0 | 11 (222) | 384 | 1 | 8 (1), 11 (767) |
| 2: 24284629-24284639 | TTC | 11 | exon | ITSN2 | 111 | 0 | 11 (222) | 376 | 1 | 8 (2), 11 (750) |
| 20: 205710-205722 | TTC | 13 | exon | C20orf96 | 111 | 0 | 13 (222) | 358 | 9 | 13 (705), 12 (1), 14 |
| | | | | | | | | | | (10) |
| 2: 238113766-238113775 | AGG | 10 | exon | MLPH | 111 | 0 | 10 (222) | 324 | 1 | 7 (2), 10 (646) |
| 1: 89424725-89424734 | TGC | 10 | exon | GBP4 | 111 | 0 | 10 (222) | 321 | 1 | 9 (2), 10 (640) |
| 7: 72359667-72359676 | AAC | 10 | exon | NSUN5 | 111 | 0 | 10 (222) | 203 | 68 | 7 (71), 10 (335) |
| 12: 48313940-48313952 | AGC | 13 | exon | PRPF40B | 111 | 0 | 13 (222) | 6 | 5 | 13 (2), 14 (10) |
| 7: 72499559-72499590 | TCC | 32 | exon | BAZ1B | 111 | 0 | 32 (222) | 3 | 3 | 14 (6) |
| 20: 23293911-23293940 | AGG | 30 | exon | GZF1 | 111 | 0 | 30 (222) | 3 | 1 | 30 (4), 9 (2) |
| 9: 130910019-130910031 | TCC | 13 | exon | CRAT | 110 | 0 | 13 (220) | 362 | 1 | 10 (2), 13 (722) |
| 1: 158179475-158179488 | CCGG | 14 | exon | IGSF9 | 110 | 0 | 14 (220) | 345 | 2 | 15 (3), 14 (687) |
| 1: 31678477-31678491 | AGC | 15 | exon | SERINC2 | 110 | 94 | 18 | 213 | 198 | 18 (392), 15 (34) |
| | | | | | | | (162), | | | |
| | | | | | | | 15 (58) | | | |
| 9: 132749311-132749326 | AAG | 16 | exon | ABL1 | 109 | 0 | 16 (218) | 387 | 1 | 13 (1), 16 (773) |
| 20: 42127973-42127983 | CCG | 11 | exon | TOX2 | 109 | 7 | 11 | 35 | 2 | 11 (66), 14 (4) |
| | | | | | | | (208), | | | |
| | | | | | | | 14 (10) | | | |
| 11: 67574568-67574586 | TGGGCC | 19 | exon | TCIRG1 | 108 | 0 | 19 (216) | 373 | 1 | 25 (1), 19 (745) |
| 3: 53504233-53504255 | ATG | 23 | exon | CACNA1D | 108 | 0 | 23 (216) | 19 | 1 | 24 (2), 23 (36) |
| 11: 65576476-65576487 | CCG | 12 | exon | SF3B2 | 107 | 2 | 12 | 383 | 1 | 12 (765), 15 (1) |
| | | | | | | | (212), | | | |
| | | | | | | | 15 (2) | | | |
| 12: 130847687-130847701 | AAG | 15 | exon | SFRS8 | 107 | 0 | 15 (214) | 320 | 1 | 12 (2), 15 (638) |
| 1: 8638909-8638934 | TTTGTC | 26 | exon | RERE | 106 | 3 | 26 | 192 | 9 | 26 (367), 20 (17) |
| | | | | | | | (208), | | | |
| | | | | | | | 20 (4) | | | |
| 7: 99795065-99795076 | TCC | 12 | exon | PILRB | 105 | 21 | 9 (28), | 339 | 98 | 9 (161), 12 (517) |
| | | | | | | | 12 (182) | | | |
| 3: 185911828-185911848 | TCC | 21 | exon | MAGEF1 | 105 | 77 | 21 (91), | 324 | 241 | 21 (208), 24 (440) |
| | | | | | | | 24 (119) | | | |
| 8: 22318174-22318187 | TGC | 14 | exon | SLC39A14 | 105 | 27 | 8 (40), | 322 | 104 | 8 (171), 14 (473) |
| | | | | | | | 14 (170) | | | |
| 11: 18084107-18084124 | TCC | 18 | exon | SAAL1 | 105 | 3 | 18 | 216 | 1 | 18 (430), 24 (2) |
| | | | | | | | (207), | | | |
| | | | | | | | 24 (3) | | | |
| 1: 221603326-221603347 | TGC | 22 | exon | SUSD4 | 104 | 2 | 22 | 286 | 3 | 25 (1), 22 (567), 19 |
| | | | | | | | (205), | | | (4) |
| | | | | | | | 19 (3) | | | |
| 19: 50603699-50603713 | AAG | 15 | exon | CD3EAP | 103 | 0 | 15 (206) | 340 | 9 | 16 (10), 17 (1), 15 |
| | | | | | | | | | | (669) |
| 12: 63290721-63290730 | TTC | 10 | exon | RASSF3 | 103 | 2 | 7 (2), 10 | 254 | 1 | 7 (2), 10 (506) |
| | | | | | | | (204) | | | |
| 12: 55960472-55960500 | TGC | 29 | exon | R3HDM2 | 102 | 0 | 29 (204) | 169 | 1 | 23 (2), 29 (336) |
| 9: 134193732-134193749 | ATC | 18 | exon | SETX | 101 | 0 | 18 (202) | 298 | 1 | 21 (1), 18 (595) |
| 1: 35976247-35976261 | TTC | 15 | exon | CLSPN | 101 | 1 | 12 (1), | 182 | 7 | 12 (11), 15 (353) |
| | | | | | | | 15 (201) | | | |
| 1: 1674208-1674235 | TCC | 28 | exon | NADK | 98 | 41 | 25 (2), | 263 | 6 | 25 (10), 28 (516) |
| | | | | | | | 28 | | | |
| | | | | | | | (137), | | | |
| | | | | | | | 31 (57) | | | |
| 19: 4768289-4768315 | AGG | 27 | exon | TICAM1 | 98 | 16 | 27 | 109 | 5 | 27 (209), 24 (1), 30 |
| | | | | | | | (177), | | | (8) |
| | | | | | | | 30 (19) | | | |
| 14: 102662628-102662655 | AAG | 28 | exon | TNFAIP2 | 96 | 0 | 28 (192) | 314 | 1 | 25 (1), 28 (627) |
| 1: 6458598-6458616 | TCC | 19 | exon | PLEKHG5 | 96 | 0 | 19 (192) | 269 | 1 | 19 (536), 17 (2) |
| 1: 21140821-21140834 | AAGG | 14 | exon | EIF4G3 | 91 | 0 | 14 (182) | 282 | 20 | 23 (22), 14 (542) |
| 7: 21434829-21434846 | AGG | 18 | exon | SP4 | 90 | 0 | 18 (180) | 33 | 3 | 18 (61), 24 (5) |
| 22: 40940517-40940538 | AGG | 22 | exon | TCF20 | 89 | 0 | 22 (178) | 236 | 1 | 22 (470), 16 (2) |
| 2: 201145537-201145546 | ACTC | 10 | exon | SGOL2 | 88 | 0 | 10 (176) | 321 | 1 | 11 (1), 10 (641) |
| 1: 44368967-44368978 | AAC | 12 | exon | KLF17 | 88 | 12 | 9 (18), | 11 | 4 | 9 (7), 12 (15) |
| | | | | | | | 12 (158) | | | |
| 1: 58910180-58910191 | TTCTC | 12 | exon | MYSM1 | 87 | 0 | 12 (174) | 305 | 1 | 11 (2), 12 (608) |
| 4: 152718473-152718482 | ATCC | 10 | exon | FAM160A1 | 87 | 0 | 10 (174) | 199 | 1 | 11 (1), 10 (397) |
| 10: 69872808-69872817 | TTC | 10 | exon | DNA2 | 84 | 0 | 10 (168) | 256 | 1 | 9 (1), 10 (511) |
| 7: 154391474-154391496 | TGC | 23 | exon | PAXIP1 | 83 | 0 | 23 (166) | 268 | 1 | 26 (2), 23 (534) |
| 10: 91487885-91487896 | AAGGAG | 12 | exon | KIF20B | 82 | 22 | 18 (34), | 346 | 100 | 18 (146), 12 (546) |
| | | | | | | | 12 (130) | | | |
| 6: 32299637-32299668 | AGC | 32 | exon | NOTCH4 | 82 | 62 | 35 (6), | 17 | 17 | 17 (2), 20 (32) |
| | | | | | | | 32 (55), | | | |
| | | | | | | | 17 (2), | | | |
| | | | | | | | 29 (72), | | | |
| | | | | | | | 20 (29) | | | |
| 4: 71773555-71773573 | AGG | 19 | exon | UTP3 | 81 | 0 | 19 (162) | 365 | 1 | 16 (1), 19 (729) |
| 22: 22893073-22893082 | ACC | 10 | exon | CABIN1 | 80 | 0 | 10 (160) | 325 | 118 | 16 (144), 10 (506) |
| 7: 138601637-138601650 | AAGG | 14 | exon | UBN2 | 80 | 0 | 14 (160) | 222 | 1 | 15 (1), 14 (443) |
| 11: 118279213-118279237 | CCCCCG | 25 | exon | BCL9L | 80 | 0 | 25 (160) | 3 | 1 | 25 (4), 13 (2) |
| 12: 88441293-88441302 | ATCC | 10 | exon | GALNT4 | 79 | 0 | 10 (158) | 327 | 1 | 9 (1), 10 (653) |
| 2: 206881623-206881632 | AGC | 10 | exon | ZDBF2 | 79 | 0 | 10 (158) | 66 | 1 | 7 (2), 10 (130) |
| 10: 5838663-5838675 | ATC | 13 | exon | C10orf18 | 78 | 0 | 13 (156) | 389 | 1 | 10 (1), 13 (777) |
| 8: 94809677-94809686 | AAG | 10 | exon | FAM92A1 | 78 | 0 | 10 (156) | 375 | 8 | 7 (10), 10 (740) |
| 12: 54909139-54909154 | ACCC | 16 | exon | OBFC2B | 77 | 0 | 16 (154) | 254 | 1 | 16 (507), 15 (1) |
| 4: 169382013-169382026 | ACAG | 14 | exon | DDX60 | 76 | 0 | 14 (152) | 377 | 1 | 13 (1), 14 (753) |
| 3: 141767687-141767703 | AGG | 17 | exon | CLSTN2 | 76 | 0 | 17 (152) | 264 | 2 | 11 (4), 17 (524) |
| 10: 97909836-97909848 | AAAAAC | 13 | exon | ZNF518A | 74 | 6 | 13 | 361 | 27 | 13 (680), 14 (42) |
| | | | | | | | (141), | | | |
| | | | | | | | 14 (7) | | | |
| 11: 10558656-10558668 | TCC | 13 | exon | MRVI1 | 74 | 0 | 13 (148) | 322 | 1 | 10 (1), 13 (643) |
| 5: 70842546-70842555 | AG | 10 | exon | BDP1 | 74 | 0 | 10 (148) | 270 | 1 | 8 (2), 10 (538) |
| 14: 22310554-22310566 | AGC | 13 | exon | OXA1L | 74 | 3 | 16 (6), | 228 | 26 | 16 (50), 13 (406) |
| | | | | | | | 13 (142) | | | |
| 11: 32580971-32580984 | TTTTC | 14 | exon | CCDC73 | 74 | 0 | 14 (148) | 73 | 1 | 15 (2), 14 (144) |
| 5: 156412022-156412033 | TTG | 12 | exon | HAVCR1 | 72 | 13 | 9 (23), | 9 | 2 | 9 (3), 12 (15) |
| | | | | | | | 12 (121) | | | |
| 12: 1932585-1932613 | TGC | 29 | exon | DCP1B | 71 | 42 | 32 (71), | 6 | 1 | 26 (2), 29 (10) |
| | | | | | | | 26 (1), | | | |
| | | | | | | | 29 (70) | | | |
| 12: 78699731-78699742 | ATTTCC | 12 | exon | PPP1R12A | 70 | 0 | 12 (140) | 10 | 1 | 13 (2), 12 (18) |
| 19: 37892029-37892038 | TC | 10 | exon | NUDT19 | 69 | 0 | 10 (138) | 381 | 1 | 10 (761), 12 (1) |
| 5: 175858598-175858614 | AAAG | 17 | exon | FAF2 | 69 | 0 | 17 (138) | 381 | 1 | 16 (1), 17 (761) |
| 11: 93101596-93101607 | AAGAG | 12 | exon | KIAA1731 | 67 | 0 | 12 (134) | 375 | 1 | 7 (1), 12 (749) |
| 11: 33587991-33588001 | AAAG | 11 | exon | C11orf41 | 67 | 0 | 11 (134) | 250 | 3 | 11 (497), 12 (3) |
| 1: 1637752-1637761 | TTTC | 10 | exon | CDC2L1 | 67 | 1 | 16 (1), | 247 | 241 | 16 (400), 10 (94) |
| | | | | | | | 10 (133) | | | |
| 11: 85052890-85052899 | TTC | 10 | exon | CREBZF | 66 | 0 | 10 (132) | 373 | 1 | 7 (1), 10 (745) |
| 14: 23726713-23726722 | TC | 10 | exon | IPO4 | 66 | 0 | 10 (132) | 5 | 1 | 19 (2), 10 (8) |
| 16: 88444381-88444396 | AGG | 16 | exon | SPIRE2 | 65 | 8 | 19 (13), | 59 | 5 | 19 (10), 16 (108) |
| | | | | | | | 16 (117) | | | |
| 4: 15798994-15799004 | TTTC | 11 | exon | TAPT1 | 64 | 0 | 11 (128) | 369 | 1 | 11 (737), 12 (1) |
| 1: 158166068-158166080 | CGG | 13 | exon | IGSF9 | 64 | 0 | 13 (128) | 351 | 1 | 19 (1), 13 (701) |
| 11: 33646246-33646256 | ACAG | 11 | exon | C11orf41 | 64 | 0 | 11 (128) | 191 | 3 | 11 (376), 12 (6) |
| 7: 69893513-69893538 | ACC | 26 | exon | AUTS2 | 57 | 2 | 32 (2), | 289 | 1 | 26 (576), 29 (2) |
| | | | | | | | 23 (2), | | | |
| | | | | | | | 26 (110) | | | |
| 13: 44937205-44937215 | CGG | 11 | exon | COG3 | 57 | 0 | 11 (114) | 203 | 1 | 11 (404), 14 (2) |
| 17: 7742582-7742596 | AAG | 15 | exon | CHD3 | 55 | 0 | 15 (110) | 386 | 1 | 12 (2), 15 (770) |
| 17: 7232598-7232611 | AGCC | 14 | exon | TNK1 | 55 | 0 | 14 (110) | 380 | 1 | 13 (1), 14 (759) |
| 5: 56213606-56213631 | AAC | 26 | exon | MAP3K1 | 55 | 47 | 23 (88), | 293 | 271 | 23 (508), 26 (78) |
| | | | | | | | 26 (22) | | | |
| 1: 20106687-20106697 | AAG | 11 | exon | OTUD3 | 55 | 0 | 11 (110) | 164 | 1 | 8 (2), 11 (326) |
| 2: 74603987-74603996 | AGGG | 10 | exon | DQX1 | 53 | 0 | 10 (106) | 112 | 1 | 16 (1), 10 (223) |
| 2: 3727027-3727036 | AAG | 10 | exon | ALLC | 53 | 28 | 7 (47), | 1 | 1 | 7 (2) |
| | | | | | | | 10 (59) | | | |
| 1: 86818484-86818517 | ACTCCT | 34 | exon | CLCA4 | 52 | 44 | 28 (81), | 3 | 3 | 28 (6) |
| | | | | | | | 34 (23) | | | |
| 3: 51952455-51952465 | AAG | 11 | exon | PARP3 | 51 | 0 | 11 (102) | 344 | 4 | 8 (4), 11 (682), 14 |
| | | | | | | | | | | (2) |
| 1: 210526078-210526090 | TCG | 13 | exon | PPP2R5A | 48 | 1 | 16 (1), | 278 | 5 | 16 (6), 13 (550) |
| | | | | | | | 13 (95) | | | |
| 20: 255202-255219 | CCG | 18 | exon | SOX12 | 46 | 0 | 18 (92) | 208 | 1 | 18 (415), 24 (1) |
| 12: 116990711-116990742 | TCC | 32 | exon | FLJ20674 | 46 | 19 | 32 (59), | 23 | 23 | 26 (44), 29 (2) |
| | | | | | | | 28 (2), | | | |
| | | | | | | | 26 (30), | | | |
| | | | | | | | 29 (1) | | | |
| 16: 87311084-87311098 | TTC | 15 | exon | FAM38A | 43 | 0 | 15 (86) | 381 | 1 | 12 (2), 15 (760) |
| 14: 102874510-102874532 | ACC | 23 | exon | EIF5 | 43 | 2 | 26 (3), | 342 | 4 | 26 (6), 23 (678) |
| | | | | | | | 23 (83) | | | |
| 20: 30410253-30410266 | AAG | 14 | exon | ASXL1 | 41 | 0 | 14 (82) | 307 | 1 | 11 (1), 14 (613) |
| 11: 587408-587421 | AGG | 14 | exon | PHRF1 | 40 | 0 | 14 (80) | 369 | 1 | 11 (2), 14 (736) |
| 12: 120731943-120731954 | TCCGGC | 12 | exon | SETD1B | 40 | 0 | 12 (80) | 347 | 1 | 9 (1), 12 (693) |
| 19: 43591342-43591359 | AAG | 18 | exon | FAM98C | 35 | 1 | 21 (2), | 341 | 15 | 21 (23), 18 (658), 15 |
| | | | | | | | 18 (68) | | | (1) |
| 17: 77250022-77250035 | AGG | 14 | exon | CCDC137 | 31 | 0 | 14 (62) | 380 | 3 | 11 (5), 14 (755) |
| 14: 92224291-92224307 | CGG | 17 | exon | RIN3 | 26 | 22 | 17 (9), | 74 | 66 | 17 (16), 14 (132) |
| | | | | | | | 14 (43) | | | |
| 9: 126601541-126601552 | CCG | 12 | exon | OLFML2A | 24 | 0 | 12 (48) | 220 | 1 | 13 (1), 12 (439) |
| 17: 17637819-17637859 | AGC | 41 | exon | RAI1 | 19 | 15 | 41 (9), | 1 | 1 | 29 (2) |
| | | | | | | | 38 (21), | | | |
| | | | | | | | 29 (8) | | | |
| 3: 40478525-40478556 | TGC | 32 | exon | RPL14 | 15 | 11 | 38 (4), | 99 | 99 | 8 (2), 11 (18), 26 |
| | | | | | | | 35 (6), | | | (10), 23 (59), 29 |
| | | | | | | | 32 (8), | | | (12), 17 (26), 20 |
| | | | | | | | 26 (4), | | | (23), 14 (48) |
| | | | | | | | 23 (2), | | | |
| | | | | | | | 41 (4), | | | |
| | | | | | | | 47 (2) | | | |
| 11: 47745240-47745251 | TGG | 12 | exon | FNBP4 | 13 | 6 | 6 (11), | 183 | 83 | 6 (147), 12 (219) |
| | | | | | | | 12 (15) | | | |
| 2: 75039317-75039334 | CGG | 18 | exon | POLE4 | 7 | 0 | 18 (14) | 197 | 1 | 21 (1), 18 (393) |
| 22: 27526500-27526511 | ACC | 12 | exon | XBP1 | 6 | 0 | 12 (12) | 293 | 1 | 12 (585), 15 (1) |
| 12: 19484228-19484239 | AGC | 12 | exon | AEBP2 | 6 | 0 | 12 (12) | 97 | 1 | 12 (192), 15 (2) |
| 6: 43005336-43005362 | TGC | 27 | exon | CNPY3 | 5 | 0 | 27 (10) | 209 | 7 | 27 (408), 24 (10) |
| 20: 226688-226707 | CGG | 20 | exon | ZCCHC3 | 3 | 3 | 17 (6) | 80 | 80 | 17 (159), 20 (1) |
| 18: 46977136-46977161 | CCG | 26 | exon | MEX3C | 3 | 3 | 17 (6) | 26 | 25 | 26 (2), 17 (50) |
| 1: 144788110-144788125 | ACCCC | 16 | exon | FAM108A3 | 2 | 0 | 16 (4) | 263 | 263 | 17 (526) |
| 2: 88707845-88707869 | AGC | 25 | exon | EIF2AK3 | 2 | 2 | 22 (4) | 9 | 8 | 22 (16), 25 (2) |
| 1: 11633367-11633377 | CGG | 11 | exon | FBXO2 | 1 | 0 | 11 (2) | 123 | 22 | 8 (2), 11 (207), 14 |
| | | | | | | | | | | (37) |
| 19: 38484848-38484866 | CCG | 19 | exon | CEBPA | 1 | 0 | 19 (2) | 31 | 1 | 19 (61), 12 (1) |
| 12: 109505123-109505142 | CCG | 20 | exon | PPTC7 | 1 | 0 | 20 (2) | 3 | 1 | 17 (2), 20 (4) |

Table 1.

TABLE 2
Breast Cancer

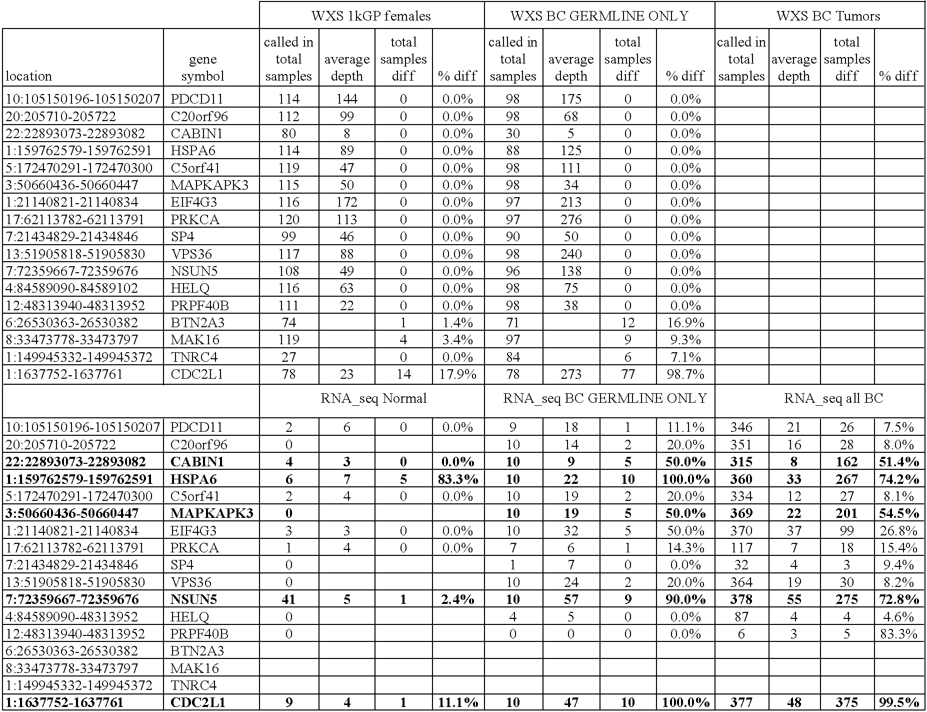

17 genes with exonic microsatellite variants associated with breast cancer. 13 of these genes (white) showed significant variation between the WXS 1kGP females and the RNA_seq of all BC tumors (P ≦ 0.05). An additional 3 loci (light grey: BTN2A3, MAK16 and TNRC4) were significantly variant between the WXS 1kGP and the WXS BC germline samples. CDC2L1 (dark grey) was significantly variant between the WXS 1kGP female and both the WXS BC germline samples and the RNA_seq BC samples. NSUN5 was the only locus that showed significance between the RNA_seq normal and RNA_seq BC samples, primarily due to the low coverage across microsatellites within the RNA_seq normal data. For 5 loci (bold), over 50% of the transcripts from both the RNA_seq BC germline only and RNA_seq all BC sets were variant.

TABLE 3
Ovarian Cancer

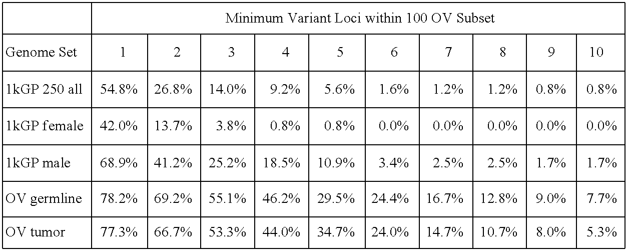

Percentage of genomes having an OV-signature with the indicated minimum variant loci. There is an inverse relationship between the minimum number of variant loci for classifying a genome as having an OV signature and the percentage of genomes classified. The grey box demarks the number of variants required to reduce OV signature calling below the expected level of 1.7% in the 1kGP female population.

TABLE 4
Ovarian Cancer

| | | | | | | | | 1kGP females | | OV germline | | OV tumors | |
|---|---|---|---|---|---|---|---|---|---|---|---|---|---|
| | Microsatellite | | | | | | | | alleles diff | | alleles diff | | alleles diff |
| | Location | | | | genome set | | consensus | | from | | from | tumor genomes | from |
| | (chromosome: nt | | | | with variant | hg18 ref | from 1kGP | genomes locus | female | genomes locus | female | locus | female |
| | position) | motif | region | gene symbol | alleles | length | females | called in | consensus | called in | consensus | called in | consensus |
| 1 | 12: 1390072-1390085 | T | intron | ABCC1 | both | 16 | 16 | 48 | 0 | 20 | 9 | 25 | 7 |
| 2 | 16: 16116003-16116018 | ATT | intron | ACSL1 | both | 13 | 13 | 54 | 2 | 32 | 10 | 28 | 5 |
| 3 | 4: 185931872-185931884 | A | intron | CMYA5 | both | 14 | 14 | 41 | 1 | 22 | 5 | 20 | 7 |
| 4 | 5: 79076734-79076747 | A | intron | COL24A1 | both | 22 | 22 | 50 | 0 | 18 | 28 | 15 | 24 |
| 5 | 1: 86081282-86081303 | AAAC | intron | DGKI | both | 13 | 13 | 41 | 1 | 47 | 9 | 41 | 6 |
| 6 | 7: 136990139-136990151 | A | intron | DOCK4 | both | 13 | 13 | 45 | 0 | 35 | 5 | 29 | 8 |
| 7 | 7: 111261986-111261998 | A | intron | PIK3IP1 | both | 17 | 17 | 103 | 4 | 55 | 12 | 57 | 15 |
| 8 | 22: 30009283-30009299 | AAAC | intron | TNIK | both | 14 | 14 | 51 | 2 | 41 | 12 | 33 | 11 |
| 9 | 3: 172326711-172326724 | A | intron | ULK4 | both | 13 | 13 | 33 | 1 | 40 | 5 | 35 | 5 |
| 10 | 3: 41852478-41852490 | A | intron | ZMYM2 | both | 13 | 12 | 50 | 2 | 47 | 9 | 36 | 5 |
| 11 | 13: 19554139-19554151 | T | 3utrl | ERC1 | both | 14 | 14 | 36 | 0 | 16 | 9 | 22 | 6 |
| 12 | 16: 49656164-49656184 | AC | intergenic | — | both | 21 | 21 | 61 | 0 | 16 | 5 | 16 | 5 |
| 13 | 3: 148477767-148477781 | A | intergenic | — | both | 15 | 15 | 66 | 2 | 30 | 6 | 27 | 5 |
| 14 | 10: 117813758-117813769 | A | 5utrI | TEAD1 | germline | 25 | 25 | 61 | 0 | 25 | 7 | 21 | 2 |
| 15 | 11: 12728672-12728696 | AGAC | 5utrI | ZNF92 | germline | 25 | 25 | 40 | 1 | 42 | 11 | 35 | 3 |
| 16 | 7: 64490218-64490242 | TG | intron | RNPEP | germline | 29 | 23 | 27 | 1 | 4 | 5 | 3 | 1 |
| 17 | 3: 55084275-55084288 | TACT | intron | TIE1 | germline | 23 | 23 | 105 | 3 | 41 | 4 | 43 | 2 |
| 18 | 1: 200230854-200230882 | TTGT | intron | PKN2 | germline | 14 | 14 | 34 | 1 | 6 | 6 | 3 | 1 |
| | | TT | | | | | | | | | | | |
| 19 | 1: 43552312-43552334 | TG | intron | ABCD3 | germline | 15 | 15 | 27 | 1 | 10 | 6 | 6 | 2 |
| 20 | 1: 88998318-88998331 | T | intron | AFAP1L2 | germline | 18 | 18 | 95 | 3 | 13 | 7 | 8 | 4 |
| 21 | 1: 94736728-94736742 | T | intron | ATP7B | germline | 13 | 13 | 47 | 1 | 13 | 6 | 15 | 0 |
| 22 | 10: 116138036-116138053 | AC | intron | TCF12 | germline | 27 | 27 | 102 | 0 | 46 | 7 | 37 | 3 |
| 23 | 13: 51413512-51413524 | A | intron | FAH | germline | 14 | 14 | 42 | 0 | 29 | 6 | 24 | 3 |
| 24 | 15: 54999521-54999547 | TTTG | intron | RIOK3 | germline | 24 | 24 | 112 | 3 | 42 | 5 | 34 | 3 |
| 25 | 15: 78247632-78247645 | T | intron | DDX18 | germline | 12 | 12 | 114 | 4 | 47 | 6 | 39 | 2 |
| 26 | 18: 19313146-19313169 | TG | intron | GPD2 | germline | 14 | 14 | 47 | 1 | 19 | 5 | 12 | 4 |
| 27 | 2: 118299153-118299164 | TGA | intron | WDSUB1 | germline | 12 | 12 | 41 | 0 | 7 | 5 | 5 | 3 |
| 28 | 2: 157078265-157078278 | T | intron | RAPGEF4 | germline | 14 | 14 | 52 | 0 | 19 | 5 | 20 | 0 |
| 29 | 2: 159800950-159800961 | A | intron | PIK3CB | germline | 13 | 13 | 30 | 0 | 19 | 6 | 8 | 2 |
| 30 | 2: 173569352-173569365 | A | intron | AGXT2 | germline | 12 | 12 | 53 | 2 | 32 | 5 | 34 | 1 |
| 31 | 3: 139883473-139883485 | A | intron | ASCC3 | germline | 13 | 13 | 32 | 0 | 34 | 6 | 25 | 1 |
| 32 | 5: 35062457-35062468 | A | intron | BAI3 | germline | 12 | 12 | 42 | 1 | 36 | 4 | 37 | 2 |
| 33 | 6: 101094988-101095000 | A | intron | LRGUK | germline | 12 | 12 | 80 | 2 | 44 | 4 | 34 | 0 |
| 34 | 6: 70097222-70097233 | A | intron | ENPP2 | germline | 15 | 14 | 55 | 1 | 12 | 5 | 17 | 1 |
| 35 | 7: 133527177-133527188 | T | intron | CLCN4 | germline | 17 | 17 | 98 | 3 | 11 | 6 | 7 | 2 |
| 36 | 8: 120700839-120700853 | A | intron | CAPN6 | germline | 14 | 14 | 28 | 0 | 31 | 5 | 25 | 2 |
| 37 | X: 10123355-10123371 | AT | intron | PLS3 | germline | 13 | 13 | 43 | 0 | 24 | 4 | 14 | 1 |
| 38 | X: 110381185-110381198 | A | intron | PRKX | germline | 13 | 13 | 32 | 1 | 45 | 4 | 48 | 2 |
| 39 | X: 114777384-114777396 | T | 3utrE | GFRA1 | germline | 12 | 12 | 79 | 0 | 30 | 8 | 26 | 4 |
| 40 | X: 3549377-3549389 | A | upstream | NSBP1 | germline | 12 | 12 | 30 | 1 | 32 | 5 | 25 | 2 |
| 41 | X: 80263832-80263843 | A | downstream | CACNA2D3 | germline | 14 | 10 | 50 | 2 | 4 | 6 | 2 | 2 |
| 42 | 1: 171695775-171695786 | AGTG | intergenic | — | germline | 12 | 12 | 62 | 2 | 7 | 5 | 4 | 1 |
| 43 | 10: 20933836-20933848 | AAA | intergenic | — | germline | 13 | 13 | 52 | 0 | 15 | 5 | 15 | 3 |
| | | GAA | | | | | | | | | | | |
| 44 | 11: 3425003-3425019 | AG | intergenic | — | germline | 17 | 17 | 43 | 0 | 6 | 6 | 3 | 4 |
| 45 | 11: 67442371-67442398 | TTTT | intergenic | — | germline | 28 | 32 | 27 | 0 | 5 | 6 | 5 | 4 |
| | | TG | | | | | | | | | | | |
| 46 | 14: 68710868-68710882 | TA | intergenic | — | germline | 15 | 15 | 31 | 0 | 9 | 6 | 6 | 1 |
| 47 | 18: 4024913-4024925 | A | intergenic | — | germline | 13 | 13 | 30 | 0 | 12 | 5 | 11 | 2 |
| 48 | 2: 96487861-96487873 | ACA | intergenic | — | germline | 13 | 13 | 69 | 0 | 6 | 6 | 5 | 4 |
| 49 | 21: 10017859-10017871 | A | intergenic | — | germline | 13 | 14 | 93 | 2 | 46 | 4 | 42 | 1 |
| 50 | 22: 26022851-26022873 | TCAT | intergenic | — | germline | 23 | 23 | 30 | 0 | 8 | 5 | 9 | 2 |
| 51 | 22: 35257862-35257873 | T | intergenic | — | germline | 12 | 12 | 27 | 1 | 7 | 5 | 4 | 3 |
| 52 | 3: 138911384-138911395 | T | intergenic | — | germline | 12 | 12 | 33 | 0 | 5 | 5 | 4 | 4 |
| 53 | 3: 148019720-148019741 | TG | intergenic | — | germline | 22 | 24 | 40 | 0 | 7 | 6 | 4 | 2 |
| 54 | 5: 145429246-145429267 | TGC | intergenic | — | germline | 22 | 25 | 70 | 0 | 12 | 6 | 14 | 4 |
| 55 | 6: 152476403-152476427 | TG | intergenic | — | germline | 25 | 25 | 55 | 1 | 7 | 5 | 4 | 0 |
| 56 | 6: 8145746-68145757 | T | intergenic | — | germline | 12 | 11 | 46 | 1 | 5 | 5 | 3 | 2 |
| 57 | 1: 114028229-114028241 | T | 5utrE | GALNT5 | tumor | 26 | 26 | 98 | 2 | 42 | 2 | 35 | 5 |
| 58 | 12: 12224304-12224316 | A | 5utrI | A2BP1 | tumor | 19 | 19 | 60 | 1 | 4 | 2 | 13 | 6 |
| 59 | 2: 157822745-157822770 | CTG | exon | PIK3AP1 | tumor | 11 | 11 | 121 | 0 | 66 | 0 | 65 | 6 |
| 60 | 16: 6890142-6890160 | TGG | exon | GZF1 | tumor | 30 | 30 | 110 | 0 | 37 | 2 | 35 | 5 |
| 61 | 10: 98401006-98401016 | TCT | exon | KDR | tumor | 12 | 12 | 117 | 0 | 51 | 0 | 45 | 5 |
| 62 | 20: 23293911-23293940 | GGA | intron | ASH1L | tumor | 12 | 12 | 86 | 2 | 55 | 2 | 50 | 8 |
| 63 | 4: 55648576-55648587 | TCC | intron | FASLG | tumor | 13 | 13 | 66 | 0 | 42 | 2 | 40 | 9 |
| 64 | 1: 153652407-153652418 | A | intron | CACNA1E | tumor | 13 | 13 | 61 | 2 | 17 | 4 | 22 | 6 |
| 65 | 1: 170895405-170895417 | T | intron | PTP4A2 | tumor | 14 | 14 | 46 | 0 | 54 | 4 | 50 | 5 |
| 66 | 1: 179957374-179957386 | T | intron | TNNI3K | tumor | 19 | 19 | 61 | 0 | 65 | 1 | 60 | 5 |
| 67 | 1: 32154180-32154193 | A | intron | NCAM1 | tumor | 14 | 14 | 57 | 1 | 47 | 0 | 34 | 6 |
| 68 | 1: 74607395-74607413 | AAAT | intron | CTNND1 | tumor | 15 | 15 | 73 | 1 | 38 | 1 | 31 | 5 |
| 69 | 11: 112618715-112618728 | TCTG | intron | PPP1CC | tumor | 12 | 12 | 106 | 3 | 51 | 0 | 43 | 5 |
| 70 | 11: 57327913-57327927 | A | intron | DYRK4 | tumor | 21 | 21 | 109 | 0 | 37 | 4 | 42 | 6 |
| 71 | 12: 109644897-109644908 | A | intron | NACA | tumor | 12 | 12 | 56 | 1 | 41 | 0 | 39 | 6 |
| 72 | 12: 4584613-4584633 | TTG | intron | KATNAL1 | tumor | 12 | 12 | 66 | 0 | 43 | 3 | 38 | 5 |
| 73 | 12: 55404464-55404475 | TTAA | intron | CROP | tumor | 19 | 19 | 43 | 0 | 6 | 0 | 7 | 6 |
| | | TT | | | | | | | | | | | |
| 74 | 13: 29752364-29752375 | A | intron | ZAK | tumor | 14 | 14 | 36 | 1 | 10 | 0 | 14 | 6 |
| 75 | 17: 46174435-46174453 | TG | intron | NRP2 | tumor | 13 | 13 | 100 | 1 | 15 | 0 | 24 | 11 |
| 76 | 2: 173812284-173812297 | A | intron | ERBB4 | tumor | 12 | 12 | 110 | 0 | 10 | 3 | 17 | 5 |
| 77 | 2: 206340548-206340560 | A | intron | MSH6 | tumor | 34 | 34 | 58 | 1 | 52 | 2 | 48 | 6 |
| 78 | 2: 211997388-211997399 | A | intron | MCM3AP | tumor | 12 | 12 | 92 | 3 | 34 | 3 | 35 | 5 |
| 79 | 2: 47871786-47871819 | TG | intron | KCNH8 | tumor | 13 | 12 | 36 | 0 | 24 | 2 | 22 | 6 |
| 80 | 21: 46527884-46527895 | A | intron | TTC23L | tumor | 22 | 22 | 40 | 0 | 12 | 1 | 18 | 5 |
| 81 | 3: 19531995-19532007 | T | intron | NOTCH4 | tumor | 13 | 13 | 40 | 1 | 19 | 2 | 25 | 6 |
| 82 | 5: 34899233-34899254 | GGT | intron | USP42 | tumor | 13 | 13 | 102 | 2 | 32 | 3 | 29 | 5 |
| 83 | 6: 32274139-32274151 | T | intron | GNAI1 | tumor | 30 | 30 | 55 | 1 | 32 | 0 | 35 | 4 |
| 84 | 7: 6155635-6155647 | A | intron | GPR112 | tumor | 13 | 13 | 59 | 2 | 25 | 2 | 23 | 6 |
| 85 | 7: 79656108-79656137 | GT | intron | MXRA5 | tumor | 13 | 13 | 115 | 0 | 31 | 2 | 26 | 5 |
| 86 | X: 135309623-135309635 | A | 3utrE | MAGI3 | tumor | 13 | 13 | 57 | 2 | 29 | 1 | 26 | 5 |
| 87 | X: 3248015-3248027 | A | 3utrI | BCL2L14 | tumor | 13 | 13 | 84 | 1 | 32 | 3 | 29 | 8 |
| 88 | 1: 108703753-108703767 | AGAT | intergenic | — | tumor | 15 | 15 | 67 | 0 | 2 | 2 | 4 | 5 |
| 89 | 1: 159723647-159723658 | GA | intergenic | — | tumor | 12 | 12 | 64 | 2 | 4 | 1 | 9 | 7 |
| 90 | 1: 166976596-166976618 | TG | intergenic | — | tumor | 23 | 23 | 26 | 0 | 2 | 0 | 5 | 6 |
| 91 | 11: 112271124-112271144 | GAG | intergenic | — | tumor | 21 | 21 | 41 | 0 | 14 | 2 | 17 | 6 |
| 92 | 11: 32965647-32965673 | AC | intergenic | — | tumor | 27 | 25 | 53 | 2 | 8 | 3 | 11 | 5 |
| 93 | 13: 102956299-102956312 | GGT | intergenic | — | tumor | 14 | 9 | 42 | 0 | 5 | 4 | 5 | 5 |
| | | GT | | | | | | | | | | | |
| 94 | 14: 76170785-76170804 | T | intergenic | — | tumor | 20 | 15 | 39 | 0 | 20 | 3 | 21 | 4 |
| 95 | 17: 14787818-14787841 | GT | intergenic | — | tumor | 24 | 24 | 31 | 0 | 6 | 2 | 5 | 6 |
| 96 | 2: 71367561-71367583 | TTA | intergenic | — | tumor | 23 | 20 | 29 | 0 | 4 | 2 | 4 | 5 |
| 97 | 4: 41479010-41479033 | AC | intergenic | — | tumor | 24 | 24 | 28 | 0 | 6 | 4 | 8 | 6 |
| 98 | 6: 170617393-170617405 | CTGA | intergenic | — | tumor | 13 | 13 | 84 | 3 | 12 | 3 | 10 | 5 |
| 99 | 6: 170617424-170617436 | CTGA | intergenic | — | tumor | 13 | 13 | 84 | 3 | 12 | 3 | 10 | 5 |
| 100 | 8: 74356421-74356455 | TTTG | intergenic | — | tumor | 35 | 35 | 25 | 0 | 4 | 4 | 3 | 6 |
| 101 | 12: 6772289-6772304 | ACA | 5utrI | CD4 | both | 16 | 16 | 57 | 0 | 20 | 3 | 27 | 4 |
| | | GAC | | | | | | | | | | | |
| 102 | 6: 16679871-16679882 | A | 5utrI | ATXN1 | both | 12 | 12 | 39 | 0 | 18 | 3 | 23 | 4 |
| 103 | 17: 39412434-39412445 | A | 5utrI | PYY | both | 12 | 12 | 26 | 0 | 6 | 3 | 5 | 4 |
| 104 | X: 53100045-53100074 | GT | 5utrI | GPR173 | both | 30 | 30 | 27 | 0 | 6 | 4 | 4 | 3 |
| 105 | 9: 90214929-90214941 | T | 5utrI | SPIN1 | both | 13 | 13 | 26 | 0 | 4 | 3 | 3 | 3 |
| 106 | 3: 182838323-182838349 | TG | 5utrI | SOX2OT | both | 27 | 27 | 27 | 0 | 4 | 3 | 5 | 3 |
| 107 | 11: 111558775-111558786 | TA | intron | BCO2 | both | 12 | 17 | 104 | 1 | 5 | 4 | 3 | 3 |
| 108 | X: 37400420-37400432 | A | intron | LANCL3 | both | 13 | 13 | 28 | 0 | 4 | 3 | 3 | 3 |
| 109 | 20: 15865317-15865333 | TA | intron | MACROD2 | both | 17 | 19 | 30 | 0 | 4 | 3 | 4 | 3 |
| 110 | 2: 178236415-178236426 | A | intron | PDE11A | both | 12 | 12 | 60 | 3 | 5 | 3 | 7 | 4 |
| 111 | 3: 50187378-50187393 | TGTA | intron | SEMA3F | both | 16 | 16 | 85 | 0 | 5 | 3 | 5 | 3 |
| 112 | 2: 17559661-17559672 | T | intron | RAD15AP2 | both | 12 | 12 | 100 | 3 | 30 | 4 | 27 | 3 |
| 113 | 15: 52107275-52107289 | T | intron | UNC13C | both | 15 | 15 | 27 | 0 | 4 | 3 | 3 | 4 |
| 114 | 11: 16926773-16926802 | AC | intron | PLEKHA7 | both | 30 | 26 | 37 | 0 | 5 | 4 | 4 | 3 |
| 115 | 21: 41509690-41509704 | GT | intron | BACE2 | both | 15 | 15 | 43 | 0 | 5 | 3 | 6 | 4 |
| 116 | 4: 148907969-148907981 | T | intro | ARHGAP10 | both | 13 | 12 | 25 | 0 | 4 | 3 | 3 | 4 |
| 117 | 18: 65998338-65998349 | A | intron | RTTN | both | 12 | 12 | 52 | 0 | 4 | 4 | 6 | 3 |
| 118 | 20: 8354518-8354529 | A | intron | PLCB1 | both | 12 | 12 | 52 | 0 | 4 | 3 | 5 | 3 |
| 119 | 10: 94367466-94367495 | TTTT | intron | KIF11 | both | 30 | 30 | 36 | 0 | 3 | 4 | 4 | 3 |
| | | TG | | | | | | | | | | | |
| 120 | 1: 109177869-109177880 | T | intron | C1orf62 | both | 12 | 12 | 28 | 1 | 4 | 3 | 3 | 4 |
| 121 | 14: 49350131-49350166 | GT | intron | SDCCAG1 | both | 36 | 30 | 31 | 0 | 4 | 3 | 3 | 3 |
| 122 | 17: 55668656-55668676 | AATT | intron | USP32 | both | 21 | 21 | 102 | 2 | 4 | 3 | 3 | 4 |
| 123 | 19: 19850268-19850282 | TG | intron | ZNF253 | both | 15 | 15 | 63 | 1 | 27 | 3 | 21 | 3 |
| 124 | 11: 109960353-109960365 | T | intron | ARHGAP20 | both | 13 | 13 | 41 | 0 | 4 | 3 | 3 | 4 |
| 125 | 2: 119718919-119718938 | TTCA | intron | STEAP3 | both | 20 | 20 | 39 | 0 | 4 | 3 | 4 | 3 |
| 126 | 7: 157690539-157690557 | AAAC | intron | PTPRN2 | both | 19 | 19 | 109 | 0 | 47 | 3 | 41 | 3 |
| 127 | 12: 23813564-23813575 | A | intron | SOX5 | both | 12 | 12 | 49 | 0 | 5 | 4 | 5 | 3 |
| 128 | 11: 73312698-73312721 | AC | intron | PAAF1 | both | 24 | 24 | 26 | 0 | 4 | 3 | 4 | 3 |
| 129 | 22: 45117761-45117775 | T | intron | TRMU | both | 15 | 15 | 52 | 2 | 30 | 4 | 23 | 3 |
| 130 | 4: 103831000-103831022 | AT | intron | MANBA | both | 23 | 23 | 73 | 1 | 17 | 3 | 13 | 4 |
| 131 | 2: 203525503-203525514 | T | intron | ALS2CR8 | both | 12 | 12 | 58 | 2 | 6 | 3 | 7 | 4 |
| 132 | 14: 63775227-63775247 | A | intron | ESR2 | both | 21 | 21 | 28 | 0 | 4 | 4 | 3 | 4 |
| 133 | 2: 60999003-60999015 | T | intron | REL | both | 13 | 13 | 33 | 1 | 30 | 4 | 29 | 4 |
| 134 | X: 110942000-110942011 | T | intron | TRPC5 | both | 12 | 12 | 36 | 0 | 5 | 4 | 4 | 3 |
| 135 | 5: 127622723-127622735 | A | 3utrE | FBN2 | both | 13 | 13 | 51 | 1 | 8 | 4 | 6 | 3 |
| 136 | 8: 146171946-146171961 | CAAA | 3utrE | ZNF252 | both | 16 | 16 | 55 | 0 | 4 | 4 | 3 | 3 |
| 137 | 7: 130349047-130349059 | A | 3utrl | FLI43663 | both | 13 | 14 | 35 | 0 | 4 | 3 | 3 | 4 |
| 138 | 6: 105721437-105721463 | TG | 3utrl | POPDC3 | both | 27 | 25 | 25 | 0 | 4 | 3 | 3 | 4 |
| 139 | 2: 145638487-145638523 | ATA | intergenic | — | both | 37 | 22 | 28 | 0 | 3 | 4 | 3 | 3 |
| 140 | 4: 164792400-164792412 | T | intergenic | — | both | 13 | 13 | 30 | 1 | 3 | 3 | 4 | 4 |
| 141 | 16: 13489606-13489618 | A | intergenic | — | both | 13 | 13 | 47 | 0 | 3 | 3 | 3 | 3 |
| 142 | 7: 97883510-97883521 | ATA | intergenic | — | both | 12 | 15 | 45 | 0 | 10 | 4 | 12 | 4 |
| 143 | 11: 10685136-10685162 | ATT | intergenic | — | both | 27 | 27 | 31 | 1 | 4 | 3 | 3 | 3 |
| 144 | 15: 40741098-40741124 | CTTT | intergenic | — | both | 27 | 27 | 30 | 0 | 17 | 3 | 17 | 4 |
| 145 | 11: 4596364-4596375 | A | intergenic | — | both | 12 | 11 | 54 | 0 | 4 | 3 | 4 | 3 |
| 146 | 6: 170617335-170617347 | CTGA | intergenic | — | both | 13 | 13 | 87 | 3 | 12 | 3 | 9 | 3 |
| 147 | 5: 4634091-4634111 | CA | intergenic | — | both | 21 | 21 | 51 | 2 | 10 | 4 | 7 | 4 |
| 148 | 9: 98862259-98862282 | TAA | intergenic | — | both | 24 | 24 | 30 | 0 | 4 | 3 | 3 | 4 |
| 149 | X: 25977786-25977810 | AC | intergenic | — | both | 25 | 25 | 31 | 0 | 4 | 3 | 6 | 4 |
| 150 | 8: 130505282-130505298 | AG | intergenic | — | both | 17 | 17 | 38 | 1 | 5 | 3 | 5 | 4 |
| 151 | 1: 176219284-176219296 | A | intergenic | — | both | 13 | 13 | 38 | 0 | 5 | 3 | 5 | 4 |
| 152 | 7: 113737802-113737815 | T | intergenic | — | both | 14 | 14 | 32 | 0 | 4 | 4 | 3 | 4 |
| 153 | 2: 33870773-33870795 | AAAC | intergenic | — | both | 23 | 23 | 36 | 0 | 8 | 4 | 7 | 3 |
| 154 | 13: 54794891-54794907 | AT | intergenic | — | both | 17 | 17 | 48 | 0 | 3 | 4 | 3 | 4 |
| 155 | 2: 192007897-192007912 | AC | intergenic | — | both | 16 | 16 | 61 | 1 | 4 | 4 | 4 | 4 |
| 156 | 8: 107323652-107323663 | A | intergenic | — | both | 12 | 12 | 38 | 0 | 7 | 3 | 12 | 3 |
| 157 | 12: 22938635-22938661 | GT | intergenic | — | both | 27 | 25 | 33 | 0 | 6 | 3 | 4 | 4 |
| 158 | X: 134739190-134739207 | TG | intergenic | — | both | 18 | 18 | 63 | 0 | 4 | 3 | 2 | 3 |
| 159 | 9: 16305659-16305683 | GT | intergenic | — | both | 25 | 25 | 26 | 0 | 5 | 3 | 4 | 4 |
| 160 | 18: 24650950-24650961 | CA | intergenic | — | both | 12 | 12 | 61 | 0 | 4 | 3 | 3 | 3 |
| 161 | 2: 54396727-54396739 | T | intergenic | — | both | 13 | 13 | 54 | 2 | 3 | 3 | 3 | 3 |
| 162 | 1: 237497587-237497605 | TG | intergenic | — | both | 19 | 19 | 35 | 0 | 4 | 4 | 3 | 3 |
| 163 | X: 94491634-94491647 | A | intergenic | — | both | 14 | 14 | 27 | 0 | 3 | 3 | 3 | 4 |
| 164 | 1: 86450570-86450582 | TTA | intergenic | — | both | 13 | 13 | 47 | 0 | 6 | 3 | 6 | 3 |
| 165 | 9: 77020098-77020110 | T | intergenic | — | both | 13 | 12 | 38 | 0 | 4 | 3 | 2 | 3 |
| 166 | 4: 121689390-121689407 | TC | intergenic | — | both | 18 | 18 | 47 | 0 | 4 | 3 | 3 | 4 |
| 167 | 11: 122744892-122744904 | AAGA | intergenic | — | both | 13 | 13 | 61 | 2 | 7 | 3 | 6 | 3 |
| 168 | 5: 87659623-87659644 | CA | intergenic | — | both | 22 | 22 | 33 | 0 | 4 | 4 | 4 | 3 |
| 169 | 2: 21040040-21040054 | A | intergenic | — | both | 15 | 15 | 26 | 1 | 4 | 4 | 3 | 4 |
| 170 | 12: 29817621-29817641 | AAA | 5utrl | TMTC1 | germline | 21 | 16 | 44 | 0 | 5 | 3 | 3 | 1 |
| | | AC | | | | | | | | | | | |
| 171 | 1: 89218696-89218709 | T | 5utrl | CCBL2 | germline | 14 | 14 | 37 | 0 | 4 | 4 | 3 | 2 |
| 172 | 12: 29818226-29818244 | GT | 5utrl | TMTC1 | germline | 19 | 17 | 58 | 1 | 5 | 3 | 6 | 2 |
| 173 | 1: 181873669-181873681 | TTTC | 5utrl | RGL1 | germline | 13 | 13 | 60 | 2 | 3 | 3 | 2 | 2 |
| | | AG | | | | | | | | | | | |
| 174 | 21: 33102478-33102490 | A | 5utrl | C21orf62 | germline | 13 | 13 | 36 | 0 | 3 | 3 | 4 | 0 |
| 175 | 19: 44142772-44142783 | T | 5utrl | FBXO17 | germline | 12 | 11 | 29 | 1 | 4 | 4 | 2 | 2 |
| 176 | 5: 115888200-115888211 | A | 5utrl | SEMA6A | germline | 12 | 12 | 59 | 1 | 6 | 3 | 3 | 2 |
| 177 | 15: 67335101-67335113 | A | 5utrl | GLCE | germline | 13 | 13 | 70 | 2 | 36 | 3 | 32 | 1 |
| 178 | 11: 71453528-71453545 | AAAC | 5utrl | NUMA1 | germline | 18 | 19 | 47 | 1 | 7 | 3 | 7 | 1 |
| 179 | 1: 2108814974-210814986 | CA | 5utrl | ATF3 | germline | 13 | 13 | 70 | 0 | 4 | 3 | 4 | 2 |
| 180 | 15: 28193381-28193394 | T | 5utrl | FAM7A3 | germline | 14 | 14 | 94 | 3 | 22 | 4 | 18 | 0 |
| 181 | 7: 5767427-5767440 | A | 5utrl | RNF216 | germline | 14 | 14 | 88 | 1 | 14 | 4 | 12 | 1 |
| 182 | 11: 98797078-98797091 | T | 5utrl | CNTN5 | germline | 14 | 14 | 29 | 0 | 3 | 3 | 2 | 2 |
| 183 | 18: 17364496-17364508 | A | intron | ESCO1 | germline | 13 | 13 | 107 | 2 | 32 | 3 | 30 | 1 |
| 184 | 12: 48281672-48281683 | T | intron | FAM186B | germline | 12 | 12 | 44 | 1 | 4 | 3 | 3 | 0 |
| 185 | 4: 47039920-47039932 | A | intron | GABRB1 | germline | 13 | 13 | 37 | 0 | 4 | 3 | 3 | 1 |
| 186 | 15: 32942592-32942603 | T | intron | AQR | germline | 12 | 12 | 66 | 2 | 35 | 3 | 27 | 0 |
| 187 | 7: 71483586-71483613 | TGGA | intron | CALN1 | germline | 28 | 28 | 39 | 0 | 5 | 3 | 3 | 1 |
| 188 | 1: 76833956-76833979 | AC | intron | ST6GALNAC3 | germline | 24 | 22 | 48 | 1 | 5 | 4 | 4 | 1 |
| 189 | X: 53646375-53646391 | AT | intron | HUWE1 | germline | 17 | 17 | 42 | 1 | 58 | 4 | 49 | 2 |
| 190 | 9: 113455017-113455043 | AAAT | intron | DNAJC25- | germline | 27 | 27 | 41 | 1 | 5 | 4 | 5 | 2 |
| | | | | GNG10 | | | | | | | | | |
| 191 | 1: 172144927-172144947 | TAA | intron | SERPINC1 | germline | 21 | 21 | 27 | 0 | 4 | 3 | 5 | 2 |
| 192 | 5: 169425047-169425060 | AAC | intron | DOCK2 | germline | 14 | 14 | 84 | 2 | 6 | 4 | 3 | 1 |
| 193 | 11: 133515991-133516003 | T | intron | JAM3 | germline | 13 | 13 | 64 | 1 | 8 | 3 | 2 | 0 |
| 194 | 19: 13184113-13184125 | GT | intron | CACNA1A | germline | 13 | 13 | 34 | 1 | 34 | 3 | 32 | 1 |
| 195 | 5: 114537119-114537140 | TG | intron | TRIM36 | germline | 22 | 22 | 25 | 0 | 4 | 3 | 4 | 2 |
| 196 | 7: 31845557-31845573 | AC | intron | PDE1C | germline | 17 | 17 | 59 | 0 | 4 | 3 | 3 | 2 |
| 197 | X: 100419148-100419160 | T | intron | TAF7L | germline | 13 | 13 | 47 | 0 | 30 | 3 | 27 | 1 |
| 198 | 4: 148967780-148967793 | T | intron | ARHGAP10 | germline | 14 | 14 | 25 | 0 | 5 | 3 | 3 | 2 |
| 199 | 1: 100382712-100382723 | A | intron | CCDC76 | germline | 12 | 12 | 57 | 2 | 4 | 4 | 3 | 1 |
| 200 | 10: 53354719-53354730 | T | intron | PRKG1 | germline | 12 | 12 | 49 | 0 | 4 | 3 | 3 | 1 |
| 201 | 9: 78682236-78682256 | AC | intron | PRUNE2 | germline | 21 | 21 | 29 | 0 | 6 | 4 | 5 | 0 |
| 202 | 12: 108208949-108208985 | GGG | intron | FOXN4 | germline | 37 | 37 | 95 | 0 | 7 | 3 | 6 | 2 |
| | | CA | | | | | | | | | | | |
| 203 | 12: 118730713-118730724 | A | intron | CIT | germline | 12 | 12 | 40 | 0 | 4 | 3 | 3 | 0 |
| 204 | 1: 117834341-117834356 | GT | intron | MAN1A2 | germline | 16 | 16 | 77 | 0 | 6 | 3 | 5 | 0 |
| 205 | 6: 83667703-83667714 | A | intron | UBE2CBP | germline | 12 | 12 | 41 | 1 | 4 | 3 | 2 | 0 |
| 206 | 20: 39258842-39258855 | A | intron | ZHX3 | germline | 14 | 14 | 28 | 0 | 23 | 3 | 15 | 2 |
| 207 | 11: 85876178-85876189 | T | intron | ME3 | germline | 12 | 12 | 55 | 1 | 22 | 4 | 11 | 2 |
| 208 | 13: 18906723-18906749 | TTTGT | intron | TPTE2 | germline | 27 | 27 | 32 | 0 | 5 | 3 | 5 | 2 |
| 209 | 5: 168306722-168306734 | AC | intron | SLIT3 | germline | 13 | 13 | 58 | 2 | 7 | 3 | 4 | 1 |
| 210 | 17: 19630095-19630106 | T | intron | ULK2 | germline | 12 | 12 | 102 | 0 | 29 | 4 | 21 | 1 |
| 211 | 13: 35367425-35367439 | A | intron | DCLK1 | germline | 15 | 15 | 30 | 0 | 4 | 3 | 3 | 2 |
| 212 | 7: 140355706-140355718 | T | intron | MRPS33 | germline | 13 | 11 | 29 | 0 | 5 | 3 | 3 | 2 |
| 213 | 17: 38010632-38010643 | A | intron | FAM134C | germline | 12 | 12 | 43 | 1 | 5 | 3 | 3 | 1 |
| 214 | 5: 74768101-74768117 | CTTT | intron | COL4A3BP | germline | 17 | 17 | 42 | 0 | 5 | 3 | 5 | 0 |
| 215 | 14: 68931216-68931227 | A | intron | ERH | germline | 12 | 12 | 47 | 0 | 40 | 4 | 35 | 0 |
| 216 | 6: 39013646-39013657 | T | intron | DNAH8 | germline | 12 | 12 | 103 | 3 | 32 | 4 | 28 | 1 |
| 217 | 15: 71205795-71205808 | T | intron | NEO1 | germline | 14 | 14 | 27 | 0 | 22 | 3 | 16 | 0 |
| 218 | 7: 129464528-129464555 | AAAC | intron | ZC3HC1 | germline | 28 | 28 | 29 | 0 | 5 | 3 | 5 | 2 |
| 219 | 18: 32789732-32789743 | T | intron | KIAA1328 | germline | 12 | 11 | 56 | 2 | 4 | 3 | 4 | 2 |
| 220 | 6: 136974297-136974308 | A | intron | MAP3K5 | germline | 12 | 12 | 69 | 0 | 49 | 3 | 47 | 0 |
| 221 | 11: 18698487-18698498 | T | intron | IGSF22 | germline | 12 | 12 | 32 | 0 | 41 | 3 | 35 | 0 |
| 222 | 5: 167681860-167681873 | A | intron | WWC1 | germline | 14 | 14 | 35 | 1 | 4 | 3 | 3 | 1 |
| 223 | X: 54074743-54074756 | A | intron | PHF8 | germline | 14 | 14 | 35 | 1 | 5 | 3 | 3 | 0 |
| 224 | 3: 103058568-103058584 | T | intron | NFKBIZ | germline | 17 | 17 | 55 | 0 | 7 | 3 | 4 | 0 |
| 225 | 7: 4875289-4875305 | ACAA | intron | RADIL | germline | 17 | 17 | 43 | 0 | 8 | 3 | 7 | 1 |
| 226 | 15: 65743895-65743907 | A | intron | MAP2K5 | germline | 13 | 13 | 45 | 0 | 40 | 3 | 35 | 2 |
| 227 | 11: 67525739-67525750 | A | intron | UNC93B1 | germline | 12 | 12 | 37 | 0 | 4 | 4 | 3 | 2 |
| 228 | 5: 80587397-80587410 | A | intron | CKMT2 | germline | 14 | 14 | 35 | 0 | 4 | 4 | 3 | 2 |
| 229 | X: 113991240-113991253 | AATT | intron | HTR2C | germline | 14 | 12 | 65 | 2 | 11 | 4 | 4 | 2 |
| 230 | 14: 90709365-90709379 | T | intron | C14orf159 | germline | 15 | 15 | 62 | 2 | 7 | 4 | 3 | 2 |
| 231 | 20: 32689455-32689468 | A | intron | PIGU | germline | 14 | 14 | 33 | 0 | 21 | 3 | 23 | 1 |
| 232 | 1: 112854845-112854856 | T | intron | WNT2B | germline | 12 | 12 | 29 | 0 | 5 | 3 | 4 | 2 |
| 233 | 5: 72221348-72221362 | T | intron | TNPO1 | germline | 15 | 15 | 31 | 0 | 35 | 3 | 27 | 1 |
| 234 | 16: 60602439-60602456 | AAAT | intron | CDH8 | germline | 18 | 18 | 32 | 1 | 4 | 3 | 3 | 0 |
| 235 | 20: 15407185-15407219 | TG | intron | MACROD2 | germline | 35 | 33 | 27 | 1 | 6 | 3 | 4 | 2 |
| 236 | 18: 27898297-27898309 | CA | intron | RNF125 | germline | 13 | 13 | 58 | 0 | 6 | 4 | 7 | 2 |
| 237 | 1: 108168496-108168514 | TTTG | intron | VAV3 | germline | 19 | 19 | 50 | 0 | 7 | 3 | 8 | 0 |
| 238 | 3: 11663031-11663043 | A | intron | VGLL4 | germline | 13 | 13 | 33 | 0 | 7 | 3 | 3 | 0 |
| 239 | 1: 181867032-181867043 | A | intron | ARPC5 | germline | 12 | 12 | 29 | 0 | 4 | 3 | 4 | 2 |
| 240 | 3: 161037594-161037605 | T | intron | SCHIP1 | germline | 12 | 12 | 40 | 0 | 4 | 3 | 3 | 1 |
| 241 | 5: 32093668-32093679 | T | intron | PDZD2 | germline | 12 | 12 | 38 | 0 | 37 | 3 | 34 | 0 |
| 242 | 8: 52529022-52529034 | AT | intron | PXDNL | germline | 13 | 13 | 57 | 2 | 5 | 3 | 3 | 0 |
| 243 | 12: 93551013-93551045 | AAA | intron | TMCC3 | germline | 33 | 33 | 28 | 0 | 4 | 3 | 4 | 2 |
| | | AG | | | | | | | | | | | |
| 244 | 3: 65403701-65403712 | A | intron | MAGI1 | germline | 12 | 12 | 102 | 2 | 21 | 3 | 21 | 1 |
| 245 | 1: 86245321-86245339 | AAT | intron | COL24A1 | germline | 19 | 19 | 33 | 0 | 8 | 3 | 7 | 0 |
| 246 | 8: 31053359-31053370 | T | intron | WRN | germline | 12 | 12 | 86 | 3 | 46 | 3 | 46 | 1 |
| 247 | 21: 37754281-37754292 | T | intron | DYRK1A | germline | 12 | 12 | 45 | 0 | 5 | 3 | 3 | 1 |
| 248 | 2: 33096724-33096735 | T | intron | LTBP1 | germline | 12 | 12 | 31 | 0 | 4 | 3 | 4 | 1 |
| 249 | 12: 63400333-63400346 | A | intron | GNS | germline | 14 | 14 | 65 | 0 | 24 | 3 | 23 | 2 |
| 250 | 1: 183116859-183116884 | CA | intron | FAM129A | germline | 26 | 26 | 39 | 1 | 4 | 3 | 5 | 2 |
| 251 | 12: 28382459-28382470 | T | intron | CCDC91 | germline | 12 | 13 | 25 | 0 | 2 | 3 | 3 | 2 |
| 252 | 6: 130060281-130060295 | T | intron | ARHGAP18 | germline | 15 | 14 | 25 | 1 | 4 | 3 | 3 | 0 |
| 253 | 6: 162495547-162495560 | A | intron | PARK2 | germline | 14 | 13 | 25 | 0 | 3 | 3 | 3 | 2 |
| 254 | 7: 110292470-110292484 | CA | intron | IMMP2L | germline | 15 | 15 | 57 | 2 | 5 | 3 | 3 | 2 |
| 255 | 1: 100722772-100722783 | A | intron | CDC14A | germline | 12 | 12 | 106 | 3 | 30 | 4 | 27 | 2 |
| 256 | 3: 159596876-159596889 | T | intron | RSRC1 | germline | 14 | 14 | 36 | 1 | 4 | 4 | 3 | 2 |
| 257 | 3: 37057037-37057065 | TTTG | intron | MLH1 | germline | 29 | 29 | 100 | 0 | 11 | 3 | 14 | 1 |
| 258 | 15: 71207635-71207649 | T | intron | NEO1 | germline | 15 | 15 | 26 | 0 | 4 | 4 | 2 | 0 |
| 259 | 14: 32110172-32110184 | T | intron | AKAP6 | germline | 13 | 13 | 31 | 0 | 4 | 4 | 3 | 2 |
| 260 | 8: 51606442-51606454 | T | intron | SNTG1 | germline | 13 | 13 | 36 | 1 | 5 | 3 | 2 | 0 |
| 261 | 6: 138599830-138599841 | T | intron | KIAA1244 | germline | 12 | 13 | 28 | 0 | 4 | 3 | 3 | 0 |
| 262 | 5: 108295563-108295583 | TG | intron | FER | germline | 21 | 21 | 35 | 0 | 4 | 4 | 4 | 1 |
| 263 | 20: 55350656-55350673 | GT | intron | SPO11 | germline | 18 | 18 | 65 | 0 | 4 | 4 | 4 | 2 |
| 264 | 12: 42968207-42968218 | CAATA | intron | TMEM117 | germline | 12 | 12 | 54 | 0 | 5 | 3 | 4 | 0 |
| 265 | 11: 113207635-113207646 | A | intron | USP28 | germline | 12 | 12 | 78 | 1 | 10 | 3 | 7 | 1 |
| 266 | 10: 106049118-106049133 | TCTTT | 3utrE | GSTO2 | germline | 16 | 16 | 111 | 0 | 45 | 3 | 39 | 0 |
| 267 | 6: 1557419-1557430 | A | 3utrE | FOXC1 | germline | 12 | 12 | 104 | 1 | 15 | 3 | 18 | 2 |
| 268 | 20: 54006163-54006174 | A | 3utrE | CBLN4 | germline | 12 | 12 | 39 | 0 | 3 | 4 | 2 | 1 |
| 269 | 8: 94018704-94018718 | AC | 3utrI | C8orf83 | germline | 15 | 15 | 53 | 0 | 3 | 3 | 2 | 1 |
| 270 | 8: 144168555-144168567 | GAG | 3utrI | LOC100133669 | germline | 13 | 13 | 67 | 2 | 3 | 4 | 6 | 0 |
| 271 | 21: 29718195-29718206 | A | 3utrI | C21orf41 | germline | 12 | 12 | 61 | 0 | 5 | 3 | 4 | 2 |
| 272 | 17: 69282667-69282680 | A | 3utrI | C17orf54 | germline | 14 | 14 | 25 | 0 | 5 | 3 | 4 | 0 |
| 273 | 3: 195572200-195572233 | TTCT | upstream | LRRC15 | germline | 34 | 34 | 29 | 0 | 4 | 3 | 4 | 2 |
| 274 | 12: 55277010-55277022 | CACCCC | downstream | RBMS2 | germline | 13 | 13 | 29 | 0 | 8 | 4 | 4 | 0 |
| 275 | X: 4433257-4433269 | T | intergenic | — | germline | 13 | 13 | 38 | 1 | 4 | 3 | 2 | 2 |
| 276 | 3: 112546677-112546696 | TAA | intergenic | — | germline | 20 | 20 | 51 | 2 | 4 | 3 | 3 | 1 |
| 277 | 11: 73962997-73963022 | AAAC | intergenic | — | germline | 26 | 26 | 28 | 0 | 4 | 4 | 7 | 2 |
| 278 | 20: 19043500-19043511 | T | intergenic | — | germline | 12 | 12 | 55 | 0 | 7 | 3 | 5 | 1 |
| 279 | X: 1131256-1131279 | GT | intergenic | — | germline | 24 | 24 | 51 | 0 | 8 | 3 | 9 | 1 |
| 280 | 4: 56247225-56247236 | A | intergenic | — | germline | 12 | 12 | 42 | 0 | 5 | 4 | 4 | 2 |
| 281 | 1: 158957356-158957370 | TTTTC | intergenic | — | germline | 15 | 16 | 29 | 1 | 6 | 4 | 4 | 2 |
| 282 | 10: 33983123-33983134 | A | intergenic | — | germline | 12 | 12 | 28 | 0 | 6 | 3 | 4 | 2 |
| 283 | 13: 61543485-61543498 | GAA | intergenic | — | germline | 14 | 14 | 38 | 0 | 4 | 3 | 4 | 2 |
| 284 | 1: 64604642-64604661 | TTGC | intergenic | — | germline | 20 | 20 | 57 | 0 | 8 | 4 | 12 | 0 |
| 285 | 1: 76906723-76906739 | AAC | intergenic | — | germline | 17 | 17 | 42 | 1 | 4 | 3 | 6 | 0 |
| 286 | 7: 19010973-19010987 | A | intergenic | — | germline | 15 | 15 | 25 | 0 | 2 | 3 | 2 | 0 |
| 287 | 1: 175589959-175589972 | AAAT | intergenic | — | germline | 14 | 14 | 25 | 0 | 9 | 4 | 12 | 2 |
| | | AA | | | | | | | | | | | |
| 288 | 12: 79175219-79175231 | T | intergenic | — | germline | 13 | 14 | 32 | 0 | 3 | 3 | 2 | 2 |
| 289 | 9: 83875067-83875081 | AC | intergenic | — | germline | 15 | 15 | 69 | 0 | 5 | 3 | 4 | 0 |
| 290 | 5: 9687506-9687520 | TTG | intergenic | — | germline | 15 | 15 | 53 | 0 | 5 | 3 | 4 | 2 |
| 291 | 3: 178605185-178605198 | A | intergenic | — | germline | 14 | 14 | 34 | 0 | 3 | 3 | 2 | 0 |
| 292 | 1: 90764331-90764342 | TTAA | intergenic | — | germline | 12 | 12 | 99 | 0 | 8 | 3 | 7 | 1 |
| | | AA | | | | | | | | | | | |
| 293 | 1: 115920401-115920417 | TG | intergenic | — | germline | 17 | 17 | 47 | 0 | 5 | 3 | 4 | 2 |
| 294 | 11: 108660886-108660917 | TG | intergenic | — | germline | 32 | 32 | 31 | 0 | 6 | 4 | 3 | 2 |
| 295 | 12: 79147904-79147916 | T | intergenic | — | germline | 13 | 13 | 28 | 0 | 4 | 3 | 3 | 0 |
| 296 | 15: 53179869-53179881 | A | intergenic | — | germline | 13 | 13 | 26 | 1 | 3 | 3 | 3 | 0 |
| 297 | 9: 22204973-22205007 | TCTG | intergenic | — | germline | 35 | 35 | 32 | 1 | 4 | 3 | 3 | 2 |
| 298 | 6: 135230419-135230443 | GTTG | intergenic | — | germline | 25 | 25 | 31 | 1 | 8 | 3 | 3 | 0 |
| 299 | 1: 14635437-14635461 | GTG | intergenic | — | germline | 25 | 25 | 31 | 0 | 9 | 4 | 9 | 2 |
| 300 | X: 6345267-6345280 | A | intergenic | — | germline | 14 | 14 | 38 | 0 | 4 | 3 | 2 | 2 |
| 301 | 4: 178099404-178099431 | GT | intergenic | — | germline | 28 | 24 | 29 | 0 | 4 | 4 | 7 | 2 |
| 302 | 1: 191090600-191090611 | A | intergenic | — | germline | 12 | 12 | 34 | 1 | 3 | 3 | 3 | 1 |
| 303 | 18: 7294429-7294442 | T | intergenic | — | germline | 14 | 14 | 28 | 0 | 4 | 3 | 3 | 0 |
| 304 | 13: 27283247-27283268 | TAAA | intergenic | — | germline | 22 | 22 | 32 | 1 | 4 | 4 | 3 | 2 |
| 305 | 4: 98061304-98061326 | TTG | intergenic | — | germline | 23 | 23 | 41 | 1 | 6 | 3 | 5 | 2 |
| 306 | 1: 52140552-52140573 | AC | intergenic | — | germline | 22 | 22 | 43 | 1 | 5 | 3 | 3 | 1 |
| 307 | 19: 6813439-6813460 | AAT | intergenic | — | germline | 22 | 22 | 30 | 0 | 4 | 3 | 4 | 2 |
| 308 | 18: 23736189-23736200 | T | intergenic | — | germline | 12 | 12 | 47 | 0 | 5 | 4 | 3 | 1 |
| 309 | 1: 173514596-173514609 | A | intergenic | — | germline | 14 | 13 | 27 | 1 | 4 | 3 | 3 | 1 |
| 310 | 19: 21350659-21350670 | A | intergenic | — | germline | 12 | 12 | 45 | 0 | 39 | 3 | 34 | 2 |
| 311 | 15: 66104876-66104892 | AC | intergenic | — | germline | 17 | 17 | 45 | 0 | 8 | 4 | 14 | 2 |
| 312 | 4: 43557024-43557052 | TTG | intergenic | — | germline | 29 | 29 | 31 | 0 | 21 | 4 | 19 | 0 |
| 313 | 10: 126036487-126036498 | T | intergenic | — | germline | 12 | 12 | 30 | 0 | 4 | 3 | 4 | 2 |
| 314 | 21: 17185005-17185016 | T | intergenic | — | germline | 12 | 12 | 33 | 0 | 5 | 3 | 3 | 0 |
| 315 | 2: 123169476-123169497 | GT | intergenic | — | germline | 22 | 18 | 29 | 1 | 4 | 3 | 3 | 2 |
| 316 | 18: 63174603-63174614 | T | intergenic | — | germline | 12 | 12 | 51 | 1 | 4 | 4 | 2 | 0 |
| 317 | 11: 122835988-122835999 | GT | intergenic | — | germline | 12 | 12 | 54 | 0 | 4 | 3 | 5 | 2 |
| 318 | 1: 234737966-234737988 | TTTT | intergenic | — | germline | 23 | 23 | 30 | 0 | 5 | 4 | 7 | 1 |
| | | TA | | | | | | | | | | | |
| 319 | 14: 96510228-96510244 | TC | intergenic | — | germline | 17 | 17 | 42 | 0 | 4 | 3 | 5 | 2 |
| 320 | 2: 103155613-103155624 | AT | intergenic | — | germline | 12 | 12 | 69 | 2 | 6 | 4 | 4 | 0 |
| 321 | 5: 148340399-148340436 | TTG | intergenic | — | germline | 38 | 38 | 27 | 1 | 5 | 3 | 4 | 2 |
| 322 | 4: 25355734-25355755 | TTTG | intergenic | — | germline | 22 | 22 | 28 | 0 | 6 | 3 | 5 | 2 |
| 323 | 9: 96058580-96058591 | T | intergenic | — | germline | 12 | 11 | 45 | 1 | 4 | 4 | 3 | 2 |
| 324 | 13: 39329635-39329662 | GCCA | intergenic | — | germline | 28 | 34 | 58 | 2 | 6 | 4 | 3 | 2 |
| | | GA | | | | | | | | | | | |
| 325 | 1: 166762596-166762610 | TA | intergenic | — | germline | 15 | 13 | 48 | 0 | 9 | 3 | 6 | 2 |
| 326 | 1: 237823405-237823416 | A | intergenic | — | germline | 12 | 13 | 58 | 2 | 5 | 3 | 3 | 0 |
| 327 | 18: 64889208-64889221 | A | intergenic | — | germline | 14 | 14 | 27 | 1 | 2 | 3 | 3 | 2 |
| 328 | 1: 43463310-43463348 | TTTG | intergenic | — | germline | 39 | 27 | 32 | 0 | 7 | 4 | 4 | 2 |
| 329 | 5: 124966313-124966342 | CA | intergenic | — | germline | 30 | 30 | 32 | 1 | 6 | 3 | 3 | 2 |
| 330 | 10: 62205866-62205878 | T | intergenic | — | germline | 13 | 12 | 30 | 0 | 4 | 3 | 3 | 2 |
| 331 | X: 65769176-65769189 | A | intergenic | — | germline | 14 | 14 | 25 | 1 | 7 | 3 | 5 | 1 |
| 332 | 5: 156268512-156268527 | AAAC | intergenic | — | germline | 16 | 16 | 62 | 2 | 12 | 3 | 14 | 2 |
| 333 | 8: 2730094-2730122 | AAAC | intergenic | — | germline | 29 | 25 | 25 | 0 | 5 | 3 | 5 | 2 |
| 334 | 3: 129716442-129716470 | GAT | intergenic | — | germline | 29 | 29 | 28 | 0 | 6 | 4 | 4 | 0 |
| 335 | 8: 79218026-79218043 | CA | intergenic | — | germline | 18 | 18 | 49 | 0 | 6 | 3 | 3 | 2 |
| 336 | 18: 59205041-59205054 | A | intergenic | — | germline | 14 | 14 | 34 | 1 | 4 | 3 | 4 | 1 |
| 337 | 10: 119532591-119532602 | T | intergenic | — | germline | 12 | 12 | 34 | 0 | 4 | 3 | 3 | 2 |
| 338 | 6: 170617571-170617583 | CTGA | intergenic | — | germline | 13 | 13 | 99 | 2 | 11 | 3 | 5 | 2 |
| 339 | 5: 66696861-66696889 | TG | intergenic | — | germline | 29 | 29 | 26 | 1 | 5 | 4 | 3 | 2 |
| 340 | 7: 15773271-15773296 | CA | intergenic | — | germline | 26 | 26 | 33 | 0 | 5 | 3 | 4 | 2 |
| 341 | 12: 73691708-73691719 | T | intergenic | — | germline | 12 | 11 | 49 | 1 | 4 | 3 | 3 | 1 |
| 342 | 6: 170617830-170617842 | CTGA | intergenic | — | germline | 13 | 13 | 96 | 3 | 7 | 4 | 3 | 2 |
| 343 | 14: 81157126-81157140 | AT | intergenic | — | germline | 15 | 15 | 51 | 0 | 7 | 4 | 3 | 1 |
| 344 | 1: 220200862-220200891 | GTTTT | intergenic | — | germline | 30 | 30 | 26 | 0 | 4 | 3 | 3 | 0 |
| 345 | 1: 44629081-44629093 | A | intergenic | — | germline | 13 | 13 | 41 | 0 | 6 | 3 | 6 | 2 |
| 346 | 14: 25679349-25679380 | CAAA | intergenic | — | germline | 32 | 32 | 32 | 0 | 8 | 3 | 10 | 1 |
| 347 | 9: 20625837-20625848 | T | intergenic | — | germline | 12 | 12 | 56 | 0 | 4 | 3 | 4 | 2 |
| 348 | 7: 117915227-117915243 | AAC | intergenic | — | germline | 17 | 20 | 54 | 1 | 5 | 3 | 6 | 0 |
| 349 | 5: 159082372-159082384 | A | intergenic | — | germline | 13 | 13 | 26 | 0 | 5 | 3 | 1 | 1 |
| 350 | 4: 93161548-93161561 | A | intergenic | — | germline | 14 | 14 | 25 | 1 | 4 | 4 | 3 | 2 |
| 351 | 14: 29042495-29042511 | AC | intergenic | — | germline | 17 | 17 | 54 | 2 | 4 | 4 | 4 | 2 |
| 352 | 4: 13267730-13267741 | T | intergenic | — | germline | 12 | 12 | 27 | 0 | 3 | 4 | 2 | 0 |
| 353 | 3: 38004298-38004317 | AC | intergenic | — | germline | 20 | 20 | 29 | 0 | 4 | 3 | 4 | 2 |
| 354 | 17: 14695510-14695532 | GTTT | intergenic | — | germline | 23 | 23 | 50 | 2 | 5 | 3 | 3 | 2 |
| 355 | X: 40030532-40030551 | AG | intergenic | — | germline | 20 | 20 | 37 | 0 | 5 | 4 | 3 | 0 |
| 356 | 16: 64398164-64398180 | A | intergenic | — | germline | 17 | 15 | 40 | 0 | 5 | 3 | 2 | 0 |
| 357 | 10: 111031041-111031059 | AAT | intergenic | — | germline | 19 | 19 | 44 | 0 | 5 | 3 | 6 | 2 |
| 358 | 8: 1055957-1055977 | GCT | intergenic | — | germline | 21 | 21 | 30 | 0 | 8 | 4 | 3 | 0 |
| 359 | 13: 96952809-96952820 | A | intergenic | — | germline | 12 | 12 | 37 | 0 | 5 | 4 | 4 | 2 |
| 360 | 11: 43532770-43532781 | A | intergenic | — | germline | 12 | 12 | 46 | 1 | 5 | 4 | 4 | 2 |
| 361 | 18: 41965925-41965938 | CAAA | intergenic | — | germline | 14 | 14 | 76 | 2 | 11 | 3 | 6 | 1 |
| 362 | 5: 81224460-81224473 | AAAT | intergenic | — | germline | 14 | 14 | 118 | 3 | 29 | 3 | 26 | 0 |
| 363 | 19: 53716193-53716216 | AC | intergenic | — | germline | 24 | 24 | 27 | 1 | 5 | 4 | 4 | 2 |
| 364 | 3: 145541904-145541915 | A | intergenic | — | germline | 12 | 12 | 59 | 2 | 6 | 4 | 3 | 0 |
| 365 | 1: 211881796-211881818 | AAAT | intergenic | — | germline | 23 | 23 | 34 | 0 | 4 | 3 | 4 | 0 |
| 366 | 12: 23163250-23163262 | T | intergenic | — | germline | 13 | 11 | 50 | 2 | 4 | 3 | 2 | 2 |
| 367 | 7: 5793036-5793048 | A | intergenic | — | germline | 13 | 13 | 46 | 0 | 4 | 3 | 3 | 1 |
| 368 | 1: 217360639-217360651 | TTTAT | intergenic | — | germline | 13 | 13 | 53 | 0 | 5 | 4 | 3 | 1 |
| 369 | 6: 14952635-14952650 | T | intergenic | — | germline | 16 | 16 | 39 | 1 | 3 | 3 | 6 | 2 |
| 370 | 2: 213201807-213201821 | AT | intergenic | — | germline | 15 | 15 | 46 | 0 | 5 | 4 | 3 | 1 |
| 371 | 5: 25875862-25875875 | AAC | intergenic | — | germline | 14 | 14 | 58 | 0 | 8 | 4 | 3 | 0 |
| 372 | 6: 9041458-9041470 | A | intergenic | — | germline | 13 | 13 | 31 | 0 | 5 | 3 | 4 | 2 |
| 373 | 16: 78151820-78151831 | A | intergenic | — | germline | 12 | 12 | 34 | 1 | 4 | 4 | 4 | 2 |
| 374 | X: 114105513-114105535 | CA | intergenic | — | germline | 23 | 21 | 27 | 0 | 6 | 3 | 4 | 1 |
| 375 | 11: 65025056-65025067 | T | 5utrE | MALAT1 | tumor | 12 | 12 | 46 | 0 | 34 | 2 | 29 | 4 |
| 376 | 11: 27546865-27546876 | T | 5utrI | BDNFOS | tumor | 12 | 12 | 37 | 0 | 4 | 2 | 3 | 3 |
| 377 | 13: 31331064-31331077 | TTCT | 5utrI | EEF1DP3 | tumor | 14 | 14 | 62 | 2 | 6 | 2 | 5 | 4 |
| | | TT | | | | | | | | | | | |
| 378 | 14: 102373732-102373743 | A | 5utrI | TRAF3 | tumor | 12 | 12 | 36 | 0 | 4 | 2 | 3 | 3 |
| 379 | 9: 9541490-9841501 | AT | 5utrl | PTPRD | tumor | 12 | 12 | 55 | 2 | 4 | 1 | 4 | 3 |
| 380 | 21: 39953532-39953544 | T | 5utrl | B3GALT5 | tumor | 13 | 13 | 31 | 0 | 6 | 0 | 7 | 3 |
| 381 | 15: 49916914-49916927 | T | 5utrl | TMOD3 | tumor | 14 | 14 | 26 | 0 | 4 | 2 | 4 | 4 |
| 382 | 3: 142450428-142450453 | GT | 5utrl | ACPL2 | tumor | 26 | 26 | 31 | 1 | 4 | 1 | 3 | 3 |
| 383 | 13: 23673170-23673205 | CA | 5utrl | SPATA13 | tumor | 36 | 12 | 37 | 0 | 4 | 0 | 6 | 4 |
| 384 | 18: 54430033-54430045 | A | 5utrl | ALPK2 | tumor | 13 | 13 | 42 | 1 | 35 | 0 | 28 | 3 |
| 385 | 4: 170791681-170791692 | T | 5utrl | CLCN3 | tumor | 12 | 11 | 38 | 0 | 4 | 2 | 4 | 4 |
| 386 | 6: 35438202-35438213 | T | 5utrl | PPARD | tumor | 12 | 12 | 26 | 0 | 4 | 2 | 3 | 3 |
| 387 | 18: 65767796-65767814 | CA | 5utrl | CD226 | tumor | 19 | 19 | 57 | 0 | 11 | 2 | 8 | 3 |
| 388 | 1: 120260181-120260190 | GTG | exon | NOTCH2 | tumor | 10 | 10 | 114 | 0 | 34 | 0 | 30 | 4 |
| 389 | 9: 133095845-133095854 | AGC | exon | NUP214 | tumor | 10 | 10 | 117 | 0 | 65 | 0 | 60 | 4 |
| 390 | 10: 76272683-76272697 | AAA | exon | MYST4 | tumor | 15 | 15 | 118 | 0 | 64 | 0 | 62 | 4 |
| | | AGC | | | | | | | | | | | |
| 391 | 5: 33718881-33718891 | TCT | exon | ADAMTS12 | tumor | 11 | 11 | 123 | 0 | 66 | 0 | 58 | 4 |
| 392 | 11: 117847960-117847970 | AGGA | exon | MLL | tumor | 11 | 11 | 116 | 0 | 65 | 0 | 53 | 4 |
| 393 | 1: 153594026-153594037 | TTCTC | exon | ASH1L | tumor | 12 | 12 | 116 | 0 | 52 | 0 | 42 | 3 |
| 394 | 1: 11213577-11213588 | TGACT | exon | FRAP1 | tumor | 12 | 12 | 114 | 0 | 56 | 2 | 51 | 4 |
| 395 | 16: 18778432-18778445 | CAAA | exon | SMG1 | tumor | 14 | 14 | 70 | 0 | 67 | 0 | 63 | 4 |
| 396 | 2: 191570844-191570853 | CAAG | exon | STAT1 | tumor | 10 | 10 | 118 | 0 | 48 | 2 | 45 | 3 |
| 397 | 12: 118756316-118756326 | TCAGC | exon | CIT | tumor | 11 | 11 | 121 | 0 | 64 | 0 | 57 | 4 |
| 398 | 1: 245654986-245654998 | AAGG | exon | NLRP3 | tumor | 13 | 13 | 117 | 0 | 54 | 1 | 43 | 3 |
| 399 | 9: 122971831-122971844 | AGAA | exon | CEP110 | tumor | 14 | 14 | 112 | 0 | 59 | 0 | 59 | 4 |
| 400 | 14: 29163357-29163369 | AT | intron | PRKD1 | tumor | 13 | 13 | 107 | 0 | 24 | 2 | 27 | 4 |
| 401 | 14: 23592112-23592130 | CA | intron | LRRC16B | tumor | 19 | 19 | 53 | 1 | 4 | 0 | 7 | 4 |
| 402 | 6: 71571355-71571367 | T | intron | SMAP1 | tumor | 13 | 13 | 40 | 0 | 9 | 1 | 11 | 3 |
| 403 | 1: 64247540-64247551 | TCCCT | intron | ROR1 | tumor | 12 | 12 | 113 | 0 | 31 | 2 | 30 | 4 |
| 404 | 2: 237073465-237073501 | GAT | intron | IQCA1 | tumor | 37 | 37 | 30 | 0 | 4 | 2 | 5 | 4 |
| 405 | 17: 64500589-64500605 | GT | intron | ABCA9 | tumor | 17 | 17 | 112 | 0 | 36 | 2 | 36 | 4 |
| 406 | 12: 9647882-9647893 | T | intron | KLRB1 | tumor | 12 | 12 | 40 | 0 | 4 | 2 | 3 | 3 |
| 407 | X: 138642412-138642426 | A | intron | ATP11C | tumor | 15 | 15 | 30 | 1 | 11 | 0 | 16 | 3 |
| 408 | 2: 172521280-172521306 | CAAA | intron | HAT1 | tumor | 27 | 23 | 33 | 0 | 4 | 2 | 6 | 4 |
| 409 | 2: 202302175-202302187 | A | intron | ALS2 | tumor | 13 | 13 | 43 | 1 | 43 | 2 | 40 | 3 |
| 410 | 2: 230361914-230361925 | A | intron | TRIP12 | tumor | 12 | 12 | 30 | 0 | 38 | 0 | 33 | 3 |
| 411 | 13: 69362768-69362799 | CA | intron | KLHL1 | tumor | 32 | 32 | 29 | 0 | 3 | 2 | 3 | 4 |
| 412 | 5: 58433294-58433307 | T | intron | PDE4D | tumor | 14 | 14 | 26 | 0 | 3 | 1 | 3 | 4 |
| 413 | 5: 112688598-112688611 | A | intron | MCC | tumor | 14 | 14 | 28 | 1 | 5 | 1 | 6 | 3 |
| 414 | 1: 232504522-232504539 | GTT | intron | SLC35F3 | tumor | 18 | 18 | 63 | 0 | 34 | 1 | 32 | 3 |
| 415 | 11: 2435621-2435634 | A | intron | KCNQ1 | tumor | 14 | 14 | 36 | 0 | 3 | 0 | 4 | 3 |
| 416 | 3: 101921488-101921499 | T | intron | TFG | tumor | 12 | 12 | 80 | 1 | 36 | 1 | 39 | 3 |
| 417 | 11: 101347498-101347515 | TTTG | intron | KIAA1377 | tumor | 18 | 18 | 32 | 0 | 6 | 1 | 8 | 4 |
| 418 | 12: 3211699-3211710 | T | intron | TSPAN9 | tumor | 12 | 12 | 36 | 0 | 4 | 2 | 4 | 3 |
| 419 | 2: 212738282-212738295 | AT | intron | ERBB4 | tumor | 14 | 14 | 62 | 0 | 4 | 1 | 4 | 3 |
| 420 | 12: 54633721-54633733 | TCCCT | intron | DGKA | tumor | 13 | 13 | 113 | 0 | 67 | 0 | 54 | 4 |
| 421 | 12: 25190475-25190486 | A | intron | CASC1 | tumor | 12 | 11 | 71 | 2 | 4 | 1 | 9 | 4 |
| 422 | 2: 121891501-1218915I4 | A | intron | CLASP1 | tumor | 14 | 14 | 35 | 1 | 3 | 1 | 4 | 4 |
| 423 | 18: 65484019-65484030 | T | intron | DOK6 | tumor | 12 | 12 | 46 | 0 | 4 | 2 | 6 | 3 |
| 424 | X: 11290692-11290716 | ATA | intron | ARHGAP6 | tumor | 25 | 25 | 41 | 0 | 4 | 1 | 4 | 3 |
| 425 | 17: 59809586-59809598 | T | intron | PECAM1 | tumor | 13 | 13 | 27 | 1 | 4 | 1 | 5 | 4 |
| 426 | 8: 139701835-139701848 | A | intron | COL22A1 | tumor | 14 | 14 | 28 | 0 | 3 | 0 | 5 | 3 |
| 427 | 21: 37767209-37767220 | T | intron | DYRK1A | tumor | 12 | 12 | 104 | 0 | 36 | 0 | 35 | 3 |
| 428 | 1: 214647891-214647903 | A | intron | USH2A | tumor | 13 | 13 | 40 | 0 | 3 | 2 | 3 | 3 |
| 429 | 1: 955848-955860 | GT | intron | AGRN | tumor | 13 | 13 | 26 | 0 | 3 | 0 | 5 | 3 |
| 430 | 2: 183540346-183540357 | A | intron | NCKAP1 | tumor | 12 | 12 | 51 | 1 | 5 | 2 | 4 | 3 |
| 431 | 2: 169826278-169826291 | A | intron | LRP2 | tumor | 14 | 14 | 27 | 1 | 5 | 2 | 4 | 3 |
| 432 | 2: 133175567-133175592 | CTG | intron | NAP5 | tumor | 26 | 26 | 73 | 1 | 14 | 1 | 13 | 4 |
| 433 | 2: 114103519-114103543 | TC | intron | RABL2A | tumor | 25 | 25 | 37 | 1 | 3 | 2 | 5 | 3 |
| 434 | 11: 73270333-73270354 | TGT | intron | PAAF1 | tumor | 22 | 22 | 58 | 2 | 4 | 0 | 5 | 4 |
| 435 | 8: 62701497-62701508 | T | intron | ASPH | tumor | 12 | 12 | 35 | 0 | 37 | 1 | 32 | 3 |
| 436 | 16: 16034570-16034597 | TGAA | intron | ABCC1 | tumor | 28 | 28 | 58 | 0 | 67 | 0 | 62 | 4 |
| 437 | 12: 47735297-47735311 | AAAC | intron | MLL2 | tumor | 15 | 15 | 73 | 0 | 55 | 2 | 54 | 4 |
| 438 | 13: 31910759-31910781 | AC | intron | N4BP2L2 | tumor | 23 | 23 | 69 | 0 | 38 | 1 | 34 | 4 |
| 439 | 13: 31972855-31972867 | A | intron | N4BP2L2 | tumor | 13 | 13 | 32 | 0 | 5 | 2 | 4 | 4 |
| 440 | 1: 38090119-38090134 | A | intron | MTF1 | tumor | 16 | 16 | 27 | 0 | 3 | 0 | 3 | 3 |
| 441 | 2: 44299109-44299120 | T | intron | PPM1B | tumor | 12 | 12 | 41 | 0 | 37 | 1 | 35 | 3 |
| 442 | 12: 101775892-101775903 | TAAA | intron | PAH | tumor | 12 | 12 | 64 | 0 | 11 | 2 | 12 | 3 |
| | | TG | | | | | | | | | | | |
| 443 | 3: 54178722-54178737 | GTGC | intron | CACNA2D3 | tumor | 16 | 16 | 49 | 0 | 27 | 2 | 32 | 3 |
| 444 | 16: 56115061-56115073 | T | intron | CCDC102A | tumor | 13 | 13 | 32 | 0 | 4 | 2 | 4 | 3 |
| 445 | 13: 40793906-40793917 | CTTA | intron | NARG1L | tumor | 12 | 8 | 78 | 3 | 5 | 2 | 5 | 4 |
| 446 | 3: 29916478-29916501 | AG | intron | RBMS3 | tumor | 24 | 24 | 101 | 3 | 8 | 0 | 10 | 4 |
| 447 | 5: 54634695-54634706 | A | intron | DHX29 | tumor | 12 | 12 | 48 | 0 | 4 | 1 | 3 | 4 |
| 448 | 17: 26710596-26710619 | GTTT | intron | NF1 | tumor | 24 | 24 | 48 | 0 | 8 | 0 | 8 | 4 |
| 449 | 11: 107537954-107537973 | AAA | intron | NPAT | tumor | 20 | 20 | 68 | 0 | 53 | 0 | 47 | 3 |
| | | AC | | | | | | | | | | | |
| 450 | 3: 176768597-176768609 | T | intron | NAALADL2 | tumor | 13 | 13 | 43 | 0 | 4 | 2 | 5 | 4 |
| 451 | 2: 178253896-178253915 | AAGA | intron | PDE11A | tumor | 20 | 20 | 85 | 0 | 45 | 2 | 36 | 3 |
| 452 | 18: 64849667-64849679 | T | intron | CCDC102B | tumor | 13 | 13 | 34 | 1 | 3 | 0 | 4 | 3 |
| 453 | 13: 93086277-93086289 | GA | intron | GPC6 | tumor | 13 | 13 | 52 | 0 | 4 | 1 | 5 | 3 |
| 454 | 16: 63946310-63946338 | TG | intron | LOC283867 | tumor | 29 | 29 | 37 | 0 | 4 | 2 | 5 | 4 |
| 455 | 10: 12474935-12474958 | AC | intron | CAMK1D | tumor | 24 | 20 | 39 | 0 | 4 | 0 | 5 | 4 |
| 456 | 11: 8442952-8442964 | A | intron | STK33 | tumor | 13 | 13 | 26 | 0 | 27 | 1 | 28 | 4 |
| 457 | 1: 100364918-100364941 | AAA | intron | SASS6 | tumor | 24 | 24 | 32 | 0 | 4 | 2 | 3 | 3 |
| | | AT | | | | | | | | | | | |
| 458 | 6: 6234664-6234694 | AC | intron | F13A1 | tumor | 31 | 31 | 33 | 0 | 5 | 0 | 4 | 4 |
| 459 | 6: 5678045-5678057 | A | intron | FARS2 | tumor | 13 | 13 | 34 | 0 | 5 | 2 | 4 | 3 |
| 460 | 6: 41877742-41877754 | T | intron | USP49 | tumor | 13 | 13 | 29 | 0 | 4 | 2 | 4 | 3 |
| 461 | 17: 34249684-34249696 | AC | intron | C17orf98 | tumor | 13 | 11 | 49 | 0 | 5 | 2 | 8 | 4 |
| 462 | 3: 31707416-31707427 | T | intron | OSBPL10 | tumor | 12 | 11 | 27 | 0 | 4 | 2 | 4 | 3 |
| 463 | 11: 95578886-95578897 | T | intron | MAML2 | tumor | 12 | 12 | 36 | 0 | 4 | 1 | 3 | 3 |
| 464 | 6: 72768730-72768742 | A | intron | RIMS1 | tumor | 13 | 13 | 40 | 1 | 4 | 0 | 4 | 4 |
| 465 | 13: 23927833-23927846 | TCAA | intron | PARP4 | tumor | 14 | 14 | 26 | 0 | 7 | 1 | 7 | 3 |
| | | CC | | | | | | | | | | | |
| 466 | 9: 19593497-19593510 | GGGA | intron | SLC24A2 | tumor | 14 | 14 | 45 | 0 | 12 | 2 | 14 | 4 |
| 467 | 2: 68582063-68582074 | A | intron | APLF | tumor | 12 | 12 | 62 | 1 | 3 | 2 | 4 | 3 |
| 468 | 22: 19431652-19431663 | A | intron | PI4KA | tumor | 12 | 12 | 86 | 2 | 13 | 1 | 15 | 3 |
| 469 | 1: 39457032-39457050 | TTTTG | intron | MACF1 | tumor | 19 | 19 | 55 | 2 | 5 | 1 | 8 | 3 |
| 470 | 1: 155364685-155364696 | T | intron | ETV3 | tumor | 12 | 12 | 80 | 1 | 32 | 0 | 36 | 3 |
| 471 | 12: 95229090-95229102 | A | intron | PCTK2 | tumor | 13 | 13 | 51 | 0 | 48 | 1 | 43 | 3 |
| 472 | 9: 77938801-77938812 | T | intron | PCSK5 | tumor | 12 | 12 | 36 | 0 | 10 | 0 | 10 | 3 |
| 473 | 1: 149052378-149052389 | ACAC | intron | ARNT | tumor | 12 | 12 | 88 | 0 | 37 | 0 | 32 | 3 |
| | | CC | | | | | | | | | | | |
| 474 | 13: 98158197-98158211 | TA | intron | SLC15A1 | tumor | 15 | 15 | 65 | 1 | 4 | 1 | 3 | 3 |
| 475 | 3: 74440458-74440469 | T | intron | CNTN3 | tumor | 12 | 12 | 41 | 0 | 4 | 1 | 4 | 3 |
| 476 | 1: 59792358-59792371 | TTTG | intron | FGGY | tumor | 14 | 14 | 111 | 0 | 53 | 0 | 41 | 4 |
| | | TT | | | | | | | | | | | |
| 477 | 7: 131790738-131790753 | AC | intron | PLXNA4 | tumor | 16 | 16 | 46 | 0 | 4 | 0 | 9 | 3 |
| 478 | 1: 100390099-100390129 | AAAC | intron | LRRC39 | tumor | 31 | 31 | 56 | 1 | 5 | 2 | 4 | 3 |
| 479 | 2: 222866669-222866683 | CT | intron | PAX3 | tumor | 15 | 15 | 82 | 0 | 30 | 0 | 33 | 3 |
| 480 | 19: 54802431-54802450 | CA | intron | PRR12 | tumor | 20 | 20 | 37 | 0 | 5 | 1 | 6 | 3 |
| 481 | 2: 149533529-149533540 | T | intron | KIF5C | tumor | 12 | 12 | 39 | 0 | 5 | 2 | 6 | 3 |
| 482 | 12: 97796138-97796150 | A | intron | ANKS1B | tumor | 13 | 13 | 34 | 0 | 4 | 0 | 5 | 3 |
| 483 | 9: 99905357-99905368 | A | intron | TRIM14 | tumor | 12 | 12 | 25 | 0 | 4 | 0 | 4 | 4 |
| 484 | 9: 124091698-124091710 | T | intron | MRRF | tumor | 13 | 13 | 57 | 2 | 5 | 1 | 4 | 3 |
| 485 | 11: 10122611-10122633 | AAA | intron | SBF2 | tumor | 23 | 23 | 43 | 0 | 4 | 2 | 7 | 3 |
| | | AT | | | | | | | | | | | |
| 486 | X: 12634127-12634138 | T | intron | FRMPD4 | tumor | 12 | 12 | 44 | 0 | 4 | 2 | 3 | 4 |
| 487 | 13: 27795312-27795323 | A | intron | FLT1 | tumor | 12 | 12 | 99 | 1 | 15 | 1 | 16 | 3 |
| 488 | 16: 70255618-70255630 | A | intron | PHLPPL | tumor | 13 | 13 | 88 | 3 | 46 | 1 | 37 | 4 |
| 489 | 3: 77696674-77696698 | GT | intron | ROBO2 | tumor | 25 | 25 | 39 | 0 | 30 | 0 | 27 | 3 |
| 490 | 11: 104377835-104377858 | AC | intron | CASP5 | tumor | 24 | 24 | 85 | 0 | 11 | 0 | 8 | 3 |
| 491 | 2: 98981028-98981040 | A | 3utrE | TSGA10 | tumor | 13 | 13 | 41 | 1 | 31 | 1 | 26 | 4 |
| 492 | 7: 136562755-136562767 | A | 3utrE | PTN | tumor | 13 | 13 | 37 | 0 | 5 | 0 | 4 | 4 |
| 493 | 12: 116068238-116068250 | AGC | 3utrE | FBXO21 | tumor | 13 | 13 | 114 | 0 | 64 | 1 | 62 | 4 |
| 494 | 21: 16713680-16713693 | A | 3utrI | C21orf34 | tumor | 14 | 14 | 28 | 1 | 2 | 2 | 4 | 4 |
| 495 | 21: 29688555-29688568 | T | 3utrI | C21orf41 | tumor | 14 | 14 | 30 | 1 | 4 | 1 | 4 | 3 |
| 496 | 12: 12223985-12223996 | TGAA | 3utrI | BCL2L14 | tumor | 12 | 12 | 82 | 0 | 52 | 0 | 43 | 4 |
| | | AA | | | | | | | | | | | |
| 497 | 17: 33283275-33283287 | A | 3utrI | LOC284100 | tumor | 13 | 13 | 41 | 0 | 6 | 1 | 4 | 3 |
| 498 | 9: 106496034-106496052 | AC | upstream | OR13D1 | tumor | 19 | 19 | 60 | 2 | 4 | 1 | 3 | 4 |
| 499 | 11: 4586527-4586555 | AAACA | upstream | TRIM68 | tumor | 29 | 24 | 38 | 0 | 5 | 2 | 4 | 4 |
| 500 | X: 138864535-138864561 | CAA | downstream | LOC347487 | tumor | 27 | 27 | 30 | 0 | 6 | 1 | 7 | 4 |
| 501 | 4: 22944879-22944890 | T | intergenic | — | tumor | 12 | 12 | 51 | 1 | 4 | 2 | 4 | 4 |
| 502 | 5: 89017822-89017833 | TC | intergenic | — | tumor | 12 | 15 | 29 | 0 | 10 | 1 | 6 | 4 |
| 503 | 7: 117536707-117536726 | AG | intergenic | — | tumor | 20 | 20 | 35 | 0 | 4 | 1 | 7 | 3 |
| 504 | 9: 84708983-84708995 | ACAT | intergenic | — | tumor | 13 | 13 | 47 | 0 | 5 | 1 | 5 | 3 |
| 505 | 1: 103011122-103011133 | TTGC | intergenic | — | tumor | 12 | 12 | 32 | 0 | 5 | 1 | 5 | 3 |
| | | TT | | | | | | | | | | | |
| 506 | 10: 113366658-113366671 | TA | intergenic | — | tumor | 14 | 14 | 62 | 2 | 4 | 2 | 3 | 3 |
| 507 | 21: 26901170-26901181 | AAAT | intergenic | — | tumor | 12 | 12 | 62 | 0 | 3 | 2 | 3 | 3 |
| 508 | 21: 18207268-18207284 | TGTA | intergenic | — | tumor | 17 | 17 | 37 | 0 | 4 | 1 | 5 | 3 |
| 509 | 10: 64181936-64181961 | GAG | intergenic | — | tumor | 26 | 26 | 32 | 1 | 24 | 1 | 23 | 4 |
| 510 | 12: 113441200-113441211 | ATTC | intergenic | — | tumor | 12 | 12 | 44 | 0 | 17 | 2 | 17 | 3 |
| | | TC | | | | | | | | | | | |
| 511 | 2: 234927411-234927424 | T | intergenic | — | tumor | 14 | 14 | 38 | 0 | 4 | 1 | 4 | 4 |
| 512 | 1: 207469326-207469339 | A | intergenic | — | tumor | 14 | 13 | 41 | 1 | 3 | 2 | 2 | 3 |
| 513 | 1: 20661739-20661764 | CTG | intergenic | — | tumor | 26 | 26 | 32 | 0 | 28 | 1 | 22 | 4 |
| 514 | 12: 79281454-79281478 | AG | intergenic | — | tumor | 25 | 23 | 40 | 0 | 5 | 1 | 7 | 3 |
| 515 | 12: 125080497-125080508 | A | intergenic | — | tumor | 12 | 12 | 26 | 0 | 4 | 2 | 4 | 3 |
| 516 | 3: 109748618-109748633 | AT | intergenic | — | tumor | 16 | 16 | 39 | 0 | 5 | 1 | 5 | 3 |
| 517 | 12: 27188726-27188748 | TTTG | intergenic | — | tumor | 23 | 23 | 34 | 0 | 5 | 0 | 7 | 4 |
| 518 | 1: 40834801-40834814 | A | intergenic | — | tumor | 14 | 14 | 38 | 1 | 4 | 1 | 4 | 3 |
| 519 | 12: 59191190-59191202 | T | intergenic | — | tumor | 13 | 13 | 38 | 1 | 2 | 0 | 3 | 3 |
| 520 | 6: 107574882-107574894 | A | intergenic | — | tumor | 13 | 12 | 32 | 0 | 5 | 2 | 6 | 3 |
| 521 | 11: 60623602-60623613 | CA | intergenic | — | tumor | 12 | 12 | 105 | 1 | 9 | 0 | 10 | 3 |
| 522 | 1: 221291902-221291917 | CTTC | intergenic | — | tumor | 16 | 16 | 32 | 0 | 5 | 2 | 5 | 4 |
| | | CA | | | | | | | | | | | |
| 523 | 10: 109161907-109161919 | T | intergenic | — | tumor | 13 | 13 | 28 | 0 | 4 | 1 | 4 | 3 |
| 524 | 1: 232694112-232694135 | GTTT | intergenic | — | tumor | 24 | 24 | 26 | 0 | 5 | 2 | 6 | 3 |
| 525 | 7: 141651782-141651794 | T | intergenic | — | tumor | 13 | 13 | 46 | 0 | 3 | 0 | 6 | 4 |
| 526 | 1: 88112010-88112037 | TTTTC | intergenic | — | tumor | 28 | 28 | 25 | 0 | 4 | 2 | 3 | 3 |
| 527 | 9: 25189911-25189940 | AC | intergenic | — | tumor | 30 | 30 | 27 | 0 | 5 | 2 | 6 | 4 |
| 528 | 9: 124127899-124127919 | TG | intergenic | — | tumor | 21 | 21 | 29 | 0 | 6 | 2 | 8 | 3 |
| 529 | X: 95595451-95595469 | AC | intergenic | — | tumor | 19 | 19 | 40 | 0 | 5 | 1 | 6 | 3 |
| 530 | 11: 60623550-60623561 | CA | intergenic | — | tumor | 12 | 12 | 105 | 1 | 9 | 0 | 10 | 3 |
| 531 | 14: 84998722-84998733 | A | intergenic | — | tumor | 12 | 12 | 40 | 0 | 4 | 2 | 4 | 3 |
| 532 | 15: 68542433-68542469 | AC | intergenic | — | tumor | 37 | 21 | 27 | 0 | 3 | 0 | 3 | 3 |
| 533 | 11: 60623479-60623490 | CA | intergenic | — | tumor | 12 | 12 | 105 | 1 | 9 | 0 | 10 | 3 |
| 534 | 10: 76229006-76229018 | AG | intergenic | — | tumor | 13 | 13 | 34 | 0 | 4 | 2 | 6 | 3 |
| 535 | 10: 77188715-77188728 | T | intergenic | — | tumor | 14 | 14 | 29 | 0 | 5 | 2 | 4 | 3 |
| 536 | 1: 146442612-146442625 | AAC | intergenic | — | tumor | 14 | 14 | 51 | 2 | 6 | 1 | 5 | 3 |
| 537 | 10: 41936144-41936158 | AAA | intergenic | — | tumor | 15 | 15 | 105 | 1 | 11 | 2 | 8 | 3 |
| | | AC | | | | | | | | | | | |
| 538 | 1: 217258209-217258226 | CACA | intergenic | — | tumor | 18 | 18 | 60 | 0 | 4 | 2 | 5 | 4 |
| | | CC | | | | | | | | | | | |
| 539 | 13: 31449787-31449798 | A | intergenic | — | tumor | 12 | 11 | 61 | 1 | 5 | 1 | 4 | 3 |
| 540 | 1: 86445838-86445849 | TGG | intergenic | — | tumor | 12 | 12 | 49 | 0 | 5 | 1 | 9 | 3 |
| | | AAG | | | | | | | | | | | |
| 541 | 3: 32588984-32589012 | TAAA | intergenic | — | tumor | 29 | 29 | 32 | 0 | 4 | 0 | 5 | 4 |
| 542 | 1: 151009970-151009983 | T | intergenic | — | tumor | 14 | 14 | 29 | 0 | 3 | 1 | 3 | 3 |
| 543 | 3: 188512753-188512766 | TC | intergenic | — | tumor | 14 | 14 | 68 | 1 | 9 | 0 | 6 | 4 |
| 544 | 10: 43317835-43317849 | GTG | intergenic | — | tumor | 15 | 15 | 26 | 0 | 9 | 0 | 14 | 4 |
| | | GG | | | | | | | | | | | |
| 545 | 3: 73127788-73127800 | TGTA | intergenic | — | tumor | 13 | 13 | 47 | 0 | 4 | 2 | 5 | 3 |
| 546 | 9: 116656866-116656879 | T | intergenic | — | tumor | 14 | 14 | 34 | 0 | 3 | 0 | 4 | 3 |
| 547 | 5: 97280161-97280172 | A | intergenic | — | tumor | 12 | 12 | 31 | 1 | 4 | 2 | 3 | 3 |
| 548 | 1: 20324652-20324663 | T | intergenic | — | tumor | 12 | 12 | 54 | 1 | 7 | 2 | 11 | 4 |
| 549 | 10: 116756625-116756636 | A | intergenic | — | tumor | 12 | 12 | 49 | 1 | 5 | 1 | 4 | 3 |
| 550 | 12: 25922213-25922233 | AC | intergenic | — | tumor | 21 | 21 | 27 | 1 | 4 | 2 | 6 | 3 |
| 551 | 4: 52942725-52942736 | A | intergenic | — | tumor | 12 | 12 | 59 | 1 | 4 | 2 | 5 | 4 |
| 552 | 15: 77843385-77843410 | AG | intergenic | — | tumor | 26 | 26 | 34 | 0 | 7 | 1 | 5 | 4 |
| 553 | 14: 85022580-85022592 | T | intergenic | — | tumor | 13 | 13 | 36 | 1 | 4 | 0 | 2 | 4 |
| 554 | 2: 49983974-49983986 | T | intergenic | — | tumor | 13 | 13 | 39 | 0 | 4 | 2 | 4 | 3 |
| 555 | 11: 86222164-86222180 | TA | intergenic | — | tumor | 17 | 17 | 83 | 1 | 3 | 1 | 4 | 3 |
| 556 | 9: 31652690-31652701 | T | intergenic | — | tumor | 12 | 12 | 38 | 0 | 5 | 2 | 5 | 4 |
| 557 | 10: 21577174-21577189 | TTTTC | intergenic | — | tumor | 16 | 16 | 95 | 0 | 9 | 2 | 15 | 3 |
| 558 | 8: 16906982-16906993 | T | intergenic | — | tumor | 12 | 12 | 48 | 0 | 3 | 2 | 3 | 3 |
| 559 | X: 39109723-39109738 | CA | intergenic | — | tumor | 16 | 16 | 53 | 0 | 8 | 2 | 8 | 3 |
| 560 | 2: 122765252-122765263 | A | intergenic | — | tumor | 12 | 12 | 50 | 0 | 8 | 1 | 12 | 3 |
| 561 | 2: 53164111-53164126 | A | intergenic | — | tumor | 16 | 16 | 25 | 0 | 3 | 2 | 3 | 4 |
| 562 | 2: 37498349-37498361 | T | intergenic | — | tumor | 13 | 13 | 33 | 0 | 4 | 1 | 4 | 4 |
| 563 | X: 65136790-65136821 | TGC | intergenic | — | tumor | 32 | 32 | 33 | 0 | 26 | 2 | 27 | 3 |
| 564 | X: 123995248-123995259 | T | intergenic | — | tumor | 12 | 12 | 42 | 1 | 2 | 1 | 3 | 3 |
| 565 | 13: 104998198-104998211 | A | intergenic | — | tumor | 14 | 14 | 29 | 0 | 3 | 1 | 3 | 3 |
| 566 | 19: 7565170-7565190 | AATC | intergenic | — | tumor | 21 | 21 | 47 | 0 | 4 | 2 | 9 | 3 |
| 567 | 18: 69015962-69015975 | T | intergenic | — | tumor | 14 | 14 | 35 | 0 | 8 | 2 | 7 | 4 |
| 568 | 11: 32085941-32085952 | T | intergenic | — | tumor | 12 | 12 | 26 | 0 | 4 | 2 | 5 | 4 |
| 569 | 12: 61667909-61667921 | AAG | intergenic | — | tumor | 13 | 13 | 65 | 0 | 10 | 1 | 10 | 3 |
| 570 | 10: 60594975-60594987 | AATA | intergenic | — | tumor | 13 | 13 | 57 | 0 | 3 | 1 | 3 | 4 |
| 571 | 10: 46461637-46461649 | CA | intergenic | — | tumor | 13 | 13 | 80 | 3 | 2 | 0 | 6 | 4 |
| 572 | 12: 72330916-72330930 | CAATA | intergenic | — | tumor | 15 | 15 | 61 | 0 | 4 | 1 | 4 | 4 |
| 573 | 2: 199595472-199595484 | AC | intergenic | — | tumor | 13 | 13 | 58 | 2 | 3 | 1 | 3 | 3 |
| 574 | 12: 36224319-36224348 | GT | intergenic | — | tumor | 30 | 30 | 25 | 0 | 7 | 0 | 6 | 3 |
| 575 | 10: 102595011-102595022 | T | intergenic | — | tumor | 12 | 12 | 25 | 0 | 5 | 0 | 5 | 3 |
| 576 | 13: 55870083-55870095 | A | intergenic | — | tumor | 13 | 13 | 25 | 0 | 2 | 0 | 3 | 3 |
| 577 | 11: 127607605-127607631 | AAAT | intergenic | — | tumor | 27 | 27 | 32 | 0 | 4 | 2 | 3 | 3 |
| 578 | 14: 23059924-23059936 | T | intergenic | — | tumor | 13 | 13 | 29 | 0 | 4 | 2 | 5 | 4 |
| 579 | 10: 4108431-4108442 | CCT | intergenic | — | tumor | 12 | 12 | 40 | 0 | 13 | 2 | 26 | 4 |
| 580 | 6: 23691135-23691151 | AC | intergenic | — | tumor | 17 | 17 | 37 | 0 | 4 | 0 | 3 | 3 |
| 581 | 5: 79691672-79691684 | A | intergenic | — | tumor | 13 | 13 | 86 | 3 | 6 | 1 | 5 | 3 |
| 582 | 2: 200097291-200097318 | GTTT | intergenic | — | tumor | 28 | 28 | 28 | 0 | 5 | 1 | 4 | 3 |
| 583 | 4: 34063105-34063122 | TA | intergenic | — | tumor | 18 | 18 | 42 | 0 | 1 | 0 | 3 | 4 |
| 584 | 4: 174560181-174560193 | A | intergenic | — | tumor | 13 | 12 | 53 | 2 | 5 | 1 | 4 | 3 |
| 585 | 8: 90069787-90069799 | A | intergenic | — | tumor | 13 | 12 | 41 | 1 | 4 | 1 | 5 | 4 |
| 586 | X: 16830736-16830758 | TTCC | intergenic | — | tumor | 23 | 23 | 39 | 0 | 7 | 0 | 8 | 3 |
| 587 | 8: 29882174-29882186 | A | intergenic | — | tumor | 13 | 13 | 40 | 0 | 4 | 2 | 3 | 4 |
| 588 | 1: 147799003-147799016 | TTG | intergenic | — | tumor | 14 | 14 | 51 | 1 | 4 | 0 | 7 | 3 |
| 589 | 14: 103826520-103826531 | TG | intergenic | — | tumor | 12 | 12 | 86 | 3 | 8 | 2 | 9 | 4 |
| 590 | 12: 121515948-121515960 | A | intergenic | — | tumor | 13 | 13 | 29 | 0 | 6 | 2 | 7 | 4 |
| 591 | 5: 105094651-105094664 | GGA | intergenic | — | tumor | 14 | 14 | 54 | 1 | 4 | 2 | 4 | 3 |
| 592 | 11: 108548098-108548110 | A | intergenic | — | tumor | 13 | 13 | 26 | 0 | 4 | 0 | 3 | 3 |
| 593 | 11: 26885069-26885081 | T | intergenic | — | tumor | 13 | 13 | 32 | 0 | 4 | 2 | 4 | 4 |
| 594 | 11: 37284835-37284855 | CA | intergenic | — | tumor | 21 | 21 | 50 | 2 | 5 | 2 | 3 | 3 |
| 595 | 16: 11366255-11366270 | CT | intergenic | — | tumor | 16 | 16 | 45 | 0 | 5 | 2 | 6 | 4 |
| 596 | 22: 26545510-26545540 | GTTT | intergenic | — | tumor | 31 | 31 | 29 | 1 | 5 | 1 | 5 | 3 |
| 597 | 4: 52893619-52893646 | TAAA | intergenic | — | tumor | 28 | 28 | 37 | 0 | 4 | 1 | 5 | 4 |
| 598 | 12: 107415122-107415138 | TAT | intergenic | — | tumor | 17 | 17 | 67 | 0 | 5 | 1 | 6 | 3 |
| 599 | 5: 106607265-106607278 | A | intergenic | — | tumor | 14 | 14 | 28 | 0 | 4 | 1 | 3 | 4 |
| 600 | 13: 65525771-65525783 | T | intergenic | — | tumor | 13 | 13 | 32 | 0 | 5 | 2 | 4 | 3 |

Table 4. Microsatellites conserved in the 1kGP female population that vary in OV. This table lists all 600 mono- to hexamer microsatellite loci that were identified as conserved in the 1kGP females but had >3% variation and ≧3 variant alleles (requires that more than one individual have the variation) in either the OV germline DNA samples, tumors, or both. Leave-one-out cross validated a set of 100 of these loci (referred to as OV-associated). The remaining 500 loci (shaded) which were dropped from the set after leave-one-out were only able to distinguish between OV signature and normal with a sensitivity of 36% and a specificity of 89% when a minimum of 4 variations within the loci setwas required. Human reference hg18 was used for all chromosomal locations, determination of gene regions, and for the reference microsatellite lengths. In 73 instances the consensus from the 1kGP females differed from the hg18 reference length, the female consensus was used as the baseline for determining variation for the OV samples. 3utrE-3′UTR exon encoded; 5utrE-5′UTR exon encoded; 3utrI-3′UTR intronic; 5utrI-5′UTR intronic; upstream and downstream boundaries were defined as 1,000 nt from the transcription start and stop sites. Microsatellites spanning a boundary between genomic regions were labeled as belonging to the region that contained the majority of the sequence. This microsatellite genotyping assumes two alleles per genome at any given microsatellite locus.

TABLE 5
Glioblastoma

| Microsatellite | | | | | 1kGP 250 samples | | | GM BL samples | | | GM TM samples | | |
|---|---|---|---|---|---|---|---|---|---|---|---|---|---|
| location (chromosome: nt position) | motif | ref length | gene region | gene symbol | total samples | consensus | alleles | total samples | consensus | alleles | total samples | consensus | alleles |
| 1: 100444455-100444467 | A | 13 | intron | DBT | 102 | 13 | 13 (200), 12 | 16 | 13 | 13 (26), 12 | 17 | 13 | 12 (1), 13 |
| | | | | | | | (2), 14 (2) | | | (6) | | | (33) |
| 1: 153652407-153652418 | A | 12 | intron | ASH1L | 158 | 12 | 12 (313), 14 | 26 | 12 | 11 (4), 12 | 31 | 12 | 11 (1), 12 |
| | | | | | | | (2), 13 (1) | | | (47), 14 (1) | | | (61) |
| 1: 182042328-182042339 | T | 12 | intron | RGL1 | 81 | 12 | 11 (1), 12 | 24 | 12 | 11 (3), 12 | 23 | 12 | 11 (1), 12 |
| | | | | | | | (161) | | | (45) | | | (45) |
| 1: 235930414-235930426 | T | 13 | intron | RYR2 | 105 | 13 | 13 (210) | 31 | 13 | 13 (54), 12 | 25 | 13 | 14 (3), 13 |
| | | | | | | | | | | (2), 14 (6) | | | (47) |
| 1: 46499455-46499476 | T | 22 | intron | RAD54L | 119 | 22 | 22 (234), 23 | 23 | 22 | 22 (46) | 20 | 22 | 22 (36), 23 |
| | | | | | | | (4) | | | | | | (4) |
| 10: 114908637-114908648 | T | 12 | intron | TCF7L2 | 184 | 12 | 11 (1), 13 | 31 | 12 | 11 (4), 13 | 25 | 12 | 12 (50) |
| | | | | | | | (4), 12 (363) | | | (2), 12 (56) | | | |
| 10: 36851713-36851736 | CA | 24 | intergenic | — | 44 | 24 | 24 (88) | 24 | 24 | 22 (1), 24 | 24 | 24 | 24 (48) |
| | | | | | | | | | | (45), 26 (2) | | | |
| 10: 74474995-74475006 | T | 12 | intron | P4HA1 | 103 | 12 | 11 (1), 12 | 7 | 12 | 13 (4), 12 | 1 | 12 | 12 (2) |
| | | | | | | | (205) | | | (10) | | | |
| 11: 65025056-65025067 | T | 12 | 5utrE | MALAT1 | 77 | 12 | 12 (154) | 24 | 12 | 11 (3), 13 | 25 | 12 | 11 (2), 12 |
| | | | | | | | | | | (2), 12 (43) | | | (46), 13 (2) |
| 13: 102055299-102055311 | T | 13 | intron | TPP2 | 27 | 13 | 13 (54) | 25 | 13 | 13 (46), 12 | 16 | 13 | 13 (32) |
| | | | | | | | | | | (3), 14 (1) | | | |
| 13: 29752364-29752375 | A | 12 | intron | KATL1 | 110 | 12 | 13 (4), 12 | 28 | 12 | 13 (4), 12 | 32 | 12 | 12 (59), 14 |
| | | | | | | | (216) | | | (51), 14 (1) | | | (1), 13 (4) |
| 14: 18641456-18641477 | T | 22 | intron | POTEG | 75 | 22 | 22 (147), 23 | 23 | 22 | 22 (46) | 21 | 22 | 22 (39), 24 |
| | | | | | | | (3) | | | | | | (2), 23 (1) |
| 14: 72076483-72076494 | T | 12 | intron | RGS6 | 91 | 12 | 12 (182) | 25 | 12 | 11 (8), 12 | 23 | 12 | 12 (46) |
| | | | | | | | | | | (42) | | | |
| 16: 52073066-52073077 | T | 12 | intron | RBL2 | 81 | 12 | 12 (162) | 26 | 12 | 11 (1), 12 | 27 | 12 | 11 (1), 12 |
| | | | | | | | | | | (51) | | | (51), 13 (2) |
| 16: 73276740-73276751 | A | 12 | intron | MLKL | 110 | 12 | 12 (220) | 21 | 12 | 11 (2), 13 | 15 | 12 | 12 (30) |
| | | | | | | | | | | (2), 12 (38) | | | |
| 16: 79623661-79623673 | T | 13 | intron | CENPN | 95 | 13 | 13 (187), 14 | 26 | 13 | 13 (49), 14 | 21 | 13 | 13 (42) |
| | | | | | | | (3) | | | (3) | | | |
| 17: 24853715-24853727 | T | 13 | intron | TAOK1 | 51 | 13 | 12 (2), 13 | 23 | 13 | 13 (42), 12 | 28 | 13 | 12 (1), 13 |
| | | | | | | | (100) | | | (4) | | | (55) |
| 17: 37621710-37621721 | T | 12 | intron | STAT5B | 64 | 12 | 11 (1), 12 | 27 | 12 | 11 (1), 12 | 29 | 12 | 11 (4), 12 |
| | | | | | | | (127) | | | (53) | | | (54) |
| 19: 13184113-13184125 | GT | 13 | intron | CAC1A | 78 | 13 | 12 (1), 13 | 28 | 13 | 13 (56) | 24 | 13 | 13 (43), 14 |
| | | | | | | | (155) | | | | | | (5) |
| 19: 21142361-21142372 | A | 12 | intron | ZNF431 | 54 | 12 | 11 (2), 12 | 31 | 12 | 11 (3), 12 | 30 | 12 | 11 (1), 12 |
| | | | | | | | (106) | | | (59) | | | (59) |
| 19: 21350659-21350670 | A | 12 | intergenic | — | 83 | 12 | 11 (1), 12 | 21 | 12 | 11 (1), 12 | 25 | 12 | 11 (3), 12 |
| | | | | | | | (165) | | | (41) | | | (47) |
| 2: 202302175-202302187 | A | 13 | intron | ALS2 | 89 | 13 | 12 (1), 13 | 27 | 13 | 13 (51), 12 | 27 | 13 | 12 (2), 13 |
| | | | | | | | (177) | | | (3) | | | (52) |
| 2: 98981028-98981040 | A | 13 | 3utrE | TSGA10 | 84 | 13 | 12 (1), 14 | 18 | 13 | 13 (32), 12 | 26 | 13 | 12 (1), 14 |
| | | | | | | | (1), 13 (166) | | | (2), 14 (2) | | | (1), 13 (50) |
| 21: 38428961-38428987 | TTCC | 27 | 5utrI | DSCR8 | 118 | 27 | 27 (234), 19 | 25 | 27 | 27 (44), 23 | 23 | 27 | 27 (46) |
| | | | | | | | (1), 23 (1) | | | (6) | | | |
| 22: 45117761-45117775 | T | 15 | intron | TRMU | 111 | 15 | 16 (2), 14 | 26 | 15 | 16 (1), 14 | 24 | 15 | 14 (3), 15 |
| | | | | | | | (2), 15 (218) | | | (3), 15 (48) | | | (44), 16 (1) |
| 3: 150385620-150385631 | T | 12 | intron | CP | 112 | 12 | 11 (2), 12 | 28 | 12 | 11 (3), 12 | 26 | 12 | 11 (6), 12 |
| | | | | | | | (222) | | | (53) | | | (46) |
| 3: 41852478-41852490 | A | 13 | intron | ULK4 | 60 | 13 | 16 (2), 13 | 15 | 13 | 16 (2), 13 | 10 | 13 | 16 (2), 13 |
| | | | | | | | (118) | | | (26), 15 (2) | | | (18) |
| 3: 48194325-48194342 | AC | 18 | intron | CDC25A | 54 | 16 | 16 (108) | 25 | 16 | 18 (4), 16 | 28 | 16 | 18 (5), 16 |
| | | | | | | | | | | (46) | | | (51) |
| 3: 67641907-67641918 | T | 12 | intron | SUCLG2 | 113 | 12 | 11 (2), 12 | 29 | 12 | 11 (4), 12 | 32 | 12 | 11 (2), 12 |
| | | | | | | | (224) | | | (54) | | | (62) |
| 4: 103831000-103831022 | AT | 23 | intron | MANBA | 140 | 23 | 21 (1), 23 | 9 | 23 | 23 (10), 17 | 6 | 23 | 17 (2), 23 |
| | | | | | | | (279) | | | (8) | | | (10) |
| 4: 43557024-43557052 | TTG | 29 | intergenic | — | 67 | 29 | 26 (2), 29 | 11 | 29 | 26 (2), 29 | 6 | 29 | 26 (3), 29 |
| | | | | | | | (132) | | | (20) | | | (9) |
| 5: 161427569-161427580 | A | 12 | 5utrE | GABRG2 | 64 | 12 | 12 (128) | 11 | 12 | 11 (2), 13 | 14 | 12 | 12 (26), 13 |
| | | | | | | | | | | (1), 12 (19) | | | (2) |
| 5: 72221348-72221362 | T | 15 | intron | TNPO1 | 56 | 15 | 15 (112) | 29 | 15 | 14 (3), 15 | 28 | 15 | 14 (3), 15 |
| | | | | | | | | | | (55) | | | (53) |
| 6: 101094988-101095000 | A | 13 | intron | ASCC3 | 65 | 13 | 11 (1), 12 | 14 | 13 | 13 (25), 12 | 13 | 13 | 12 (5), 13 |
| | | | | | | | (1), 13 (128) | | | (3) | | | (21) |
| 6: 152769773-152769785 | T | 13 | intron | SYNE1 | 67 | 13 | 12 (1), 13 | 20 | 13 | 11 (1), 13 | 28 | 13 | 12 (4), 13 |
| | | | | | | | (133) | | | (36), 12 (3) | | | (52) |
| 6: 256798-256810 | T | 13 | intron | DUSP22 | 78 | 13 | 13 (153), 12 | 24 | 13 | 13 (47), 14 | 26 | 13 | 12 (5), 14 |
| | | | | | | | (1), 14 (2) | | | (1) | | | (1), 13 (46) |
| 6: 43622506-43622518 | A | 13 | intron | XPO5 | 116 | 13 | 12 (4), 13 | 29 | 13 | 13 (53), 12 | 30 | 13 | 13 (55), 12 |
| | | | | | | | (228) | | | (5) | | | (4), 14 (1) |
| 6: 64347898-64347912 | T | 15 | intron | PTP4A1 | 29 | 15 | 14 (1), 15 | 23 | 15 | 14 (6), 15 | 22 | 15 | 14 (6), 15 |
| | | | | | | | (57) | | | (40) | | | (37), 13 (1) |
| 7: 102905960-102905974 | T | 15 | intron | RELN | 88 | 15 | 14 (2), 15 | 22 | 15 | 14 (6), 15 | 21 | 15 | 14 (2), 15 |
| | | | | | | | (174) | | | (38) | | | (38), 16 (2) |
| 7: 111261986-111261998 | A | 13 | intron | DOCK4 | 84 | 13 | 13 (165), 12 | 29 | 13 | 13 (55), 12 | 29 | 13 | 13 (56), 12 |
| | | | | | | | (2), 4 (1) | | | (3) | | | (2) |
| 7: 134906568-134906580 | T | 13 | intron | NUP205 | 88 | 13 | 13 (174), 12 | 32 | 13 | 13 (63), 14 | 29 | 13 | 12 (1), 14 |
| | | | | | | | (1), 14 (1) | | | (1) | | | (2), 13 (55) |
| 7: 136990139-136990151 | A | 13 | intron | DGKI | 87 | 13 | 12 (3), 13 | 22 | 13 | 13 (41), 12 | 24 | 13 | 12 (4), 13 |
| | | | | | | | (171) | | | (3) | | | (44) |
| 9: 14787414-14787425 | AC | 12 | intron | FREM1 | 142 | 12 | 12 (281), 14 | 29 | 12 | 12 (53), 14 | 19 | 12 | 12 (33), 14 |
| | | | | | | | (3) | | | (5) | | | (5) |
| 9: 84549183-84549196 | A | 14 | intergenic | — | 62 | 14 | 14 (124) | 30 | 14 | 13 (6), 14 | 29 | 14 | 14 (54), 13 |
| | | | | | | | | | | (54) | | | (4) |
| X: 110381185-110381198 | A | 14 | intron | CAPN6 | 83 | 14 | 14 (166) | 23 | 14 | 13 (4), 15 | 26 | 14 | 14 (46), 15 |
| | | | | | | | | | | (5), 14 (37) | | | (6) |
| X: 132665972-132665984 | A | 13 | intron | GPC3 | 50 | 13 | 12 (1), 13 | 22 | 13 | 13 (44) | 15 | 13 | 12 (2), 14 |
| | | | | | | | (99) | | | | | | (2), 13 (26) |
| X: 48155256-48155269 | A | 14 | intron | SSX4B | 26 | 14 | 14 (51), 13 | 17 | 14 | 13 (3), 14 | 14 | 14 | 14 (27), 13 |
| | | | | | | | (1) | | | (31) | | | (1) |
| X: 80263832-80263843 | A | 12 | upstream | NSBP1 | 74 | 12 | 12 (146), 13 | 27 | 12 | 11 (2), 12 | 29 | 12 | 11 (4), 12 |
| | | | | | | | (2) | | | (52) | | | (53), 13 (1) |

Table 5. Informative loci as identified using a leave-one-out strategy following the comparison of the allelic distribution at each loci for ‘normal’ genomes and those genomes from patients with Glioblastoma.

TABLE 6
Glioblastoma

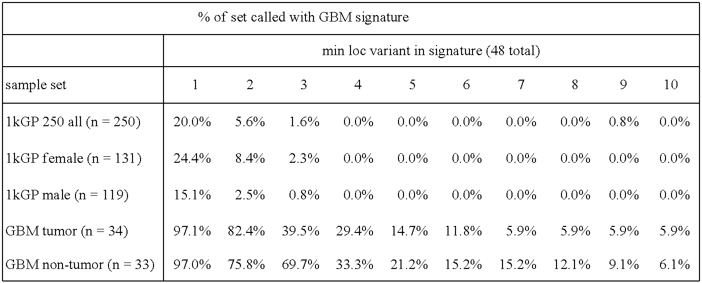

Percentage of genomes having a GBM-signature with the indicated minimum variant loci. There is an inverse relationship between the minimum number of variant loci for classifying a genome as having a GBM signature and the percentage of genomes classified. The grey box demarks the number of variants required to reduce GBM signature calling below the expected level of 0.65% and 0.5% in the 1kGP male and female population, respectively.

TABLE 7
Colon Cancer

| Microsatellite location | | | | ref | TUMOR allele lengths |
|---|---|---|---|---|---|
| (chromosome: nt position) | region | gene symbol | motif family | length | (calls) |
| 10: 119034325-119034334 | exon | PDZD8 | TTGC | 10 | 9 (2), 10 (236) |
| 22: 37211898-37211924 | exon | DDX17 | AGG | 27 | 27 (237), 24 (1) |
| 16: 68340479-68340495 | exon | NOB1 | TCC | 17 | 17 (237), 14 (1) |
| 11: 76747638-76747662 | exon | PAK1 | ATC | 25 | 22 (1), 25 (237) |
| 9: 138148265-138148281 | exon | C9orf69 | AGC | 17 | 17 (235), 14 (1) |
| 1: 224101463-224101481 | exon | TMEM63A | TGC | 19 | 22 (1), 19 (233) |
| 11: 64563765-64563774 | exon | SNX15 | AAG | 10 | 7 (1), 10 (231) |
| 12: 122516716-122516726 | exon | SNRNP35 | AG | 11 | 11 (229), 9 (1) |
| 3: 51405862-51405880 | exon | RBM15B | ACC | 19 | 22 (1), 19 (229) |
| X: 153658283-153658305 | exon | DKC1 | AAG | 23 | 26 (2), 23 (226) |
| 15: 79028302-79028314 | exon | KIAA1199 | AAG | 13 | 10 (4), 13 (222) |
| 3: 50660436-50660447 | exon | MAPKAPK3 | AGGC | 12 | 13 (8), 12 (214) |
| 5: 137116828-137116846 | exon | HNRNPA0 | CCG | 19 | 22 (3), 19 (219) |
| 4: 71773555-71773573 | exon | UTP3 | AGG | 19 | 16 (3), 19 (217) |
| 19: 17021706-17021716 | exon | HICE1 | AG | 11 | 11 (216), 9 (2) |
| 13: 95237338-95237353 | exon | DNAJC3 | AAAAG | 16 | 16 (210), 17 (2) |
| 13: 19118717-19118728 | exon | MPHOSPH8 | AAAAAG | 12 | 13 (1), 12 (209) |
| 6: 74267164-74267173 | exon | MTO1 | AG | 10 | 11 (1), 10 (205) |
| 6: 32256050-32256059 | exon | RNF5 | TTC | 10 | 9 (1), 10 (203) |
| 1: 154832117-154832135 | exon | GPATCH4 | TTTTTC | 19 | 18 (1), 19 (194), 20 (7) |
| 13: 19118663-19118680 | exon | MPHOSPH8 | AAAAAG | 18 | 18 (201), 19 (1) |
| 6: 108478982-108478991 | exon | OSTM1 | ATTC | 10 | 11 (2), 10 (196) |
| 1: 109126581-109126591 | exon | STXBP3 | AAAAG | 11 | 11 (196), 9 (2) |
| 7: 42916048-42916058 | exon | C7orf25 | TC | 11 | 11 (194), 9 (4) |
| 19: 50603699-50603713 | exon | CD3EAP | AAG | 15 | 16 (2), 17 (1), 14 (2), 15 (185) |
| 1: 1261533-1261548 | exon | DVL1 | TGGGG | 16 | 16 (189), 15 (1) |
| 15: 48561172-48561185 | exon | USP8 | AAAC | 14 | 15 (2), 14 (186) |
| X: 46915411-46915425 | exon | RBM10 | CGG | 15 | 12 (2), 15 (186) |
| 7: 107943140-107943149 | exon | PNPLA8 | AT | 10 | 10 (172), 12 (2) |
| 2: 43305244-43305269 | exon | ZFP36L2 | TGC | 26 | 26 (171), 29 (1) |
| 12: 95141621-95141633 | exon | ELK3 | AAAAC | 13 | 13 (145), 14 (1) |
| 11: 124000974-124000985 | exon | TBRG1 | AAAAAG | 12 | 13 (6), 12 (134) |
| 13: 51905818-51905830 | exon | VPS36 | TTTTC | 13 | 13 (118), 14 (2) |
| 1: 55278141-55278167 | exon | PCSK9 | TGC | 27 | 27 (97), 30 (7) |
| 17: 62113782-62113791 | exon | PRKCA | AAGC | 10 | 11 (9), 10 (93) |
| 20: 36988734-36988756 | exon | FAM83D | CGG | 23 | 26 (6), 23 (84) |
| 17: 68717454-68717478 | exon | FAM104A | TGC | 25 | 22 (2), 25 (82) |
| 10: 8046398-8046409 | exon | TAF3 | AAAAG | 12 | 11 (2), 12 (80) |
| 18: 18006071-18006101 | exon | GATA6 | ACC | 31 | 28 (2), 31 (74) |
| 9: 134193732-134193749 | exon | SETX | ATC | 18 | 18 (67), 15 (1) |
| 15: 72006957-72006974 | exon | LOXL1 | CCG | 18 | 18 (57), 15 (1) |
| 1: 234812967-234812976 | exon | HEATR1 | AAAT | 10 | 11 (2), 10 (46) |
| 12: 116990711-116990742 | exon | FLJ20674 | TCC | 32 | 32 (42), 29 (2) |
| 17: 6868744-6868773 | exon | BCL6B | AGC | 30 | 33 (2) |
| 14: 102874510-102874532 | exon | EIF5 | ACC | 23 | 26 (1), 23 (239) |
| 6: 33763867-33763879 | exon | ITPR3 | AGG | 13 | 10 (2), 13 (236) |
| 11: 118403640-118403650 | exon | SLC37A4 | ACACC | 11 | 10 (238) |
| 16: 1989884-1989899 | exon | ZNF598 | TCC | 16 | 13 (1), 19 (24), 16 (207) |
| 1: 1674208-1674235 | exon | NADK | TCC | 28 | 28 (145), 31 (85) |
| 2: 237909603-237909616 | exon | COL6A3 | AGC | 14 | 11 (10), 14 (218) |
| 14: 22860695-22860704 | exon | PABPN1 | TGC | 10 | 22 (4), 10 (224) |
| 11: 108293845-108293870 | exon | DDX10 | ATG | 26 | 26 (213), 29 (3) |
| 10: 70445822-70445835 | exon | KIAA1279 | AAAT | 14 | 13 (1), 15 (1), 14 (210) |
| 11: 18084135-18084148 | exon | SAAL1 | CGG | 14 | 17 (37), 14 (175) |
| 14: 99775541-99775575 | exon | YY1 | ACC | 35 | 38 (1), 35 (200), 32 (9) |
| 3: 185911828-185911848 | exon | MAGEF1 | TCC | 21 | 21 (55), 24 (151) |
| 16: 88444381-88444396 | exon | SPIRE2 | AGG | 16 | 19 (5), 16 (181) |
| 7: 99795065-99795076 | exon | PILRB | TCC | 12 | 9 (24), 12 (160) |
| 18: 75576176-75576196 | exon | CTDP1 | AGG | 21 | 18 (2), 21 (162) |
| 19: 4768289-4768315 | exon | TICAM1 | AGG | 27 | 27 (152), 30 (8), 24 (4) |
| 14: 22310554-22310566 | exon | OXA1L | AGC | 13 | 16 (23), 13 (141) |
| 19: 43591342-43591359 | exon | FAM98C | AAG | 18 | 21 (3), 18 (149), 15 (2) |
| 1: 31678477-31678491 | exon | SERINC2 | AGC | 15 | 18 (147), 15 (5) |
| 10: 103444348-103444370 | exon | FBXW4 | TCC | 23 | 23 (151), 20 (1) |
| 20: 4628049-4628061 | exon | PRNP | TGG | 13 | 37 (2), 13 (140) |
| 20: 4628073-4628085 | exon | PRNP | TGG | 13 | 37 (2), 13 (140) |
| X: 119271862-119271881 | exon | ZBTB33 | ATG | 20 | 23 (68), 20 (40) |
| 14: 22619719-22619750 | exon | ACIN1 | TCC | 32 | 32 (98), 29 (8) |
| 10: 97909836-97909848 | exon | ZNF518A | AAAAAC | 13 | 13 (98), 14 (8) |
| 17: 16980287-16980321 | exon | MPRIP | AGC | 35 | 35 (20), 32 (86) |
| 3: 40478525-40478556 | exon | RPL14 | TGC | 32 | 35 (39), 32 (45), 29 (18) |
| 2: 227369640-227369662 | exon | IRS1 | TGC | 23 | 26 (1), 23 (91) |
| 12: 1932585-1932613 | exon | DCP1B | TGC | 29 | 32 (33), 29 (47) |
| 14: 92224291-92224307 | exon | RIN3 | CGG | 17 | 17 (20), 14 (58) |
| 5: 56213606-56213631 | exon | MAP3K1 | AAC | 26 | 23 (66), 26 (8) |
| 4: 15122103-15122114 | exon | CC2D2A | AAG | 12 | 9 (4), 12 (68) |
| 11: 119040888-119040912 | exon | PVRL1 | TCC | 25 | 25 (60), 28 (4) |
| 5: 156412022-156412033 | exon | HAVCR1 | TTG | 12 | 9 (22), 12 (42) |
| 12: 6808275-6808285 | exon | LEPREL2 | CGCGG | 11 | 12 (56) |
| 20: 226688-226707 | exon | ZCCHC3 | CGG | 20 | 17 (48) |
| 5: 140933741-140933781 | exon | DIAPH1 | AGG | 41 | 38 (1), 44 (4), 41 (23) |
| 14: 23839690-23839719 | exon | C14orf21 | AGG | 30 | 33 (10), 30 (10) |
| 3: 155440981-155440990 | exon | SGEF | AGTC | 10 | 6 (12) |
| 21: 46546414-46546436 | exon | C21orf58 | TGG | 23 | 26 (3), 23 (9) |
| 7: 142272174-142272207 | exon | EPHB6 | TCC | 34 | 34 (4), 31 (2) |
| 9: 130060617-130060654 | exon | GOLGA2 | TCC | 38 | 35 (2), 38 (4) |
| 4: 140871035-140871062 | exon | MAML3 | TGC | 28 | 25 (4) |
| 2: 88707845-88707869 | exon | EIF2AK3 | AGC | 25 | 22 (2) |

Table 7. Table of loci that varied in colon cancer genomes relative to the highly conserved loci found in ‘normal’ individuals.

TABLE 8
Lung Squamous Cell Carcinoma

| Microsatellite location | gene | | motif family | ref | UNKNOWN allele lengths |
|---|---|---|---|---|---|
| (chromosome: nt position) | symbol | region | cyclic | length | (calls) |
| 1: 144788110-144788125 | FAM108A3 | exon | ACCCC | 16 | 17 (314) |
| 22: 22893073-22893082 | CABIN1 | exon | ACC | 10 | 16 (36), 10 (242) |
| 16: 1989884-1989899 | ZNF598 | exon | TCC | 16 | 19 (49), 16 (265) |
| 7: 72359667-72359676 | NSUN5 | exon | AAC | 10 | 7 (25), 10 (129) |
| 18: 46977136-46977161 | MEX3C | exon | CCG | 26 | 26 (6), 17 (42) |
| 10: 97909836-97909848 | ZNF518A | exon | AAAAAC | 13 | 13 (274), 14 (34) |
| 3: 50660436-50660447 | MAPKAPK3 | exon | AGGC | 12 | 13 (17), 12 (303) |
| 17: 62113782-62113791 | PRKCA | exon | AAGC | 10 | 11 (15), 10 (183) |
| 10: 105150196-105150207 | PDCD11 | exon | AAAAAC | 12 | 13 (10), 12 (293), 14 (1) |
| 1: 11633367-11633377 | FBXO2 | exon | CGG | 11 | 11 (100), 14 (16) |
| 1: 21140821-21140834 | EIF4G3 | exon | AAGG | 14 | 23 (9), 14 (283) |
| 5: 172470291-172470300 | C5orf41 | exon | AAGG | 10 | 11 (8), 10 (230) |
| 1: 35976247-35976261 | CLSPN | exon | TTC | 15 | 12 (11), 15 (197) |
| 19: 50603699-50603713 | CD3EAP | exon | AAG | 15 | 16 (5), 15 (305) |
| 20: 205710-205722 | C20orf96 | exon | TTC | 13 | 13 (254), 12 (1), 14 (2), 15 (1) |
| 13: 51905818-51905830 | VPS36 | exon | TTTTC | 13 | 13 (327), 14 (3) |
| 15: 79028302-79028314 | KIAA1199 | exon | AAG | 13 | 10 (4), 13 (296) |
| 12: 48313940-48313952 | PRPF40B | exon | AGC | 13 | 14 (4) |
| 10: 115653292-115653303 | NHLRC2 | exon | AAAAAC | 12 | 13 (2), 12 (304) |
| 6: 43005336-43005362 | CNPY3 | exon | TGC | 27 | 27 (210), 24 (2) |
| 5: 6808013-6808026 | POLS | exon | AC | 14 | 15 (2), 14 (312) |
| 1: 210526078-210526090 | PPP2R5A | exon | TCG | 13 | 16 (2), 13 (282) |
| 12: 32025985-32025999 | C12orf35 | exon | TCC | 15 | 12 (2), 15 (288) |
| 2: 75039317-75039334 | POLE4 | exon | CGG | 18 | 21 (1), 18 (257) |
| 1: 52599801-52599821 | CC2D1B | exon | TCC | 21 | 21 (38), 15 (2) |
| 2: 74603987-74603996 | DQX1 | exon | AGGG | 10 | 11 (1), 10 (251) |
| 1: 75002330-75002346 | TYW3 | exon | ATG | 17 | 17 (328), 14 (2) |
| 10: 119034325-119034334 | PDZD8 | exon | TTGC | 10 | 11 (1), 10 (317) |
| 16: 87311084-87311098 | FAM38A | exon | TTC | 15 | 12 (1), 15 (331) |
| 11: 33646246-33646256 | C11orf41 | exon | ACAG | 11 | 11 (123), 12 (1) |
| 13: 47779490-47779499 | RB1 | exon | AG | 10 | 10 (302), 12 (2) |
| 11: 33587991-33588001 | C11orf41 | exon | AAAG | 11 | 11 (151), 12 (1) |
| 7: 72499559-72499590 | BAZ1B | exon | TCC | 32 | 14 (2) |
| 7: 21434829-21434846 | SP4 | exon | AGG | 18 | 18 (39), 24 (1) |
| 5: 168950721-168950731 | CCDC99 | exon | AAC | 11 | 11 (323), 12 (1) |
| 1: 232623159-232623170 | TARBP1 | exon | ACTTGG | 12 | 12 (311), 14 (1) |
| 13: 27795047-27795059 | FLT1 | exon | TTTC | 13 | 13 (125), 14 (1) |
| 19: 44635873-44635882 | SUPT5H | exon | AAG | 10 | 7 (1), 10 (331) |
| 1: 59020712-59020727 | JUN | exon | TGC | 16 | 19 (1), 16 (313) |
| 22: 40940288-40940298 | TCF20 | exon | TTG | 11 | 8 (2), 11 (286) |
| 21: 33783206-33783219 | DNAJC28 | exon | TTC | 14 | 8 (2), 14 (68) |
| 4: 6343932-6343943 | WFS1 | exon | AAG | 12 | 9 (1), 12 (313) |
| 7: 137864475-137864488 | TRIM24 | exon | AAAT | 14 | 15 (1), 14 (273) |
| 3: 57517808-57517819 | PDE12 | exon | TTC | 12 | 9 (1), 12 (305) |
| 3: 48468151-48468160 | ATRIP | exon | AAG | 10 | 7 (2), 10 (282) |
| 11: 117932958-117932969 | C11orf60 | exon | TTC | 12 | 9 (2), 12 (10) |
| 12: 95141621-95141633 | ELK3 | exon | AAAAC | 13 | 13 (295), 14 (1) |
| 1: 153715235-153715245 | ASH1L | exon | TTTTC | 11 | 11 (285), 12 (1) |
| 7: 27179627-27179636 | HOXA10 | exon | CGG | 10 | 11 (1), 10 (27) |
| 2: 230842516-230842528 | SP140 | exon | AATG | 13 | 13 (124), 14 (2) |
| 13: 95237338-95237353 | DNAJC3 | exon | AAAAG | 16 | 16 (331), 17 (1) |
| 2: 227369052-227369072 | IRS1 | exon | TGC | 21 | 18 (2), 21 (198) |
| 22: 39145088-39145098 | MKL1 | exon | ACC | 11 | 8 (1), 11 (315) |
| 10: 105171250-105171261 | PDCD11 | exon | TCC | 12 | 10 (1), 12 (315) |
| 19: 48866075-48866098 | PLAUR | exon | AGC | 24 | 24 (223), 12 (1) |
| 19: 10292432-10292446 | RAVER1 | exon | TGC | 15 | 12 (2), 15 (324) |
| 12: 120364831-120364841 | FBXL10 | exon | TTC | 11 | 8 (1), 11 (321) |
| 19: 960186-960205 | GRIN3B | exon | AGC | 20 | 17 (2), 20 (12) |
| 14: 102662628-102662655 | TNFAIP2 | exon | AAG | 28 | 25 (2), 28 (246) |
| 1: 221603326-221603347 | SUSD4 | exon | TGC | 22 | 25 (1), 22 (261) |
| 1: 1637752-1637761 | CDC2L1 | exon | TTTC | 10 | 16 (197), 10 (69) |
| 3: 185911828-185911848 | MAGEF1 | exon | TCC | 21 | 21 (73), 24 (211) |
| 11: 47745240-47745251 | FNBP4 | exon | TGG | 12 | 6 (78), 12 (142) |
| 10: 91487885-91487896 | KIF20B | exon | AAGGAG | 12 | 18 (52), 12 (188) |
| 3: 40478525-40478556 | RPL14 | exon | TGC | 32 | 23 (2), 29 (2), 17 (4), 20 (5), 14 (9) |
| 19: 43591342-43591359 | FAM98C | exon | AAG | 18 | 21 (8), 18 (296) |
| 1: 8638909-8638934 | RERE | exon | TTTGTC | 26 | 26 (46), 20 (8) |
| 20: 42127973-42127983 | TOX2 | exon | CCG | 11 | 11 (108), 14 (8) |
| 14: 102874510-102874532 | EIF5 | exon | ACC | 23 | 26 (4), 23 (324) |
| 16: 88444381-88444396 | SPIRE2 | exon | AGG | 16 | 19 (6), 16 (50) |
| 1: 1674208-1674235 | NADK | exon | TCC | 28 | 25 (3), 28 (211) |
| 1: 215860189-215860199 | GPATCH2 | exon | ATT | 11 | 11 (309), 12 (1) |
| 3: 51952455-51952465 | PARP3 | exon | AAG | 11 | 8 (1), 11 (261) |
| 10: 99116512-99116545 | RRP12 | exon | TCC | 34 | 19 (2) |
| 1: 159762579-159762591 | HSPA6 | exon | ATCACC | 13 | 7 (52), 13 (206) |
| 7: 99795065-99795076 | PILRB | exon | TCC | 12 | 9 (71), 12 (231) |
| 8: 22318174-22318187 | SLC39A14 | exon | TGC | 14 | 8 (58), 14 (226) |
| 12: 116990711-116990742 | FU20674 | exon | TCC | 32 | 26 (26) |
| 14: 22310554-22310566 | OXA1L | exon | AGC | 13 | 16 (22), 13 (152) |
| 2: 237909603-237909616 | COL6A3 | exon | AGC | 14 | 11 (14), 14 (256) |
| 2: 88707845-88707869 | EIF2AK3 | exon | AGC | 25 | 22 (8), 25 (2) |
| 18: 75576176-75576196 | CTDP1 | exon | AGG | 21 | 21 (264), 24 (6) |
| 12: 109505123-109505142 | PPTC7 | exon | CCG | 20 | 17 (6), 20 (24) |
| 1: 55278141-55278167 | PCSK9 | exon | TGC | 27 | 27 (26), 30 (2) |
| 14: 105067095-105067114 | TMEM121 | exon | CCG | 20 | 17 (2) |
| 6: 44078478-44078509 | C6orf223 | exon | CGG | 32 | 26 (2) |
| 19: 4768289-4768315 | TICAM1 | exon | AGG | 27 | 27 (86), 30 (2) |
| 5: 56213606-56213631 | MAP3K1 | exon | AAC | 26 | 23 (132), 26 (14) |
| 14: 92224291-92224307 | RIN3 | exon | CGG | 17 | 17 (10), 14 (98) |
| 17: 77250022-77250035 | CCDC137 | exon | AGG | 14 | 11 (1), 14 (323) |
| 12: 1932585-1932613 | DCP1B | exon | TGC | 29 | 29 (4), 20 (2) |
| 1: 31678477-31678491 | SERINC2 | exon | AGC | 15 | 18 (213), 15 (15) |
| 20: 226688-226707 | ZCCHC3 | exon | CGG | 20 | 17 (90), 20 (2) |
| 1: 86818484-86818517 | CLCA4 | exon | ACTCCT | 34 | 28 (50) |
| 6: 32299637-32299668 | NOTCH4 | exon | AGC | 32 | 17 (2), 20 (4) |

Table 8. Table of loci that varied in lung cancer (Lung Squamous Cell Carcinoma) genomes relative to the highly conserved loci found in ‘normal’ individuals. The right hand column is labeled UNKNOWN because the meta data associated with these samples did not indicate whether they were from tumors or from germline.

TABLE 9
Lung Adenocarcinoma

| | | | | 1 kGP | | |
|---|---|---|---|---|---|---|
| Microsatellite location | | | motif family | average | ref | UNKNOWN allele lengths |
| (chromosome: nt position) | gene symbol | region | cyclic | length | length | (calls) |
| 1: 144788110-144788125 | FAM108A3 | exon | ACCCC | 16 | 16 | 17 (36) |
| 22: 22893073-22893082 | CABIN1 | exon | ACC | 10 | 10 | 16 (18), 10 (18) |
| 18: 46977136-46977161 | MEX3C | exon | CCG | 17 | 26 | 26 (4), 17 (18) |
| 12: 48313940-48313952 | PRPF40B | exon | AGC | 13 | 13 | 14 (4) |
| 3: 50660436-50660447 | MAPKAPK3 | exon | AGGC | 12 | 12 | 13 (2), 12 (34) |
| 1: 11633367-11633377 | FBXO2 | exon | CGG | 11 | 11 | 8 (2), 11 (20), 14 (2) |
| 12: 32025985-32025999 | C12orf35 | exon | TCC | 15 | 15 | 12 (1), 15 (33) |
| 11: 32580971-32580984 | CCDC73 | exon | TTTTC | 14 | 14 | 15 (2), 14 (2) |
| 6: 43005336-43005362 | CNPY3 | exon | TGC | 27 | 27 | 27 (31), 24 (1) |
| 7: 72359667-72359676 | NSUN5 | exon | AAC | 10 | 10 | 7 (1), 10 (1) |
| 17: 62113782-62113791 | PRKCA | exon | AAGC | 10 | 10 | 11 (1), 10 (29) |
| 7: 21434829-21434846 | SP4 | exon | AGG | 18 | 18 | 18 (12), 24 (2) |
| 10: 57788416-57788438 | ZWINT | exon | AGCCTC | 23 | 23 | 23 (31), 29 (1) |
| 12: 131113109-131113120 | EP400 | exon | ACG | 12 | 12 | 9 (1), 12 (33) |
| 15: 79028302-79028314 | KIAA1199 | exon | AAG | 13 | 13 | 10 (1), 13 (27) |
| 8: 118019906-118019930 | C8orf85 | exon | CGG | 25 | 25 | 19 (2) |
| 12: 120364831-120364841 | FBXL10 | exon | TTC | 11 | 11 | 8 (1), 11 (35) |
| 17: 63252843-63252858 | BPTF | exon | ACG | 16 | 16 | 13 (1), 16 (29) |
| 10: 97909836-97909848 | ZNF518A | exon | AAAAAC | 13 | 13 | 13 (34), 14 (2) |
| 1: 1637752-1637761 | CDC2L1 | exon | TTTC | 10.1 | 10 | 16 (15), 10 (9) |
| 3: 185911828-185911848 | MAGEF1 | exon | TCC | 22.7 | 21 | 21 (15), 24 (21) |
| 11: 47745240-47745251 | FNBP4 | exon | TGG | 9.3 | 12 | 6 (12), 12 (20) |
| 3: 40478525-40478556 | RPL14 | exon | TGC | 35.2 | 32 | 11 (2), 23 (10) |
| 10: 91487885-91487896 | KIF20B | exon | AAGGAG | 13.3 | 12 | 18 (10), 12 (18) |
| 5: 156412022-156412033 | HAVCR1 | exon | TTG | 11.5 | 12 | 9 (5), 12 (7) |
| 19: 43591342-43591359 | FAM98C | exon | AAG | 18.1 | 18 | 21 (3), 18 (29) |
| 14: 102874510-102874532 | EIF5 | exon | ACC | 23.1 | 23 | 26 (1), 23 (35) |
| 1: 1674208-1674235 | NADK | exon | TCC | 29 | 28 | 25 (2), 28 (30) |
| 2: 88707845-88707869 | EIF2AK3 | exon | AGC | 22 | 25 | 22 (12) |
| 8: 22318174-22318187 | SLC39A14 | exon | TGC | 12.8 | 14 | 8 (7), 14 (27) |
| 12: 116990711-116990742 | FU20674 | exon | TCC | 30.3 | 32 | 26 (6) |
| 7: 99795065-99795076 | PILRB | exon | TCC | 11.6 | 12 | 9 (3), 12 (23) |
| 1: 159762579-159762591 | HSPA6 | exon | ATCACC | 13 | 13 | 7 (1), 13 (3) |
| 14: 105067095-105067114 | TMEM121 | exon | CCG | 20 | 20 | 17 (2), 20 (2) |
| 12: 109505123-109505142 | PPTC7 | exon | CCG | 19.3 | 20 | 17 (2), 20 (6) |
| 14: 22310554-22310566 | OXA1L | exon | AGC | 13.1 | 13 | 16 (2), 13 (18) |
| 14: 92224291-92224307 | RIN3 | exon | CGG | 14.4 | 17 | 17 (4), 14 (22) |
| 5: 56213606-56213631 | MAP3K1 | exon | AAC | 23.8 | 26 | 23 (14), 26 (6) |
| 1: 31678477-31678491 | SERINC2 | exon | AGC | 17.2 | 15 | 18 (26), 15 (2) |
| 20: 226688-226707 | ZCCHC3 | exon | CGG | 17 | 20 | 17 (10) |

Table 9. Table of loci that varied in lung cancer (Lung Adenocarcinoma) genomes relative to the highly conserved loci found in ‘normal’ individuals. The right hand column is labeled UNKNOWN because the meta data associated with these samples did not indicate whether they were from tumors or from germline.

TABLE 10
Prostate Cancer

| | | | | 1 kGP | | |
|---|---|---|---|---|---|---|
| Microsatellite location | | | Motif family | average | ref | TUMOR alleles |
| (chromosome: nt position) | gene symbol | region | cyclic | length | length | (calls) |
| 1: 234032885-234032894 | LYST | exon | TTC | 10.0 | 10 | 7 (1), 10 (45) |
| 6: 44327897-44327908 | HSP90AB1 | exon | AAG | 12.0 | 12 | 13 (1), 12 (45) |
| 17: 78291999-78292009 | FN3K | exon | AGG | 11.0 | 11 | 8 (1), 11 (1) |
| 12: 6508178-6508191 | NCAPD2 | exon | AAGGTG | 14.0 | 14 | 15 (2), 14 (40) |
| 9: 127043189-127043201 | HSPA5 | exon | AGC | 13.0 | 13 | 16 (3), 13 (21) |
| 7: 72359667-72359676 | NSUN5 | exon | AAC | 10.0 | 10 | 7 (4), 10 (4) |
| 9: 130060617-130060654 | GOLGA2 | exon | TCC | 37.3 | 38 | 35 (5), 38 (33) |
| 11: 85052890-85052899 | CREBZF | exon | TTC | 10.0 | 10 | 7 (2), 10 (28) |
| 10: 97909836-97909848 | ZNF518A | exon | AAAAAC | 13.0 | 13 | 13 (18), 14 (2) |
| 19: 54618343-54618370 | PTH2 | exon | AGC | 28.0 | 28 | 25 (2), 28 (20) |
| 1: 6423367-6423381 | ESPN | exon | TGC | 15.0 | 15 | 19 (2), 15 (30) |
| 13: 78074485-78074513 | POU4F1 | exon | TGG | 29.0 | 29 | 32 (1), 29 (25) |
| 1: 11633367-11633377 | FBXO2 | exon | CGG | 11.0 | 11 | 14 (2) |
| 20: 42127973-42127983 | TOX2 | exon | CCG | 11.1 | 11 | 11 (38), 14 (2) |
| 1: 8638909-8638934 | RERE | exon | TTTGTC | 25.9 | 26 | 26 (35), 20 (1) |
| 3: 185911828-185911848 | MAGEF1 | exon | TCC | 22.7 | 21 | 21 (13), 24 (29) |
| 11: 119040888-119040912 | PVRL1 | exon | TCC | 25.1 | 25 | 22 (2), 25 (39), 28 (1) |
| 1: 1674208-1674235 | NADK | exon | TCC | 29.1 | 28 | 28 (15), 31 (23) |
| 7: 150515200-150515217 | ASB10 | exon | AG | 18.3 | 18 | 18 (14), 20 (4) |
| 4: 77284331-77284344 | NUP54 | exon | TGC | 14.3 | 14 | 17 (6), 14 (34) |
| 5: 156412022-156412033 | HAVCR1 | exon | TTG | 11.6 | 12 | 9 (10), 12 (16) |
| 1: 44368967-44368978 | KLF17 | exon | AAC | 11.7 | 12 | 9 (2), 12 (30) |
| 10: 91487885-91487896 | KIF20B | exon | AAGGAG | 13.3 | 12 | 18 (7), 12 (29) |
| 16: 88444381-88444396 | SPIRE2 | exon | AGG | 16.3 | 16 | 19 (6), 16 (28) |
| 11: 6619322-6619347 | DCHS1 | exon | AGC | 26.1 | 26 | 26 (37), 29 (1) |
| 19: 43591342-43591359 | FAM98C | exon | AAG | 18.0 | 18 | 21 (3), 18 (27) |
| 1: 149945332-149945372 | TNRC4 | exon | TGC | 40.9 | 41 | 38 (1), 41 (21) |
| 3: 40478525-40478556 | RPL14 | exon | TGC | 35.8 | 32 | 32 (1), 26 (37) |
| 11: 47745240-47745251 | FNBP4 | exon | TGG | 9.2 | 12 | 6 (6), 12 (10) |
| 1: 17637569-17637583 | RCC2 | exon | CCG | 15.0 | 15 | 18 (1), 15 (3) |
| 19: 50259447-50259470 | SFRS16 | exon | TCC | 24.0 | 24 | 21 (1), 24 (29), 15 (2) |
| 15: 36564099-36564136 | FAM98B | exon | TGG | 38.0 | 38 | 38 (18), 29 (4) |
| 2: 237909603-237909616 | COL6A3 | exon | AGC | 13.8 | 14 | 11 (2), 14 (40) |
| 1: 159762579-159762591 | HSPA6 | exon | ATCACC | 13.0 | 13 | 7 (4) |
| 18: 75576176-75576196 | CTDP1 | exon | AGG | 21.2 | 21 | 21 (30), 24 (6) |
| 19: 4768289-4768315 | TICAM1 | exon | AGG | 27.2 | 27 | 27 (33), 30 (5) |
| 8: 22318174-22318187 | SLC39A14 | exon | TGC | 12.8 | 14 | 8 (8), 14 (36) |
| 14: 22310554-22310566 | OXA1L | exon | AGC | 13.2 | 13 | 16 (8), 13 (22) |
| 12: 116990711-116990742 | FLJ20674 | exon | TCC | 30.7 | 32 | 32 (16), 26 (2) |
| 3: 46726078-46726104 | TMIE | exon | AAG | 24.3 | 27 | 27 (2), 24 (6) |
| 5: 140933741-140933781 | DIAPH1 | exon | AGG | 40.9 | 41 | 38 (1), 44 (1), 41 (24), 47 (2) |
| 1: 55278141-55278167 | PCSK9 | exon | TGC | 27.0 | 27 | 27 (31), 30 (3) |
| 12: 1932585-1932613 | DCP1B | exon | TGC | 30.4 | 29 | 32 (28), 29 (14) |
| 5: 56213606-56213631 | MAP3K1 | exon | AAC | 23.9 | 26 | 23 (23), 26 (5) |
| 1: 238322192-238322208 | FMN2 | exon | CGG | 14.7 | 17 | 17 (2), 14 (4) |
| 14: 92224291-92224307 | RIN3 | exon | CGG | 14.3 | 17 | 17 (4), 14 (22) |
| 12: 6916141-6916199 | ATN1 | exon | AGC | 45.1 | 59 | 59 (1), 38 (10), 44 (3) |
| 1: 31678477-31678491 | SERINC2 | exon | AGC | 17.2 | 15 | 18 (36), 15 (2) |
| 17: 17637819-17637859 | RAI1 | exon | AGC | 38.7 | 41 | 38 (12), 29 (2), 41 (2) |
| 20: 226688-226707 | ZCCHC3 | exon | CGG | 17.0 | 20 | 17 (4) |
| 7: 142272174-142272207 | EPHB6 | exon | TCC | 34.4 | 34 | 34 (39), 40 (1), 31 (2) |
| 19: 54349523-54349579 | HRC | exon | ATC | 55.8 | 57 | 60 (7), 57 (19), 54 (8) |
| 1: 86818484-86818517 | CLCA4 | exon | ACTCCT | 29.5 | 34 | 28 (24) |
| 6: 32299637-32299668 | NOTCH4 | exon | AGC | 27.6 | 32 | 32 (12), 29 (6), 20 (4) |
| 11: 6368504-6368551 | SMPD1 | exon | TGGCGC | 41.7 | 48 | 36 (8), 48 (16) |
| 2: 96144698-96144721 | ADRA2B | exon | TCC | 26.6 | 24 | 33 (13), 24 (9) |

Table 10. Table of loci that varied in prostate cancer genomes relative to the highly conserved loci found in ‘normal’ individuals.

TABLE 11
Table 11. Changes in protein sequence due to microsatellite variation at 11
BC-associated genes. The red amino acids (which are also bolded and underlined)
illustrate thealterations in protein sequence caused by variant microsatellites.

| | | | nt variation | ref amino | variant | frame- |
|---|---|---|---|---|---|---|
| Locus | | motif | from ref | acids | amino acids | shift |
| 3:50660436-50660447 | MAPKAPK3 | GCAG | 1 | KKQAGSSS | KKAGRQLLCLTGLQQP | yes |
| | | | | | VAHGALEEPGLSACITD | |
| 22:22893073- | CABIN1 | CCA | 6 | PATTTGT | PAPATTTGT | no |
| 22893082 | | | | | | |
| 7:72359667-72359676 | NSUN5 | CAA | -3 | YELLLGKG | YELLGKG | no |
| 17:62113782- | PRKCA | AAGC | 1 | NESKQKT | NESKQKNQ | yes |
| 62113791 | | | | | | |
| 1:21140821-21140834 | EIF4G3 | AGGA | 9 | TVPSFPPTP | TVPSFPPTPPTP | no |
| 1:8638909-8638934 | RERE | TCTTTG | -6 | TADKDKDKDKEKDR | TADKDKDKEKDR | no |
| 7:21434829-21434846 | SP4 | AGG | 6 | KKEEEEEAAA | KKEEEEEAAAAA | no |
| 1:1637752-1637761 | CDC2L1 | TCTT | 6 | RVKEREHE | RVKEKEREHE | no |
| 4:84589090-84589102 | HELQ | TTTC | 1 | VQERKNLIY | VQERKKFNI | yes |
| 1:35976247-35976261 | CLSPN | TTC | -3 | TAEEEEEIGE | TAEEEEIGE | no |
| 1:159762579- | HSPA6 | ATCACC | -6 | TRSPSPMT | TRSPMT | no |
| 159762591 | | | | | | |

TABLE 12

| | Exome/exome equivalent | | | | WGS | | | |
|---|---|---|---|---|---|---|---|---|
| Groups | Count | Average | Stdev | p value | Count | Average | Stdev | p value |
| 1kGP | 131 | 1.0% | 0.2% | — | 111 | 1.5% | 0.4% | — |
| OV Germline | 72 | 1.4% | 0.6% | 3.6E−09 | 4 | 4.7% | 1.2% | 9.4E−29 |
| OV Tumor | 67 | 1.4% | 0.6% | 5.1E−09 | 4 | 4.0% | 2.0% | 4.1E−17 |

Table 12. Overall levels of microsatellite variation were greater in OV patient genomes than in the normal female population. For the 1kGP females, genomes were considered whole genome sequenced (WGS) if ≧200,000 microsatellite loci were called.

TABLE 13
Table 13. Primer pairs which can be used to amplify informative
microsatellite loci disclosed herein.

| | Allele | | | |
|---|---|---|---|---|
| | length in | | | |
| Micro- | human | | | |
| satellite | reference | Other allele | | |
| Locus | (nt) | length (nt) | FWD primer | REV primer |
| C5orf41 | 10 | 11 | TGCAGTAAAGAAGTCACGGAGA | CCTGGAAGCCAGCTTATTTTT |
| PRKCA | 10 | 11 | ACGCCATTCTGACGTCTCTT | ATTTAGTGTGGAGCGGATGG |
| MAPKAPK3 | 12 | 13 | CTTAGTGCCCACCATCCTGT | CCCCATGAGCTACTGGTTGT |
| NSUN5 | 10 | 7 | TTCCAACAGGTCCTCATTCC | GCTTCATGCTTAGGGCATTT |
| EIF4G3 | 14 | 23 | GGAGGAGAAGCTGGAGGAGT | ACGGAGAGCATTGTGGAAAT |
| CABIN1 | 10 | 16 | GGAGGAGCTGAGCATCAGTG | ACGGTAGGCATCCAACAGAA |
| CDC2L1 | 10 | 16 | CAGCCCACTCACCTTTCTCT | GGCCTCGTGAAATTTTTGAA |
| RPL14 | 32 | 8, 11, 14, 17, | CCTGAAAGCTTCTCCCAAAA | TGCCACTTATGCTTTCTTGC |
| | | 20, 23, 26, 29 | | |
| HSPA6 | 13 | 7 | GGGGTCTTCATCCAGGTGTA | AACCATCCTCTCCACCTCCT |

TABLE 14

| | | | | | 1kGP- | BC | |
|---|---|---|---|---|---|---|---|
| | | | | | EUF % | Germline | |
| | | | | Modal | Non- | % Non- | Relative |
| Microsatellite Locus | Gene | Region | Motif | Genotype | Modal | Modal | Risk |
| 2: 198334597-198334608 | COQ10B | intron | A | 12 12 | 2% | 27% | 14.64 |
| 13: 45517483-45517512 | NUFIP1 | intron | AC | 30 30 | 4% | 17% | 4.44 |
| 1: 23408924-23408939 | KDM1A | intron | T | 16 16 | 11% | 44% | 4.16 |
| 19: 49123876-49123893 | SPHK2 | intron | A | 18 18 | 24% | 91% | 3.81 |
| 8: 23709570-23709595 | STC1 | intron | TG | 26 26 | 11% | 41% | 3.77 |
| 20: 20018883-20018904 | CRNKL1 | intron | A | 22 22 | 22% | 81% | 3.63 |
| 18: 44392305-44392320 | PIAS2 | 3′utr | A | 16 16 | 18% | 61% | 3.47 |
| 11: 118353038-118353053 | MLL | intron | T | 16 16 | 14% | 43% | 3.15 |
| 5: 133944044-133944059 | SAR1B | intron | T | 16 16 | 29% | 91% | 3.09 |
| 16: 20956099-20956124 | DNAH3 | intron | AC | 26 26 | 20% | 53% | 2.61 |
| 16: 28842258-28842274 | ATXN2L | intron | A | 17 17 | 28% | 72% | 2.57 |
| X: 10109659-10109674 | WWC3 | 3′utr | A | 16 16 | 34% | 83% | 2.42 |
| 15: 63040517-63040532 | TLN2 | intron | A | 16 16 | 22% | 53% | 2.43 |
| 16: 56718016-56718035 | MT1X | 3′utr | T | 19 20 | 34% | 83% | 2.39 |
| 17: 57663597-57663614 | DHX40 | intron | A | 18 17 | 32% | 72% | 2.27 |
| 7: 148494795-148494811 | CUL1 | intron | T | 17 17 | 42% | 90% | 2.14 |
| 19: 30106131-30106147 | POP4 | intron | T | 17 17 | 53% | 93% | 1.75 |
| 4: 55131002-55131018 | PDGFRA | intron | A | 17 17 | 51% | 85% | 1.66 |
| 10: 45568537-45568553 | — | intergenic | T | 17 17 | 60% | 100% | 1.67 |
| X: 13775753-13775768 | OFD1 | intron | T | 16 16 | 53% | 80% | 1.51 |
| 1: 114372333-114372344 | PTPN22 | intron | A | 11 12 | 50% | 69% | 1.4 |
| 22: 38308043-38308071 | MICALL1 | intron | TG | 25 29 | 59% | 80% | 1.34 |
| 4: 77065477-77065491 | NUP54 | intron | A | 14 15 | 75% | 99% | 1.32 |
| 8: 39607084-39607119 | ADAM2 | intron | GT | 40 36 | 62% | 81% | 1.31 |
| 7: 38282131-38282150 | TRG | intron | GT | 22 20 | 58% | 78% | 1.35 |
| 6: 49815874-49815887 | CRISP1 | intron | T | 14 14 | 41% | 13% | 0.32 |
| 3: 197880131-197880172 | FAM157A | exon | GCA | 42 42 | 57% | 17% | 0.3 |
| 1: 10357207-10357223 | KIF1B | intron | T | 16 17 | 49% | 14% | 0.3 |
| 3: 154834380-154834396 | MME | intron | TA | 17 17 | 23% | 7% | 0.3 |
| 2: 75919273-75919297 | C2orf3 | intron | AT | 21 21 | 46% | 13% | 0.29 |
| 4: 47746603-47746615 | CORIN | intron | A | 13 13 | 19% | 5% | 0.28 |
| 17: 15973418-15973434 | NCOR1 | intron | T | 16 17 | 55% | 14% | 0.26 |
| 5: 86679496-86679513 | RASA1 | intron | A | 18 17 | 43% | 11% | 0.25 |
| 12: 110834031-110834048 | ANAPC7 | intron | A | 18 17 | 52% | 13% | 0.24 |
| 14: 102550070-102550087 | HSP90AA1 | intron | A | 18 17 | 48% | 11% | 0.22 |
| 17: 63747018-63747031 | CCDC46 | intron | A | 14 14 | 40% | 8% | 0.2 |
| 3: 33877501-33877512 | PDCD6IP | intron | T | 12 12 | 21% | 4% | 0.18 |
| 9: 5798652-5798666 | ERMP1 | intron | A | 15 15 | 45% | 7% | 0.16 |
| 15: 84473326-84473342 | ADAMTSL3 | intron | T | 16 17 | 41% | 6% | 0.13 |
| 14: 51348282-51348298 | ABHD12B | intron | T | 18 19 | 32% | 4% | 0.13 |
| 2: 203630103-203630123 | FAM117B | intron | T | 21 21 | 24% | 3% | 0.12 |
| 3: 98299708-98299720 | CPOX | intron | A | 13 13 | 46% | 6% | 0.13 |
| X: 70812449-70812463 | ACRC | intron | T | 15 15 | 10% | 1% | 0.11 |
| 2: 203680555-203680567 | ICA1L | intron | A | 13 13 | 24% | 3% | 0.11 |
| 15: 89811883-89811895 | FANCI | intron | T | 13 13 | 19% | 2% | 0.11 |
| 11: 62565909-62565944 | NXF1 | intron | AAA | 36 36 | 38% | 4% | 0.11 |
| | | | AGA | | | | |
| 11: 110128926-110128940 | RDX | intron | A | 15 15 | 37% | 4% | 0.11 |
| 20: 5167156-5167168 | CDS2 | intron | T | 13 13 | 23% | 2% | 0.10 |
| 8: 30933817-30933828 | WRN | intron | T | 12 12 | 10% | 1% | 0.09 |
| 3: 113079774-113079785 | WDR52 | intron | A | 12 12 | 15% | 1% | 0.07 |
| 8: 107704941-107704954 | OXR1 | intron | A | 14 14 | 13% | 1% | 0.07 |
| 3: 195984819-195984830 | PCYT1A | intron | A | 12 12 | 13% | 1% | 0.06 |
| 15: 81637358-81637378 | TMC3 | intron | GA | 21 21 | 12% | 0% | 0.03 |
| 7: 122757720-122757732 | SLC13A1 | intron | A | 13 13 | 9% | 0% | 0.03 |
| 6: 170881390-170881402 | TBP | 3′utr | T | 13 13 | 13% | 0% | 0.00 |

Table 4. 55 BC-Associated Informative Loci.

TABLE 15

| | |
|---|---|
| Cancer | NUFIP1, KDM1A, SPHK2, STC1, PIAS2, MLL, |
| | TLN2, CUL1, POP4, PDGFRA, NCOR1, MME, |
| | RASA1, ANAPC7, HSP90AA1, FANCI, WRN, |
| | TBP, DNAH3, MT1X, PTPN22, NUP54, |
| | ADAM2, KIF1B, CORIN, ADAMTSL3, CPOX, |
| | ACRC, NXF1, RDX, CDS2, SLC13A1 |
| Breast Cancer | NUFIP1, KDM1A, SPHK2, STC1, PIAS2, MLL, |
| | TLN2, CUL1, POP4, PDGFRA, NCOR1, MME, |
| | RASA1, ANAPC7, HSP90AA1, FANCI, WRN, TBP |
| Cell Cycle | CUL1, PTPN22, KIF1B, DNAH3, PDGFA, |
| | CCDC46, WRN, MICALL1, ANAPC7 |
| Apoptosis | CUL1, SPHK2, ADAM2, PDGFRA, PDCD6IP |

Table 15. Many of the genes associated with our 55 signature microsatellite loci are known to be associated with cancer generally, specifically with BC, or are involved in other cellular pathways associated with cancer.

TABLE 16

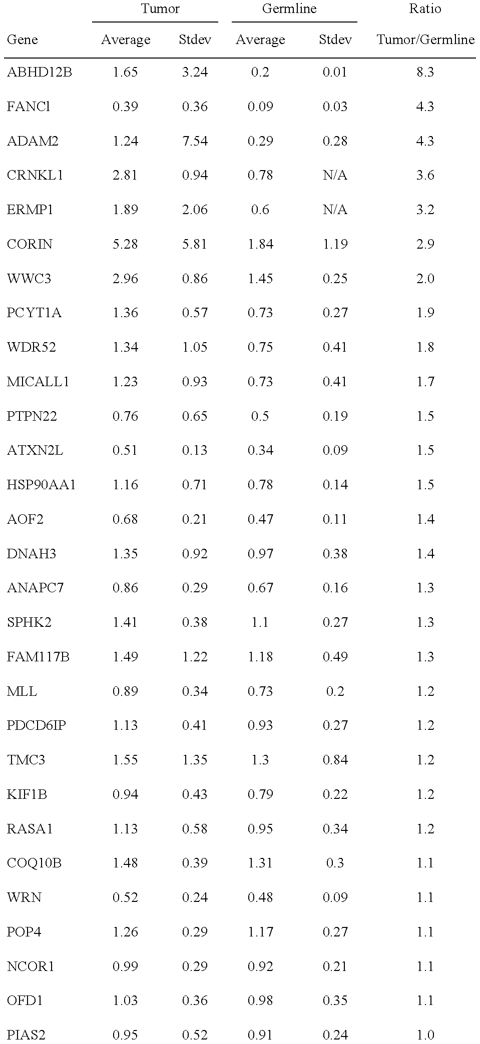

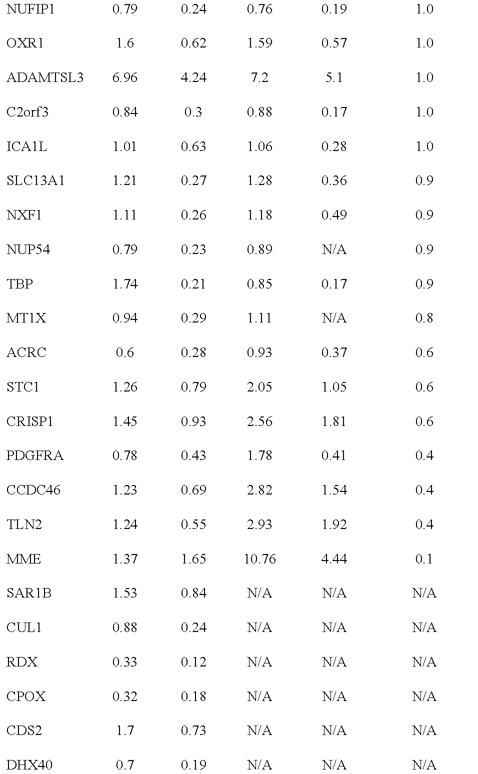

Expression data. Gene Expression levels in tumor and germline at the 55-BC associated informative loci from RNASeq. Gray highlighting indicates loci with ≧2-fold change in gene expression.

TABLE 17

| | | | Modal | |
|---|---|---|---|---|
| | | | genotype | |
| | | | in corre- | |
| | | | sponding | |
| Microsatellite locus | | | 1 kGP- | |
| (hg19) | Region | Motif | EU set | Gene |
| 1: 112305407-112305422 | intron | A | 16 15 | DDX20 |
| 1: 117605131-117605144 | intron | T | 14 14 | TTF2 |
| 1: 16890815-16890826 | intron | A | 12 12 | NBPF1 |
| 1: 225707272-225707287 | intron | A | 16 16 | ENAH |
| 10: 122648751-122648767 | intron | TTTTG | 17 17 | BRWD2 |
| 10: 123256330-123256345 | intron | T | 16 16 | FGFR2 |
| 10: 33471762-33471790 | intron | CA | 29 29 | NRP1 |
| 10: 88817579-88817594 | intron | A | 16 16 | GLUD1 |
| 11: 119144792-119144808 | intron | T | 16 17 | CBL |
| 11: 89502008-89502035 | inter- | GA | 28 28 | — |
| | genic | | | |
| 12: 33578998-33579044 | intron | CA | 47 47 | SYT10 |
| 13: 113964899-113964910 | intron | T | 12 12 | LAMP1 |
| 13: 45517483-45517512 | intron | AC | 30 30 | NUFIP1 |
| 14: 36334906-36334920 | intron | T | 15 15 | BRMS1L |
| 14: 95566069-95566109 | intron | AC | 37 37 | DICER1 |
| 15: 43910867-43910899 | exon | CAG | 33 33 | STRC |
| 15: 85056104-85056118 | 3utr | A | 15 15 | FLJ40113 |
| 16: 70873867-70873881 | intron | T | 15 15 | HYDIN |
| 17: 40986455-40986486 | intron | GA | 32 32 | PSME3 |
| 17: 54981572-54981587 | intron | A | 16 15 | TRIM25 |
| 19: 39077896-39077911 | intron | AT | 16 16 | RYR1 |
| 2: 139308384-139308419 | intron | TC | 42 42 | SPOPL |
| 2: 203680555-203680567 | intron | A | 13 13 | ICA1L |
| 2: 87122106-87122120 | inter- | T | 15 15 | — |
| | genic | | | |
| 2: 91886031-91886042 | inter- | A | 10 12 | — |
| | genic | | | |
| 21: 10995988-10996000 | inter- | A | 14 14 | — |
| | genic | | | |
| 3: 112253194-112253207 | intron | A | 15 15 | ATG3 |
| 3: 112719792-112719807 | 3utr | A | 16 15 | GTPBP8 |
| 3: 121202434-121202458 | intron | A | 25 24 | POLQ |
| 3: 154002358-154002369 | intron | T | 12 12 | DHX36 |
| 3: 170844017-170844030 | intron | A | 14 14 | TNIK |
| 3: 93754287-93754302 | intron | T | 16 16 | ARL13B |
| 4: 169197064-169197079 | intron | A | 16 16 | DDX60 |
| 4: 189063362-189063397 | intron | GT | 30 30 | TRIML1 |
| 4: 47746603-47746615 | intron | A | 13 13 | CORIN |
| 4: 5746907-5746928 | intron | TTC | 22 22 | EVC |
| 6: 31832357-31832371 | intron | A | 15 15 | SLC44A4 |
| 6: 36452604-36452619 | intron | A | 16 15 | KCTD20 |
| 6: 70950282-70950298 | intron | AT | 15 15 | COL9A1 |
| 7: 102825988-102826000 | 3utr | A | 13 13 | DPY19L2P2 |
| 7: 72721731-72721740 | exon | CAA | 10 10 | NSUN5 |
| 7: 83021800-83021817 | intron | A | 14 15 | SEMA3E |
| 8: 107704941-107704954 | intron | A | 14 14 | OXR1 |
| 9: 133498230-133498244 | intron | A | 15 15 | FUBP3 |
| 9: 52626-52640 | inter- | A | 16 15 | — |
| | genic | | | |
| X: 131231431-131231468 | intron | AC | 38 38 | FRMD7 |
| X: 13775753-13775768 | intron | T | 16 16 | OFD1 |
| X: 70812449-70812463 | intron | T | 15 15 | ACRC |

Table 17. 48 GBM-associated informative loci.

TABLE 18

| | | | Modal | |
|---|---|---|---|---|
| | | | genotype | |
| | | | in corres- | |
| | | | ponding | |
| Microsatellite locus | | | 1 kGP- | |
| (hg19) | Region | Motif | EU set | Gene |
| 1: 10357207-10357223 | intron | T | 16 17 | KIF1B |
| 1: 112305407-112305422 | intron | A | 16 15 | DDX20 |
| 1: 145456733-145456746 | intron | A | 14 14 | POLR3GL |
| 1: 153617511-153617525 | intron | T | 15 15 | C1orf77 |
| 1: 231094051-231094066 | intron | A | 16 15 | TTC13 |
| 11: 108058770-108058784 | intron | T | 15 15 | NPAT |
| 11: 108141956-108141970 | intron | T | 15 15 | ATM |
| 11: 134072617-134072631 | intron | A | 15 15 | NCAPD3 |
| 12: 51053874-51053888 | intron | T | 15 15 | DIP2B |
| 12: 95488340-95488353 | intron | A | 14 14 | FGD6 |
| 12: 989801-989814 | intron | T | 13 14 | WNK1 |
| 13: 113964899-113964910 | intron | T | 12 12 | LAMP1 |
| 13: 115002098-115002110 | intron | T | 13 13 | CDC16 |
| 13: 28133957-28133971 | intron | A | 15 15 | LNX2 |
| 13: 77792100-77792112 | intron | A | 13 13 | MYCBP2 |
| 14: 21936763-21936775 | intron | A | 13 13 | RAB2B |
| 14: 51062237-51062261 | intron | TC | 23 23 | ATL1 |
| 14: 76198819-76198830 | intron | T | 11 11 | TTLL5 |
| 15: 44002671-44002699 | inter- | TG | 29 29 | — |
| | genic | | | |
| 15: 63040517-63040532 | intron | A | 16 16 | TLN2 |
| 15: 73418742-73418755 | intron | T | 14 14 | NEO1 |
| 16: 66946895-66946926 | intron | GT | 32 32 | CDH16 |
| 16: 70176322-70176335 | intron | T | 14 14 | PDPR |
| 17: 15517061-15517072 | intron | A | 12 12 | CDRT1 |
| 17: 15973418-15973434 | intron | T | 16 17 | NCOR1 |
| 17: 3968150-3968161 | intron | A | 12 12 | ZZEF1 |
| 17: 40986455-40986486 | intron | GA | 32 32 | PSME3 |
| 19: 21558016-21558032 | inter- | TG | 19 19 | — |
| | genic | | | |
| 2: 111721143-111721181 | intron | TG | 19 19 | ACOXL |
| 2: 48688259-48688272 | intron | T | 14 14 | KLRAQ1 |
| 2: 61145499-61145511 | intron | T | 13 13 | REL |
| 2: 87122106-87122120 | inter- | T | 15 15 | — |
| | genic | | | |
| 21: 19628810-19628822 | intron | T | 13 13 | CHODL |
| 21: 44488756-44488769 | intron | A | 15 15 | CBS |
| 3: 112253194-112253207 | intron | A | 15 15 | ATG3 |
| 3: 132166149-132166161 | intron | T | 13 13 | DNAJC13 |
| 3: 172052898-172052918 | intron | T | 21 21 | FNDC3B |
| 3: 196088810-196088825 | intron | A | 16 16 | UBXN7 |
| 3: 50155884-50155909 | 3utr | GA | 26 26 | RBM5 |
| 4: 113107830-113107844 | intron | T | 15 15 | C4orf32 |
| 4: 128621145-128621157 | intron | T | 13 13 | INTU |
| 4: 186188374-186188387 | intron | A | 14 14 | SNX25 |
| 4: 22444252-22444266 | intron | A | 15 15 | GPR125 |
| 4: 5746907-5746928 | intron | TTC | 22 22 | EVC |
| 4: 71114677-71114688 | intron | ATA | 12 12 | CSN3 |
| 5: 112903586-112903597 | intron | T | 12 12 | YTHDC2 |
| 5: 137013351-137013364 | intron | A | 14 14 | KLHL3 |
| 5: 156525921-156525942 | intron | AG | 22 22 | HAVCR2 |
| 5: 72185592-72185606 | intron | T | 15 15 | TNPO1 |
| 6: 126249756-126249770 | intron | T | 14 15 | NCOA7 |
| 6: 157495952-157495965 | intron | T | 14 14 | ARID1B |
| 6: 31832357-31832371 | intron | A | 15 15 | SLC44A4 |
| 6: 36452604-36452619 | intron | A | 16 15 | KCTD20 |
| 6: 49815874-49815887 | intron | T | 14 14 | CRISP1 |
| 7: 65426055-65426068 | intron | A | 14 14 | GUSB |
| 7: 95775849-95775862 | intron | A | 14 14 | SLC25A13 |
| 7: 95818865-95818882 | intron | A | 18 17 | SLC25A13 |
| 8: 38839303-38839315 | intron | T | 13 13 | HTRA4 |
| 8: 96047807-96047819 | intron | A | 14 14 | C8orf38 |
| 9: 118164376-118164387 | intron | T | 12 12 | Dec1 |
| 9: 133498230-133498244 | intron | A | 15 15 | FUBP3 |
| 9: 52626-52640 | inter- | A | 16 15 | — |
| | genic | | | |
| X: 134853047-134853059 | intron | T | 13 13 | CT45-1 |
| X: 18183098-18183112 | 3utr | A | 15 15 | BEND2 |
| X: 52734297-52734310 | intron | A | 14 14 | SSX2 |
| X: 52895580-52895606 | intron | GT | 25 25 | XAGE3 |

Table 18. 66 LGG-Associated Informative Loci.

TABLE 19

| | | | Modal | |
|---|---|---|---|---|
| Microsatellite locus | | | genotype | |
| (hg19) | Region | Motif | in LGG | Gene |
| 11: 116691512-116691528 | 3utr | GACA | 13 17 | APOA4 |
| 14: 88651827-88651847 | 3utr | AC | 21 23 | KCNK10 |
| 21: 30925854-30925868 | 3utr | T | 14 15 | C21orf41 |
| 15: 20666398-20666410 | inter- | A | 13 13 | — |
| | genic | | | |
| 15: 44002671-44002699 | inter- | TG | 29 29 | — |
| | genic | | | |
| 2: 91886031-91886042 | inter- | A | 10 12 | — |
| | genic | | | |
| 9: 52626-52640 | inter- | A | 14 15 | — |
| | genic | | | |
| 1: 151384053-151384066 | intron | A | 14 14 | POGZ |
| 1: 181714467-181714480 | intron | T | 14 14 | CACNA1E |
| 11: 16117685-16117697 | intron | A | 13 13 | SOX6 |
| 13: 115002098-115002110 | intron | T | 13 12 | CDC16 |
| 13: 77792100-77792112 | intron | A | 13 13 | MYCBP2 |
| 15: 73418742-73418755 | intron | T | 14 14 | NEO1 |
| 16: 70176322-70176335 | intron | T | 13 14 | PDPR |
| 16: 7703786-7703806 | intron | CT | 23 23 | A2BP1 |
| 20: 37146132-37146145 | intron | T | 14 14 | KIAA1219 |
| 3: 132363753-132363764 | intron | A | 12 12 | ACAD11 |
| 3: 45776876-45776888 | intron | T | 13 13 | SACM1L |
| 4: 128621145-128621157 | intron | T | 13 13 | INTU |
| 4: 141448596-141448609 | intron | T | 14 14 | ELMOD2 |
| 4: 166388826-166388837 | intron | T | 12 12 | CPE |
| 4: 22444252-22444266 | intron | A | 15 14 | GPR125 |
| 5: 137013351-137013364 | intron | A | 14 14 | KLHL3 |
| 6: 126249756-126249770 | intron | T | 15 14 | NCOA7 |
| 6: 42611937-42611950 | intron | A | 14 14 | UBR2 |
| 9: 118164376-118164387 | intron | T | 12 12 | |
| X: 52734297-52734310 | intron | A | 14 14 | SSX2 |

Table 19. Loci that can be used to differentiate GBM from LGG.

TABLE 20

| | | | Modal | |
|---|---|---|---|---|
| Microsatellite locus | | | Genotype | |
| (hg19) | Region | Motif | in LGG G2 | Gene |
| 9: 52626-52640 | inter- | A | 14 15 | — |
| | genic | | | |
| 13: 115002098-115002110 | intron | T | 13 12 | CDC16 |
| 13: 77792100-77792112 | intron | A | 13 13 | MYCBP2 |
| 2: 27597191-27597203 | intron | T | 13 13 | SNX17 |
| 20: 37146132-37146145 | intron | T | 14 14 | KIAA1219 |
| 3: 158407931-158407944 | intron | T | 14 14 | GFM1 |
| 3: 45776876-45776888 | intron | T | 13 13 | SACM1L |
| 4: 83970298-83970311 | intron | T | 14 14 | COPS4 |

Table 20. Loci that can be used to differentiate GBM from Grade II LGG.

TABLE 21

| | | | | Samples | | |
|---|---|---|---|---|---|---|
| | | | Samples | with min 4 | Average | |
| Gene | Region | Motif | Called | Alleles | Alleles | Stdev |
| CLIP1 | intron | A | 640 | 511 | 4.30 | 1.0 |
| RAP1A | intron | T | 650 | 460 | 3.99 | 1.1 |
| RIT2 | intron | A | 645 | 402 | 3.84 | 1.1 |
| SGIP1 | intron | A | 648 | 401 | 3.84 | 1.1 |
| RNF5 | intron | T | 638 | 384 | 3.77 | 1.2 |
| CATSPER2 | intron | A | 649 | 383 | 3.51 | 0.9 |
| ANO6 | intron | T | 649 | 369 | 3.55 | 1.1 |
| OSBP | intron | A | 649 | 366 | 3.82 | 1.1 |
| ARMC10 | intron | T | 649 | 351 | 3.48 | 1.2 |
| APBB1IP | intron | A | 650 | 345 | 3.62 | 1.0 |
| MFSD11 | intron | T | 647 | 338 | 3.35 | 1.2 |
| IL3RA | intron | A | 648 | 328 | 3.54 | 1.2 |
| TPTE | intron | T | 620 | 327 | 3.51 | 1.9 |
| NUP54 | intron | A | 640 | 326 | 3.64 | 1.1 |
| EDNRA | intron | T | 649 | 309 | 3.24 | 1.2 |
| OR4K2 | upstream | T | 574 | 303 | 3.39 | 1.6 |
| PTP4A1 | intron | T | 650 | 297 | 3.34 | 1.1 |
| GNAQ | intron | A | 650 | 296 | 3.33 | 0.9 |
| ALG8 | intron | A | 525 | 295 | 3.60 | 2.0 |
| C14orf133 | intron | A | 641 | 291 | 3.20 | 1.3 |
| CT45-4 | intron | T | 453 | 289 | 3.54 | 0.9 |

Table 21. Variant Microsatellite Loci.

TABLE 22

| | | | | 1kGP- | | | | BC | | | |
|---|---|---|---|---|---|---|---|---|---|---|---|
| | | | | EUF | | | | Germline | | | |
| | | | | Genotype | Hardy- | | | Genotype | | | |
| | | | | (# of | Wein- | BC | | (# of | Hardy- | | Ben- |
| | Modal | 1kGP- | | exomes | berg | Germ- | BC | exomes | Weinberg | | jamini- |
| | Genotype | EUF | 1kGP- | having | Chi- | line | Germ- | having | Chi- | | Hochberg |
| Microsatellite | in 1kGP- | exomes | EUF | specified | square p | exomes | line | specified | square p | Fisher's | adjusted |
| Locus | EUF | called | % diff | genotype) | value | called | % diff | genotype) | value | p-value | p-value |
| 2: 198042842-198042853 | 12 12 | 54 | 2% | 11 12 | 0.998 | 107 | 27% | 12 12 | 0.757 | 2.69E−05 | 2.97E−03 |
| | | | | (1), | | | | (78), | | | |
| | | | | 12 12 | | | | 10 12 | | | |
| | | | | (53) | | | | (1), | | | |
| | | | | | | | | 11 12 | | | |
| | | | | | | | | (28) | | | |
| 13: 44415483-44415512 | 30 30 | 159 | 4% | 28 30 | 1.000 | 430 | 17% | 34 30 | 0.050 | 8.69E−06 | 1.42E−03 |
| | | | | (2), | | | | (1), | | | |
| | | | | 32 30 | | | | 32 32 | | | |
| | | | | (4), | | | | (7), | | | |
| | | | | 30 30 | | | | 28 30 | | | |
| | | | | (153) | | | | (14), | | | |
| | | | | | | | | 32 30 | | | |
| | | | | | | | | (49), | | | |
| | | | | | | | | 30 30 | | | |
| | | | | | | | | (358), | | | |
| | | | | | | | | 28 28 (1) | | | |
| 1: 23281511-23281526 | 16 16 | 38 | 11% | 16 16 | 0.943 | 185 | 44% | 16 16 | 0.013 | 7.92E−05 | 6.60E−03 |
| | | | | (34), | | | | (104), | | | |
| | | | | 16 15 (4) | | | | 16 15 | | | |
| | | | | | | | | (77), | | | |
| | | | | | | | | 16 17 (4) | | | |
| 19: 53815688-53815705 | 18 18 | 21 | 24% | 18 18 | 0.826 | 65 | 91% | 18 18 | 1.53E−08 | 1.02E−08 | 1.91E−05 |
| | | | | (16), | | | | (6), | | | |
| | | | | 18 19 (5) | | | | 18 19 | | | |
| | | | | | | | | (4), | | | |
| | | | | | | | | 18 17 | | | |
| | | | | | | | | (55) | | | |
| 8: 23765515-23765540 | 26 26 | 82 | 11% | 24 26 | 1.000 | 70 | 41% | 24 26 | 0.444 | 2.35E−05 | 2.67E−03 |
| | | | | (3), | | | | (28), | | | |
| | | | | 30 26 | | | | 28 26 | | | |
| | | | | (1), | | | | (1), | | | |
| | | | | 28 26 | | | | 26 26 | | | |
| | | | | (5), | | | | (41) | | | |
| | | | | 26 26 | | | | | | | |
| | | | | (73) | | | | | | | |
| 20: 19966883-19966904 | 22 22 | 36 | 22% | 22 21 | 0.801 | 31 | 81% | 22 22 | 0.147 | 2.05E−06 | 5.49E−04 |
| | | | | (7), | | | | (6), | | | |
| | | | | 22 22 | | | | 22 21 | | | |
| | | | | (28), | | | | (9), | | | |
| | | | | 21 21 (1) | | | | 21 21 | | | |
| | | | | | | | | (16) | | | |
| 18: 42646303-42646318 | 16 16 | 40 | 18% | 16 17 | 0.000 | 150 | 61% | 17 15 | 8.18E−06 | 9.10E−07 | 2.84E−04 |
| | | | | (1), | | | | (1), | | | |
| | | | | 16 16 | | | | 16 16 | | | |
| | | | | (33), | | | | (59), | | | |
| | | | | 16 15 | | | | 16 15 | | | |
| | | | | (5), | | | | (70), | | | |
| | | | | 14 14 (1) | | | | 14 14 | | | |
| | | | | | | | | (4), | | | |
| | | | | | | | | 14 15 | | | |
| | | | | | | | | (1), | | | |
| | | | | | | | | 15 15 | | | |
| | | | | | | | | (1), | | | |
| | | | | | | | | 16 17 | | | |
| | | | | | | | | (4), | | | |
| | | | | | | | | 16 14 | | | |
| | | | | | | | | (10) | | | |
| 11: 117858248-117858263 | 16 16 | 58 | 14% | 16 17 | 0.997 | 92 | 43% | 16 16 | 0.213 | 1.39E−04 | 9.46E−03 |
| | | | | (6), | | | | (52), | | | |
| | | | | 16 16 | | | | 16 15 | | | |
| | | | | (50), | | | | (32), | | | |
| | | | | 16 15 (2) | | | | 16 17 (8) | | | |
| 5: 133971943-133971958 | 16 16 | 17 | 29% | 15 15 | 0.735 | 99 | 91% | 16 16 | 1.11E−08 | 1.73E−07 | 8.11E−05 |
| | | | | (1), | | | | (9), | | | |
| | | | | 16 15 | | | | 16 15 | | | |
| | | | | (4), | | | | (82), | | | |
| | | | | 16 16 | | | | 14 15 | | | |
| | | | | (12) | | | | (1), | | | |
| | | | | | | | | 15 15 (7) | | | |
| 16: 20863600-20863625 | 26 26 | 59 | 20% | 26 26 | 0.113 | 81 | 53% | 24 26 | 6.04E−06 | 1.03E−04 | 8.05E−03 |
| | | | | (47), | | | | (30), | | | |
| | | | | 24 26 | | | | 30 26 | | | |
| | | | | (7), | | | | (6), | | | |
| | | | | 30 26 | | | | 28 26 | | | |
| | | | | (2), | | | | (3), | | | |
| | | | | 28 26 | | | | 28 30 | | | |
| | | | | (2), | | | | (4), | | | |
| | | | | 30 30 (1) | | | | 26 26 | | | |
| | | | | | | | | (38) | | | |
| 16: 28749759-28749775 | 17 17 | 32 | 28% | 18 17 | 0.973 | 54 | 72% | 18 17 | 0.004 | 1.07E−04 | 8.17E−03 |
| | | | | (8), | | | | (8), | | | |
| | | | | 17 17 | | | | 16 17 | | | |
| | | | | (23), | | | | (31), | | | |
| | | | | 16 17 (1) | | | | 17 17 | | | |
| | | | | | | | | (15) | | | |
| 15: 60827809-60827824 | 16 16 | 69 | 22% | 16 17 | 0.960 | 104 | 53% | 16 16 | 0.059 | 3.98E−05 | 4.04E−03 |
| | | | | (5), | | | | (49), | | | |
| | | | | 16 16 | | | | 16 15 | | | |
| | | | | (54), | | | | (51), | | | |
| | | | | 16 15 | | | | 15 15 | | | |
| | | | | (10) | | | | (1), | | | |
| | | | | | | | | 16 17 (3) | | | |
| X: 10069659-10069674 | 16 16 | 38 | 34% | 16 15 | 0.899 | 111 | 83% | 16 16 | 2.85E−33 | 5.29E−08 | 4.96E−05 |
| | | | | (11), | | | | (19), | | | |
| | | | | 16 17 | | | | 15 16 | | | |
| | | | | (2), | | | | (90), | | | |
| | | | | 16 16 | | | | 15 15 | | | |
| | | | | (25) | | | | (1), | | | |
| | | | | | | | | 17 17 (1) | | | |
| 16: 55275517-55275536 | 19 20 | 29 | 34% | 19 19 | 0.007 | 40 | 83% | 18 18 | 0.001 | 1.09E−04 | 8.18E−03 |
| | | | | (2), | | | | (1), | | | |
| | | | | 18 19 | | | | 18 19 | | | |
| | | | | (7), | | | | (28), | | | |
| | | | | 21 20 | | | | 18 17 | | | |
| | | | | (1), | | | | (1), | | | |
| | | | | 19 20 | | | | 18 20 | | | |
| | | | | (19) | | | | (2), | | | |
| | | | | | | | | 19 19 | | | |
| | | | | | | | | (1), | | | |
| | | | | | | | | 19 20 (7) | | | |
| 17: 55018379-55018396 | 18 17 | 38 | 32% | 18 17 | 0.002 | 85 | 72% | 18 18 | 3.24E−10 | 5.10E−05 | 4.78E−03 |
| | | | | (26), | | | | (1), | | | |
| | | | | 18 18 | | | | 16 16 | | | |
| | | | | (8), | | | | (2), | | | |
| | | | | 17 16 (4) | | | | 19 17 | | | |
| | | | | | | | | (1), | | | |
| | | | | | | | | 18 17 | | | |
| | | | | | | | | (24), | | | |
| | | | | | | | | 16 17 | | | |
| | | | | | | | | (54), | | | |
| | | | | | | | | 17 17 (3) | | | |
| 7: 148125728-148125744 | 17 17 | 26 | 42% | 16 17 | 0.000 | 63 | 90% | 16 16 | 1.01E−11 | 4.33E−06 | 9.02E−04 |
| | | | | (10), | | | | (3), | | | |
| | | | | 17 17 | | | | 16 15 | | | |
| | | | | (15), | | | | (7), | | | |
| | | | | 14 14 (1) | | | | 14 14 | | | |
| | | | | | | | | (2), | | | |
| | | | | | | | | 15 15 | | | |
| | | | | | | | | (1), | | | |
| | | | | | | | | 16 17 | | | |
| | | | | | | | | (43), | | | |
| | | | | | | | | 17 17 | | | |
| | | | | | | | | (6), | | | |
| | | | | | | | | 16 14 (1) | | | |
| 19: 34797971-34797987 | 17 17 | 30 | 53% | 16 17 | 0.628 | 105 | 93% | 16 16 | 0.005 | 1.73E−06 | 4.98E−04 |
| | | | | (10), | | | | (25), | | | |
| | | | | 16 16 | | | | 16 15 | | | |
| | | | | (5), | | | | (12), | | | |
| | | | | 17 17 | | | | 18 17 | | | |
| | | | | (14), | | | | (2), | | | |
| | | | | 16 15 (1) | | | | 16 17 | | | |
| | | | | | | | | (59), | | | |
| | | | | | | | | 17 17 (7) | | | |
| 10: 44888543-44888559 | 17 17 | 15 | 60% | 17 17 | 0.005 | 46 | 100% | 17 15 | 7.79E−10 | 9.01E−05 | 7.35E−03 |
| | | | | (6), | | | | (2), | | | |
| | | | | 15 15 | | | | 15 14 | | | |
| | | | | (3), | | | | (10), | | | |
| | | | | 16 17 (6) | | | | 16 15 | | | |
| | | | | | | | | (6), | | | |
| | | | | | | | | 15 15 | | | |
| | | | | | | | | (7), | | | |
| | | | | | | | | 16 17 | | | |
| | | | | | | | | (21) | | | |
| 4: 54825759-54825775 | 17 17 | 39 | 51% | 18 17 | 0.999 | 113 | 85% | 16 16 | 3.81E−32 | 5.45E−05 | 4.99E−03 |
| | | | | (1), | | | | (5), | | | |
| | | | | 17 15 | | | | 15 15 | | | |
| | | | | (1), | | | | (1), | | | |
| | | | | 16 17 | | | | 16 17 | | | |
| | | | | (15), | | | | (90), | | | |
| | | | | 16 16 | | | | 17 17 | | | |
| | | | | (2), | | | | (17) | | | |
| | | | | 17 17 | | | | | | | |
| | | | | (19), | | | | | | | |
| | | | | 16 15 (1) | | | | | | | |
| X: 13685674-13685689 | 16 16 | 79 | 53% | 15 15 | 0.172 | 166 | 80% | 16 16 | 0.007 | 2.06E−05 | 2.41E−03 |
| | | | | (5), | | | | (33), | | | |
| | | | | 16 16 | | | | 15 14 | | | |
| | | | | (37), | | | | (2), | | | |
| | | | | 16 15 | | | | 16 15 | | | |
| | | | | (34), | | | | (109), | | | |
| | | | | 14 15 (3) | | | | 15 15 | | | |
| | | | | | | | | (21), | | | |
| | | | | | | | | 16 17 (1) | | | |
| 1: 114173856-114173867 | 11 12 | 123 | 50% | 11 12 | 0.849 | 380 | 69% | 12 12 | 1.30E−11 | 1.35E−04 | 9.38E−03 |
| | | | | (62), | | | | (97), | | | |
| | | | | 11 11 | | | | 11 11 | | | |
| | | | | (43), | | | | (166), | | | |
| | | | | 12 12 | | | | 11 12 | | | |
| | | | | (18) | | | | (117) | | | |
| 7: 38248656-38248675 | 22 20 | 137 | 58% | 22 22 | 0.496 | 410 | 78% | 22 20 | 6.42E−12 | 8.32E−06 | 1.42E−03 |
| | | | | (23), | | | | (91), | | | |
| | | | | 20 20 | | | | 22 22 | | | |
| | | | | (56), | | | | (60), | | | |
| | | | | 22 20 | | | | 20 20 | | | |
| | | | | (58) | | | | (256), | | | |
| | | | | | | | | 24 20 | | | |
| | | | | | | | | (1), | | | |
| | | | | | | | | 22 24 | | | |
| | | | | | | | | (1), | | | |
| | | | | | | | | 18 20 (1) | | | |
| 22: 36637989-36638017 | 25 29 | 177 | 59% | 27 29 | 0.000 | 420 | 80% | 29 29 | 1.44E−22 | 8.36E−07 | 3.14E−04 |
| | | | | (1), | | | | (211), 25 | | | |
| | | | | 25 25 | | | | 25 (110), | | | |
| | | | | (36), | | | | 25 31 | | | |
| | | | | 29 31 | | | | (4), | | | |
| | | | | (3), | | | | 29 31 | | | |
| | | | | 25 31 | | | | (5), | | | |
| | | | | (3), | | | | 25 29 | | | |
| | | | | 25 29 | | | | (86), | | | |
| | | | | (72), | | | | 25 33 | | | |
| | | | | 27 27 | | | | (1), | | | |
| | | | | (1), | | | | 27 29 | | | |
| | | | | 29 29 | | | | (2), | | | |
| | | | | (61) | | | | 31 31 (1) | | | |
| 4: 77284501-77284515 | 14 15 | 28 | 75% | 13 15 | 0.072 | 105 | 99% | 13 15 | 3.31E−12 | 6.50E−05 | 5.67E−03 |
| | | | | (3), | | | | (4), | | | |
| | | | | 15 15 | | | | 12 15 | | | |
| | | | | (6), | | | | (2), | | | |
| | | | | 12 15 | | | | 12 12 | | | |
| | | | | (5), | | | | (19), | | | |
| | | | | 12 12 | | | | 13 13 | | | |
| | | | | (3), | | | | (37), | | | |
| | | | | 13 13 | | | | 15 14 | | | |
| | | | | (3), | | | | (1), | | | |
| | | | | 13 12 | | | | 13 12 | | | |
| | | | | (1), | | | | (25), | | | |
| | | | | 14 15 (7) | | | | 16 15 | | | |
| | | | | | | | | (3), | | | |
| | | | | | | | | 15 15 | | | |
| | | | | | | | | (14) | | | |
| 8: 39726241-39726276 | 40 36 | 152 | 62% | 38 36 | 0.089 | 411 | 81% | 36 40 | 1.08E−24 | 7.78E−06 | 1.46E−03 |
| | | | | (4), | | | | (79), | | | |
| | | | | 38 40 | | | | 34 36 | | | |
| | | | | (2), | | | | (1), | | | |
| | | | | 40 40 | | | | 38 40 | | | |
| | | | | (52), | | | | (9), | | | |
| | | | | 36 36 | | | | 38 36 | | | |
| | | | | (34), | | | | (5), | | | |
| | | | | 38 38 | | | | 42 40 | | | |
| | | | | (1), | | | | (2), | | | |
| | | | | 40 36 | | | | 40 40 | | | |
| | | | | (58), | | | | (204), | | | |
| | | | | 34 36 (1) | | | | 38 38 | | | |
| | | | | | | | | (2), | | | |
| | | | | | | | | 36 36 | | | |
| | | | | | | | | (109) | | | |
| 6: 49923833-49923846 | 14 14 | 54 | 41% | 13 14 | 0.618 | 255 | 13% | 13 14 | 4.75E−63 | 8.03E−06 | 1.43E−03 |
| | | | | (20), | | | | (26), | | | |
| | | | | 14 14 | | | | 14 14 | | | |
| | | | | (32), | | | | (222), | | | |
| | | | | 14 15 (2) | | | | 14 15 | | | |
| | | | | | | | | (4), | | | |
| | | | | | | | | 15 15 | | | |
| | | | | | | | | (2), | | | |
| | | | | | | | | 17 17 (1) | | | |
| 3: 199364528-199364569 | 42 42 | 42 | 57% | 42 42 | 0.000 | 81 | 17% | 33 36 | 7.27E−20 | 1.06E−05 | 1.59E−03 |
| | | | | (18), | | | | (1), | | | |
| | | | | 33 36 | | | | 45 45 | | | |
| | | | | (10), | | | | (1), | | | |
| | | | | 36 36 | | | | 42 36 | | | |
| | | | | (3), | | | | (3), | | | |
| | | | | 33 33 | | | | 42 42 | | | |
| | | | | (11) | | | | (67), | | | |
| | | | | | | | | 42 33 | | | |
| | | | | | | | | (5), | | | |
| | | | | | | | | 36 36 | | | |
| | | | | | | | | (2), | | | |
| | | | | | | | | 33 33 (2) | | | |
| 1: 10279794-10279810 | 16 17 | 45 | 49% | 18 17 | 0.191 | 104 | 14% | 16 16 | 5.58E−12 | 2.05E−05 | 2.47E−03 |
| | | | | (3), | | | | (1), | | | |
| | | | | 17 17 | | | | 18 17 | | | |
| | | | | (19), | | | | (2), | | | |
| | | | | 16 17 | | | | 16 17 | | | |
| | | | | (23) | | | | (89), | | | |
| | | | | | | | | 17 17 | | | |
| | | | | | | | | (12) | | | |
| 3: 156317074-156317090 | 17 17 | 98 | 23% | 17 15 | 0.000 | 409 | 7% | 17 15 | 1.61E−241 | 1.85E−05 | 2.40E−03 |
| | | | | (15), | | | | (24), | | | |
| | | | | 27 27 | | | | 21 19 | | | |
| | | | | (2), | | | | (1), | | | |
| | | | | 27 17 | | | | 19 17 | | | |
| | | | | (1), | | | | (1), | | | |
| | | | | 27 25 | | | | 17 17 | | | |
| | | | | (5), | | | | (380), | | | |
| | | | | 17 17 | | | | 27 23 | | | |
| | | | | (75) | | | | (1), | | | |
| | | | | | | | | 25 27 (2) | | | |
| 2: 75772781-75772805 | 21 21 | 41 | 46% | 25 21 | 0.000 | 142 | 13% | 25 23 | 3.41E−50 | 1.86E−05 | 2.32E−03 |
| | | | | (1), | | | | (1), | | | |
| | | | | 25 23 | | | | 25 25 | | | |
| | | | | (3), | | | | (7), | | | |
| | | | | 25 25 | | | | 23 23 | | | |
| | | | | (13), | | | | (1), | | | |
| | | | | 23 23 | | | | 21 19 | | | |
| | | | | (2), | | | | (3), | | | |
| | | | | 21 21 | | | | 21 23 | | | |
| | | | | (22) | | | | (3), | | | |
| | | | | | | | | 17 17 | | | |
| | | | | | | | | (1), | | | |
| | | | | | | | | 21 21 | | | |
| | | | | | | | | (123), | | | |
| | | | | | | | | 25 27 (3) | | | |
| 4: 47441360-47441372 | 13 13 | 113 | 19% | 13 13 | 0.933 | 407 | 5% | 13 14 | 0.147 | 1.31E−05 | 1.89E−03 |
| | | | | (91), | | | | (11), | | | |
| | | | | 13 12 | | | | 13 13 | | | |
| | | | | (20), | | | | (385), | | | |
| | | | | 13 14 (2) | | | | 13 12 | | | |
| | | | | | | | | (10), | | | |
| | | | | | | | | 14 14 (1) | | | |
| 17: 15914143-15914159 | 16 17 | 44 | 55% | 18 17 | 0.288 | 71 | 14% | 18 17 | 4.36E−08 | 6.37E−06 | 1.26E−03 |
| | | | | (4), | | | | (1), | | | |
| | | | | 16 17 | | | | 16 17 | | | |
| | | | | (20), | | | | (61), | | | |
| | | | | 17 17 | | | | 17 17 (9) | | | |
| | | | | (20) | | | | | | | |
| 5: 86715252-86715269 | 18 17 | 42 | 43% | 18 17 | 0.035 | 122 | 11% | 18 18 | 1.80E−18 | 1.69E−05 | 2.26E−03 |
| | | | | (24), | | | | (6), | | | |
| | | | | 18 18 | | | | 18 17 | | | |
| | | | | (18) | | | | (109), | | | |
| | | | | | | | | 16 17 | | | |
| | | | | | | | | (5), | | | |
| | | | | | | | | 17 17 (2) | | | |
| 12: 109318414-109318431 | 18 17 | 23 | 52% | 18 17 | 0.721 | 88 | 13% | 18 18 | 2.64E−11 | 1.33E−04 | 9.42E−03 |
| | | | | (11), | | | | (9), | | | |
| | | | | 18 18 | | | | 18 19 | | | |
| | | | | (11), | | | | (2), | | | |
| | | | | 18 19 (1) | | | | 18 17 | | | |
| | | | | | | | | (77) | | | |
| 14: 101619823-101619840 | 18 17 | 42 | 48% | 18 17 | 0.134 | 141 | 11% | 18 18 | 3.07E−19 | 7.80E−07 | 3.25E−04 |
| | | | | (22), | | | | (12), | | | |
| | | | | 18 18 | | | | 18 19 | | | |
| | | | | (16), | | | | (2), | | | |
| | | | | 18 19 (4) | | | | 18 17 | | | |
| | | | | | | | | (126), | | | |
| | | | | | | | | 17 17 (1) | | | |
| 17: 61177480-61177493 | 14 14 | 48 | 40% | 13 14 | 0.232 | 173 | 8% | 13 14 | 0.857 | 8.79E−07 | 3.00E−04 |
| | | | | (19), | | | | (14), | | | |
| | | | | 14 14 | | | | 14 14 | | | |
| | | | | (29) | | | | (159) | | | |
| 3: 33852505-33852516 | 12 12 | 106 | 21% | 13 13 | 0.585 | 370 | 4% | 12 12 | 1.000 | 1.69E−07 | 9.03E−05 |
| | | | | (1), | | | | (356), | | | |
| | | | | 11 12 | | | | 13 12 | | | |
| | | | | (13), | | | | (13), | | | |
| | | | | 13 12 | | | | 11 12 (1) | | | |
| | | | | (8), | | | | | | | |
| | | | | 12 12 | | | | | | | |
| | | | | (84) | | | | | | | |
| 9: 5788652-5788666 | 15 15 | 22 | 45% | 15 15 | 0.386 | 82 | 7% | 15 14 | 1.000 | 9.30E−05 | 7.42E−03 |
| | | | | (12), | | | | (5), | | | |
| | | | | 15 14 | | | | 16 15 | | | |
| | | | | (10) | | | | (1), | | | |
| | | | | | | | | 15 15 | | | |
| | | | | | | | | (76) | | | |
| 14: 50418032-50418048 | 18 19 | 37 | 32% | 19 19 | 0.008 | 72 | 4% | 18 19 | 1.41E−13 | 1.18E−04 | 8.69E−03 |
| | | | | (12), | | | | (69), | | | |
| | | | | 18 19 | | | | 19 17 | | | |
| | | | | (25) | | | | (2), | | | |
| | | | | | | | | 18 17 (1) | | | |
| 15: 82264330-82264346 | 16 17 | 29 | 41% | 16 17 | 0.083 | 90 | 6% | 16 17 | 2.26E−16 | 1.51E−05 | 2.10E−03 |
| | | | | (17), | | | | (85), | | | |
| | | | | 17 17 | | | | 17 17 (5) | | | |
| | | | | (12) | | | | | | | |
| 3: 99782398-99782410 | 13 13 | 56 | 46% | 13 13 | 0.077 | 34 | 6% | 13 13 | 0.985 | 4.00E−05 | 3.95E−03 |
| | | | | (30), | | | | (32), | | | |
| | | | | 13 12 | | | | 13 12 (2) | | | |
| | | | | (26) | | | | | | | |
| 2: 203338348-203338368 | 21 21 | 49 | 24% | 21 20 | 0.621 | 135 | 3% | 21 20 | 1.000 | 3.16E−05 | 3.39E−03 |
| | | | | (12), | | | | (2), | | | |
| | | | | 21 21 | | | | 22 21 | | | |
| | | | | (37) | | | | (2), | | | |
| | | | | | | | | 21 21 | | | |
| | | | | | | | | (131) | | | |
| X: 70729174-70729188 | 15 15 | 92 | 10% | 15 15 | 0.885 | 539 | 1% | 14 15 | 0.992 | 4.89E−05 | 4.70E−03 |
| | | | | (83), | | | | (6), | | | |
| | | | | 14 15 (9) | | | | 15 15 | | | |
| | | | | | | | | (533) | | | |
| 15: 87612887-87612899 | 13 13 | 47 | 19% | 13 13 | 0.768 | 182 | 2% | 13 13 | 0.989 | 1.22E−04 | 8.76E−03 |
| | | | | (38), | | | | (178), | | | |
| | | | | 13 12 (9) | | | | 13 12 (4) | | | |
| 2: 203388800-203388812 | 13 13 | 99 | 24% | 13 13 | 0.390 | 324 | 3% | 13 13 | 0.968 | 4.30E−10 | 1.61E−06 |
| | | | | (75), | | | | (315), | | | |
| | | | | 13 12 | | | | 13 12 (9) | | | |
| | | | | (24) | | | | | | | |
| 11: 62322485-62322520 | 36 36 | 37 | 38% | 36 37 | 0.847 | 198 | 4% | 36 36 | 0.959 | 7.04E−08 | 4.40E−05 |
| | | | | (1), | | | | (190), | | | |
| | | | | 36 36 | | | | 35 36 (8) | | | |
| | | | | (23), | | | | | | | |
| | | | | 35 36 | | | | | | | |
| | | | | (13) | | | | | | | |
| 11: 109634136-109634150 | 15 15 | 49 | 37% | 15 14 | 0.289 | 50 | 4% | 14 15 | 0.990 | 3.89E−05 | 4.05E−03 |
| | | | | (18), | | | | (2), | | | |
| | | | | 15 15 | | | | 15 15 | | | |
| | | | | (31) | | | | (48) | | | |
| 20: 5115156-5115168 | 13 13 | 61 | 23% | 13 14 | 0.961 | 91 | 2% | 13 13 | 0.994 | 5.77E−05 | 5.15E−03 |
| | | | | (1), | | | | (89), | | | |
| | | | | 13 13 | | | | 13 12 (2) | | | |
| | | | | (47), | | | | | | | |
| | | | | 13 12 | | | | | | | |
| | | | | (13) | | | | | | | |
| 8: 31053359-31053370 | 12 12 | 132 | 10% | 11 12 | 0.838 | 456 | 1% | 12 12 | 0.996 | 2.31E−06 | 5.78E−04 |
| | | | | (13), | | | | (452), | | | |
| | | | | 12 12 | | | | 11 12 (4) | | | |
| | | | | (119) | | | | | | | |
| 8: 107774117-107774130 | 14 14 | 119 | 13% | 13 14 | 0.991 | 443 | 1% | 13 14 | 7.41E−16 | 6.55E−08 | 4.91E−05 |
| | | | | (14), | | | | (3), | | | |
| | | | | 14 14 | | | | 13 13 | | | |
| | | | | (104), | | | | (1), | | | |
| | | | | 14 15 (1) | | | | 14 14 | | | |
| | | | | | | | | (439) | | | |
| 3: 114562464-114562475 | 12 12 | 40 | 15% | 11 12 | 0.998 | 454 | 1% | 12 12 | 2.17E−11 | 6.66E−05 | 5.67E−03 |
| | | | | (4), | | | | (449), | | | |
| | | | | 13 12 | | | | 11 11 | | | |
| | | | | (2), | | | | (1), | | | |
| | | | | 12 12 | | | | 11 12 (4) | | | |
| | | | | (34) | | | | | | | |
| 3: 197469216-197469227 | 12 12 | 71 | 13% | 11 12 | 0.997 | 411 | 1% | 12 12 | 0.997 | 3.13E−06 | 6.91E−04 |
| | | | | (8), | | | | (408), | | | |
| | | | | 12 12 | | | | 11 12 (3) | | | |
| | | | | (62), | | | | | | | |
| | | | | 13 12 (1) | | | | | | | |
| 7: 122544956-122544968 | 13 13 | 92 | 9% | 13 13 | 0.909 | 396 | 0% | 13 13 | 1.000 | 9.40E−06 | 1.47E−03 |
| | | | | (84), | | | | (395), | | | |
| | | | | 13 12 (8) | | | | 13 12 (1) | | | |
| 15: 79424413-79424433 | 21 21 | 60 | 12% | 21 23 | 0.891 | 525 | 0% | 21 19 | 1.000 | 2.62E−06 | 6.14E−04 |
| | | | | (7), | | | | (1), | | | |
| | | | | 21 21 | | | | 21 23 | | | |
| | | | | (53) | | | | (1), | | | |
| | | | | | | | | 21 21 | | | |
| | | | | | | | | (523) | | | |
| 6: 170723315-170723327 | 13 13 | 78 | 13% | 13 13 | 0.833 | 358 | 0% | 13 13 | N/A | 2.04E−08 | 2.55E−05 |
| | | | | (68), | | | | (358) | | | |
| | | | | 13 12 | | | | | | | |
| | | | | (10) | | | | | | | |

Table 22. BC Microsatellite Loci Distribution.

1-84. (canceled)

85. A kit comprising:
a) one or more solid supports comprising immobilized nucleic acid probes, wherein each nucleic acid probe is hybridizable to a target nucleic acid sequence, wherein the target nucleic acid sequence comprises a microsatellite loci selected from the group consisting of the loci listed in any of tables 14, 17, 18, 19, or 20; and
b) one or more reagents for performing hybridizations, washes, and/or elution of target nucleic acid sequences.

86. The kit of claim 85 comprising:
a) one or more solid supports comprising immobilized nucleic acid probes hybridizable to a plurality of target nucleic acid sequences, wherein said target nucleic acid sequences comprise at least 2, 5, 10, 15, 25, 30, 35, 40, 45, 50 or all of the microsatellite loci listed in table 14; and
b) one or more reagents for performing hybridizations, washes, and/or elution of target nucleic acid sequences.

87. The kit of claim 85 comprising:
a) one or more solid supports comprising immobilized nucleic acid probes hybridizable to a plurality of target nucleic acid sequences, wherein said target nucleic acid sequences comprise at least 2, 5, 10, 15, 25, 30, 35, 40, 45 or all of the microsatellite loci listed in table 17; and
b) one or more reagents for performing hybridizations, washes, and/or elution of target nucleic acid sequences.

88. The kit of claim 85 comprising:
a) one or more solid supports comprising immobilized nucleic acid probes hybridizable to a plurality of target nucleic acid sequences, wherein said target nucleic acid sequences comprise at least 2, 5, 10, 15, 25, 30, 35, 40, 45, 50, 55, 60 or all of the microsatellite loci listed in table 18; and
b) one or more reagents for performing hybridizations, washes, and/or elution of target nucleic acid sequences.

89. The kit of claim 85 comprising:
a) one or more solid supports comprising immobilized nucleic acid probes hybridizable to a plurality of target nucleic acid sequences, wherein said target nucleic acid sequences comprise at least 2, 5, 10, 15, 20, 25 or all of the microsatellite loci listed in table 19; and
b) one or more reagents for performing hybridizations, washes, and/or elution of target nucleic acid sequences.

90. The kit of claim 85 comprising:
a) one or more solid supports comprising immobilized nucleic acid probes hybridizable to a plurality of target nucleic acid sequences, wherein said target nucleic acid sequences comprise at least 1, 2, 3, 4, 5, 6, 7, or 8 of the microsatellite loci listed in table 20; and
b) one or more reagents for performing hybridizations, washes, and/or elution of target nucleic acid sequences.

91. The kit of claim 85, wherein the target nucleic acid sequences comprise, for a particular microsatellite loci, the nucleotide sequence corresponding to one or both alleles of a modal genotype of a reference population identified as healthy.

92. A kit comprising:
a) one or more solid supports comprising immobilized nucleic acid probes hybridizable to a plurality of target nucleic acid sequences, wherein said target nucleic acid sequences comprise all or a subset of 1- to 6-mer microsatellite motifs; and
b) one or more reagents for performing hybridizations, washes, and/or elution of target nucleic acid sequences.

93. The kit of claim 85, wherein said one or more solid supports is a microarray slide.

94. The kit of claim 85, wherein said one or more solid supports comprises one or more beads.

95. The kit of claim 85, wherein the target nucleic acid sequences comprise the microsatellite loci with at least 5-10 nucleotides of flanking sequence 5′ and/or 3′ to the microsatellite loci.

96. The kit of claim 95, wherein the target nucleic acid sequences comprise the microsatellite loci with at least 5-10 nucleotides of flanking sequence 5′ to the microsatellite loci and at least 5-10 nucleotides of flanking sequence 3′ to the microsatellite loci, wherein the number of nucleotides of flanking sequence is independently selected for the 5′ and 3′ flanking sequence.

97. The kit of claim 95, wherein the nucleic acid probes are hybridizable to both target nucleic acid sequence corresponding to the microsatellite loci and target nucleic acid sequence corresponding to the flanking sequence.

98. The kit of claim 85, wherein the kit comprises a plurality of solid supports, and wherein each solid support comprises probes hybridizable to more than one target nucleic acid sequence.

99. The kit of claim 85, wherein the nucleic acid probes are microsatellite-specific enrichment probes.

100. (canceled)

101. The kit of claim 85, wherein the nucleic acid probes are complementary to the target nucleic acid sequence, with two or fewer mismatches.

102-108. (canceled)

109. A computer-implemented method of identifying variant microsatellite loci comprising:
(a) receiving, at a computer, a library of sequence reads for subsequences in the nucleic acid from a sample obtained using a Next Generation sequencing platform;
(b) aligning a first sequence read from said library to a reference sequence by an alignment method, wherein the alignment method comprises:
(i) selecting a microsatellite locus and sequence portion flanking the selected microsatellite locus from said sequence read, wherein the flanking sequence comprises at least 1, 2, 3, 4, 5, 6, 7, 8, 9 or 10 nucleotide bases; and
(ii) identifying a similarity between said reference sequence and the selected microsatellite locus and sequence portion flanking the microsatellite locus;
(c) determining the sequence and/or length of the microsatellite locus to which a similarity is identified in (ii);
(d) repeating (a)-(c) for all the sequence reads in the library of sequence reads;
(e) forming a distribution of sequence and/or lengths associated with each microsatellite locus whose length is determined in (c); and
(f) assigning a genotype or allelotype for each microsatellite locus based on its distribution of sequence and/or lengths.

110-245. (canceled)

246. The kit of claim 92, wherein the kit comprises a plurality of solid supports, and wherein each solid support comprises probes hybridizable to more than one target nucleic acid sequence.

247. The kit of claim 92, wherein the nucleic acid probes are microsatellite-specific enrichment probes.

248. The kit of claim 92, wherein said one or more solid supports is a microarray slide.

249. The kit of claim 92, wherein said one or more solid supports comprises one or more beads.
